SEQUENCE LISTING (27 sequences)

SEQ ID NO: 1 - DNA, 2211 bp, Artificial Sequence
  AAVhu68 vp1 capsid
  CDS (1)..(2211)
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 2 - protein, 736 aa, Artificial Sequence
  Synthetic Construct
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - DNA, 2208 bp, Artificial Sequence
  AAV9 VP1 capsid
  CDS (1)..(2208): AAV9 VP1 Capsid
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 4 - protein, 736 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - DNA, 2058 bp, Artificial sequence
  Human GALC coding sequence
  sig_peptide (1)..(126)
  CDS (1)..(2058)
atggctgagtggctactctcggcttcctggcaacgccgagcgaaagctatgactgcggccgcgggttcggcgggccgcgccgcggtgcccttgctgctgtgtgcgctgctggcgcccggcggcgcgtacgtgctcgacgactccgacgggctgggccgggagttcgacggcatcggcgcggtcagcggcggcggggcaacctcccgacttctagtaaattacccagagccctatcgttctcagatattggattatctctttaagccgaattttggtgcctctttgcatattttaaaagtggaaataggtggtgatgggcagacaacagacggcactgagccctcccacatgcattatgcactagatgagaattatttccgaggatacgagtggtggttgatgaaagaagctaagaagaggaatcccaatattacactcattgggttgccatggtcattccctggatggctgggaaaaggtttcgactggccttatgtcaatcttcagctgactgcctattatgtcgtgacctggattgtgggcgccaagcgttaccatgatttggacattgattatattggaatttggaatgagaggtcatataatgccaattatattaagatattaagaaaaatgctgaattatcaaggtctccagcgagtgaaaatcatagcaagtgataatctctgggagtccatctctgcatccatgctccttgatgccgaactcttcaaggtggttgatgttataggggctcattatcctggaacccattcagcaaaagatgcaaagttgactgggaagaagctttggtcttctgaagactttagcactttaaatagtgacatgggtgcaggctgctggggtcgcattttaaatcagaattatatcaatggctatatgacttccacaatcgcatggaatttagtggctagttactatgaacagttgccttatgggagatgcgggttgatgacggcccaggagccatggagtgggcactacgtggtagaatctcctgtctgggtatcagctcataccactcagtttactcaacctggctggtattacctgaagacagttggccatttagagaaaggaggaagctacgtagctctgactgatggcttagggaacctcaccatcatcattgaaaccatgagtcataaacattctaagtgcatacggccatttcttccttatttcaatgtgtcacaacaatttgccacctttgttcttaagggatcttttagtgaaataccagagctacaggtatggtataccaaacttggaaaaacatccgaaagatttctttttaagcagctggattctctatggctccttgacagcgatggcagtttcacactgagcctgcatgaagatgagctgttcacactcaccactctcaccactggtcgcaaaggcagctacccgcttcctccaaaatcccagcccttcccaagtacctataaggatgatttcaatgttgattacccattttttagtgaagctccaaactttgctgatcaaactggtgtatttgaatattttacaaatattgaagaccctggcgagcatcacttcacgctacgccaagttctcaaccagagacccattacgtgggctgccgatgcatccaacacaatcagtattataggagactacaactggaccaatctgactataaagtgtgatgtatacatagagacccctgacacaggaggtgtgttcattgcaggaagagtaaataaaggtggtattttgattagaagtgccagaggaattttcttctggatttttgcaaatggatcttacagggttacaggtgatttagctggatggattatatatgctttaggacgtgttgaagttacagcaaaaaaatggtatacactcacgttaactattaagggtcatttcacctctggcatgctgaatgacaagtctctgtggacagacatccctgtgaattttccaaagaatggctgggctgcaattggaactcactcctttgaatttgcacagtttgacaactttcttgtggaagccacacgctaa

SEQ ID NO: 6 - protein, 685 aa, Artificial sequence
  Synthetic Construct
MAEWLLSASWQRRAKAMTAAAGSAGRAAVPLLLCALLAPGGAYVLDDSDGLGREFDGIGAVSGGGATSRLLVNYPEPYRSQILDYLFKPNFGASLHILKVEIGGDGQTTDGTEPSHMHYALDENYFRGYEWWLMKEAKKRNPNITLIGLPWSFPGWLGKGFDWPYVNLQLTAYYVVTWIVGAKRYHDLDIDYIGIWNERSYNANYIKILRKMLNYQGLQRVKIIASDNLWESISASMLLDAELFKVVDVIGAHYPGTHSAKDAKLTGKKLWSSEDFSTLNSDMGAGCWGRILNQNYINGYMTSTIAWNLVASYYEQLPYGRCGLMTAQEPWSGHYVVESPVWVSAHTTQFTQPGWYYLKTVGHLEKGGSYVALTDGLGNLTIIIETMSHKHSKCIRPFLPYFNVSQQFATFVLKGSFSEIPELQVWYTKLGKTSERFLFKQLDSLWLLDSDGSFTLSLHEDELFTLTTLTTGRKGSYPLPPKSQPFPSTYKDDFNVDYPFFSEAPNFADQTGVFEYFTNIEDPGEHHFTLRQVLNQRPITWAADASNTISIIGDYNWTNLTIKCDVYIETPDTGGVFIAGRVNKGGILIRSARGIFFWIFANGSYRVTGDLAGWIIYALGRVEVTAKKWYTLTLTIKGHFTSGMLNDKSLWTDIPVNFPKNGWAAIGTHSFEFAQFDNFLVEATR

SEQ ID NO: 7 - DNA, 2007 bp, Artificial sequence
  Engineered canine GALC
  CDS (1)..(2007)
atgacagccgctgctggatctgctggacacgctgctgttcctctgctgctgtgtgcactgctggtgcctggcggagcttacgtgctggatgattctgatggactaggccgcgagttcgacggcgtgggagctgtttctggtggcggagccacatctaggctgctggtcaattaccccgagccttacaggtcccagatcctggactacctgttcaagcccaacttcggcgccagcctgcacatcctgaaggtggaaattggcggcgacggccagaccaccgacggaacagaacctagccacatgcactacgccctggacgagaacttcttcaggggctacgagtggtggctgatgaaggaagccaagaagaggaaccccaacatcatcctgatgggcctgccttggtctttccctggctggatcggcaagggcttcaactggccttacgtgaacctgcagctgaccgcctactacatcatgacctggatcgtgggcgccaagcactaccacgacctggacatcgactacatcggcatctggaacgagcggagcttcgacatcaattacatcaaggtgctgcggcggatgctgaactaccagggcctcgacagagtgaagatcattgccagcgacaacctgtgggagcccatcagcgcttctatgctgctggactccgagctgctgaaagtgatcgacgtgatcggcgctcactaccccggaacacacaccgtgaaggacgccaaactgaccaagaagaagctgtggtccagcgaggacttcagcaccctgaatagtgacgtcggagccggctgcctgggcagaatcctgaaccagaactacgtgaacggctacatgaccgccacaatcgcctggaatctggtggccagctactacgagcagctgccctacggaaggtgcggcctgatgacagcccaagagccttggagcggacactacgtggtggaaagccctatctgggtgtcagcccacaccacacagttcacccagcctggctggtactacctgaaaaccgtgggccacctggaaaaaggcggctcttatgtggccctgaccgacggcctgggaaacctgacaatcatcgtggaaaccatgagccacaagcagagcgcctgcatcagaccctttctgccctacttcaacgtgtccaggcagttcgccaccttcgtgctgaagggcagcttcagcgagatccccgaactgcaagtgtggtacaccaagctgggcaagcccagcgagagatacctgtttaagcagctggacagcctgtggctgctcgacagcagctctaccttcacactggaactccaagaggacgagatcttcaccctgaccacactgaccgtgggcagcaagggatcttaccctctgcctcctaagagcgagccctttccacagatctacgaggacgacttcgacgtggactacccattctttagcgaggcccctaacttcgccgatcagaccggcgtgttcgagtacttcaccaacatcgaggaccccggcgagcacaggtttaccctgagacaggtgctgaaccagcggcctattacctgggccgctgacgcctacaacaccatctctatcatcggcgactacaagtggtccaacctgaccgtcagatgcgacgtgtacatcgagacacccgagaaaggcggagtgtttatcgccggcagagtgaacaaaggcggcatcctgatcagaagcgccaggggaatcttcttctggatcttcgccaacggcacctacagagtgacaggcgatctggccggatgggtcatctatgccctgggaagagtggacgtgaccgccaagaagtggtacactctgaccctgatcatcaagggcagactgagcagcggcatgctgaatggcaagaccgtgtggaagaacatccccgtgtctttccccaaaaacggctgggccgccatcggaacccacagcttcgaatttgcccagttcgacaactttcacgtggaagccaccaga

SEQ ID NO: 8 - protein, 669 aa, Artificial sequence
  Synthetic Construct
MTAAAGSAGHAAVPLLLCALLVPGGAYVLDDSDGLGREFDGVGAVSGGGATSRLLVNYPEPYRSQILDYLFKPNFGASLHILKVEIGGDGQTTDGTEPSHMHYALDENFFRGYEWWLMKEAKKRNPNIILMGLPWSFPGWIGKGFNWPYVNLQLTAYYIMTWIVGAKHYHDLDIDYIGIWNERSFDINYIKVLRRMLNYQGLDRVKIIASDNLWEPISASMLLDSELLKVIDVIGAHYPGTHTVKDAKLTKKKLWSSEDFSTLNSDVGAGCLGRILNQNYVNGYMTATIAWNLVASYYEQLPYGRCGLMTAQEPWSGHYVVESPIWVSAHTTQFTQPGWYYLKTVGHLEKGGSYVALTDGLGNLTIIVETMSHKQSACIRPFLPYFNVSRQFATFVLKGSFSEIPELQVWYTKLGKPSERYLFKQLDSLWLLDSSSTFTLELQEDEIFTLTTLTVGSKGSYPLPPKSEPFPQIYEDDFDVDYPFFSEAPNFADQTGVFEYFTNIEDPGEHRFTLRQVLNQRPITWAADAYNTISIIGDYKWSNLTVRCDVYIETPEKGGVFIAGRVNKGGILIRSARGIFFWIFANGTYRVTGDLAGWVIYALGRVDVTAKKWYTLTLIIKGRLSSGMLNGKTVWKNIPVSFPKNGWAAIGTHSFEFAQFDNFHVEATR

SEQ ID NO: 9 - DNA, 2055 bp, Artificial sequence
  Engineered human GALC coding sequence
  CDS (1)..(2055)
  sig_peptide (1)..(126)
  mat (127)..(2055)
atggccgagtggctgctgtctgctagctggcagagaagggccaaggccatgacagccgccgctggatctgcaggcagagctgctgtgcctctgctgctgtgtgcactgctggcacctggcggagcctacgtgctggatgattctgacggcctgggcagagagttcgacggcatcggagctgtgtctggcggcggagccacaagcagactgctcgtgaactaccccgagccctacagaagccagatcctggactacctgttcaagcccaacttcggcgccagcctgcacatcctgaaggtggaaatcggcggcgacggccagaccaccgatggcacagaacctagccacatgcactacgccctggacgagaactacttccggggctacgagtggtggctgatgaaggaagccaagaagcggaaccccaacatcaccctgatcggcctgccttggagcttccctggctggctgggcaagggcttcgactggccttacgtgaacctgcagctgaccgcctactacgtcgtgacctggatcgtgggcgccaagagataccacgacctggacatcgactacatcggcatctggaacgagcggagctacaacgccaactacatcaagatcctgcggaagatgctgaactaccagggcctgcagagagtgaagatcattgccagcgacaacctgtgggagagcatcagcgcctccatgctgctggacgccgagctgttcaaggtggtggatgtgatcggcgcccactaccctggcacacactctgccaaggacgccaagctgaccggcaagaagctgtggtccagcgaggacttcagcaccctgaacagcgacatgggagccggctgctggggcagaatcctgaaccagaattacatcaacggctacatgaccagcacaatcgcctggaacctggtggccagctactacgagcagctgccctacggcagatgcggcctgatgacagcccaggaaccttggagcggccactacgtggtggaaagccctgtgtgggtgtccgcccacaccacccagtttacacagcccggctggtactacctgaaaaccgtgggccacctggaaaagggcggcagctatgtggccctgacagacggactgggcaacctgaccatcatcatcgagacaatgtcccacaagcacagcaagtgcatcagaccctttctgccctacttcaacgtgtcccagcagttcgccaccttcgtgctgaagggcagcttcagcgagatccccgaactgcaagtgtggtacaccaagctgggaaagaccagcgagcggttcctgtttaagcagctggacagcctgtggctgctggacagcgacggcagctttaccctgagcctgcacgaggacgagctgtttacactgaccaccctgaccacaggccggaagggctcttaccccctgcctcctaagagccagcccttcccaagcacctacaaggacgacttcaatgtggactacccattctttagcgaggcccccaatttcgccgaccagaccggcgtgttcgagtacttcaccaacatcgaggaccccggcgagcaccacttcaccctgagacaggtgctgaaccagcggcccatcacctgggctgccgatgccagcaacaccatcagcatcatcggcgactacaactggaccaatctgacaatcaagtgcgacgtgtacatcgaaacccccgacaccggcggagtgtttatcgccggcagagtgaacaagggcgggatcctgatcagaagcgccagaggcatcttcttttggatcttcgccaacggcagctacagagtgaccggcgatctggccggctggatcatctacgctctgggcagggtggaagtgaccgccaagaaatggtacaccctgacactgactatcaagggccacttcgcctccggcatgctgaacgacaagagcctgtggaccgacatccccgtgaacttccccaagaatggctgggccgccatcggcacccacagctttgagttcgcccagttcgacaacttcctggtggaagccaccaga

SEQ ID NO: 10 - protein, 685 aa, Artificial sequence [sequence omitted]

SEQ ID NO: 11 - DNA, 130 bp, Artificial sequence
  AAV2 - 5' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 12 - DNA, 382 bp, Artificial sequence
  CMV IE enhancer
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 13 - DNA, 282 bp, Artificial sequence
  CB promoter
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 14 - DNA, 666 bp, Artificial sequence
  CB7 promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 15 - DNA, 972 bp, Artificial Sequence
  chimeric intron
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 16 - DNA, 127 bp, Artificial sequence
  Rabbit globin polyA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 17 - DNA, 130 bp, Artificial sequence
  AAV2 - 3' ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 18 - DNA, 4337 bp, Artificial sequence
  Vector genome with canine GALC
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgacagccgctgctggatctgctggacacgctgctgttcctctgctgctgtgtgcactgctggtgcctggcggagcttacgtgctggatgattctgatggactaggccgcgagttcgacggcgtgggagctgtttctggtggcggagccacatctaggctgctggtcaattaccccgagccttacaggtcccagatcctggactacctgttcaagcccaacttcggcgccagcctgcacatcctgaaggtggaaattggcggcgacggccagaccaccgacggaacagaacctagccacatgcactacgccctggacgagaacttcttcaggggctacgagtggtggctgatgaaggaagccaagaagaggaaccccaacatcatcctgatgggcctgccttggtctttccctggctggatcggcaagggcttcaactggccttacgtgaacctgcagctgaccgcctactacatcatgacctggatcgtgggcgccaagcactaccacgacctggacatcgactacatcggcatctggaacgagcggagcttcgacatcaattacatcaaggtgctgcggcggatgctgaactaccagggcctcgacagagtgaagatcattgccagcgacaacctgtgggagcccatcagcgcttctatgctgctggactccgagctgctgaaagtgatcgacgtgatcggcgctcactaccccggaacacacaccgtgaaggacgccaaactgaccaagaagaagctgtggtccagcgaggacttcagcaccctgaatagtgacgtcggagccggctgcctgggcagaatcctgaaccagaactacgtgaacggctacatgaccgccacaatcgcctggaatctggtggccagctactacgagcagctgccctacggaaggtgcggcctgatgacagcccaagagccttggagcggacactacgtggtggaaagccctatctgggtgtcagcccacaccacacagttcacccagcctggctggtactacctgaaaaccgtgggccacctggaaaaaggcggctcttatgtggccctgaccgacggcctgggaaacctgacaatcatcgtggaaaccatgagccacaagcagagcgcctgcatcagaccctttctgccctacttcaacgtgtccaggcagttcgccaccttcgtgctgaagggcagcttcagcgagatccccgaactgcaagtgtggtacaccaagctgggcaagcccagcgagagatacctgtttaagcagctggacagcctgtggctgctcgacagcagctctaccttcacactggaactccaagaggacgagatcttcaccctgaccacactgaccgtgggcagcaagggatcttaccctctgcctcctaagagcgagccctttccacagatctacgaggacgacttcgacgtggactacccattctttagcgaggcccctaacttcgccgatcagaccggcgtgttcgagtacttcaccaacatcgaggaccccggcgagcacaggtttaccctgagacaggtgctgaaccagcggcctattacctgggccgctgacgcctacaacaccatctctatcatcggcgactacaagtggtccaacctgaccgtcagatgcgacgtgtacatcgagacacccgagaaaggcggagtgtttatcgccggcagagtgaacaaaggcggcatcctgatcagaagcgccaggggaatcttcttctggatcttcgccaacggcacctacagagtgacaggcgatctggccggatgggtcatctatgccctgggaagagtggacgtgaccgccaagaagtggtacactctgaccctgatcatcaagggcagactgagcagcggcatgctgaatggcaagaccgtgtggaagaacatccccgtgtctttccccaaaaacggctgggccgccatcggaacccacagcttcgaatttgcccagttcgacaactttcacgtggaagccaccagatgataacctcaggctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 19 - DNA, 4386 bp, Artificial sequence
  CB7.CI.hGALC.rBG
  misc_feature (1)..(130): 5' ITR
  misc_feature (198)..(579): CMV IE Enhancer
  misc_feature (582)..(863): CB promoter
  misc_feature (836)..(839): TATA
  misc_feature (958)..(1930): chimeric intron
  misc_feature (1948)..(4002): hGALCco
  misc_feature (4042)..(4168): Rabbit globin poly A
  misc_feature (4257)..(4386): 3' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgccaccatggccgagtggctgctgtctgctagctggcagagaagggccaaggccatgacagccgccgctggatctgcaggcagagctgctgtgcctctgctgctgtgtgcactgctggcacctggcggagcctacgtgctggatgattctgacggcctgggcagagagttcgacggcatcggagctgtgtctggcggcggagccacaagcagactgctcgtgaactaccccgagccctacagaagccagatcctggactacctgttcaagcccaacttcggcgccagcctgcacatcctgaaggtggaaatcggcggcgacggccagaccaccgatggcacagaacctagccacatgcactacgccctggacgagaactacttccggggctacgagtggtggctgatgaaggaagccaagaagcggaaccccaacatcaccctgatcggcctgccttggagcttccctggctggctgggcaagggcttcgactggccttacgtgaacctgcagctgaccgcctactacgtcgtgacctggatcgtgggcgccaagagataccacgacctggacatcgactacatcggcatctggaacgagcggagctacaacgccaactacatcaagatcctgcggaagatgctgaactaccagggcctgcagagagtgaagatcattgccagcgacaacctgtgggagagcatcagcgcctccatgctgctggacgccgagctgttcaaggtggtggatgtgatcggcgcccactaccctggcacacactctgccaaggacgccaagctgaccggcaagaagctgtggtccagcgaggacttcagcaccctgaacagcgacatgggagccggctgctggggcagaatcctgaaccagaattacatcaacggctacatgaccagcacaatcgcctggaacctggtggccagctactacgagcagctgccctacggcagatgcggcctgatgacagcccaggaaccttggagcggccactacgtggtggaaagccctgtgtgggtgtccgcccacaccacccagtttacacagcccggctggtactacctgaaaaccgtgggccacctggaaaagggcggcagctatgtggccctgacagacggactgggcaacctgaccatcatcatcgagacaatgtcccacaagcacagcaagtgcatcagaccctttctgccctacttcaacgtgtcccagcagttcgccaccttcgtgctgaagggcagcttcagcgagatccccgaactgcaagtgtggtacaccaagctgggaaagaccagcgagcggttcctgtttaagcagctggacagcctgtggctgctggacagcgacggcagctttaccctgagcctgcacgaggacgagctgtttacactgaccaccctgaccacaggccggaagggctcttaccccctgcctcctaagagccagcccttcccaagcacctacaaggacgacttcaatgtggactacccattctttagcgaggcccccaatttcgccgaccagaccggcgtgttcgagtacttcaccaacatcgaggaccccggcgagcaccacttcaccctgagacaggtgctgaaccagcggcccatcacctgggctgccgatgccagcaacaccatcagcatcatcggcgactacaactggaccaatctgacaatcaagtgcgacgtgtacatcgaaacccccgacaccggcggagtgtttatcgccggcagagtgaacaagggcgggatcctgatcagaagcgccagaggcatcttcttttggatcttcgccaacggcagctacagagtgaccggcgatctggccggctggatcatctacgctctgggcagggtggaagtgaccgccaagaaatggtacaccctgacactgactatcaagggccacttcgcctccggcatgctgaacgacaagagcctgtggaccgacatccccgtgaacttccccaagaatggctgggccgccatcggcacccacagctttgagttcgcccagttcgacaacttcctggtggaagccaccagatgatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 20 - DNA, 2208 bp, Artificial sequence
  AAV1 VP1
  CDS (1)..(2208)
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctg

SEQ ID NO: 21 - protein, 736 aa, Artificial sequence
  Synthetic Construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 22 - DNA, 2172 bp, Artificial sequence
  AAV5 capsid VP1
  CDS (1)..(2172)
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggcccggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctt

SEQ ID NO: 23 - protein, 724 aa, Artificial sequence
  Synthetic Construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 24 - protein, 736 aa, Artificial sequence
  AAV3B VP1 Capsid
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 25 - DNA, 1227 bp, Artificial sequence
  UbC promoter
ggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggta

SEQ ID NO: 26 - DNA, 232 bp, Artificial sequence
  SV40 late polyA
cgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcg

SEQ ID NO: 27 - DNA, 1184 bp, Artificial sequence
  EF-1a promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

(19) United States
(12) Patent Application Publication
(10) Pub. No.: US 2023/0210941 A1
(43) Pub. Date: Jul. 6, 2023

(54) COMPOSITIONS USEFUL IN TREATMENT OF KRABBE DISEASE

(71) Applicant: The Trustees of the University of Pennsylvania, Philadelphia, PA (US)

(72) Inventors: James M. Wilson, Philadelphia, PA (US); Juliette Hordeaux, Philadelphia, PA (US); Nathan Katz, Stamford, CT (US)

(21) Appl. No.: 17/998,330

(22) PCT Filed: May 11, 2021
(86) PCT No.: PCT/US2021/031802

§ 371 Date: Nov. 9, 2022

Related U.S. Application Data
(60) Provisional application No. 63/073,756, filed on Sep. 2, 2020.
(60) Provisional application No. 63/070,653, filed on Aug. 26, 2020.
(60) Provisional application No. 63/023,459, filed on May 12, 2020.

(51) Int. Cl. A61K 38/16; C12N 15/86; C12N 9/24; A61K 9/00

(52) U.S. Cl. CPC A61K 38/162; A61K 9/0085; C12N 9/2402; C12N 15/86; C12Y 302/01046; C12N 2750/14122; C12N 2750/14143

(57) ABSTRACT

A pharmaceutical composition formulated for delivery of a recombinant adeno-associated virus (rAAV) vector comprising an AAV capsid and a vector genome having human galactosylceramidase (GALC) coding sequence is provided. Also provided are 5 methods and uses of a pharmaceutical composition comprising a rAAV for the treatment of Krabbe disease.

### BACKGROUND OF THE INVENTION

Adeno-associated virus (AAV), a member of the Parvovirus family, is a small non-enveloped, icosahedral virus with single-stranded linear DNA (ssDNA) genomes of about 4.7 kilobases (kb) long. The wild-type genome comprises inverted terminal repeats (ITRs) at both ends of the DNA strand, and two open reading frames (ORFs): rep and cap. Rep is composed of four overlapping genes encoding rep proteins required for the AAV life cycle, and cap contains overlapping nucleotide sequences of capsid proteins: VP1, VP2 and VP3, which self-assemble to form a capsid of an icosahedral symmetry.

Recombinant adeno-associated virus (rAAV) vectors derived from the replication defective human parvovirus have been described as suitable vehicles for gene delivery. Typically, functional rep genes and the cap gene are removed from the vector, resulting in a replication-incompetent vector. These functions are provided during the vector production system but absent in the final vector.

To date, there have been several different well-characterized AAVs isolated from human or non-human primates (NHP). It has been found that AAVs of different serotypes exhibit different transfection efficiencies, and exhibit tropism for different cells or tissues. Many different AAV clades have been described in WO 2005/033321, including clade F which is identified therein as having just three members, AAV9, AAVhu31 and AAVhu32. A structural analysis of AAV9 is provided in M. A. DiMattia et al, J. Virol. (June 2012) vol. 86 no. 12 6947-6958. This paper reports that AAV9 has 60 copies (in total) of the three variable proteins (vps) that are encoded by the cap gene and have overlapping sequences. These include VP1 (87 kDa), VP2 (73 kDa), and VP3 (62 kDa), which are present in a predicted ratio of 1:1:10, respectively. The entire sequence of VP3 is within VP2, and all of VP2 is within VP1. VP1 has a unique N-terminal domain. The refined coordinates and structure factors are available under accession no. 3UX1 from the RCSB PDB database.

Several different AAV9 variants have been engineered in order to detarget or target different tissue. See, e.g., N. Pulicheria, “Engineering Liver-detargeted AAV9 Vectors for Cardiac and Musculoskeletal Gene Transfer”, Molecular Therapy, Vol, 19, no. 6, p. 1070-1078 (June 2011). The development of AAV9 variants to deliver gene across the blood-brain barrier has also been reported. See, e.g., B.E. Deverman et al, Nature Biotech, Vol. 34, No. 2, p 204 - 211 (published online 1 Feb. 2016) and Caltech press release, A. Wetherston, www.neurology-central.com/2016/02/10/successful-delivery-of-genes-through-the-blood-brain-barrier/, accessed Oct. 05, 2016. See, also, WO 2016/0492301 and US 8,734,809.

Recently, AAVhu68, which was identified following amplification of the capsid gene from a natural source, was identified as a new AAV capsid. See, e.g., WO 2018/160582. This AAV is within Clade F, as is AAV9.

Krabbe disease (globoid cell leukodystrophy; GLD) is an autosomal recessive lysosomal storage disease (LSD) caused by mutations in the gene encoding the hydrolytic enzyme galactosylceramidase (GALC) (Wenger D.A., et al. (2000) Mol Genet Metab. 70(1): 1-9). This enzyme is responsible for the degradation of certain galactolipids, including galactosylceramide (ceramide) and galactosylsphingosine (psychosine), which are found almost exclusively in the myelin sheath. In Krabbe disease, GALC deficiency causes toxic accumulation of psychosine (but not galactosylceramide) in the lysosomes (Svennerholm et al., 1980). The accumulation of psychosine is particularly toxic to myelin-producing oligodendrocytes in the CNS and Schwann cells in the PNS, resulting in rapid and widespread death of these cell types. Myelin breakdown in both the CNS and PNS is accompanied by reactive astroytic gliosis and the infiltration of giant multinucleated macrophages (“globoid cells”) (Suzuki K. (2003) J Child Neurol. 18(9):595-603). Galactosylceramide does not accumulate in the absence of GALC activity due primarily to hydrolysis by another enzyme, GM1 ganglioside β-galactosidase (Kobayashi T., et al. (1985) J Biol Chem. 260(28): 14982-7) and the death of oligodendrocytes contributing to an arrest in the galactosylceramide synthesis (Svennerholm L., et al. (1980) J Lipid Res. 21(1):53-64).

The only disease-modifying treatment currently available for Krabbe disease is hematopoietic stem cell transplant (HSCT), which is often provided by umbilical cord blood transplant (UCBT), allogeneic peripheral blood stem cells, or allogeneic bone marrow. There has been only modest success using HSCT to treat patients with infantile Krabbe disease, who typically present with symptoms before their first birthday. When performed after the onset of overt symptoms in infantile Krabbe disease, HSCT provides only minimal neurologic improvement and does not substantially improve survival (Escolar M.L., et al. (2005) N Engl J Med. 352(20):2069-81). HSCT can be efficacious when performed in presymptomatic patients, but even then, motor outcomes are poor (Escolar M.L., et al. (2005) N Engl J Med. 352(20):2069-81; Wright M.D., et al. (2017) Neurology. 89(13): 1365-1372; van den Broek B.T.A., et al. (2018) Blood Adv. 2(1):49-60). Infants transplanted before 30 days of age had better survival and functional outcomes compared with those transplanted later (Allewelt H., et al. (2018) Biol Blood Marrow Transplant. 24(11):2233-2238). Presymptomatic transplantation is reported to result in significantly better outcomes with progressive central myelination, normal receptive language, attenuation of symptom severity, and longer survival compared with infantile Krabbe disease patients who were either untreated or treated after symptom onset (Escolar M.L., et al. (2005) N Engl J Med. 352(20):2069-81; Duffner P.K., et al. (2009) Genet Med. 11(6):450-4; Wright M.D., et al. (2017) Neurology. 89(13): 1365-1372). Even so, most children treated before the emergence of symptoms remain well below average for height and weight, and have progressive gross motor delays ranging from mild spasticity to inability to walk independently (Escolar M.L., et al. (2005) N Engl J Med. 352(20):2069-81; Duffner P.K., et al. (2009) Genet Med. 11(6):450-4). Some children also have residual impairments, including acquired microcephaly, the need for gastrostomy, and dysarthria (Duffner P.K., et al. (2009) Genet Med. 11(6):450-4). Moreover, HSCT only appears to influence the CNS-specific disease pathology. Clinical features associated with the PNS pathology, such as peripheral neuropathy, remain unaffected by HSCT. These results highlight the limitations of HSCT, especially in early onset forms where rapid disease progression outpaces the time needed for hematopoietic stem cells to engraft, migrate to the CNS, differentiate, and provide therapeutic effect through GALC secretion and cross-correction (i.e., the process by which enzyme secreted by corrective cells is taken up by GALC-deficient cells).

There remains a need in the art for improved treatments for Krabbe disease patients.

### SUMMARY OF THE INVENTION

These and other aspects of the invention will be apparent from the following detailed description of the invention.

In one aspect, a pharmaceutical composition comprising a stock of recombinant AAV (rAAV) having an AAV capsid and a vector genome packaged therein is provided. The vector genome comprises: (a) a 5′ inverted terminal repeat (ITR); (b) a CB7 promoter; (c) an intron; (d) a galactosylceramidase (GALC) coding sequence comprising nucleotides 1 to 2055 of SEQ ID NO: 9, or a sequence at least 95% identical thereto that encodes amino acids 1 to 685 of SEQ ID NO: 10; (e) a polyA; and (f) a 3′ ITR. In certain embodiments, the composition is formulated for administration of a dose of about 1.7 × 10¹⁰ genome copies (GC)/g brain mass to about 5.0 × 10¹¹ GC/g brain mass. In certain embodiments, the AAV capsid is an AAVhu68 capsid. In further embodiments, the vector genome comprises nucleotides 198 to 4168 of SEQ ID NO: 19.

In one aspect, a method of treating Krabbe disease in a patient in need thereof, wherein the method comprises intracisternal magna (ICM) administration of the pharmaceutical composition described herein. In certain embodiments, the method comprises hematopoietic stem cell transplant before or after administration of the pharmaceutical composition. The hematopoietic stem cell transplant may permit a reduced dose of the rAAV to be administered to the patient.

In one aspect, a method for increasing GALC expression and enzyme activity in the serum and/or cerebral spinal fluid (CSF) of a patient having Krabbe disease is provided, wherein the method comprises administering a pharmaceutical composition described herein to the patient.

In one aspect, a method for reducing neuroinflammation in peripheral nerves of a patient having Krabbe disease is provided, wherein the method comprises administering a pharmaceutical composition described herein to the patient.

In one aspect, a method for increasing GALC expression and activity in neurons of the cortex and/or hippocampus of a patient having Krabbe disease is provided, wherein the method comprises administering a pharmaceutical composition described herein to the patient.

In one aspect, a pharmaceutical composition for use in the treatment of a patient with Krabbe disease is provided. In certain embodiments, the treatment i) increases GALC expression and enzyme activity in the serum and/or cerebral spinal fluid (CSF), ii) increases GALC expression and activity in neurons of the cortex and/or hippocampus, and/or iii) increases psychosine in the serum and/or cerebral spinal fluid (CSF).

In one aspect, the use of a pharmaceutical composition described herein for treating Krabbe disease in a patient in need thereof is provided, optionally followed by bone marrow transplant.

### BRIEF DESCRIPTION OF THE DRAWINGS

FIG. 1 provides an alignment of AAV9 (SEQ ID NO: 4) and AAVhu68 (SEQ ID NO: 2) capsid sequences. The two amino acids that differ between the AAV9 and AAVhu68 capsids are located in the VP1 (67, 157) and VP2 (157) regions of the capsid. Abbreviations: AAV9, adeno-associated virus serotype 9; AAVhu68; adeno-associated virus serotype hu68; VP1, viral protein 1; VP2, viral protein.

FIG. 2 shows a schematic of the CB7.CI.hGALC.rBG vector genome. The linear map depicts the vector genome, which is designed to express human GALC under the control of the ubiquitous CB7 promoter. CB7 is composed of hybrid between a CMV IE enhancer and a chicken β-actin (CB) promoter. Abbreviations: CMV IE, cytomegalovirus immediate-early; GALC, galctosylceramidase; ITR, inverted terminal repeats; PolyA, polyadenylation; rBG, rabbit β-globin.

FIG. 3 shows a vector map for pENN.AAV.CB7.CI.RBG (p1044) with an engineered cGALC gene (cGALCco) inserted.

FIG. 4 shows Linear vector map of the trans plasmid pAAV2/hu68.KanR (p0068). Abbreviations: AAV2, adeno-associated virus serotype 2; AAVhu68, adeno-associated virus serotype hu68; bp, base pairs; Cap, capsid; KanR, kanamycin resistance; Ori, origin of replication; Rep, replicase.

FIG. 5A and FIG. 5B show the adenovirus helper plasmid pAdDeltaF6(KanR). (FIG. 5A) Derivation of the helper plasmid pAdΔF6 from parental plasmid pBHG10 through intermediates pAdΔF1 and pAdΔF5. (FIG. 5B) The ampicillin resistance gene in pAdΔF6 was replaced by the kanamycin resistance gene to generate pAdΔF6(Kan).

FIG. 6A shows the progression of neuropathological and behavioral phenotypes for the Twitcher mouse (twi/twi). Mice display an accumulation of cytotoxic psychosine followed by the infiltration of the PNS and CNS white matter by phagocytic globoid cells. Following an initial period of myelination, demyelination is observed in the PNS followed by the CNS to a lesser extent due to the death of myelin-forming Schwann cells and oligodendrocytes, respectively. Behavioral phenotypes manifest around PND 20 consisting of tremors, twitching, hind limb weakness, followed by subsequent paralysis and weight loss necessitating euthanasia around PND 40. Adapted from (Nicaise A.M., et al. (2016) J Neurosci Res. 94(11): 1049-61). Abbreviations: CNS, central nervous system; PND, postnatal day; PNS, peripheral nervous system; twi, twitcher loss-of-function allele.

FIG. 6B shows a study design for evaluation of AAV.CB7.cGALCco.rBG gene therapy using the Twitcher mouse model.

FIG. 7 shows survival of twi/twi mice administered rAAVhu68.hGALC or vehicle. On PND 0, twi/twi mice were IV-administered rAAVhu68.hGALC at a dose of 1.0 × 10¹¹ GC. Age-matched twi/twi mice and WT mice were IV-administered PBS as controls. Survival was monitored. P= p=0.0006 based on a comparison of each group to the vehicle-treated twi/twi control group using a Log-rank (Mantel-Cox) test. Abbreviations: GC, genome copies; PBS, phosphate-buffered saline; twi, Twitcher allele (consisting of a loss-of-function mutation in the Galc gene).

FIG. 8 shows transgene expression (GALC Activity) in the brain of twi/twi mice administered rAAVhu68.hGALC or vehicle. On PND 0, twi/twi mice were IV-administered rAAVhu68.hGALC at a dose of 1.0 × 10¹¹ GC. Age-matched twi/twi mice and WT mice were IV-administered PBS as controls. Brain GALC enzyme activity was measured. A one-way ANOVA post hoc multiple comparison Tukey’s comparing each group to twi/twi PBS. Dashed line indicates mean of wild type PBS group. Abbreviations: GC, genome copies; PBS, phosphate-buffered saline; twi, Twitcher allele (consisting of a loss-of-function mutation in the Galc gene).

FIG. 9 shows survival of twi/twi mice administered rAAVhu68.hGALC or vehicle. On PND 0, twi/twi mice were ICV-administered rAAVhu68.hGALC at one of three doses, 2.0 × 10¹⁰, 5.0 × 10¹⁰, 1.0 × 10¹¹ GC. Age-matched twi/twi mice and WT mice were ICV-administered PBS as controls. Survival was monitored. p=0.0001 based on a comparison of each group to the vehicle-treated twi/twi control group using a Log-rank (Mantel-Cox) test. Abbreviations: GC, genome copies; PBS, phosphate-buffered saline; twi, Twitcher allele (consisting of a loss-of-function mutation in the Galc gene).

FIG. 10 shows body weights of twi/twi mice administered rAAVhu68.hGALC or vehicle. On PND 0, twi/twi mice were ICV-administered rAAVhu68.hGALC at one of three doses, 2.0 × 10¹⁰, 5.0 × 10¹⁰, 1.0 × 10¹¹ GC. Age-matched twi/twi mice and WT mice were ICV-administered PBS as controls. Animals were weighed three times per week after weaning. Error bars represent the standard deviation. p=0.0001 based on statistical analysis of the longitudinal data using linear mixed effect modeling to compare each group to the PBS-treated twi/twi control group. Abbreviations: GC, genome copies; PBS, phosphate-buffered saline; twi, Twitcher allele (consisting of a loss-of-function mutation in the Galc gene).

FIG. 11 shows neuromotor function of twi/twi mice administered rAAVhu68.hGALC or vehicle. On PND 0, twi/twi mice were ICV-administered rAAVhu68.hGALC at one of three doses, 2.0 × 10¹⁰, 5.0 × 10¹⁰, 1.0 × 10¹¹ GC. Age-matched twi/twi mice and WT mice were ICV-administered PBS as controls. On PND 35, neuromotor function was assessed by the time to fall (seconds) for mice running on an accelerating rod initially spinning at 5 RPM and increasing to 40 RPM over 120 seconds. Error bars represent the standard deviation. **p<0.007, ****p<0.0001 based on a comparison of each group to the PBS-treated twi/twi control group using a 1-way ANOVA and post hoc Dunn’s multiple comparisons test. Abbreviations: GC, genome copies; PBS, phosphate-buffered saline; twi, Twitcher allele (consisting of a loss-of-function mutation in the Galc gene).

FIG. 12 shows transgene expression (GALC Activity) in the brain of twi/twi mice administered rAAVhu68.hGALC or vehicle. On PND 0, twi/twi mice were ICV-administered rAAVhu68.hGALC at one of three doses, 2.0 × 10¹⁰, 5.0 × 10¹⁰, 1.0 × 10¹¹ GC. Age-matched twi/twi mice and WT mice were ICV-administered PBS as controls. At necropsy, the brain was collected for a GALC enzyme activity assay to evaluate transgene expression. Error bars represent the standard deviation. The dashed line indicates the mean wild type PBS-treated group. ****p<0.0001 based on a comparison of each group to the PBS-treated twi/twi control group using a 1-way ANOVA and post hoc Dunn’s multiple comparisons test. Abbreviations: GC, genome copies; PBS, phosphate-buffered saline; twi, Twitcher allele (consisting of a loss-of-function mutation in the Galc gene).

FIG. 13 shows transgene expression (GALC Activity) in the liver of twi/twi mice administered rAAVhu68.hGALC or vehicle. On PND 0, twi/twi mice were ICV-administered rAAVhu68.hGALC at one of three doses, 2.0 × 10¹⁰, 5.0 × 10¹⁰, 1.0 × 10¹¹ GC. Age-matched twi/twi mice and WT mice were ICV-administered PBS as controls. At necropsy, the liver was collected for a GALC enzyme activity assay to evaluate transgene expression. Error bars represent the standard deviation. Abbreviations: GC, genome copies; PBS, phosphate-buffered saline; twi, Twitcher allele (consisting of a loss-of-function mutation in the Galc gene).

FIG. 14 shows transgene expression (GALC Activity) in the serum of twi/twi mice administered rAAVhu68.hGALC or vehicle. On PND 0, twi/twi mice were ICV-administered rAAVhu68.hGALC at one of three doses, 2.0 × 10¹⁰, 5.0 × 10¹⁰, 1.0 × 10¹¹ GC. Age-matched twi/twi mice and WT mice were ICV-administered PBS as controls. At PND28, blood was collected for a GALC enzyme activity assay to evaluate transgene expression. Error bars represent the standard deviation. Abbreviations: GC, genome copies; PBS, phosphate-buffered saline; twi, Twitcher allele (consisting of a loss-of-function mutation in the Galc gene).

FIG. 15 shows brain myelination in twi/twi mice administered rAAVhu68.hGALC or vehicle. On PND 0, twi/twi mice were ICV-administered rAAVhu68.hGALC at one of three doses, 2.0 × 10¹⁰, 5.0 × 10¹⁰, 1.0 × 10¹¹ GC. Age-matched twi/twi mice and WT mice were ICV-administered PBS as controls. At necropsy, the brain was collected, processed, and stained with LFB/PAS for myelination and globoid cell infiltration assessments. Pictures were taken at low magnification. Arrows show the corpus callosum. Scale bar 2 mm. Abbreviations: GC, genome copies; LFB, Luxol Fast Blue; PAS, Periodic Acid-Schiff; PBS, phosphate-buffered saline; twi, Twitcher allele (consisting of a loss-of-function mutation in the Galc gene).

FIG. 16 shows brain myelination in twi/twi mice administered rAAVhu68.hGALC or vehicle - high magnification. On PND 0, twi/twi mice were ICV-administered rAAVhu68.hGALC at one of three doses, 2.0 × 10¹⁰, 5.0 × 10¹⁰, 1.0 × 10¹¹ GC. Age-matched twi/twi mice and WT mice were ICV-administered PBS as controls. At necropsy, the brain was collected, processed, and stained with LFB/PAS for myelination and globoid cell infiltration assessments. Pictures were taken at high magnification (20×). Yellow arrows point globoid cells. Star: central white matter cerebellum. Scale bars 100 um. Abbreviations: GC, genome copies; LFB, Luxol Fast Blue; PAS, Periodic Acid-Schiff; PBS, phosphate-buffered saline; twi, Twitcher allele (consisting of a loss-of-function mutation in the Galc gene).

FIG. 17 shows sciatic nerve myelination of twi/twi mice administered rAAVhu68.hGALC or vehicle. On PND 0, twi/twi mice were ICV-administered rAAVhu68.hGALC at one of three doses, 2.0 × 10¹⁰, 5.0 × 10¹⁰, 1.0 × 10¹¹ GC. Age-matched twi/twi mice and WT mice were ICV-administered PBS as controls. At necropsy, the sciatic nerve was collected, processed, and stained with Toluidine blue for myelination and globoid cell infiltration assessments. Pictures were taken at a 40× magnification. Arrows indicate myelinated nerve fibers. Abbreviations: GC, genome copies; phosphate-buffered saline; twi, Twitcher allele (consisting of a loss-of-function mutation in the Galc gene).

FIG. 18 shows neuroinflammation (IBA1) in the brain of twi/twi mice administered rAAVhu68.hGALC or vehicle. On PND 0, twi/twi mice were ICV-administered rAAVhu68.hGALC at one of three doses, 2.0 × 10¹⁰, 5.0 × 10¹⁰, 1.0 × 10¹¹ GC. Age-matched twi/twi mice and WT mice were ICV-administered PBS as controls. At necropsy, the brain was collected, processed, and stained for neuroinflammation (IBA1 staining). Dark staining indicated activated microglial cells and globoid cells (arrows). Central cerebellar white matter is indicated with the black stars and there is an absence of globoid cells. Globoid cells were present in the cerebellum folia and brainstem of rAAVhu68.hGALC -treated mice. Scale bar is 100 µm. Abbreviations: GC, genome copies; PBS, phosphate-buffered saline; twi, Twitcher allele (consisting of a loss-of-function mutation in the Galc gene).

FIG. 19 shows hGALC expression in the brain of twi/twi mice administered rAAVhu68.hGALC or vehicle. On PND 0, twi/twi mice were ICV-administered rAAVhu68.hGALC at one of three doses, 2.0 × 10¹⁰, 5.0 × 10¹⁰, 1.0 × 10¹¹ GC. Age-matched twi/twi mice and WT mice were ICV-administered PBS as controls. At necropsy, the brain was collected, processed, and stained for hGALC detection. Scale bar is 200 µm. Abbreviations: GC, genome copies; PBS, phosphate-buffered saline; twi, Twitcher allele (consisting of a loss-of-function mutation in the Galc gene).

FIG. 20A and FIG. 20B show survival of twi/twi mice. On PND 0, twi/twi mice were ICV-administered either AAVhu68.hGALC, AAV1.hGALC, AAV3B.hGALC, or AAV5.hGALC at a dose of 2.0 ×10¹⁰. Survival was monitored. Abbreviations: GC, genome copies; PBS, phosphate-buffered saline; twi, Twitcher allele (consisting of a loss-of-function mutation in the Galc gene).

FIG. 21 shows body weights of twi/twi mice. On PND 0, twi/twi mice were ICV-administered either AAV1.hGALC, AAV3B.hGALC, or AAV5.hGALC at a dose of 2.0 ×10¹⁰ GC. Animals were weighted three times per week after weaning. Abbreviations: GC, genome copies; PBS, phosphate-buffered saline; twi, Twitcher allele (consisting of a loss-of-function mutation in the Galc gene).

FIG. 22 shows neuromotor function of twi/twi mice. On PND 0, twi/twi mice were ICV-administered either AAV1.hGALC, AAV3B.hGALC, or AAV5.hGALC at a dose of 2.0 ×10¹⁰. On PND 35, neuromotor function was assessed by the time to fall (seconds) for mice running on an accelerating rod initially spinning at 5 RPM and increasing to 40 RPM over 120 seconds. Error bars represent the standard deviation. Abbreviations: GC, genome copies; PBS, phosphate-buffered saline; twi, Twitcher allele (consisting of a loss-of-function mutation in the Galc gene).

FIG. 23 shows transgene expression (GALC Activity) in the brain of twi/twi mice. On PND 0, twi/twi mice were ICV-administered either AAV1.hGALC, AAV3B.hGALC, or AAV5.hGALC at a dose of 2.0 ×10¹⁰. At necropsy, the brain was collected for a GALC enzyme activity assay to evaluate transgene expression. Error bars represent the standard deviation. The dashed line indicates the mean wild type PBS-treated group. Abbreviations: GC, genome copies; PBS, phosphate-buffered saline; twi, Twitcher allele (consisting of a loss-of-function mutation in the Galc gene).

FIG. 24 shows transgene expression (GALC Activity) in the liver of twi/twi mice. On PND 0, twi/twi mice were ICV-administered either AAV1.hGALC, AAV3B.hGALC, or AAV5.hGALC at a dose of 2.0 ×10¹⁰. At necropsy, the liver was collected for a GALC enzyme activity assay to evaluate transgene expression. Error bars represent the standard deviation. Abbreviations: GC, genome copies; PBS, phosphate-buffered saline; twi, Twitcher allele (consisting of a loss-of-function mutation in the Galc gene).

FIG. 25 shows transgene expression (GALC Activity) in the serum of twi/twi mice. On PND 0, twi/twi mice were ICV-administered either AAV1.hGALC, AAV3B.hGALC, or AAV5.hGALC at a dose of 2.0 ×10¹⁰. On PND28, blood was collected for a GALC enzyme activity assay to evaluate transgene expression. Error bars represent the standard deviation. Abbreviations: GC, genome copies; PBS, phosphate-buffered saline; twi, Twitcher allele (consisting of a loss-of-function mutation in the Galc gene).

FIG. 26 shows sciatic nerve myelination in twi/twi mice. On PND 0, twi/twi mice were ICV-administered either AAV1.hGALC, AAV3B.hGALC, or AAV5.hGALC at a dose of 2.0 ×10¹⁰. At necropsy, the sciatic nerve was collected, processed and stained with Toluidine blue for myelination and globoid cell infiltration assessments. Pictures were taken at a 40× magnification. Abbreviations: GC, genome copies; PBS, phosphate-buffered saline; twi, Twitcher allele (consisting of a loss-of-function mutation in the Galc gene).

FIG. 27 shows neuroinflammation (IBA1) in the brain of twi/twi mice. On PND 0, twi/twi mice were ICV-administered either AAV1.hGALC, AAV3B.hGALC, or AAV5.hGALC at a dose of 2.0 ×10¹⁰. At necropsy, the brain was collected, processed and stained for neuroinflammation (IBA1 staining). Dark staining indicated activated microglial cells and globoid cells. Scale bar is 100 µm. Abbreviations: GC, genome copies; PBS, phosphate-buffered saline; twi, Twitcher allele (consisting of a loss-of-function mutation in the Galc gene).

FIG. 28 shows hGALC expression in the brain of twi/twi mice. On PND 0, twi/twi mice were ICV-administered either AAV1.hGALC, AAV3B.hGALC, or AAV5.hGALC at a dose of 2.0 ×10¹⁰. At necropsy, the brain was collected, processed and stained for hGALC detection. Scale bar is 200 µm. Abbreviations: GC, genome copies; PBS, phosphate-buffered saline; twi, Twitcher allele (consisting of a loss-of-function mutation in the Galc gene).

FIG. 29A and FIG. 29B show survival of twi/twi Mice Administered rAAVhu68.hGALC or Vehicle on PND12 or PND21 On PND 12 or PND 21, twi/twi mice were ICV-administered rAAVhu68.hGALC at one of two doses, 1.0 × 10¹¹ GC or 2.0 × 10¹¹ GC. Age-matched twi/twi mice and WT mice were ICV-administered PBS as controls. Survival was monitored. p<0.0001 for rAAVhu68.hGALC groups on PND12 compared to PBS and p=0.0008 for PND21 group compared to PBS based on a comparison of each group to the vehicle-treated twi/twi control group using a Log-rank (Mantel-Cox) test. Abbreviations: GC, genome copies; PBS, phosphate-buffered saline; twi, Twitcher allele (consisting of a loss-of-function mutation in the Galc gene).

FIG. 30 shows survival of twi/twi mice administered rAAVhu68.hGALC at a dose of 2.0 × 10¹¹ GC on PND12 or PND21. On PND 12 or PND 21, twi/twi mice were ICV-administered rAAVhu68.hGALC at one of two doses, 1.0 × 10¹¹ GC or 2.0 × 10¹¹ GC. Age-matched twi/twi mice and WT mice were ICV-administered PBS as controls. Survival was monitored. p<0.0001 based on a comparison of each group to the vehicle-treated twi/twi control group using a Log-rank (Mantel-Cox) test. Abbreviations: GC, genome copies; PBS, phosphate-buffered saline; twi, Twitcher allele (consisting of a loss-of-function mutation in the Galc gene).

FIG. 31 shows body weights of twi/twi mice administered rAAVhu68.hGALC or vehicle on PND12. On PND 12, twi/twi mice were ICV-administered rAAVhu68.hGALC at one of two doses, 1.0 × 10¹¹ GC or 2.0 × 10¹¹ GC. Age-matched twi/twi mice and WT mice were ICV-administered PBS as controls. Animals were weighed three times per week. Error bars represent the standard deviation. p=0.0001 based comparison of groups using a 1-way ANOVA and post hoc Dunn’s multiple comparisons test. Abbreviations: GC, genome copies; PBS, phosphate-buffered saline; twi, Twitcher allele (consisting of a loss-of-function mutation in the Galc gene).

FIG. 32 shows body weights of twi/twi mice administered rAAVhu68.hGALC or vehicle on PND21. On PND 12 or PND 21, twi/twi mice were ICV-administered rAAVhu68.hGALC at one of two doses, 1.0 × 10¹¹ GC or 2.0 × 10¹¹ GC. Age-matched twi/twi mice and WT mice were ICV-administered PBS as controls. Animals were weighed three times per week. Error bars represent the standard deviation. p=0.0001 based comparison of groups using a 1-way ANOVA and post hoc Dunn’s multiple comparisons test. Abbreviations: GC, genome copies; PBS, phosphate-buffered saline; twi, Twitcher allele (consisting of a loss-of-function mutation in the Galc gene).

FIG. 33 shows neuromotor function of twi/twi mice administered rAAVhu68.hGALC or vehicle on PND12. On PND 12, twi/twi mice were ICV-administered rAAVhu68.hGALC at one of two doses, 1.0 × 10¹¹ GC or 2.0 × 10¹¹ GC. Age-matched twi/twi mice and WT mice were ICV-administered PBS as controls. On PND 35, neuromotor function was assessed by the time to fall (seconds) for mice running on an accelerating rod initially spinning at 5 RPM and increasing to 40 RPM over 120 seconds. Error bars represent the standard deviation. **p=0.0004 for low dose and ***p=0.0006 for the high dose based on a comparison of each group to the PBS-treated twi/twi control group using a 1-way ANOVA and post hoc Dunn’s multiple comparisons test. Abbreviations: GC, genome copies; PBS, phosphate-buffered saline; twi, Twitcher allele (consisting of a loss-of-function mutation in the Galc gene).

FIG. 34 shows neuromotor function of twi/twi mice administered rAAVhu68.hGALC or vehicle on PND12 or PND21. On PND 12 or PND 21, twi/twi mice were ICV-administered rAAVhu68.hGALC at one of two doses, 1.0 × 10¹¹ GC or 2.0 × 10¹¹ GC. Age-matched twi/twi mice and WT mice were ICV-administered PBS as controls. On PND 35, neuromotor function was assessed by the time to fall (seconds) for mice running on an accelerating rod initially spinning at 5 RPM and increasing to 40 RPM over 120 seconds. Error bars represent the standard deviation. Abbreviations: GC, genome copies; PBS, phosphate-buffered saline; twi, Twitcher allele (consisting of a loss-of-function mutation in the Galc gene).

FIG. 35 shows transgene expression (GALC Activity) in the brain of twi/twi mice administered rAAVhu68.hGALC or vehicle on PND12 or PND21. On PND 12 or PND 21, twi/twi mice were ICV-administered rAAVhu68.hGALC at one of two doses, 1.0 × 10¹¹ GC or 2.0 × 10¹¹ GC. Age-matched twi/twi mice and WT mice were ICV-administered PBS as controls. At necropsy, the brain was collected for a GALC enzyme activity assay to evaluate transgene expression. Error bars represent the standard deviation. The dashed line indicates the mean value from PBS-treated wild type mice. **p=0.002 based on a comparison of each group to the PBS-treated twi/twi control group using a 1-way ANOVA and post hoc Dunn’s multiple comparisons test.

FIG. 36 shows transgene expression (GALC Activity) in the liver of twi/twi mice administered rAAVhu68.hGALC or vehicle on PND12 or PND21. On PND 12 or PND 21, twi/twi mice were ICV-administered rAAVhu68.hGALC at one of two doses, 1.0 × 10¹¹ GC or 2.0 × 10¹¹ GC. Age-matched twi/twi mice and WT mice were ICV-administered PBS as controls. At necropsy, the liver was collected for a GALC enzyme activity assay to evaluate transgene expression. Error bars represent the standard deviation. The dashed line indicates the mean value from PBS-treated wild type mice . ****p<0.0001 based on a comparison of each group to the PBS-treated twi/twi control group using a 1-way ANOVA and post hoc Dunn’s multiple comparisons test.

FIG. 37 shows transgene expression (GALC Activity) in the serum of twi/twi mice administered rAAVhu68.hGALC or vehicle on PND12 or PND21. On PND 12 or PND 21, twi/twi mice were ICV-administered rAAVhu68.hGALC at one of two doses, 1.0 × 10¹¹ GC or 2.0 × 10¹¹ GC. Age-matched twi/twi mice and WT mice were ICV-administered PBS as controls. On PND18, blood was collected was collected for a GALC enzyme activity assay to evaluate transgene expression. Error bars represent the standard deviation. The dashed line indicates the mean value from PBS-treated wild type mice. ** p<0.01; **** p<0.0001 based on a comparison of each group to the PBS-treated twi/twi control group using a 1-way ANOVA and post hoc Dunn’s multiple comparisons test.

FIG. 38 shows brain myelination in twi/twi mice administered rAAVhu68.hGALC or vehicle on PND12 or PND21. On PND 12 or PND 21, twi/twi mice were ICV-administered rAAVhu68.hGALC at one of two doses, 1.0 × 1011 GC or 2.0 × 1011 GC. Age-matched twi/twi mice and WT mice were ICV-administered PBS as controls. At necropsy, the brain was collected, processed and stained with LFB/PAS for myelination and globoid cell infiltration assessments. Pictures were taken at high magnification. Arrows point to globoid cells in the cerebellar folia and the star shows the corpus callosum. Scale bars 100 um.

FIG. 39 shows sciatic nerve myelination in twi/twi mice administered rAAVhu68.hGALC on PND12 or PND21. On PND 12 or PND 21, twi/twi mice were ICV-administered rAAVhu68.hGALC at one of two doses, 1.0 × 10¹¹ GC or 2.0 × 10¹¹ GC. Age-matched twi/twi mice and WT mice were ICV-administered PBS as controls. At necropsy, the sciatic nerve was collected, processed and stained with Toluidine blue for myelination and globoid cell infiltration assessments. More myelinated fibers are seen in the mouse that survived the longest (middle picture). Pictures were taken at a 40× magnification. Arrows indicate myelinated nerve fibers.

FIG. 40 shows neuroinflammation (IBA1) in the brain of twi/twi mice administered rAAVhu68.hGALC or vehicle on PND12 or PND21. On PND 12 or PND 21, twi/twi mice were ICV-administered rAAVhu68.hGALC at one of two doses, 1.0 × 10¹¹ GC or 2.0 × 10¹¹ GC. Age-matched twi/twi mice and WT mice were ICV-administered PBS as controls. At necropsy, the brain was collected, processed and stained for neuroinflammation (IBA1 staining). Dark staining indicated activated microglial cells and globoid cells (arrows). Central cerebellar white matter is indicated with the black stars and there is an absence of globoid cells. Globoid cells were present in the cerebellum folia and brainstem of rAAVhu68.hGALC -treated mice. Scale bar is 100 µm.

FIG. 41 shows hGALC expression in the brain of twi/twi mice administered rAAVhu68.hGALC or vehicle on PND12 or PND21. On PND 12 or PND 21, twi/twi mice were ICV-administered rAAVhu68.hGALC at one of two doses, 1.0 × 10¹¹ GC or 2.0 × 10¹¹ GC. Age-matched twi/twi mice and WT mice were ICV-administered PBS as controls. At necropsy, the brain was collected, processed and stained for hGALC detection. Scale bar is 200 µm.

FIG. 42 shows body weights of twi/twi mice administered rAAVhu68.hGALC or vehicle. On PND 40, twi/twi mice were ICV-administered rAAVhu68.hGALC at a dose of 2.0 × 10¹¹ GC. Age-matched twi/twi mice and WT mice were ICV-administered PBS as controls. Animals were weighed three times per week. Error bars represent the standard deviation. Due to the limited number of male and female mice per group, male and female body weight data were combined.

FIG. 43 shows neuromotor function of twi/twi mice administered rAAVhu68.hGALC or vehicle. On PND 40, twi/twi mice were ICV-administered rAAVhu68.hGALC at a dose of 2.0 × 10¹¹ GC. Age-matched twi/twi mice and WT mice were ICV-administered PBS as controls. On PND 35, neuromotor function was assessed by the time to fall (seconds) for mice running on an accelerating rod initially spinning at 5 RPM and increasing to 40 RPM over 120 seconds. Error bars represent the standard deviation. **p=0.0001 based on a comparison of each group to the PBS-treated twi/twi control group using a 1-way ANOVA and post hoc Dunn’s multiple comparisons test.

FIG. 44 shows clinical scoring assessments of twi/twi mice administered rAAVhu68.hGALC or vehicle. On PND 40, twi/twi mice were ICV-administered rAAVhu68.hGALC at a dose of 2.0 × 10¹¹ GC. Age-matched twi/twi mice and WT mice were ICV-administered PBS as controls. A standardized clinical assessment was performed three times per week. Error bars represent the standard deviation.

FIG. 45 shows transgene expression (GALC Activity) in the brain of twi/twi mice administered rAAVhu68.hGALC or vehicle. On PND 40, twi/twi mice were ICV-administered rAAVhu68.hGALC at a dose of 2.0 × 10¹¹ GC. Age-matched twi/twi mice and WT mice were ICV-administered PBS as controls. At necropsy, the brain was collected for a GALC enzyme activity assay to evaluate transgene expression. Error bars represent the standard deviation.

FIG. 46 shows transgene expression (GALC Activity) in the liver of twi/twi mice administered rAAVhu68.hGALC or vehicle. On PND 40, twi/twi mice were ICV-administered rAAVhu68.hGALC at a dose of 2.0 × 10¹¹ GC. Age-matched twi/twi mice and WT mice were ICV-administered PBS as controls. At necropsy, the liver was collected for a GALC enzyme activity assay to evaluate transgene expression. Error bars represent the standard deviation.

FIG. 47A and FIG. 47B show transgene expression (GALC Activity) in the serum of twi/twi mice Administered rAAVhu68.hGALC or vehicle. On PND 40, twi/twi mice were ICV-administered rAAVhu68.hGALC at a dose of 2.0 × 10¹¹ GC. Age-matched twi/twi mice and WT mice were ICV-administered PBS as controls. On PND28 (FIG. 47A) and at necropsy (PND40) (FIG. 47B), blood was collected for a GALC enzyme activity assay to evaluate transgene expression. Error bars represent the standard deviation. ** p<0.01; **** p<0.0001 based on a comparison of each group to the PBS-treated twi/twi control group using a 1-way ANOVA and post hoc Dunn’s multiple comparisons test.

FIG. 48A - FIG. 48C show brain myelination in twi/twi mice administered rAAVhu68.hGALC or vehicle. On PND 40, twi/twi mice were ICV-administered rAAVhu68.hGALC at a dose of 2.0 × 10¹¹ GC (results in FIG. 48C). Age-matched twi/twi mice (FIG. 48B) and WT mice (FIG. 48A) were ICV-administered PBS as controls. At necropsy, the brain was collected, processed, and stained with LFB/PAS for myelination and globoid cell infiltration assessments. Pictures were taken at low magnification. Staining intensity represents myelination extent. Paler myelin can be seen in the corpus callosum of twi/twi PBS mice. rAAVhu68.hGALC -treatment groups show normal WT-like corpus callosum myelin intensity. Scale bar 2 mm. Abbreviations are as defined above. LFB, Luxol Fast Blue; PAS, Periodic Acid-Schiff;

FIG. 49 is a series of nine photos which shows brain myelination in twi/twi mice administered rAAVhu68.hGALC (results in third columns) or vehicle (result shown in second column) higher magnification. On PND 40, twi/twi mice were ICV-administered rAAVhu68.hGALC at a dose of 2.0 × 10¹¹ GC. Age-matched twi/twi mice and WT mice (results in first column) were ICV-administered PBS as controls. At necropsy, the brain was collected, processed, and stained with LFB/PAS for myelination and globoid cell infiltration assessments. Pictures were taken at high magnification (20×). Arrows indicate globoid cells. Scale bars 100 um. Row 1 is from brain stem. Row 2 is from cerebellum. Row 3 is from corpus collosum.

FIG. 50 is a series of twelve photos which shows peripheral nerve myelination in twi/twi mice administered rAAVhu68.hGALC (results in column 3) or vehicle (results in column 2). On PND 40, twi/twi mice were ICV-administered rAAVhu68.hGALC at a dose of 2.0 × 10¹¹ GC. Age-matched twi/twi mice and WT mice (results in column 1) were ICV-administered PBS as controls. At necropsy, the nerves were collected, processed, and stained with LFB/PAS or Toluidine Blue for myelination and globoid cell infiltration assessments. Rows 1 and 2 provided results of sciatic nerve and axillary nerve, respectively with LBS staining. Row 3 is high magnification of sciatic nerve with toluidine blue staining. Row 4 is high magnification of sciatic nerve with IBA1 IHC staining. Scale bar is 100 nm.

FIG. 51 is a series of nine photos which show spinal cord myelination in twi/twi mice administered rAAVhu68.hGALC or vehicle. On PND 40, twi/twi mice were ICV-administered rAAVhu68.hGALC at a dose of 2.0 × 10¹¹ GC (results in column 3). Age-matched twi/twi mice and WT mice were ICV-administered PBS as controls (results in columns 2 and 1, respectively). At necropsy, the spinal cord was collected, processed, and stained with LFB/PAS for myelination and globoid cell infiltration assessments. Pictures were taken at low magnification. Globoid cells are indicated by the arrows. Scale bar 100 nm. Row 1 provides a sample from the cervical (C)-spine, Row 2 provides a sample from thoracis (T)-spine, Row 3 provides results from lumbar (L)-spine.

FIG. 52A - FIG. 52C show neuroinflammation (IBA1) in the brain of twi/twi mice administered rAAVhu68.hGALC (FIG. 52C) or vehicle (FIG. 52B) On PND 40, twi/twi mice were ICV-administered rAAVhu68.hGALC at a dose of 2.0 × 10¹¹ GC. Age-matched twi/twi mice and WT mice (FIG. 52A) were ICV-administered PBS as controls. At necropsy, the brain was collected, processed, and stained for neuroinflammation (IBA1 staining). Brown staining indicated activated microglial cells and globoid cells. Microglia cells are large and give a patchy coarse staining, especially in cortical cortex, corpus callosum, brain stem, cerebellum. In rAAVhu68.hGALC -treated twi/twi mice, the patchy staining of IBA1 is cleared in cortical cortex, corpus callosum, but it remains in cerebellum and brain stem. Pictures were taken at low magnification. Scale bars 2 mm.

FIG. 53 is a series of 15 photos which shows neuroinflammation (IBA1) in the brain of twi/twi mice administered rAAVhu68.hGALC (column 3) or vehicle (column 2) - high magnification. On PND 40, twi/twi mice were ICV-administered rAAVhu68.hGALC at a dose of 2.0 × 10¹¹ GC. Age-matched twi/twi mice and WT mice (column 1) were ICV-administered PBS as controls. At necropsy, the brain was collected, processed and stained for neuroinflammation (IBA1 staining). Dark staining indicated activated microglial cells and globoid cells. Pictures were taken at high magnification. Row 1 is from the cortical cortex. Row 2 is from the hippocampus. Row 3 is from the corpus collosum. Row 4 is from the cerebellum. Row 5 is from the brain stem. Scale bars 300 µm.

FIG. 54 is a series of nine pictures which show neuroinflammation (IBA1) in the spinal cord of twi/twi mice administered rAAVhu68.hGALC (column 3) or vehicle (column 2). On PND 40, twi/twi mice were ICV-administered rAAVhu68.hGALC at a dose of 2.0 × 10¹¹ GC. Age-matched twi/twi mice and WT mice (column 1) were ICV-administered PBS as controls. At necropsy, the brain was collected, processed and stained for neuroinflammation (IBA1 staining). Dark staining indicated activated microglial cells and globoid cells. Pictures were taken at high magnification). Row 1 is C-spine. Row 2 is T-spine. Row 3 is L-spine. Scale bars 200 um. Abbreviations: C-spine, cervical spinal cord; GC, genome copies; L-spine, lumbar spinal cord; PBS, phosphate-buffered saline; T-spine, lumbar spinal cord; twi, Twitcher allele (consisting of a loss-of-function mutation in the Galc gene).

FIG. 55 is a series of 12 pictures which show hGALC expression in the brain of twi/twi mice administered rAAVhu68.hGALC (column 3) or Vehicle (column 2). On PND 40, twi/twi mice were ICV-administered rAAVhu68.hGALC at a dose of 2.0 × 10¹¹ GC. Age-matched twi/twi mice and WT mice (column 1) were ICV-administered PBS as controls. At necropsy, the brain was collected, processed and stained for hGALC expression. Dark staining indicated activated microglial cells and globoid cells. Pictures were taken at high magnification. Scale bars 300 µm. Row 1 is from the cortex, Row 2 is from the hippocampus. Row 3 is from cerebellum. Row 4 is from brainstem.

FIG. 56A - FIG. 56C show results following combination therapy of rAAVhu68.hGALC and bone marrow transplant. Twitcher mice (twi/twi) were treated with a BMT only (N=13, PND 10), rAAVhu68.hGALC only (N=12, PND 0 or N=13, PND 12; ICV; 1.00 × 10¹¹ GC), rAAVhu68.hGALC followed by a BMT (N=7; PND 0 and PND 10, respectively), or a BMT followed by rAAVhu68.hGALC (N=7; PND 10 and PND 12, respectively). Twitcher mice (twi/twi) administered PBS only served as historical controls (N=8, Study 1, PND 0; N=4, Study 2, PND 12). Interim survival results are shown, and the experiment is still ongoing. Abbreviations: BMT, bone marrow transplant; GC, genome copies; ICV, intracerebroventricular; N, number of animals; PBS, phosphate-buffered saline; PND, postnatal day. FIG. 56C shows brain engraftment of GFP+ donor cells in cerebellum of wildtype and Twitcher (Krabbe) mice 8 weeks post HSCT.

FIG. 57 shows survival of twi/twi mice administered rAAVhu68.hGALC or vehicle. On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. Survival was monitored. Data for both the PND 40 necropsy cohort and the survival cohort are combined by treatment and genotype. *p<0.05, **p<0.01, ***p<0.001 based on a comparison of each group to the vehicle-treated twi/twi control group using a Log-rank (Mantel-Cox) test. Abbreviations: Galc, galactosylceramidase (gene, mouse); GC, genome copies; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 58A - FIG. 58B shows body weights of male or female twi/twi mice administered rAAVhu68.hGALC or vehicle (PND 40 and Survival Cohorts). FIG. 58A provides results of males and FIG. 58B provides results for females. On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. Animals were weighed three times per week. Data for both the PND 40 necropsy cohort and the survival cohort are combined by treatment and genotype, and average body weights are presented from weaning to the necropsy of the PND 40 cohort. Error bars represent the standard deviation. **p<0.01, ****p<0.0001 based on a statistical analysis of the longitudinal data using linear mixed effect modeling to compare each group to the vehicle-treated twi/twi control group. Abbreviations: Galc, galactosylceramidase (gene, mouse); GC, genome copies; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 59A and FIG. 59B shows body weights of male or female twi/twi mice administered rAAVhu68.hGALC or vehicle (PND 40 and Survival Cohorts). FIG. 59A provides results of males and FIG. 59B provides results for females. On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Twi/twi mice and WT mice were ICV-administered vehicle (ITFFB) as controls. Animals were weighed three times per week after weaning. Data for both the PND 40 necropsy cohort and the survival cohort are combined by treatment and genotype, and average body weights are presented from weaning until the necropsy of the last surviving twi/twi mice. Error bars represent the standard deviation. Abbreviations: Galc, galactosylceramidase (gene, mouse); GC, genome copies; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 60 shows clinical scoring assessments of twi/twi mice administered rAAVhu68.hGALC or vehicle (PND 40 and Survival Cohorts). On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. A standardized clinical assessment was performed on the indicated postnatal days. Data for both the PND 40 necropsy cohort and the survival cohort are combined by treatment and genotype, and average total clinical scores are presented from weaning to the necropsy of the PND 40 cohort. Error bars represent the standard deviation. **p<0.01, ****p<0.0001 based on a statistical analysis of the longitudinal data using linear mixed effect modeling to compare each group to the vehicle-treated twi/twi control group. Abbreviations: Galc, galactosylceramidase (gene, mouse); GC, genome copies; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 61 shows clinical scoring assessments of twi/twi mice administered rAAVhu68.hGALC or vehicle (PND 40 and Survival Cohorts). On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. A standardized clinical assessment was performed on the indicated postnatal days. Data for both the PND 40 necropsy cohort and the survival cohort are combined by treatment and genotype, and average total clinical severity scores are presented from weaning until the necropsy of the last surviving twi/twi mice. Error bars represent the standard deviation. **p<0.01, ****p<0.0001 based on a statistical analysis of the longitudinal data using linear mixed effect modeling to compare each group to the vehicle-treated twi/twi control group. Abbreviations: Galc, galactosylceramidase (gene, mouse); GC, genome copies; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 62 shows neuromotor function of twi/twi mice administered rAAVhu68.hGALC or vehicle (PND 40 and Survival Cohorts). On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. On PND 35-37, neuromotor function was assessed by the time to fall (seconds) for mice running on an accelerating rod initially spinning at 5 RPM and increasing to 40 RPM over 120 seconds. Data for both the PND 40 necropsy cohort and the survival cohort are combined by treatment and genotype. Error bars represent the standard deviation. **p<0.01, ****p<0.0001 based on a comparison of each group to the vehicle-treated twi/twi control group using a 1-way ANOVA and post hoc Dunn’s multiple comparisons test. Abbreviations: Galc, galactosylceramidase (gene, mouse); GC, genome copies; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 63 shows transgene expression in serum of twi/twi mice administered rAAVhu68.hGALC or vehicle (PND 40 and Survival Cohorts). On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. Serum was collected on PND 35-37 for a GALC enzyme activity assay to evaluate transgene expression. Data for both the PND 40 necropsy cohort and the survival cohort are combined by treatment and genotype. The y-axis is split to illustrate the difference between the data points in the 0-1000 RFU range. Error bars represent the standard deviation. **p<0.01, ****p<0.0001 based on a comparison of each group to the vehicle-treated twi/twi control group using a 1-way ANOVA and post hoc Dunn’s multiple comparisons test. Abbreviations: Galc, galactosylceramidase (gene, mouse); GALC, galactosylceramidase (protein, mouse); GC, genome copies; ICV, intracerebroventricular; ITFFB, intrathecal final formulation buffer; N, number of animals; PND, postnatal day; RFU, relative fluorescence units; twi, Twitcher allele (consisting of a loss-of-function mutation in the Galc gene); WT, wild type.

FIG. 64 shows transgene expression in the brain of twi/twi mice administered rAAVhu68.hGALC or Vehicle (PND 40 and Survival Cohorts). On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. At necropsy, the brain was collected for a GALC enzyme activity assay to evaluate transgene expression. Data for both the PND 40 necropsy cohort and the survival cohort are combined by treatment and genotype. Error bars represent the standard deviation. **p<0.01, ****p<0.0001 based on a comparison of each group to the vehicle-treated twi/twi control group using a 1-way ANOVA and post hoc Dunn’s multiple comparisons test. Abbreviations: Galc, galactosylceramidase (gene, mouse); GALC, galactosylceramidase (protein, mouse); GC, genome copies; ICV, intracerebroventricular; ITFFB, intrathecal final formulation buffer; N, number of animals; PND, postnatal day; RFU, relative fluorescence units; twi, Twitcher allele (consisting of a loss-of-function mutation in the Galc gene); WT, wild type.

FIG. 65A - FIG. 65D show transgene expression in the heart, kidney, liver, and spleen of twi/twi mice administered rAAVhu68.hGALC or vehicle (PND 40 and Suvival Cohorts). On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. At necropsy, heart (FIG. 65A), kidney (FIG. 65B), liver (FIG. 65C), and spleen (FIG. 65D) were collected for a GALC enzyme activity assay to evaluate transgene expression. Data for both the PND 40 necropsy cohort and the survival cohort are combined by treatment and genotype. Error bars represent the standard deviation. *p<0.05, ****p<0.0001 based on a comparison of each group to the vehicle-treated twi/twi control group using a 1-way ANOVA and post hoc Dunn’s multiple comparisons test. Abbreviations: Galc, galactosylceramidase (gene, mouse); GALC, galactosylceramidase (protein, mouse); GC, genome copies; ICV, intracerebroventricular; ITFFB, intrathecal final formulation buffer; N, number of animals; PND, postnatal day; RFU, relative fluorescence units; twi, Twitcher allele (consisting of a loss-of-function mutation in the Galc gene); WT, wild type.

FIG. 66A - FIG. 66C shows transgene expression in the lung, quadriceps muscle, and diaphragm of twi/twi mice administered rAAVhu68.hGALC or vehicle (PND 40 and Suvival Cohorts). On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. At necropsy, lung (FIG. 66A), quadriceps muscle (FIG. 66B), and diaphragm (FIG. 66C) were collected for a GALC enzyme activity assay to evaluate transgene expression. Data for both the PND 40 necropsy cohort and the survival cohort are combined by treatment and genotype. Error bars represent the standard deviation. **p<0.01, ****p<0.0001 based on a comparison of each group to the vehicle-treated twi/twi control group using a 1-way ANOVA and post hoc Dunn’s multiple comparisons test. Abbreviations: Galc, galactosylceramidase (gene, mouse); GALC, galactosylceramidase (protein, mouse); GC, genome copies; ICV, intracerebroventricular; ITFFB, intrathecal final formulation buffer; N, number of animals; PND, postnatal day; RFU, relative fluorescence units; twi, Twitcher allele (consisting of a loss-of-function mutation in the Galc gene); WT, wild type.

FIG. 67A and FIG. 67B show lymphocyte counts in twi/twi mice administered rAAVhu68.hGALC or vehicle. On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. At necropsy on PND 40-42 (FIG. 67A) and at humane euthanasia for the survival cohort (FIG. 67B), blood was collected to quantify lymphocytes. Blood collected from untreated twi/twi and WT mice on the day of dosing served as baseline controls. Error bars represent the standard deviation. *p<0.05, **p<0.01, ***p<0.001 based on a comparison of each group to the vehicle-treated WT control group using a 1-way ANOVA and post hoc Dunn’s multiple comparisons test. Abbreviations: Galc, galactosylceramidase (gene, mouse); GC, genome copies; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 68A - FIG. 68D show aspartate aminotransferase levels (FIG. 68A and FIG. 68B) and bilirubin levels (FIG. 68C and FIG. 68D) in twi/twi mice administered rAAVhu68.hGALC or vehicle. On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. At necropsy on PND 40-42 and at humane euthanasia for the survival cohort, serum was collected to evaluate AST and bilirubin levels as part of a serum chemistry panel. FIG. 68A and FIG. 68B show AST levels for PND cohort and survival cohort, respectively. FIG. 68C and FIG. 68D show total bilirubin levels for PND cohort and survival cohort, respectively Serum collected from untreated twi/twi and WT mice on the day of dosing served as baseline controls. Error bars represent the standard deviation. **p<0.01, ***p<0.001 based on a comparison of each group to the vehicle-treated WT control group using a 1-way ANOVA and post hoc Dunn’s multiple comparisons test. Untreated WT mice (baseline cohort, Group 2) and twi/twi mice administered rAAVhu68.hGALC at a dose of 6.8 × 10⁹ GC (PND 40 cohort, Group 8a) were excluded from statistical analysis due to insufficient sample numbers. Abbreviations: AST, aspartate aminotransferase; Galc, galactosylceramidase (gene, mouse); GC, genome copies; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 69A and FIG. 69B show alanine aminotransferase levels in twi/twi mice administered rAAVhu68.hGALC or vehicle. On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. At necropsy on PND 40-42 (FIG. 69A) and at humane euthanasia for the survival cohort (FIG. 69B), serum was collected to evaluate ALT levels as part of a serum chemistry panel. Serum collected from untreated twi/twi and WT mice on the day of dosing served as baseline controls. Error bars represent the standard deviation. *p<0.05, **p<0.01, ***p<0.001 based on a comparison of each group to the vehicle-treated WT control group using a 1-way ANOVA and post hoc Dunn’s multiple comparisons test. Untreated WT mice (baseline cohort, Group 2) and twi/twi mice administered rAAVhu68.hGALC at a dose of 6.8 × 10⁹ GC (PND 40 cohort, Group 8a) were excluded from statistical analysis due to insufficient sample numbers. Abbreviations: ALT, alanine aminotransferase; Galc, galactosylceramidase (gene, mouse); GC, genome copies; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 70A - FIG. 70D show glucose (FIG. 70A and FIG. 70B) and amylase levels (FIG. 70C and FIG. 70D) in twi/twi mice administered rAAVhu68.hGALC or vehicle. On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. At necropsy on PND 40-42 and at humane euthanasia for the survival cohort, serum was collected to evaluate glucose and amylase levels as part of a serum chemistry panel. Serum collected from untreated twi/twi and WT mice on the day of dosing served as baseline controls. Error bars represent the standard deviation. *p<0.05, **p<0.01 based on a comparison of each group to the vehicle-treated WT control group using a 1-way ANOVA and post hoc Dunn’s multiple comparisons test. Untreated WT mice (baseline cohort, Group 2) and twi/twi mice administered rAAVhu68.hGALC at a dose of 6.8 × 10⁹ GC (PND 40 cohort, Group 8a) were excluded from statistical analysis due to insufficient sample numbers. Abbreviations: Galc, galactosylceramidase (gene, mouse); GC, genome copies; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 71 shows semi-quantitative scoring of liver microvacuolation in twi/twi mice administered rAAVhu68.hGALC or vehicle. On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. The survival cohort was necropsied at the point of humane euthanasia, and livers were collected for histopathology. Hepatocellular vacuolation was semi-quantitatively scored as follows: Grade 0, no vacuoles; Grade 1, minimal vacuoles; Grade 2, mild vacuoles; Grade 3, moderate vacuoles. Abbreviations: Galc, galactosylceramidase (gene, mouse); GC, genome copies; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 72A - FIG. 72F show quantification of IBA 1-positive cell size in the cortex and cerebellum of twi/twi mice administered rAAVhu68.hGALC or vehicle. On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. At necropsy on PND 40-42 and at humane euthanasia for the survival cohort, the brain was collected. Brains collected from untreated twi/twi and WT mice on the day of dosing served as baseline controls. IBA1 immunohistochemistry was performed on tissue sections from the cortex - FIG. 72A (baseline), FIG. 72B (PND 40 cohort), FIG. 72C (survival) and cerebellum - (FIG. 72D (baseline), FIG. 72E (PND 40 cohort), FIG. 72F (survival). The size of individual IBA1-positive globoid cells (mean object area) was quantified using image analysis software. Error bars represent the standard deviation. *p<0.05, **p<0.01, ***p<0.001, ****p<0.0001 based on a comparison of each group to the vehicle-treated twi/twi control group using a 1-way ANOVA and post hoc Dunn’s multiple comparisons test. Abbreviations: Galc, galactosylceramidase (gene, mouse); GC, genome copies; IBAl, ionized calcium-binding adaptor molecule 1; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 73A - FIG. 73F show quantification of IBA1-postive cell size in the brainstem and spinal cord of twi/twi mice administered rAAVhu68.hGALC or vehicle. FIG. 73A, FIG. 73B, and FIG. 73C provide baseline, PND40 cohort and survival cohort, respectively for brainstem. FIG. 73D, FIG. 73E, and FIG. 73F provide baseline, PND40 cohort and survival cohort, respectively for spinal cord. On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. At necropsy on PND 40-42 and at humane euthanasia for the survival cohort, the brain and spinal cord were collected. Brains and spinal cords collected from untreated twi/twi and WT mice on the day of dosing served as baseline controls. IBA1 immunohistochemistry was performed on tissue sections from the brain stem and the spinal cord (cervical, lumbar, thoracic).. The size of individual IBA1-positive globoid cells (mean object area) was quantified using image analysis software. Error bars represent the standard deviation. **p<0.01, ***p<0.001, ****p<0.0001 based on a comparison of each group to the vehicle-treated twi/twi control group using a 1-way ANOVA and post hoc Dunn’s multiple comparisons test. Abbreviations: Galc, galactosylceramidase (gene, mouse); GC, genome copies; IBA1, ionized calcium-binding adaptor molecule 1; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 74A - FIG. 74C show quantification of IBA 1-postive cell size in the sciatic nerve of twi/twi mice administered rAAVhu68.hGALC or vehicle. FIG. 74A provides baseline results. FIG. 74B provides results from the PND40 cohort. FIG. 74C provides results from the survival cohort. On PND 12-14, twi/twi mice were ICV-administered rAAVhu68.hGALC at a dose of 6.8 × 10⁹ GC, 2.0 × 10¹⁰ GC, 6.8 × 10¹⁰ GC, or 2.0 × 10¹¹ GC. Age-matched twi/twi mice and WT mice were ICV-administered vehicle (ITFFB) as controls. At necropsy on PND 40-42 and at humane euthanasia for the survival cohort, sciatic nerves were collected. Sciatic nerves collected from untreated twi/twi and WT mice on the day of dosing served as baseline controls. IBA1 immunohistochemistry was performed on tissue sections from the sciatic nerves. The size of individual IBA1-positive globoid cells (mean object area) was quantified using image analysis software. Error bars represent the standard deviation. **p<0.01, ****p<0.0001 based on a comparison of each group to the vehicle-treated twi/twi control group using a 1-way ANOVA and post hoc Dunn’s multiple comparisons test. Abbreviations: Galc, galactosylceramidase (gene, mouse); GC, genome copies; IBA1, ionized calcium-binding adaptor molecule 1; ICV, intracerebroventricular; ITFFB, intrathecal final formulation buffer; N, number of animals; PND, postnatal day; twi, Twitcher allele (consisting of a loss-of-function mutation in the Galc gene); WT, wild type.

FIG. 75 shows a comparison of serum GALC activity in twitcher mice administered a rAAVhu68 having either an engineered GALC (cGALCco) or the native canine GALC (cGALnat) sequence. Improved survival was observed in twitcher mice administered the rAAVhu.cGALCco, compared to a rAAVhu68 having the native sequence.

FIG. 76 shows the progression of the neuropathological and behavioral phenotypes for the Krabbe dog is presented (Wenger D.A., et al. (1999) J Hered. 90(1): 138-42; Bradbury A., et al. (2016) Neuroradiol J. 29(6):417-424; Bradbury A.M., et al. (2016b) 94(11): 1007-17; Bradbury A.M., et al. (2018) Hum Gene Ther. 29(7):785-801). Dashed lines indicate that data for earlier time points for the specified phenotype have not been described. *Asterisk refers to demyelination that is observed by histology. Abbreviations: BAER, brainstem auditory evoked response; CNS, central nervous system; MRI, magnetic resonance imaging; NCV, nerve conduction velocity; PNS, peripheral nervous system.

FIG. 77 shows a study design for evaluation of AAV.CB7.cGALCco.rBG gene therapy in Krabbe dogs.

FIG. 78 shows body weights of Krabbe dogs following ICM administration of rAAVhu68.cGALCco. At 2-3 weeks old, Krabbe dogs received a single ICM administration of either rAAVhu68.cGALCco (AAV) at a dose of 3.0 × 10¹³ GC (N=4) or vehicle (ITFFB; N=2). A healthy wild type littermate dog was administered vehicle as a control (N=1). Animals were weighed weekly. Abbreviations: AAVhu68.cGALCco, AAVhu68.CB7.CI.cGALCco.rBG; GC, genome copies; ICM, intra-cisterna magna; ITFFB, intrathecal final formulation buffer; N, number of animals; NCV, nerve conduction velocity.

FIG. 79A - FIG. 79D show nerve conduction velocities in Krabbe dogs following ICM administration of rAAVhu68.cGALCco. FIG. 79A and FIG. 79B provide radial sensory NSV and sciatic motor NCV. FIG. 79C and FIG. 79D provide ulnar motor NCV and tibial motor NCV. At 2-3 weeks old, Krabbe dogs received a single ICM administration of either rAAVhu68.cGALCco (AAV) at a dose of 3.0 × 10¹³ GC (N=4) or vehicle (ITFFB; N=2). A healthy wild type littermate dog was administered vehicle as a control (N=1). Nerve conduction studies were performed at the indicated ages. NCVs for the radial nerve (sensory nerve) and the sciatic, ulnar, and tibial nerves (motor nerves) are presented. Abbreviations: AAVhu68.cGALCco, AAVhu68.CB7.CI.cGALCco.rBG; GC, genome copies; ICM, intra-cisterna magna; ITFFB, intrathecal final formulation buffer; N, number of animals; NCV, nerve conduction velocity.

FIG. 80 shows BAER interpeak latency in Krabbe dogs following ICM administration of rAAVhu68.cGALCco. At 2-3 weeks old, Krabbe dogs received a single ICM administration of either rAAVhu68.cGALCco (AAV) at a dose of 3.0 × 10¹³ GC (N=4) or vehicle (ITFFB; N=2). A healthy wild type littermate dog was administered vehicle as a control (N=1). BAER was performed at the indicated ages. Central conduction time was defined as the time between the first and the fifth peak. A value of 0 indicates no response measured. Abbreviations: AAVhu68.cGALCco, AAVhu68.CB7.CI.cGALCco.rBG; BAER, brainstem auditory evoked response; GC, genome copies; ICM, intra-cisterna magna; ITFFB, intrathecal final formulation buffer; N, number of animals

FIG. 81 shows BAER hearing threshold in Krabbe dogs following ICM administration of rAAVhu68.cGALCco. At 2-3 weeks old, Krabbe dogs received a single ICM administration of either rAAVhu68.cGALCco (AAV) at a dose of 3.0 × 10¹³ GC (N=4) or vehicle (ITFFB; N=2). A healthy wild type littermate dog was administered vehicle as a control (N=1). BAER hearing was assessed performed at the indicated ages. Hearing threshold was defined as the sound intensity at which an evoked waveform was first visible. A value of 0 indicates no response measured. Abbreviations: AAVhu68.cGALCco, AAVhu68.CB7.CI.cGALCco.rBG; BAER, brainstem auditory evoked response; GC, genome copies; ICM, intra-cisterna magna; ITFFB, intrathecal final formulation buffer; N, number of animals.

FIG. 82A - FIG. 82D show results from brain MRI studies in Krabbe dogs following ICM administration of rAAVhu68.cGALCco. At 2-3 weeks old, Krabbe dogs received a single ICM administration of either rAAVhu68.cGALCco (AAV) at a dose of 3.0 × 10¹³ GC (N=4) or vehicle (ITFFB; N=2). A healthy wild type littermate dog was administered vehicle as a control (N=1). Brain MRIs were performed when all animals were 8-10 weeks old and at 61 weeks of age in the long-term cohort (K933 and K928). Semi-quantitative brain white matter intensity scores were assigned to internal capsule, corona radiata, corpus callosum, and occipital and cerebellar white matter as follows: 0 = normal myelination (hypointense signal), 1 = suboptimal myelination (isointense signal), 2 = demyelination (hyperintense signal). FIG. 82A provides the brain MRI score - white matter hyperintensity for the individual treated animals and vehicle controls. FIG. 82B provides an MRI image from a Krabbe animal treated with vehicle showing hyperintense white matter. FIG. 82C provides an MRI image from a rAAVhu68.cGalCco treated Krabbe showing isointense white matter. Cumulative white matter intensity scores across all brain regions are presented for each animal. FIG. 82D provides measurements of mass intermedia diameter. Abbreviations: AAVhu68.cGALCco, AAVhu68.CB7.CI.cGALCco.rBG; GC, genome copies; ICM, intra-cisterna magna; ITFFB, intrathecal final formulation buffer; MRI, magnetic resonance imaging; N, number of animals.

FIG. 83 shows psychosine levels in the CSF in Krabbe dogs following ICM administration of rAAVhu68.cGALCco. At 2-3 weeks old, Krabbe dogs received a single ICM administration of either rAAVhu68.cGALCco (AAV) at a dose of 3.0 × 10¹³ GC (N=4) or vehicle (ITFFB; N=2). A healthy wild type littermate dog was administered vehicle as a control (N=1). CSF samples were collected throughout the study for glycosphingolipid quantification. Abbreviations: AAVhu68.cGALCco, AAVhu68.CB7.CI.cGALCco.rBG; GC, genome copies; ICM, intra-cisterna magna; ITFFB, intrathecal final formulation buffer; MRI, magnetic resonance imaging; N, number of animals.

FIG. 84 shows twelve staining images from myelination and globoid cells in the brain and peripheral nerves of Krabbe dogs following ICM administration of rAAVhu68.cGALCco. Column 1 is from a Krabbe dog treated with vehicle at 2 months. Column 2 is from a treated Krabbe dog at nine months. Column 3 is from a treated Krabbe dog at 6 months. Row 1 is from the corpus callosum. Row 2 is cerebellum. Row 3 is spinal cord. Row 4 is peripheral nerve. At 2-3 weeks old, Krabbe dogs received a single ICM administration of either AAVhu68.cGALCco (AAV) at a dose of 3.0 × 10¹³ GC (Animals K937, K938) or vehicle (ITFFB; Animal K930). At necropsy, CNS and PNS tissues were collected for LFB/PAS staining to visualize myelin (blue staining) and globoid cells. Representative images of the brain (corpus callosum), cerebellum folia, spinal cord, and sciatic nerves from one vehicle-treated animal (Animal K930, necropsied Day 35) and two AAV-treated animals (Animal K938, necropsied Day 181; Animal K937, necropsied Day 261) are presented. Abbreviations: AAVhu68.cGALCco, AAVhu68.CB7.CI.cGALCco.rBG; CNS, central nervous system; GC, genome copies; ICM, intra-cisterna magna; ITFFB, intrathecal final formulation buffer; N, number of animals; PAS, periodic acid-Schiff; PNS, peripheral nervous system.

FIG. 85A - FIG. 85F show semi-quantitative scoring of demyelination and globoid cell infiltration in the nervous system of Krabbe dogs following ICM administration of rAAVhu68.cGALCco. FIG. 85A, FIG. 85B. and FIG. 85C show demyelination in brain, spinal cord and peripheral nerves, respectively. FIG. 85D, FIG. 85E, and FIG. 85F show globoid cell infiltration in brain, spinal cord and peripheral nerves respectively. At 2-3 weeks old, Krabbe dogs received a single ICM administration of either rAAVhu68.cGALCco (AAV) at a dose of 3.0 × 10¹³ GC (Animals K937, K938, K939) or vehicle (ITFFB; Animals K930, K948). Necropsies for vehicle-treated animals were performed on Day 35 (Animal K930) and Day 66 (Animal K948). Necropsies for AAV-treated animals were performed on Day 180±3 (Animals K938, K939) and Day 261 (Animal K937). At necropsy, the brain, spinal cord (cervical, lumbar, thoracic), and peripheral nerves (optic, sciatic, medial, peroneal, radial, tibial, ulnar) were collected for LFB/PAS staining. Tissues were scored on a 4-point graded severity scale ranging from a score of 1 (normal myelination and absence of globoid cells) to a score of 4 (minimal to no myelination and diffuse globoid cell infiltration). Abbreviations: AAVhu68.cGALCco, AAVhu68.CB7.CI.cGALCco.rBG; GC, genome copies; ICM, intra-cisterna magna; ITFFB, intrathecal final formulation buffer; N, number of animals; PAS, periodic acid-Schiff.

FIG. 86A and FIG. 86B show neuro-inflammation and globoid cell storage in the nervous system of Krabbe dogs following ICM administration of rAAVhu68.cGALCco. At 2-3 weeks old, Krabbe dogs received a single ICM administration of either AAVhu68.cGALCco (AAV) at a dose of 3.0 × 10¹³ GC (Animals K937, K938, K939) or vehicle (ITFFB; Animals K930, K948). Necropsies for vehicle-treated animals were performed on Day 35 (Animal K930) and Day 66 (Animal K948). Necropsies for AAV-treated animals were performed on Day 180±3 (Animals K938, K939) and Day 261 (Animal K937). At necropsy, the brain was collected IBA1 immunohistochemistry. The size of globoid cells was measured using image analysis software. Abbreviations: AAVhu68.cGALCco, AAVhu68.CB7.CI.cGALCco.rBG; GC, genome copies; ICM, intra-cisterna magna; ITFFB, intrathecal final formulation buffer; N, number of animals; PAS, periodic acid-Schiff.

FIG. 87A and FIG. 87B show neuro-inflammation and globoid cell storage in the spinal cord of Krabbe dogs following ICM administration of rAAVhu68.cGALCco. At 2-3 weeks old, Krabbe dogs received a single ICM administration of either rAAVhu68.cGALCco (AAV) at a dose of 3.0 × 10¹³ GC (Animals K937, K938, K939) or vehicle (ITFFB; Animals K930, K948). Necropsies for vehicle-treated animals were performed on Day 35 (Animal K930) and Day 66 (Animal K948). Necropsies for AAV-treated animals were performed on Day 180±3 (Animals K938, K939) and Day 261 (Animal K937). At necropsy, the spinal cord (cervical, lumbar, thoracic) was collected for LFB/PAS staining. The size of globoid cells was measured using image analysis software. Abbreviations: AAVhu68.cGALCco, AAVhu68.CB7.CI.cGALCco.rBG; GC, genome copies; ICM, intra-cisterna magna; ITFFB, intrathecal final formulation buffer; N, number of animals; PAS, periodic acid-Schiff.

FIG. 88A - FIG. 88D show GALC activity in CSF and serum of Krabbe dogs following ICM administration of rAAVhu68.cGALCco. FIG. 88A and FIG. 88B show GALC activity in the CSF in AAV-Treated or Vehicle-treated animals, respectively. FIG. 88C and FIG. 88D show GALC activity in the serum in AAV-Treated or Vehicle-treated animals, respectively. At 2-3 weeks old, Krabbe dogs received a single ICM administration of either rAAVhu68.cGALCco (AAV) at a dose of 3.0 × 10¹³ GC (N=4) or vehicle (ITFFB; N=2). A healthy wild type littermate dog was administered vehicle as a control (N=1). At the indicated timepoints post treatment, CSF and serum were collected for all animals and a fluorescence-based GALC activity assay was performed to evaluate transgene product expression. The dotted line in each graph represents the average wild type GALC activity level in CSF (top graphs - FIG. 88A and FIG. 88B) or serum (bottom graphs - FIG. 88C and FIG. 88D) for Animal K928 during the 18-month follow-up. Abbreviations: AAVhu68.cGALCco, AAVhu68.CB7.CI.cGALCco.rBG; CSF, cerebrospinal fluid; FU, fluorescence units; GALC, galactosylceramidase (protein); GC, genome copies; ICM, intra-cisterna magna; ITFFB, intrathecal final formulation buffer; N, number of animals.

FIG. 89A - FIG. 89G show GALC activity in central nervous system of Krabbe dogs following ICM administration of rAAVhu68.cGALCco. FIG. 89A -FIG. 89D provide results of GALC activity in brain: cerebellum (FIG. 89A), frontal cortex (FIG. 89B), medulla (FIG. 89C) or occipital cortex (FIG. 89D). FIG. 89E -FIG. 89G provide GALC activity in spinal cord: cervical (FIG. 89E), thoracic (FIG. 89F) or lumbar (FIG. 89G). At 2-3 weeks old, Krabbe dogs received a single ICM administration of either rAAVhu68.cGALCco (AAV) at a dose of 3.0 × 10¹³ GC (Animals K937, K938, K939) or vehicle (ITFFB; Animals K930, K948). Necropsies for vehicle-treated animals were performed on Day 35 (Animal K930) and Day 66 (Animal K948). Necropsies for AAV-treated animals were performed on Day 180±3 (Animals K938, K939) and Day 261 (Animal K937). At necropsy, the indicated brain and spinal cord tissues were collected for a fluorescence-based GALC activity assay to evaluate transgene product expression. Abbreviations: AAVhu68.cGALCco, AAVhu68.CB7.CI.cGALCco.rBG; FU, fluorescence units; GALC, galactosylceramidase (protein); GC, genome copies; ICM, intra-cisterna magna; ITFFB, intrathecal final formulation buffer; N, number of animals.

FIG. 90A - FIG. 90D show GALC activity in the peripheral nervous system of Krabbe dogs following ICM administration of rAAVhu68.cGALCco as measured by fluorescent units (FU)/50 µg. FIG. 90A shows GALC activity in the dorsal root ganglion (DRG) - cervical spine. FIG. 90B shows GALC activity in the DRG -lumbar spine. FIG. 90C provides GALC activity in sciatic nerve. FIG. 90D provides GALC activity in median nerve. At 2-3 weeks old, Krabbe dogs received a single ICM administration of either rAAVhu68.cGALCco (AAV) at a dose of 3.0 × 10¹³ GC (Animals K937, K938, K939) or vehicle (ITFFB; Animals K930, K948). Necropsies for vehicle-treated animals were performed on Day 35 (Animal K930) and Day 66 (Animal K948). Necropsies for AAV-treated animals were performed on Day 180±3 (Animals K938, K939) and Day 261 (Animal K937). At necropsy, the indicated peripheral nervous system tissues were collected for a fluorescence-based GALC activity assay to evaluate transgene product expression. Abbreviations: AAVhu68.cGALCco, AAVhu68.CB7.CI.cGALCco.rBG; DRG, dorsal root ganglia; FU, fluorescence units; GALC, galactosylceramidase (protein); GC, genome copies; ICM, intra-cisterna magna; ITFFB, intrathecal final formulation buffer; N, number of animals.

FIG. 91A - FIG. 91E show GALC activity in peripheral organs of Krabbe dogs following ICM administration of rAAVhu68.cGALCco as measured by fluorescent units (FU)/50 µg. FIG. 91A provides results in heart. FIG. 91B provides results in kidney. FIG. 91C provides results in liver. FIG. 91D provides results in diaphragm. FIG. 91E provides results in skeletal muscle (quadriceps femoris). At 2-3 weeks old, Krabbe dogs received a single ICM administration of either rAAVhu68.cGALCco (AAV) at a dose of 3.0 × 10¹³ GC (Animals K937, K938, K939) or vehicle (ITFFB; Animals K930, K948). Necropsies for vehicle-treated animals were performed on Day 35 (Animal K930) and Day 66 (Animal K948). Necropsies for AAV-treated animals were performed on Day 180±3 (Animals K938, K939) and Day 261 (Animal K937). At necropsy, the indicated peripheral nervous system tissues were collected for a fluorescence-based GALC activity assay to evaluate transgene product expression. Abbreviations: AAVhu68.cGALCco, AAVhu68.CB7.CI.cGALCco.rBG; DRG, dorsal root ganglia; FU, fluorescence units; GALC, galactosylceramidase (protein); GC, genome copies; ICM, intra-cisterna magna; ITFFB, intrathecal final formulation buffer; N, number of animals.

FIG. 92 shows tissue biodistribution of Krabbe dogs following ICM administration of rAAVhu68.cGALCco. At 2-3 weeks old, Krabbe dogs received a single ICM administration of either rAAVhu68.cGALCco (AAV) at a dose of 3.0 × 10¹³ GC (Animals K937, K938, K939) or vehicle (ITFFB; Animals K930, K948). Necropsies for vehicle-treated animals were performed on Day 35 (Animal K930) and Day 66 (Animal K948). Necropsies for AAV-treated animals were performed on Day 180±3 (Animals K938, K939) and Day 261 (Animal K937). At necropsy, tissues were collected for biodistribution. Abbreviations: AAVhu68.cGALCco, AAVhu68.CB7.CI.cGALCco.rBG; DRG, dorsal root ganglia; FU, fluorescence units; GALC, galactosylceramidase (protein); GC, genome copies; ICM, intra-cisterna magna; ITFFB, intrathecal final formulation buffer; N, number of animals.

FIG. 93 shows a typical median nerve SNAP recorded from digit II of a healthy NHP. Sensory nerve conduction velocity was calculated by dividing the physical distance between the stimulation cathode and the recording site at digit II by the onset latency (i.e., the time between the stimulus and the onset of the SNAP). The SNAP amplitude was calculated as the difference in electrical voltage at the SNAP onset versus the SNAP peak. Abbreviations: NHP, nonhuman primate; SNAP, sensory nerve action potential.

FIG. 94A and FIG. 94B show SNAP amplitudes and nerve conduction velocities following ICM administration of rAAVhu68.hGALC to NHPs (Day 90 Cohort) with results sow as graphs of µV over the number of study day. FIG. 94A provides SNAP amplitude results with graphs from the right median nerve (on the left) and the left median nerve (on the right). FIG. 94B provides the nerve conducting velocity with the graph of the right median nerve shown on the left and the graph from the left median nerve shown on the right. Juvenile NHPs received a single ICM administration of either vehicle (ITFFB; N=1) or rAAVhu68.hGALC at a dose of 4.5 × 10¹² GC (low dose), 1.5 × 10¹³ GC (mid-dose), or 4.5 × 10¹³ GC (high dose) (N=3/group). Sensory nerve conduction testing was performed at BL and on Days 28±3, 60±3, and 90±4. SNAP amplitudes and conduction velocities of the right and left median nerves are presented. For SNAP amplitudes, the shaded area (17.1-92.3 µV) indicates values within two standard deviations of the baseline average of all animals in the study. Abbreviations: BL, baseline; GC, genome copies; ICM, intra-cisterna magna; ITFFB, intrathecal final formulation buffer; N, number of animals; NHP, non-human primate; SNAP, sensory nerve action potential.

FIG. 95A and FIG. 95B show SNAP amplitudes and nerve conduction velocities following ICM administration of rAAVhu68.hGALC to NHPs (Day 180 Cohort). with results sow as graphs of µV over the number of study day. FIG. 95A provides SNAP amplitude results with graphs from the right median nerve (on the left) and the left median nerve (on the right). FIG. 95B provides the nerve conducting velocity with the graph of the right median nerve shown on the left and the graph from the left median nerve shown on the right. Juvenile NHPs received a single ICM administration of either vehicle (ITFFB; N=1) or rAAVhu68.hGALC at a dose of 4.5 × 10¹² GC (low dose), 1.5 × 10¹³ GC (mid-dose), or 4.5 × 10¹³ GC (high dose) (N=3/group). Sensory nerve conduction testing was performed at BL and on Days 28±3, 60±3, 90±4, 120±4, 150±4, and 180±5. SNAP amplitudes and conduction velocities of the right and left median nerves are presented. For SNAP amplitudes, the shaded area (17.1-92.3 µV) indicates values within two standard deviations of the baseline average of all animals in the study. Abbreviations: BL, baseline; GC, genome copies; ICM, intra-cisterna magna; ITFFB, intrathecal final formulation buffer; N, number of animals; NHP, non-human primate; SNAP, sensory nerve action potential.

FIG. 96A and FIG. 96B show leukocyte counts in cerebrospinal fluid of NHPs following ICM administration of rAAVhu68.hGALC or vehicle. Juvenile NHPs received a single ICM administration of either vehicle (ITFFB; N=l/group) or rAAVhu68.hGALC at a dose of 4.5 × 10¹² GC (low dose), 1.5 × 10¹³ GC (mid-dose), or 4.5 × 10¹³ GC (high dose) (N=3/group). CSF was collected on Days 0, 7±1, 14±2, 29±3, 60±3, 90±4, 120±4, 150±4, and 180±5. Leukocytes were quantified as the number of WBCs per µl of CSF. Abbreviations: CSF, cerebrospinal fluid; GC, genome copies; ID, identification number; ICM, intra-cisterna magna; ITFFB, intrathecal final formulation buffer; N, number of animals; NHP, non-human primate; WBCs, white blood cells.

FIG. 96C and FIG. 96D show CSF and sensory neuron safety monitoring in sham treated Krabbe and wildtype dogs, and Krabbe dogs administered AAVhu68.cGALC. (FIG. 96C) CSF pleocytosis. (FIG. 96D) Dorsal root ganglia histology from an AAVhu68.cGALC treated Krabbe dog.

FIG. 97A and FIG. 97B show body weights following ICM administration of rAAVhu68.hGALC to NHPs at day 90 (FIG. 97A) or day 180 (FIG. 97B). Juvenile NHPs received a single ICM administration of either vehicle (ITFFB; N=1group) or rAAVhu68.hGALC at a dose of 4.5 × 10¹² GC (low dose), 1.5 × 10¹³ GC (mid-dose), or 4.5 × 10¹³ GC (high dose) (N=3/group). Body weights were monitored at BL and on Days 0, 7±1, 14±2, 29±3, 60±3, 90±4, 120±4, 150±4, and 180±5. Abbreviations: BL, baseline; GC, genome copies; ICM, intra-cisterna magna; ITFFB, intrathecal final formulation buffer; N, number of animals; NHP, non-human primate; SNAP, sensory nerve action potential.

FIG. 98A - FIG. 98C show DRG neuronal degeneration severity scores after ICM administration of rAAVhu68.hGALC to NHPs at day 90 (FIG. 98A), day 180 (FIG. 98B), or combined (FIG. 98C). Juvenile NHPs received a single ICM administration of either vehicle (ITFFB; N=1group) or rAAVhu68.hGALC at a dose of 4.5 × 10¹² GC (low dose), 1.5 × 10¹³ GC (mid-dose), or 4.5 × 10¹³ GC (high dose) (N=3/group). Severity grade scores for all ITFFB- and rAAVhu68.hGALC-treated animals necropsied on Day 90 and Day 180 are presented in each DRG segment (cervical, thoracic, and lumbar) for findings of neuronal cell body degeneration with mononuclear cell infiltrates. For each DRG segment, the following scores were assigned: Severity Grade 1 = minimal, Severity Grade 2 = mild, Severity Grade 3 = moderate, Severity Grade 4 = marked; Severity Grade 5 = severe. Abbreviations: DRG, dorsal root ganglia; GC, genome copies; ICM, intra-cisterna magna; ITFFB, intrathecal final formulation buffer; N, number of animals; NHP, non-human primate; TRG, trigeminal ganglia.

FIG. 99A - FIG. 99C show spinal cord axonopathy severity scores after ICM administration of rAAVhu68.hGALC to NHPs at day 90 (FIG. 99A), day 180 (FIG. 99B), or all scores combined (FIG. 99C). Juvenile NHPs received a single ICM administration of either vehicle (ITFFB; N=l/group) or rAAVhu68.hGALC at a dose of 4.5 × 10¹² GC (low dose), 1.5 × 10¹³ GC (mid-dose), or 4.5 × 10¹³ GC (high dose) (N=3/group). Severity grade scores for all ITFFB- and rAAVhu68.hGALC-treated animals necropsied on Day 90 and Day 180 are presented for axonopathy in the dorsal white matter tracts of the spinal cord (cervical, thoracic, and lumbar segments). For each finding, the following scores were assigned: Severity Grade 1 = minimal, Severity Grade 2 = mild, Severity Grade 3 = moderate, Severity Grade 4 = marked; Severity Grade 5 = severe. *p<0.05 based on a Kriskall Wallis test followed by a multiple comparisons Dunn’s test comparing each group to the vehicle-treated control group. Abbreviations: GC, genome copies; ICM, intra-cisterna magna; ITFFB, intrathecal final formulation buffer; N, number of animals; NHP, non-human primate.

FIG. 100A - FIG. 100D show GALC enzyme activity in serum and CSF of NHPs treated with rAAVhu68.hGALC or vehicle. FIG. 100A graphs GALC serum levels over 180 days of the study. FIG. 100B shows an enlarged view of day 14 at the various doses. FIG. 100C graphs GALC CSF levels over 180 days of the study. FIG. 100D shows an enlarged view of day 7 at the various doses. Juvenile NHPs received a single ICM administration of either vehicle (ITFFB; N=l/group) or rAAVhu68.hGALC at a dose of 4.5 × 10¹² GC (low dose), 1.5 × 10¹³ GC (mid-dose), or 4.5 × 10¹³ GC (high dose) (N=3/group). CSF and serum were collected at the indicated days and analyzed for transgene product expression (GALC enzyme activity). In the Day 14 graph for serum (FIG. 100B) and Day 7 graph for CSF (FIG. 100D), empty shapes indicate animals that were negative for serum-circulating NAbs against the vector capsid at the time of treatment. Empty shapes denote animals that were negative for serum-circulating NAbs against the vector capsid at the time of treatment. Error bars represent the standard deviation. Abbreviations: BL, baseline; GALC, galactosylceramidase (protein); GC, genome copies; ICM, intra-cisterna magna; ITFFB, intrathecal final formulation buffer; N, number of animals; NAb, neutralizing antibody; NHP, non-human primate.

FIG. 101A shows anti-human GALC antibodies in CSF of NHPs following ICM administration of rAAVhu68.hGALC. FIG. 101B shows anti-human GALC antibodies in serum of NHPs following ICM administration of rAAVhu68.hGALC. Juvenile NHPs received a single ICM administration of either vehicle (ITFFB; N= 1/group) or rAAVhu68.hGALC at a dose of 4.5 × 10¹² GC (low dose), 1.5 × 10¹³ GC (mid-dose), or 4.5 × 10¹³ GC (high dose) (N=3/group). CSF and serum were collected on the indicated days, and antibodies against the transgene product (anti-human GALC antibodies) were measured by ELISA. Error bars represent the standard deviation. Abbreviations: BL, baseline; ELISA, enzyme-linked immunosorbent assay; GALC, galactosylceramidase (protein); GC, genome copies; ICM, intra-cisterna magna; ITFFB, intrathecal final formulation buffer; N, number of animals; NHP, non-human primate.

### DETAILED DESCRIPTION OF THE INVENTION

A recombinant adeno-associated virus (rAAV) which expresses a human galactosylceramidase (GALC) protein is provided, as are compositions containing the rAAV and uses thereof. In certain embodiments, the rAAV.hGALC provides for the first time, a disease-modifying treatment for symptomatic infantile Krabbe patients (early infantile Krabbe disease, EIKD). In certain embodiments, the rAAV.hGALC provides a treatment for presymptomatic infantile patients. In certain embodiments, the rAAV.hGALC provides a therapy that can correct peripheral nerves which cause respiratory failure and motor function loss. In certain embodiments, the rAAV.hGALC provides additional options for treatment of later-onset patients for whom the benefit-risk ratio is not in favor of hematopoietic stem cell transplant (HSCT), which is currently the only disease-modifying treatment.

As used herein, a “rAAV.GALC” refers to a rAAV having an AAV capsid which has packaged therein a vector genome containing, at a minimum, a coding sequence for the galactosylceramidase protein (enzyme). rAAVhu68.GALC refers to a rAAV in which the AAV capsid is an AAVhu68 capsid, which is defined herein. The examples below also illustrate other AAV capsids.

The term “cGALC” refers to a coding sequence which expresses a canine GALC, which as used in the examples below for studies in dogs. Canine GALC has a 26 bp signal peptide and a total length of the protein of 669 amino acids.

The term “hGALC” refers to a coding sequence for a human GALC.

Isoform 1 of hGALC is the canonical sequence and is 685 amino acids in length. This amino acid sequence is reproduced in SEQ ID NO: 6. The mature protein is located at about amino acid 43 to about 685 and a signal peptide is located in positions 1 to 42, although there is some suggestion that the initiating Met is at position 17 rather than at position 1. Although multiple isoforms of GALC are known (isoforms 1-5), and over three dozen natural variants have been described, the present inventors have discovered that a variation having a threonine (T) to Alanine (A) mutation at position 641 is particularly desirable. This sequence is provided in SEQ ID NO: 10. This variant is the protein sequence encoded by the human galactosylceramidase (hGALC) coding sequence illustrated in the examples in the rAAV and vector genomes provided herein. Galactosylceramidase (GALC) is also known as galactocerebrosidase and these names are used interchangeably. In certain embodiments, this variant may be used in enzyme replacement therapy or co-therapies.

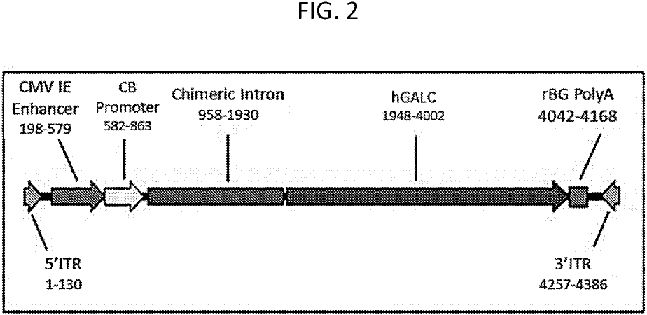

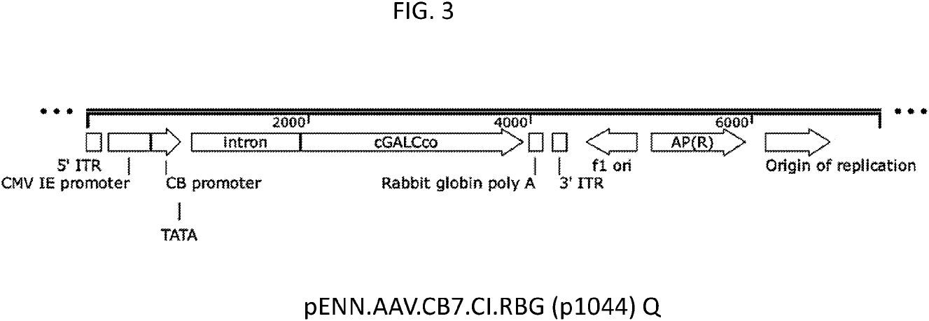

As used herein, “CB7.CI.hGALC.rBG” refers to a vector genome (e.g., as depicted in FIG. 2) that contains a coding sequence for human GALC under the control of the ubiquitous CB7 promoter and includes at least a CMV IE (cytomegalovirus immediate-early) enhancer, a chimeric intron, and a rabbit β-globin (rBG) polyA sequence, all of which are flanked by a 5′ITR and a 3′ITR. In certain embodiments, the CB7.CI.hGALC.rBG includes a GALC coding sequence encoding a mature GALC protein having the amino acid sequence of SEQ ID NO: 10. In certain embodiments, the CB7.CI.hGALC.rBG includes a coding sequence for GALC that contains the nucleic acid sequence of SEQ ID NO: 9 or a sequence 95% to 99.9% identical thereto. In yet another embodiment, the CB7.CI.hGALC.rBG vector genome includes SEQ ID NO: 19. In certain embodiments, the CB7.CI.hGALC.rBG contains a coding sequence for the mature protein of SEQ ID NO: 10 and an exogenous signal peptide.

In certain embodiments, a fusion protein is contemplated which contains at least the mature GALC with all or a portion of the native signal peptide removed (aa 1-17, or aa 1-42) and substituted with an exogenous signal peptide. Such a fusion protein may contain an exogenous signal peptide and at least the mature human GALC protein (e.g., amino acid 43 to 695 of SEQ ID NO: 6 or SEQ ID NO: 10). In certain embodiments, the fusion protein contains an exogenous signal peptide suitable for human cells in the CNS, i.e. a signal peptide that is substituted for a native signal peptide to improve production, intracellular transport, and/or secretion of the protein (i.e. hGALC) in cells present in the human CNS. Exogenous signal peptides suitable for human cells in the CNS, include, but are not limited to those natively found in an immunoglobulin (e.g., IgG), a cytokine (e.g., IL-2, IL12, IL18, or the like), insulin, albumin, β-glucuronidase, alkaline protease, von Willebrand factor (VWF), or the fibronectin secretory signal peptides (See, also, e.g., www.signalpeptide.de/index.php?m=listspdb_mammalia).

Also encompassed by the present invention are nucleic acid sequences which encode the GALC protein(s) provided herein (e.g., SEQ ID NO: 6, SEQ ID NO: 10, or fusion proteins comprising the mature GALC). In certain embodiments, a coding sequence is a cDNA sequence encoding the protein. However, also encompassed are the corresponding RNA sequences.

In certain embodiments, a nucleic acid coding sequence has the cDNA sequence of SEQ ID NO: 5 or a sequence 95% to 99.9% identical thereto, or a fragment thereof. Suitable fragments include the coding sequence for the mature protein (about nt 127 to about nt 2058), or the coding sequence for the mature protein with a fragment of the signal peptide (e.g., about nt 54 to about nt 2058). In certain embodiments, the coding sequence has the nucleic acid sequence encoding the mature hGALC of SEQ ID NO: 5 (nt 127 to 2058) or a fusion protein comprising the same and an exogenous leader, or a sequence 95% to 99.9% identical thereto. In certain embodiments, the coding sequence has the nucleic acid sequence encoding the mature hGALC of SEQ ID NO: 5 (nt 127 to 2058) or a sequence 95% to 99.9% identical thereto, or a fragment thereof comprising a fragment of the leader sequence and the mature hGALC. In certain embodiments, the coding sequence encodes a full-length human GALC protein having the amino acid sequence of SEQ ID NO: 10. In certain embodiments, the coding sequence encodes the hGALC leader (nucleic acids 1 to 126) and mature protein (encoded by nucleic acids 127 to 2058) of SEQ ID NO: 5.

In certain embodiments, the expression cassette comprises one or more miRNA target sequences that repress expression of hGALC in dorsal root ganglion (drg). Such miRNA target sequences can be operably linked to the hGALC coding sequence. Suitable miRNA target sequences are described in PCT/US19/67872, filed Dec. 20, 2019, and entitled “Compositions for DRG-specific reduction of transgene expression”. International Patent Application No. PCT/US19/67872 is incorporated herein by reference.

As used herein, Krabbe disease, also known as globoid cell leukodystrophy (GLD) is a lysosomal storage disease caused by mutation affecting the activity of galactosylceramidase (GALC), an enzyme responsible for the degradation of myelin galactolipids. Several types of Krabbe disease have been described which depend on the severity of the enzymatic deficit. From the most severe to least severe enzymatic deficit are: early infantile Krabbe disease (EIKD) defined by onset ≤ 6 months of age; late infantile Krabbe disease (LIKD) defined by onset from 7 to 12 months; juvenile Krabbe disease (JKD) defined by onset from 13 months to 10 years; and adolescent/adult onset Krabbe disease.

In certain embodiments, an effective amount of a rAAV.GALC vector increases GALC enzyme levels in the CSF to within about 30% to about 100% of normal levels. In other embodiments, an effective amount of a rAAV. GALC vector increases GALC enzyme levels in the plasma to within about 30% to about 100% of normal levels. In certain embodiments, lower amounts of increased CSF or plasma levels of GALC are observed, but an improvement is observed in one or more of the symptoms associated with Krabbe disease, as described herein.

A “recombinant AAV” or “rAAV” is a DNAse-resistant viral particle containing two elements, an AAV capsid and a vector genome containing at least non-AAV coding sequences packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs may be used. Further, the vector genome contains regulatory sequences which direct expression of the gene products. Suitable components of a vector genome are discussed in more detail herein.

### AAVhu68

As described in the examples below, the rAAV provided herein comprises an AAVhu68 capsid. See, e.g., WO 2018/160582, which is incorporated herein by reference. AAVhu68 is within clade F. AAVhu68 (SEQ ID NO: 2) varies from another Clade F virus AAV9 (SEQ ID NO: 4) by two encoded amino acids at positions 67 and 157 of vp1. In contrast, the other Clade F AAV (AAV9, hu31, hu31) has an Ala at position 67 and an Ala at position 157.

A rAAVhu68 is composed of an AAVhu68 capsid and a vector genome. In one embodiment, a composition comprising rAAVhu68 comprises an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences. SEQ ID NO: 2 provides the encoded amino acid sequence of the AAVhu68 vp1 protein. The AAVhu68 capsid contains subpopulations within the vp1 proteins, within the vp2 proteins and within the vp3 proteins which have modifications from the predicted amino acid residues in SEQ ID NO: 2. These subpopulations include, at a minimum, certain deamidated asparagine (N or Asn) residues. For example, certain subpopulations comprise at least one, two, three or four highly deamidated asparagines (N) positions in asparagine - glycine pairs in SEQ ID NO: 2 and optionally further comprising other deamidated amino acids, wherein the deamidation results in an amino acid change and other optional modifications. The various combinations of these and other modifications are described herein.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine - glycine pairs.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, 97%, 99%, up to about 100% deamidated at a referenced amino acid position, as compared to the predicted amino acid sequence at the reference amino acid position (e.g., at least 80% of the asparagines at amino acid 57 of SEQ ID NO: 2 may be deamidated based on the total vp1 proteins or 20% of the asparagines at amino acid 409 of SEQ ID NO: 2 may be deamidated based on the total vp1, vp2 and vp3 proteins). Such percentages may be determined using 2D-gel, mass spectrometry techniques, or other suitable techniques.

As provided herein, each deamidated N of SEQ ID NO: 2 may independently be aspartic acid (Asp), isoaspartic acid (isoAsp), aspartate, and/or an interconverting blend of Asp and isoAsp, or combinations thereof. Any suitable ratio of α- and isoaspartic acid may be present. For example, in certain embodiments, the ratio may be from 10:1 to 1:10 aspartic to isoaspartic, about 50:50 aspartic: isoaspartic, or about 1:3 aspartic: isoaspartic, or another selected ratio. In certain embodiments, one or more glutamine (Q) in SEQ ID NO: 2 deamidates to glutamic acid (Glu), i.e., α-glutamic acid, γ-glutamic acid (Glu), or a blend of α- and γ-glutamic acid, which may interconvert through a common glutarinimide intermediate. Any suitable ratio of α- and γ-glutamic acid may be present. For example, in certain embodiments, the ratio may be from 10:1 to 1:10 α to y, about 50:50 α: γ, or about 1:3 α : γ, or another selected ratio.

Thus, an rAAVhu68 includes subpopulations within the rAAVhu68 capsid of vp1, vp2 and/or vp3 proteins with deamidated amino acids, including at a minimum, at least one subpopulation comprising at least one highly deamidated asparagine. In addition, other modifications may include isomerization, particularly at selected aspartic acid (D or Asp) residue positions. In still other embodiments, modifications may include an amidation at an Asp position.

In certain embodiments, an AAVhu68 capsid contains subpopulations of vp1, vp2 and vp3 having at least 4 to at least about 25 deamidated amino acid residue positions, of which at least 1 to 10% are deamidated as compared to the encoded amino acid sequence of SEQ ID NO: 2. The majority of these may be N residues. However, Q residues may also be deamidated.

In certain embodiments, an AAVhu68 capsid is further characterized by one or more of the following. AAVhu68 capsid proteins that comprise: AAVhu68 vp1 proteins produced by expression from a nucleic acid sequence which encodes the predicted amino acid sequence of 1 to 736 of SEQ ID NO: 2, vp1 proteins produced from SEQ ID NO: 1, or vp1 proteins produced from a nucleic acid sequence at least 70% identical to SEQ ID NO: 1 which encodes the predicted amino acid sequence of 1 to 736 of SEQ ID NO: 2; AAVhu68 vp2 proteins produced by expression from a nucleic acid sequence which encodes the predicted amino acid sequence of at least about amino acids 138 to 736 of SEQ ID NO:2, vp2 proteins produced from a sequence comprising at least nucleotides 412 to 2211 of SEQ ID NO:1, or vp2 proteins produced from a nucleic acid sequence at least 70% identical to at least nucleotides 412 to 2211 of SEQ ID NO: 1 which encodes the predicted amino acid sequence of at least about amino acids 138 to 736 of SEQ ID NO: 2, and/or AAVhu68 vp3 proteins produced by expression from a nucleic acid sequence which encodes the predicted amino acid sequence of at least about amino acids 203 to 736 of SEQ ID NO: 2, vp3 proteins produced from a sequence comprising at least nucleotides 607 to 2211 of SEQ ID NO: 1, or vp3 proteins produced from a nucleic acid sequence at least 70% identical to at least nucleotides 607 to 2211 of SEQ ID NO: 1 which encodes the predicted amino acid sequence of at least about amino acids 203 to 736 of SEQ ID NO: 2.

Additionally or alternatively, an AAV capsid is provided which comprises a heterogeneous population of vp1 proteins optionally comprising a valine at position 157, a heterogeneous population of vp2 proteins optionally comprising a valine at position 157, and a heterogeneous population of vp3 proteins, wherein at least a subpopulation of the vp 1 and vp2 proteins comprise a valine at position 157 and optionally further comprising a glutamic acid at position 67 based on the numbering of the vp1 capsid of SEQ ID NO:2. Additionally or alternatively, an AAVhu68 capsid is provided which comprises a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 736 of SEQ ID NO: 2, and a heterogeneous population of vp3 proteins which are the product of a nucleic acid sequence encoding at least amino acids 203 to 736 of SEQ ID NO: 2, wherein: the vp1, vp2 and vp3 proteins contain subpopulations with amino acid modifications.

The AAVhu68 vp1, vp2 and vp3 proteins are typically expressed as alternative splice variants encoded by the same nucleic acid sequence which encodes the full-length vp1 amino acid sequence of SEQ ID NO: 2 (amino acid 1 to 736). Optionally the vp1-encoding sequence is used alone to express the vp1, vp2 and vp3 proteins. Alternatively, this sequence may be co-expressed with one or more of a nucleic acid sequence which encodes the AAVhu68 vp3 amino acid sequence of SEQ ID NO: 2 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 1), or a sequence at least 70% to at least 99% (e.g., at least 85%, at least 90%, at least 95%, at least 97%, at least 98% or at least 99%) identical to SEQ ID NO: 1 which encodes aa 203 to 736 of SEQ ID NO: 2. Additionally, or alternatively, the vp1-encoding and/or the vp2-encoding sequence may be co-expressed with the nucleic acid sequence which encodes the AAVhu68 vp2 amino acid sequence of SEQ ID NO: 2 (about aa 138 to 736) without the vp1-unique region (about aa 1 to about 137), or a strand complementary thereto, the corresponding mRNA or tRNA (nt 412 to 22121 of SEQ ID NO: 1), or a sequence at least 70% to at least 99% (e.g., at least 85%, at least 90%, at least 95%, at least 97%, at least 98% or at least 99%) identical to SEQ ID NO: 1 which encodes about aa 138 to 736 of SEQ ID NO: 2.

As described herein, a rAAVhu68 has a rAAVhu68 capsid produced in a production system expressing capsids from an AAVhu68 nucleic acid which encodes the vp1 amino acid sequence of SEQ ID NO: 2, and optionally additional nucleic acid sequences, e.g., encoding a vp3 protein free of the vp1 and/or vp2-unique regions. The rAAVhu68 resulting from production using a single nucleic acid sequence vp1 produces the heterogeneous populations of vp1 proteins, vp2 proteins and vp3 proteins. More particularly, the rAAVhu68 capsid contains subpopulations within the vp1 proteins, within the vp2 proteins and within the vp3 proteins which have modifications from the predicted amino acid residues in SEQ ID NO:2. These subpopulations include, at a minimum, deamidated asparagine (N or Asn) residues. For example, asparagines in asparagine - glycine pairs are highly deamidated.

In one embodiment, the AAVhu68 vp1 nucleic acid sequence has the sequence of SEQ ID NO: 1, or a strand complementary thereto, e.g., the corresponding mRNA or tRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 2 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 1). In certain embodiments, also provided is a nucleic acid sequence which encodes the AAVhu68 vp2 amino acid sequence of SEQ ID NO: 2 (about aa 138 to 736) without the vp1-unique region (about aa 1 to about 137), or a strand complementary thereto, the corresponding mRNA or tRNA (nt 412 to 2211 of SEQ ID NO: 1).

However, other nucleic acid sequences which encode the amino acid sequence of SEQ ID NO: 2 may be selected for use in producing rAAVhu68 capsids. In certain embodiments, the nucleic acid sequence has the nucleic acid sequence of SEQ ID NO: 1 or a sequence at least 70% to 99% identical, at least 75%, at least 80%, at least 85%, at least 90%, at least 95%, at least 97%, or at least 99% identical to SEQ ID NO: 1 which encodes SEQ ID NO: 2. In certain embodiments, the nucleic acid sequence has the nucleic acid sequence of SEQ ID NO: 1 or a sequence at least 70% to 99%, at least 75%, at least 80%, at least 85%, at least 90%, at least 95%, at least 97%, or at least 99% identical to about nt 412 to about nt 2211 of SEQ ID NO: 1 which encodes the vp2 capsid protein (about aa 138 to 736) of SEQ ID NO: 2. In certain embodiments, the nucleic acid sequence has the nucleic acid sequence of about nt 607 to about nt 2211 of SEQ ID NO: 1 or a sequence at least 70% to 99.%, at least 75%, at least 80%, at least 85%, at least 90%, at least 95%, at least 97%, or at least 99% identical to about nt 607 to about nt 2211 SEQ ID NO: 1 which encodes the vp3 capsid protein (about aa 203 to 736) of SEQ ID NO: 2.

It is within the skill in the art to design nucleic acid sequences encoding this rAAVhu68 capsid, including DNA (genomic or cDNA), or RNA (e.g., mRNA). In certain embodiments, the nucleic acid sequence encoding the AAVhu68 vp1 capsid protein is provided in SEQ ID NO: 1. In other embodiments, a nucleic acid sequence of 70% to 99.9% identity to SEQ ID NO: 1 may be selected to express the AAVhu68 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 1. Such nucleic acid sequences may be codon-optimized for expression in a selected system (i.e., cell type) can be designed by various methods. This optimization may be performed using methods which are available on-line (e.g., GeneArt), published methods, or a company which provides codon optimizing services, e.g., DNA2.0 (Menlo Park, CA). One codon optimizing method is described, e.g., in U.S. International Patent Publication No. WO 2015/012924, which is incorporated by reference herein in its entirety. See also, e.g., U.S. Pat. Publication No. 2014/0032186 and U.S. Pat. Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence and produce a nucleic acid fragment of a codon-optimized coding region which encodes the polypeptide. A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In certain embodiments, the asparagine (N) in N-G pairs in the rAAVhu68 vp1, vp2 and vp3 proteins are highly deamidated. In the case of the rAAVhu68 capsid protein, 4 residues (N57, N329, N452, N512) routinely display levels of deamidation >70% and it most cases >90% across various lots. Additional asparagine residues (N94, N253, N270, N304, N409, N477, and Q599) also display deamidation levels up to ~20% across various lots. The deamidation levels were initially identified using a trypsin digest and verified with a chymotrypsin digestion.

In certain embodiments, an rAAVhu68 capsid contains subpopulations of AAV vp1, vp2 and/or vp3 capsid proteins having at least four asparagine (N) positions in the rAAVhu68 capsid proteins which are highly deamidated. In certain embodiments, about 20 to 50% of the N-N pairs (exclusive of N-N-N triplets) show deamidation. In certain embodiments, the first N is deamidated. In certain embodiments, the second N is deamidated. In certain embodiments, the deamidation is between about 15% to about 25% deamidation. Deamidation at the Q at position 259 of SEQ ID NO: 2 is about 8% to about 42% of the AAVhu68 vp1, vp2 and vp3 capsid proteins of an AAVhu68 protein.

In certain embodiments, the rAAVhu68 capsid is further characterized by an amidation in D297 the vp1, vp2 and vp3 proteins. In certain embodiments, about 70% to about 75% of the D at position 297 of the vp1, vp2 and/or vp3 proteins in a AAVhu68 capsid are amidated, based on the numbering of SEQ ID NO: 2. In certain embodiments, at least one Asp in the vp1, vp2 and/or vp3 of the capsid is isomerized to D-Asp. Such isomers are generally present in an amount of less than about 1% of the Asp at one or more of residue positions 97, 107, 384, based on the numbering of SEQ ID NO: 2.

In certain embodiments, a rAAVhu68 has an AAVhu68 capsid having vp1, vp2 and vp3 proteins having subpopulations comprising combinations of one, two, three, four or more deamidated residues at the positions set forth in the table below. Deamidation in the rAAV may be determined using 2D gel electrophoresis, and/or mass spectrometry, and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra. Examples of suitable proteases may include, e.g., trypsin or chymotrypsin. Mass spectrometric identification of deamidated peptides is relatively straightforward, as deamidation adds to the mass of intact molecule +0.984 Da (the mass difference between —OH and —NH₂ groups). The percent deamidation of a particular peptide is determined mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It will be understood by one of skill in the art that a number of variations on these illustrative methods can be used. For example, suitable mass spectrometers may include, e.g, a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

| Deamidation Based on Predicted AAVHu68 (SEQ ID NO: 2) | Average % Based on VP1/VP2/VP3 Proteins in AAVhu68 Capsid | |
|---|---|---|
| Deamidated Residue + 1 (Neighboring AA) | Broad Range of Percentages (%) | Narrow Ranges (%) |
| N57 (N-G) | 78 to 100% | 80 to 100, 85 to 97 |
| N66 (N-E) | 0 to 5 | 0, 1 to 5 |
| N94 (N-H) | 0 to 15, | 0, 1 to 15, 5 to 12, 8 |
| N113 (N-L) | 0 to 2 | 0, 1 to 2 |
| ~N253 (N-N) | 10 to 25 | 15 to 22 |
| Q259 (Q-I) | 8 to 42 | 10 to 40, 20 to 35 |
| ~N270 (N-D) | 12 to 30 | 15 to 28 |
| ~N304 (N-N) (position 303 also N) | 0 to 5 | 1 to 4 |
| N319 (N-I) | 0 to 5 | 0, 1 to 5, 1 to 3 |
| N329 * (N-G)*(position 328 also N) | 65 to 100 | 70 to 95, 85 to 95, 80 to 100, 85 to 100, |
| N336 (N-N) | 0 to 100 | 0, 1 to 10, 25 to 100, 30 to 100, 30 to 95 |
| ~409 (N-N) | 15 to 30 | 20 to 25 |
| N452 (N-G) | 75 to 100 | 80 to 100, 90 to 100, 95 to 100, |
| 477 (N-Y) | 0 to 8 | 0, 1 to 5 |
| N512 (N-G) | 65 to 100 | 70 to 95, 85 to 95, 80 to 100, 85 to 100, |
| N515 (N-S) | 0 to 25 | 0, 1 to 10, 5 to 25, 15 to 25 |
| ~Q599 (Asn-Q-Gly) | 1 to 20 | 2 to 20, 5 to 15 |
| N628 (N-F) | 0 to 10 | 0, 1 to 10, 2 to 8 |
| N651 (N-T) | 0 to 3 | 0, 1 to 3 |
| N663 (N-K) | 0 to 5 | 0, 1 to 5, 2 to 4 |
| N709 (N-N) | 0 to 25 | 0,1 to 22, 15 to 25 |
| N735 | 0 to 40 | 0. 1 to 35, 5 to 50, 20 to 35 |

In certain embodiments, the AAVhu68 capsid is characterized by having capsid proteins in which at least 45% of N residues are deamidated at least one of positions N57, N329, N452, and/or N512 based on the numbering of amino acid sequence of SEQ ID NO: 2. In certain embodiments, at least about 60%, at least about 70%, at least about 80%, or at least 90% of the N residues at one or more of these N-G positions (i.e., N57, N329, N452, and/or N512, based on the numbering of amino acid sequence of SEQ ID NO: 2) are deamidated. In these and other embodiments, an AAVhu68 capsid is further characterized by having a population of proteins in which about 1% to about 20% of the N residues have deamidations at one or more of positions: N94, N253, N270, N304, N409, N477, and/or Q599, based on the numbering of amino acid sequence of SEQ ID NO: 2. In certain embodiments, the AAVhu68 comprises at least a subpopulation of vp1, vp2 and/or vp3 proteins which are deamidated at one or more of positions N35, N57, N66, N94, N113, N252, N253, Q259, N270, N303, N304, N305, N319, N328, N329, N336, N409, N410, N452, N477, N515, N598, Q599, N628, N651, N663, N709, N735, based on the numbering of amino acid sequence of SEQ ID NO: 2, or combinations thereof. In certain embodiments, the capsid proteins may have one or more amidated amino acids.

Still other modifications are observed, most of which do not result in conversion of one amino acid to a different amino acid residue. Optionally, at least one Lys in the vp1, vp2 and vp3 of the capsid are acetylated. Optionally, at least one Asp in the vp1, vp2 and/or vp3 of the capsid is isomerized to D-Asp. Optionally, at least one S (Ser, Serine) in the vp1, vp2 and/or vp3 of the capsid is phosphorylated. Optionally, at least one T (Thr, Threonine) in the vp1, vp2 and/or vp3 of the capsid is phosphorylated. Optionally, at least one W (trp, tryptophan) in the vp1, vp2 and/or vp3 of the capsid is oxidized. Optionally, at least one M (Met, Methionine) in the vp1, vp2 and/or vp3 of the capsid is oxidized. In certain embodiments, the capsid proteins have one or more phosphorylations. For example, certain vp1 capsid proteins may be phosphorylated at position 149.

In certain embodiments, an rAAVhu68 capsid comprises a heterogeneous population of vp 1 proteins which are the product of a nucleic acid sequence encoding the amino acid sequence of SEQ ID NO: 2, wherein the vp1 proteins comprise a Glutamic acid (Glu) at position 67 and/or a valine (Val)at position 157; a heterogeneous population of vp2 proteins optionally comprising a valine (Val) at position 157; and a heterogeneous population of vp3 proteins. The AAVhu68 capsid contains at least one subpopulation in which at least 65% of asparagines (N) in asparagine - glycine pairs located at position 57 of the vp1 proteins and at least 70% of asparagines (N) in asparagine - glycine pairs at positions 329, 452 and/or 512 of the vp1, v2 and vp3 proteins are deamidated, based on the residue numbering of the amino acid sequence of SEQ ID NO: 2, wherein the deamidation results in an amino acid change.

As discussed in more detail herein, the deamidated asparagines may be deamidated to aspartic acid, isoaspartic acid, an interconverting aspartic acid/isoaspartic acid pair, or combinations thereof. In certain embodiments, the rAAVhu68 are further characterized by one or more of: (a) each of the vp2 proteins is independently the product of a nucleic acid sequence encoding at least the vp2 protein of SEQ ID NO: 2; (b) each of the vp3 proteins is independently the product of a nucleic acid sequence encoding at least the vp3 protein of SEQ ID NO: 2; (c) the nucleic acid sequence encoding the vp1 proteins is SEQ ID NO: 1, or a sequence at least 70% to at least 99% (e.g., at least 85%, at least 90%, at least 95%, at least 97%, at least 98% or at least 99%) identical to SEQ ID NO: 1 which encodes the amino acid sequence of SEQ ID NO: 2. Optionally that sequence is used alone to express the vp1, vp2 and vp3 proteins. Alternatively, this sequence may be co-expressed with one or more of a nucleic acid sequence which encodes the AAVhu68 vp3 amino acid sequence of SEQ ID NO: 2 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 1), or a sequence at least 70% to at least 99% (e.g., at least 85%, at least 90%, at least 95%, at least 97%, at least 98% or at least 99%) identical to SEQ ID NO: 1 which encodes aa 203 to 736 of SEQ ID NO: 2. Additionally, or alternatively, the vp1-encoding and/or the vp2-encoding sequence may be co-expressed with the nucleic acid sequence which encodes the AAVhu68 vp2 amino acid sequence of SEQ ID NO: 2 (about aa 138 to 736) without the vp1-unique region (about aa 1 to about 137), or a strand complementary thereto, the corresponding mRNA or tRNA (nt 412 to 2211 of SEQ ID NO: 1), or a sequence at least 70% to at least 99% (e.g., at least 85%, at least 90%, at least 95%, at least 97%, at least 98% or at least 99%) identical to SEQ ID NO: 1 which encodes about aa 138 to 736 of SEQ ID NO: 2.

Additionally or alternatively, the rAAVhu68 capsid comprises at least a subpopulation of vp1, vp2 and/or vp3 proteins which are deamidated at one or more of positions N57, N66, N94, N113, N252, N253, Q259, N270, N303, N304, N305, N319, N328, N329, N336, N409, N410, N452, N477, N512, N515, N598, Q599, N628, N651, N663, N709, based on the numbering of SEQ ID NO:2, or combinations thereof; (e) rAAVhu68 capsid comprises a subpopulation of vp1, vp2 and/or vp3 proteins which comprise 1% to 20% deamidation at one or more of positions N66, N94, N113, N252, N253, Q259, N270, N303, N304, N305, N319, N328, N336, N409, N410, N477, N515, N598, Q599, N628, N651, N663, N709, based on the numbering of SEQ ID NO:2, or combinations thereof; (f) the rAAVhu68 capsid comprises a subpopulation of vp1 in which 65% to 100 % of the N at position 57 of the vp1 proteins, based on the numbering of SEQ ID NO:2, are deamidated; (g) the rAAVhu68 capsid comprises subpopulation of vp1 proteins in which 75% to 100% of the N at position 57 of the vp1 proteins are deamidated; (h) the rAAVhu68 capsid comprises subpopulation of vp1 proteins, vp2 proteins, and/or vp3 proteins in which 80% to 100% of the N at position 329, based on the numbering of SEQ ID NO:2, are deamidated; (i) the rAAVhu68 capsid comprises subpopulation of vp1 proteins, vp2 proteins, and/or vp3 proteins in which 80% to 100% of the N at position 452, based on the numbering of SEQ ID NO:2, are deamidated; (j) the rAAVhu68 capsid comprises subpopulation of vp1 proteins, vp2 proteins, and/or vp3 proteins in which 80% to 100% of the N at position 512, based on the numbering of SEQ ID NO: 2, are deamidated; (k) the rAAV comprises about 60 total capsid proteins in a ratio of about 1 vp1 to about 1 to 1.5 vp2 to 3 to 10 vp3 proteins; (1) the rAAV comprises about 60 total capsid proteins in a ratio of about 1 vp1 to about 1 vp2 to 3 to 9 vp3 proteins.

In certain embodiments, the AAVhu68 is modified to change the glycine in an asparagine-glycine pair, in order to reduce deamidation. In other embodiments, the asparagine is altered to a different amino acid, e.g., a glutamine which deamidates at a slower rate; or to an amino acid which lacks amide groups (e.g., glutamine and asparagine contain amide groups); and/or to an amino acid which lacks amine groups (e.g., lysine, arginine and histidine contain amide groups). As used herein, amino acids lacking amide or amine side groups refer to, e.g., glycine, alanine, valine, leucine, isoleucine, serine, threonine, cystine, phenylalanine, tyrosine, or tryptophan, and/or proline. Modifications such as described may be in one, two, or three of the asparagine-glycine pairs found in the encoded AAVhu68 amino acid sequence. In certain embodiments, such modifications are not made in all four of the asparagine - glycine pairs. Thus, a method is provided for reducing deamidation of rAAVhu68 and/or engineered rAAVhu68 variants having lower deamidation rates. Additionally, one or more other amide amino acids may be changed to a non-amide amino acid to reduce deamidation of the rAAVhu68.

These amino acid modifications may be made by conventional genetic engineering techniques. For example, a nucleic acid sequence containing modified AAVhu68 vp codons may be generated in which one to three of the codons encoding glycine at position 58, 330, 453 and/or 513 in SEQ ID NO: 2 (asparagine - glycine pairs) are modified to encode an amino acid other than glycine. In certain embodiments, a nucleic acid sequence containing modified asparagine codons may be engineered at one to three of the asparagine-glycine pairs located at position 57, 329, 452 and/or 512 in SEQ ID NO: 2, such that the modified codon encodes an amino acid other than asparagine. Each modified codon may encode a different amino acid. Alternatively, one or more of the altered codons may encode the same amino acid. In certain embodiments, these modified AAVhu68 nucleic acid sequences may be used to generate a mutant rAAVhu68 having a capsid with lower deamidation than the native hu68 capsid. Such mutant rAAVhu68 may have reduced immunogenicity and/or increase stability on storage, particularly storage in suspension form. As used herein, a “codon” refers to three nucleotides in a sequence which encodes an amino acid.

As used herein, “encoded amino acid sequence” refers to the amino acid which is predicted based on the translation of a known DNA codon of a referenced nucleic acid sequence being translated to an amino acid. The following table illustrates DNA codons and twenty common amino acids, showing both the single letter code (SLC) and three letter code (3LC).

| | | | |
|---|---|---|---|
| Amino Acid | SLC | 3LC | DNA codons |
| Isoleucine | I | Ile | ATT, ATC, ATA |
| Leucine | L | Leu | CTT, CTC, CTA, CTG, TTA, TTG |
| Valine | V | Val | GTT, GTC, GTA, GTG |
| Phenylalanine | F | Phe | TTT, TTC |
| Methionine | M | Met | ATG |
| Cysteine | C | Cys | TGT, TGC |
| Alanine | A | Ala | GCT, GCC, GCA, GCG |
| Glycine | G | Gly | GGT, GGC, GGA, GGG |
| Proline | P | Pro | CCT, CCC, CCA, CCG |
| Threonine | T | Thr | ACT, ACC, ACA, ACG |
| Serine | S | Ser | TCT, TCC, TCA, TCG, AGT, AGC |
| Tyrosine | Y | Tyr | TAT, TAC |
| Tryptophan | W | Trp | TGG |
| Glutamine | Q | Gln | CAA, CAG |
| Asparagine | N | Asn | AAT, AAC |
| Histidine | H | His | CAT, CAC |
| Glutamic acid | E | Glu | GAA, GAG |
| Aspartic acid | D | Asp | GAT, GAC |
| Lysine | K | Lys | AAA, AAG |
| Arginine | R | Arg | CGT, CGC, CGA, CGG, AGA, AGG |
| Stop codons | Stop | | TAA, TAG, TGA |

rAAVhu68 capsids may be useful in certain embodiments. For example, such capsids may be used in generating monoclonal antibodies and/or generating reagents useful in assays for monitoring AAVhu68 concentration levels in gene therapy patients. Techniques for generating useful anti-AAVhu68 antibodies, labelling such antibodies or empty capsids, and suitable assay formats are known to those of skill in the art.

In certain embodiments, provided herein is a nucleic acid sequence of SEQ ID NO: 1 or a sequence at least 70%, at least 75%, at least 80%, at least 85%, at least 90%, at least 95%, at least 97%, at least 99%, which encodes the vp1 amino acid sequence of SEQ ID NO: 2 with a modification (e.g., deamidated amino acid) as described herein. In certain embodiments, the vp1 amino acid sequence is reproduced in SEQ ID NO: 2.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 Jun; 78(10: 6381-6388, which identifies Clades A, B, C, D, E and F, GenBank Accession Numbers AY530553 to AY530629. See, also, WO 2005/033321.

As used herein, an “AAV9 capsid” is a self-assembled AAV capsid composed of multiple AAV9 vp proteins. The AAV9 vp proteins are typically expressed as alternative splice variants encoded by a nucleic acid sequence of SEQ ID NO: 3 which encodes the vp1 amino acid sequence of SEQ ID NO: 4 (GenBank accession: AAS99264). These splice variants result in proteins of different length of SEQ ID NO: 4. In certain embodiments, “AAV9 capsid” includes an AAV having an amino acid sequence which is 99% identical to AAS99264 or 99% identical to SEQ ID NO: 4. See, also US7906111 and WO 2005/033321. As used herein “AAV9 variants” include those described in, e.g., WO2016/049230, US 8,927,514, US 2015/0344911, and US 8,734,809.

Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. In the examples, AAV alignments are performed using the published AAV9 sequences as a reference point. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

### rAAV Vectors

As indicated above, the AAVhu68 sequences and proteins are useful in production of rAAV, and are also useful in recombinant AAV vectors which may be antisense delivery vectors, gene therapy vectors, or vaccine vectors. Additionally, the engineered AAV capsids described herein, e.g., those having mutant amino acids at position 67, 157, or both relative to the numbering of the vp1 capsid protein in SEQ ID NO: 2, may be used to engineer rAAV vectors for delivery of a number of suitable nucleic acid molecules to target cells and tissues.

Genomic sequences which are packaged into an AAV capsid and delivered to a host cell are typically composed of, at a minimum, a transgene and its regulatory sequences, and AAV inverted terminal repeats (ITRs). Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest.

In particular, the present disclosure provides rAAV comprising a coding sequence of human galactosylceramidase (GALC). In some embodiments, the coding sequence is an engineered GALC coding sequence. In some embodiments, the coding sequence is the sequence of GALC gene (GALCco) of SEQ ID NO: 9. In certain embodiments, the GALC coding sequence comprises a sequence at least 95% identical to SEQ ID NO: 9.

The nucleic acid coding sequence is operatively linked to regulatory components in a manner which permits transgene transcription, translation, and/or expression in a cell of a target tissue. In some embodiments, the regulatory sequences comprise a beta-actin promoter, an intron, and a rabbit globin polyA. In some embodiments, the regulatory sequences comprise SEQ ID NO: 13. In some embodiments, the regulatory sequences comprise SEQ ID NO: 15. In some embodiments, the regulatory sequences comprise SEQ ID NO: 16.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences from AAV2. A shortened version of the 5′ ITR, termed ΔITR, has been described in which the D-sequence and terminal resolution site (trs) are deleted. In other embodiments, the full-length AAV 5′ and 3′ ITRs are used. However, ITRs from other AAV sources may be selected. Where the source of the ITRs is from AAV2 and the AAV capsid is from another AAV source, the resulting vector may be termed pseudotyped. However, other configurations of these elements may be suitable. In certain embodiments, the vector genome includes a shortened AAV2 ITR of 130 base pairs, wherein the external “a” element is deleted. The shortened ITR is reverted back to the wild-type length of 145 base pairs during vector DNA amplification using the internal A element as a template. In other embodiments, the full-length or other AAV 5′ and 3′ ITRs are used.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

The regulatory control elements typically contain a promoter sequence as part of the expression control sequences, e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be used may be utilized in the vectors described herein. The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter. In addition to a promoter a vector may contain one or more other appropriate transcription initiation, termination, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. An example of a suitable enhancer is the CMV enhancer. Other suitable enhancers include those that are appropriate for desired target tissue indications. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g, the chicken beta-actin intron. Other suitable introns include those known in the art, e.g., such as are described in WO 2011/126808. Examples of suitable polyA sequences include, e.g., SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence [see, e.g., MA Zanta-Boussif, et al, Gene Therapy (2009) 16: 605-619.

These rAAVs are particularly well suited to gene delivery for therapeutic purposes and for immunization, including inducing protective immunity. Further, the compositions of the invention may also be used for production of a desired gene product in vitro. For in vitro production, a desired product (e.g., a protein) may be obtained from a desired culture following transfection of host cells with a rAAV containing the molecule encoding the desired product and culturing the cell culture under conditions which permit expression. The expressed product may then be purified and isolated, as desired. Suitable techniques for transfection, cell culturing, purification, and isolation are known to those of skill in the art.

### rAAV Vector Production

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. For packaging a gene into virions, the ITRs are the only AAV components required in cis in the same construct as the nucleic acid molecule containing the expression cassette(s). The cap and rep genes can be supplied in trans.

In one embodiment, the expression cassettes described herein are engineered into a genetic element (e.g., a shuttle plasmid) which transfers the immunoglobulin construct sequences carried thereon into a packaging host cell for production a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; US 7588772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant rAAVhu68 is provided. Such a cell culture contains a nucleic acid which expresses the rAAVhu68 capsid protein in the host cell; a nucleic acid molecule suitable for packaging into the rAAVhu68 capsid, e.g., a vector genome which contains AAV ITRs and a non-AAV nucleic acid sequence encoding a gene product operably linked to sequences which direct expression of the product in a host cell; and sufficient AAV rep functions and adenovirus helper functions to permit packaging of the nucleic acid molecule into the recombinant AAVhu68 capsid. In one embodiment, the cell culture is composed of mammalian cells (e.g., human embryonic kidney 293 cells, among others) or insect cells (e.g., baculovirus).

Suitably, the rep functions are provided by an AAV which is from the same source as the ITRs which are present in the vector genome, or from another source which packages the vector genome into the AAV capsid (e.g., AAVhu68). In certain embodiments, the rep protein is from AAV2. However, in other embodiments In another embodiment, the rep protein is a heterologous rep protein other than AAVhu68rep, for example but not limited to, AAV1 rep protein, AAV2 rep protein, AAV3 rep protein, AAV4 rep protein, AAV5 rep protein, AAV6 rep protein, AAV7 rep protein, AAV8 rep protein; or rep 78, rep 68, rep 52, rep 40, rep68/78 and rep40/52; or a fragment thereof; or another source. Any of these AAVhu68 or mutant AAV capsid sequences may be under the control of exogenous regulatory control sequences which direct expression thereof in a production cell.

In one embodiment, cells are manufactured in a suitable cell culture (e.g., HEK 293) cells. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

In certain embodiments, the manufacturing process for rAAV.hGALC involves transient transfection of HEK293 cells with plasmid DNA. A single batch or multiple batches are produced by PEI-mediated triple transfection of HEK293 cells in PALL iCELLis bioreactors. Harvested AAV material are purified sequentially by clarification, TFF, affinity chromatography, and anion exchange chromatography in disposable, closed bioprocessing systems where possible.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Application No. PCT/US2016/065970, filed Dec. 9, 2016 and its priority documents, U.S. Pat. Application Nos. 62/322,071, filed Apr. 13, 2016 and 62/226,357, filed Dec. 11, 2015 and entitled “Scalable Purification Method for AAV9”, which is incorporated by reference herein. Purification methods for AAV8, International Patent Application No. PCT/US2016/065976, filed Dec. 9, 2016 and its priority documents U.S. Pat. Application Nos. 62/322,098, filed Apr. 13, 2016 and 62/266,341, filed Dec. 11, 2015, and rh10, International Patent Application No. PCT/US16/66013, filed Dec. 9, 2016 and its priority documents, U.S. Pat. Application No. 62/322,055, filed Apr. 13, 2016 and 62/266,347, entitled “Scalable Purification Method for AAVrh10”, also filed Dec. 11, 2015, and for AAV1, International Patent Application No. PCT/US2016/065974, filed Dec. 9, 2016 and its priority documents U.S. Pat. Application Nos. 62/322,083, filed Apr. 13, 2016 and 62/26,351, for “Scalable Purification Method for AAV1”, filed Dec. 11, 2015, are all incorporated by reference herein.

To calculate empty and full particle content, VP3 band volumes for a selected sample (e.g., in examples herein an iodixanol gradient-purified preparation where # of GC = # of particles) are plotted against GC particles loaded. The resulting linear equation (y = mx+c) is used to calculate the number of particles in the band volumes of the test article peaks. The number of particles (pt) per 20 µL loaded is then multiplied by 50 to give particles (pt) /mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles.

Generally, methods for assaying for empty capsids and AAV vector particles with packaged genomes have been known in the art. See, e.g., Grimm et al., Gene Therapy (1999) 6: 1322-1330; Sommer et al., Molec. Ther. (2003) 7: 122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer’s instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods. 2014 Apr;25(2):115-25. doi: 10.1089/hgtb.2013.131. Epub 2014 Feb 14.

In brief, the method for separating rAAVhu68 particles having packaged genomic sequences from genome-deficient AAVhu68 intermediates involves subjecting a suspension comprising recombinant AAVhu68 viral particles and AAVhu689 capsid intermediates to fast performance liquid chromatography, wherein the AAVhu68 viral particles and AAVhu68 intermediates are bound to a strong anion exchange resin equilibrated at a pH of 10.2, and subjected to a salt gradient while monitoring eluate for ultraviolet absorbance at about 260 and about 280. Although less optimal for rAAV9hu68, the pH may be in the range of about 10.0 to 10.4. In this method, the AAVhu68 full capsids are collected from a fraction which is eluted when the ratio of A260/A280 reaches an inflection point. In one example, for the Affinity Chromatography step, the diafiltered product may be applied to a Capture Select™ Poros- AAV2/9 affinity resin (Life Technologies) that efficiently captures the AAV2/hu68 serotype. Under these ionic conditions, a significant percentage of residual cellular DNA and proteins flow through the column, while AAV particles are efficiently captured.

### Compositions and Uses

Provided herein are compositions containing at least one rAAV.hGALC stock (e.g., an rAAVhu68 stock or a mutant rAAV stock) and an optional carrier, excipient and/or preservative. An rAAV stock refers to a plurality of rAAV vectors which are the same, e.g., such as in the amounts described below in the discussion of concentrations and dosage units. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered vector genomes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits x 100 give the approximate molecular mass of the polyoxypropylene core, and the last digit x 10 gives the percentage polyoxyethylene content. In one embodiment Poloxamer 188 is selected. The surfactant may be present in an amount up to about 0.0005% to about 0.001% of the suspension.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery), lung, heart, eye, kidney,), oral, inhalation, intranasal, intrathecal, intratracheal, intraarterial, intraocular, intravenous, intramuscular, subcutaneous, intradermal, and other parental routes of administration. Routes of administration may be combined, if desired.

Dosages of the viral vector depend primarily on factors such as the condition being treated, the age, weight and health of the patient, and may thus vary among patients. For example, a therapeutically effective human dosage of the viral vector is generally in the range of from about 25 to about 1000 microliters to about 100 mL of solution containing concentrations of from about 1 × 10⁹ to 1 × 10¹⁶ genomes virus vector. The dosage is adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 1.0 × 10⁹ GC to about 1.0 × 10¹⁶ GC (to treat an average subject of 70 kg in body weight) including all integers or fractional amounts within the range, and preferably 1.0 × 10¹² GC to 4.0 × 10¹⁴ GC for a human patient. In some embodiments, the compositions are formulated to contain 1.4 × 10¹³ to 4 × 10¹⁴ GC of the replication-defective virus. In some embodiments, the compositions are formulated to contain 4 × 10¹³ to 4 × 10¹⁴ GC of the replication-defective virus. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range. In one embodiment, for human application the dose can range from 1.4×10¹³ to about 4×10¹⁴ GC per dose including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 µL. In one embodiment, the volume is about 50 µL. In another embodiment, the volume is about 75 µL. In another embodiment, the volume is about 100 µL. In another embodiment, the volume is about 125 µL. In another embodiment, the volume is about 150 µL. In another embodiment, the volume is about 175 µL. In yet another embodiment, the volume is about 200 µL. In another embodiment, the volume is about 225 µL. In yet another embodiment, the volume is about 250 µL. In yet another embodiment, the volume is about 275 µL. In yet another embodiment, the volume is about 300 µL. In yet another embodiment, the volume is about 325 µL. In another embodiment, the volume is about 350 µL. In another embodiment, the volume is about 375 µL. In another embodiment, the volume is about 400 µL. In another embodiment, the volume is about 450 µL. In another embodiment, the volume is about 500 µL. In another embodiment, the volume is about 550 µL. In another embodiment, the volume is about 600 µL. In another embodiment, the volume is about 650 µL. In another embodiment, the volume is about 700 µL. In another embodiment, the volume is between about 700 and 1000 µL.

Therapeutically effective intrathecal/intracisternal doses of the rAAV.hGALC range from about 1 × 10¹¹ to 7.0 × 10¹⁴ GC (flat doses) - the equivalent of about 10⁸ to 7 × 10¹¹ GC/g brain mass of the patient. Alternatively, the following therapeutically effective flat doses can be administered to patients of the indicated age group:
Newborns: about 1 × 10¹¹ to about 3 × 10¹⁴ GC;
3 - 9 months: about 6 × 10¹² to about 3 × 10¹⁴ GC;
9 months - 6 years: about 6 × 10¹² to about 3 × 10¹⁴ GC;
3 - 6 years: about 1.2 × 10¹³ to about 6 × 10¹⁴ GC;
6 - 12 years: about 1.2 × 10¹³ to about 6 × 10¹⁴ GC;
12+ years: about 1.4 × 10¹³ to about 7.0 × 10¹⁴ GC;
18+ years (adult): about 1.4 × 10¹³ to about 7.0 × 10¹⁴ GC.

In certain embodiments, the dose may be in the range of about 1 × 10⁹ GC/g brain mass to about 1 × 10¹² GC/g brain mass. In certain embodiments, the dose may be in the range of about 3 × 10¹⁰ GC/g brain mass to about 3 × 10¹¹ GC/g brain mass. In certain embodiments, the dose may be in the range of about 1.70 × 10¹⁰ GC/g brain mass to about 5 × 10¹¹ GC/g brain mass. In certain embodiments, the dose may be in the range of about 5 × 10¹⁰ GC/g brain mass to about 5 × 10¹¹ GC/g brain mass. In certain embodiments, the dose may be in the range of about 5 × 10¹⁰ GC/g brain mass to about 1.85 × 10¹¹ GC/g brain mass. In certain embodiments, the dose may be in the range of about 1.70 × 10¹⁰ GC/g brain mass to about 1.70 × 10¹¹ GC/g brain mass. In certain embodiments, the dose may be in the range of about 5.00 × 10¹⁰ GC/g brain mass to about 1.70 × 10¹¹ GC/g brain mass. In certain embodiments, the dose may be in the range of about 1.70 × 10¹¹ GC/g brain mass to about 5 × 10¹¹ GC/g brain mass. In certain embodiments, the dose may be in the range of about 5 × 10¹⁰ GC/g brain mass to about 10¹² GC/g brain mass. In certain embodiments, the dose may be in the range of about 10¹⁰ GC/g brain mass to about 10¹² GC/g brain mass. In certain embodiments, the dose may be at least 1.70 × 10¹⁰ GC/g brain mass. In certain embodiments, the dose may be at least 5 × 10¹⁰ GC/g brain mass. In certain embodiments, the dose may be at least 1.70 × 10¹¹ GC/g brain mass.

For scaling between infants and adolescent/adults, brain mass is in some instances estimated to be about 600 g to about 800 g for a four to 12 month old; about 800 g to about 1000 g for a nine month to 18 month old, about 1000 g to about 1100 g for an 18 month old to a three year old; 1100 g to about 1300 g an adolescent or adult humans, or about 1300 g for an adult human.

In one embodiment, the viral constructs may be delivered in doses of from at least about least 1×10⁹ GCs to about 1 × 10¹⁵, or about 1 × 10¹¹ to 5 × 10¹³ GC. Suitable volumes for delivery of these doses and concentrations may be determined by one of skill in the art. For example, volumes of about 1 µL to 150 mL may be selected, with the higher volumes being selected for adults. In certain embodiments, the volume delivered is about 2.0 ml, about 2.5 mL, about 3.0 ml, about 3.5 mL, about 4.0 ml, about 4.5 mL, about 5.0 ml, about 5.5 mL, about 6.0 ml, about 6.5 mL, about 7.0 ml, about 7.5 mL, about 8.0 ml, about 8.5 mL, about 9.0 ml, about 9.5 mL, about 10.0 mL, about 10.5 ml, about 11.0 mL, about 11.5 ml, about 12.0 mL, about 12.5 ml, about 13.0 mL, about 13.5 ml, about 14.0 mL, about 14.5 mL, or about 15.0 mL. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In certain embodiments, a patient receives an intrathecal administration in a volume of about 2 mL to about 4 mL, about 3 mL to about 5 mL, about 4 mL to about 6 mL, about 5 mL to about 7 mL, about 6 mL to about 8 mL, about 7 mL to about 9 mL, or about 8 mL to about 10 mL. In still other embodiments, a patient may receive an intrathecal administration in a volume of about 5 mL to about 15 mL, or about 7.5 mL to about 10 mL. In certain embodiments, the volume administered intrathecally is about 5.0 mL. In certain embodiments, the volume administered intrathecally is about 5.6 mL. Other suitable volumes and dosages may be determined. The dosage is adjusted to balance the therapeutic benefit against any side effects and such dosages may vary depending upon the therapeutic application for which the recombinant vector is employed.

The above-described recombinant vectors may be delivered to host cells according to published methods. The rAAV, preferably suspended in a physiologically compatible carrier, may be administered to a human or non-human mammalian patient. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of about 6.8 to about 7.2 may be desired. However, other pHs within the broadest ranges and these subranges may be selected for other routes of delivery.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo.

In one example, the formulation may contain, e.g., buffered saline solution comprising one or more of sodium chloride, sodium bicarbonate, dextrose, magnesium sulfate (e.g., magnesium sulfate ·7H₂O), potassium chloride, calcium chloride (e.g., calcium chloride ·2H₂O), dibasic sodium phosphate, and mixtures thereof, in water. Suitably, for intrathecal delivery, the osmolarity is within a range compatible with cerebrospinal fluid (e.g., about 275 to about 290); see, e.g., emedicine.medscape.com/article/2093316-overview. Optionally, for intrathecal delivery, a commercially available diluent may be used as a suspending agent, or in combination with another suspending agent and other optional excipients. See, e.g., Elliotts B® solution [Lukare Medical].

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard’s buffer. The aqueous solution may further contain Kolliphor® P188, a poloxamer which is commercially available from BASF which was formerly sold under the trade name Lutrol® F68. The aqueous solution may have a pH of 7.2.

In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intrathecal delivery.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna.

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube. In certain embodiments, a composition is delivered using an Ommaya reservoir for CNS-targeted administration.

As used herein, the term Computed Tomography (CT) refers to radiography in which a three-dimensional image of a body structure is constructed by computer from a series of plane cross-sectional images made along an axis.

The rAAV.GALC vectors and compositions provided herein are useful for correcting conditions associated with deficient levels of GALC enzymatic activity. In certain embodiments, the rAAV.GALC vectors and compositions provided herein are useful for treating dysfunction of peripheral nerves caused by deficiencies in GALC, useful in treating respiratory failure and/or motor function loss caused by GALC deficiencies, useful in treating Krabbe disease, and/or useful in treating symptoms associated with Krabbe disease in patients.

In certain embodiments, a composition comprising an effective amount of rAAV.hGALC is administered to a patient who is less than 6 months of age who has early infantile Krabbe disease (EIKD). In certain embodiments, the patient is less than 6 months of age and has GALC enzymatic deficiencies which are less severe than EIKD.

In certain embodiments, a composition comprising an effective amount of rAAV.hGALC is administered to a patient who is older than 6 months of age, e.g., 7 months to about 12 months who has late infantile Krabbe disease (LIKD). In certain embodiments, the patient is older than 6 months, or about 7 months to 12 months of age, and has GALC enzymatic deficiencies which are less severe than LIKD.

In certain embodiments, the patient is over a year old (e.g., from 13 months to 10 years) of age and has juvenile Krabbe disease (JKD). In certain embodiments, the patient is from 13 months to 10 years of age and has GALC enzymatic deficiencies which are less severe than JKD.

In certain embodiments, the patient is over 10 years of age (e.g., from over 10 years to 12 years, or from 10 years to 18 years or older) of age and has adolescent or adult onset Krabbe disease.

In any of the embodiments described above, the rAAV.hGALC therapy provided herein may be administered as a co-therapy with hematopoietic stem cell replacement therapy, bone marrow transplant (BMT), and/or substrate reduction therapy (SRT). In certain embodiments, the rAAV.hGALC therapy (e.g., EIKD) is followed by a co-therapy such as HSCT or BMT, or enzyme replacement therapy. In certain embodiments, the therapy results in rapid enzyme production following administration of the vector, including within 1 week post-treatment.

In certain embodiments, enzyme replacement therapy involves administration of the human GALC protein of SEQ ID NO: 10. In other embodiments, other hGALC protein variants (e.g., such as the canonical sequence identified herein, or an engineered protein), may be used in enzyme replacement therapy. Combinations of different hGALC proteins may be used in enzyme replacement therapy. In such embodiments, the hGALC protein may be produced in vitro using a suitable production system See, e.g., C. Lee et al, 2005/10/01, Enzyme replacement therapy results in substantial improvements in early clinical phenotype in a mouse model of globoid cell leukodystrophy, FASEB journal, The FASEB Journal 19(11): 1549-51, October 2005]. The hGALC proteins may be formulated for delivery (e.g., suspended in a physiologically compatible saline solution) by any suitable route including, but not limited to intravenous, intraperitoneal, or an intrathecal route. Suitable doses may range from 1 mg/kg to 20 mg/kg, or 5 mg/kg to 10 mg/kg and may be readministered once a week, or more or less frequently, as needed (e.g., once every other day, once every two weeks, etc). Using CSF administration of the hAAVhu68.GALC vector, GALC levels in brain and serum can be supraphysiological without toxicity and improved neuromotor function and myelination in CNS and PNS may be observed. When newborn CSF administration is followed by bone marrow transplant in a postnatal conditioned animal model, survival can be extended (e.g., to >300 days) in the absence of overt signs. In a presymptomatic Krabbe patient, a single cisterna magna injection of AAV.cGALC may provide phenotypic correction, survival increase, nerve conduction normalization, and/or improved brain MRI.

In certain embodiments, a combination therapy or regimen is provided involving treating a patient with rAAVhu68.hGALC and BMT. In certain embodiments, the rAAV.hGALC therapy is provided following HSCT or BMT (e.g., LIKD or JKD). However, in certain embodiments, the rAAV.hGALC provides sufficient GALC levels that HSCT or BMT are not required. In certain embodiment, the gene therapy treatment precedes use of BMT. In certain embodiments, this combination therapy provides enhanced transgene expression in skeletal muscle (e.g., quadriceps, lung, diaphragm, heart, liver, kidney), or in certain cells of the peripheral nervous system or central nervous system. In other embodiments, BMT may precede use of the gene therapy treatment described herein.

The goal of treatment is to functionally replace the patient’s defective GALC via rAAV-based CNS- and PNS-directed gene therapy. Efficacy of the therapy for EIKD or LIKD patients can be measured by assessing improvement in one or more of the symptoms of EIKD or LIKD: crying and irritability, spasticity, fisted hands, loss of smiling, poor head control and feeding difficulties; mental and motor deterioration, hyper or hypotonicity, seizures, blindness, deafness, and increased survival (for EIKD, without treatment, death typically occurs before the age of 2; for LIKD, survival may increase to 3-5 years of age). Additionally, for these and other Krabbe patients, efficacy of treatment may be assessed by: a decrease in demyelination and demyelination affecting both peripheral nerves and CNS white matter (deep cerebral white matter and dentate/cerebellar white matter) which can be monitored via imaging (e.g., magnetic resonance imaging (MRI)); a decrease in abnormal nerve conduction velocity (NCV) and/or brainstem auditory evoked potentials (BAEPs); increased levels of GALC may be observed in cerebrospinal fluid and/or plasma; and/or decreased accumulation of psychosine.

A composition comprising a recombinant adeno-associated virus (rAAV) is provided which comprises an AAV capsid which targets cells in the central nervous system and which has packaged therein a vector genome comprising a galactosylceramidase coding sequence encoding a mature galactosylceramidase protein having the amino acid sequence in SEQ ID NO: 10 under the control of regulatory sequences which direct expression of the protein, said vector genome further comprising AAV inverted terminal repeats necessary for packaging the vector genome in an AAV capsid.

In certain embodiments, a composition useful for treating Krabbe disease is provided which comprises rAAVhu68 having a vector genome of CB7.CI.hGALC.rBG. In one embodiment, the vector genome has the coding sequence of (SEQ ID NO: 19).

In certain embodiments, use of a composition is provided in a method for correcting dysfunction of peripheral nerves caused by a GALC deficiency and/or a method for treating respiratory failure and motor function loss caused by a GALC deficiency. In certain embodiments, the method comprises administering a composition comprising a stock of recombinant adeno-associated virus (rAAV) which comprises: (a) an AAV capsid which targets cells in the central nervous system and which has a vector genome of (b) packaged therein; and (b) a vector genome comprising a galactosylceramidase coding sequence encoding a mature galactosylceramidase protein having the amino acid sequence in SEQ ID NO: 10 under the control of regulatory sequences which direct expression of the protein, wherein the vector genome further comprises AAV inverted terminal repeats necessary for packaging the vector genome in an AAV capsid.

In certain embodiments, a rAAV.hGALC composition as provided herein is delivered intrathecally for treatment of a patient with early infantile Krabbe disease. In certain embodiments, a composition as provided herein is delivered intrathecally for treatment of a patient with late infantile Krabbe disease (LIKD). In certain embodiments, a rAAV.hGALC composition as provided herein is delivered intrathecally for treatment of a patient with Juvenile Krabbe disease (JKD). In certain embodiments, rAAV.hGALC composition as provided herein is delivered intrathecally for treatment of a patient with adolescent or adult onset Krabbe disease. In certain embodiments, the rAAV.hGALC composition is administered as a co-therapy to hematopoietic stem cell transplant (HSCT), bone marrow transplant, and/or substrate reduction therapy. In certain embodiments, the rAAV.hGALC composition is administered as a single dose via a computed tomography-(CT-) guided sub-occipital injection into the cisterna magna (intra-cisterna magna).

Administration of rAAV.hGALC stabilizes disease progression as measured by survival, preventing loss of developmental and motor milestone potentially supporting acquisition of new milestones, onset and frequency of seizures. Thus, in certain embodiments, methods for monitoring treatment are provided wherein endpoints are measured at, for example, 30 days, 90 days and/or 6 months, and then, for example, every 6 months during the 2-year short-term follow-up period. In certain embodiments, measurement frequency decreases to once every 12 months during the long-term extension. Given the severity of disease in the target population, subjects may have achieved motor skills by enrollment, developed and subsequently lost other motor milestones, or not yet shown signs of motor milestone development. Assessments therefore track age-at-achievement and age-at-loss for all milestones. In certain embodiments, milestones include, for example, one or more of sitting without support, hand-and-knees crawling, standing with assistance, walking with assistance, standing alone, and/or walking alone. In certain embodiments, treatment results in a delayed onset of seizure activity and/or a decrease in the frequency of seizure events.

In certain embodiments, methods of monitoring treatment in a subject uses clinical scales to quantify the effects of treatment on development and changes in adaptive behaviors, cognition, language, motor function, and/or health-related quality of life. Scales and domains include, for example, the Bayley Scales of Infant and Toddler Development (assesses development of infant and toddlers across five domains: cognitive, language, motor, social-emotional, and adaptive behavior), the Vineland Adaptive Behavior Scales (Edition III) (assesses adaptive behavior from birth through adulthood (0-90 years) across five domains: communication, daily living skills, socialization, motor skills, and maladaptive behavior), the Peabody Developmental Motor Scales- Second Edition (measures interrelated motor function from birth to children five years of age; assessments focus on six domains: reflexes, stationary, locomotion, object manipulation, grasping, and visual-motor integration), the Infant Toddler Quality of Life Questionnaire (ITQOL) (parent-reported measure of health-related quality of life designed for infants 2 months of age up to toddlers 5 years of age), and the Mullen Scales of Early Learning (assesses language, motor, and perceptual abilities in infants and toddlers up to 68 months of age). In certain embodiments, the effects of treatments are monitored or measured by evaluating changes in myelination, functional outcomes related to myelination, and potential disease biomarkers. In certain embodiments, central and peripheral demyelination slow or cease in progression following treatment of a subject. Central demyelination may be tracked by diffusion-tensor magnetic resonance imaging (DT-MRI) anisotropy measurements of white matter regions and fiber tracking of corticospinal motors tracts, changes in which are indicators of disease state and progression. Peripheral demyelination may be measured indirectly via nerve conduction velocity (NCV) studies on the motor nerves (deep peroneal, tibial, and ulnar nerves) and sensory nerves (sural, and median nerves) to monitor for fluctuations indicative of a change in biologically active myelin (i.e., F-wave and distal latencies, amplitude or presence or absence of a response).

In certain embodiments, a method of monitoring treatment following rAAV.hGALC administration is provided wherein the subject is evaluated for a delay in vision loss or absence of vision loss for those subjects that have not developed significant vision loss prior to treatment. Measurement of visual evoked potentials (VEPs) is therefore used to objectively measure responses to visual stimuli as an indicator of central visual impairment or loss. In certain embodiments, the subject is monitored for hearing loss following treatment using, for example, brainstem auditory evoked response (BAER) testing. In certain embodiments, a method of monitoring treatment following rAAV.hGALC administration is provided wherein a subject’s psychosine levels are measured.

It is to be noted that the term “a” or “an” refers to one or more. As such, the terms “a” (or “an”), “one or more”, and “at least one” are used interchangeably herein.

The words “comprise”, “comprises”, “comprising”, “containing”, and “including” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% (±10%) from the reference given, unless otherwise specified.

As used herein, “disease”, “disorder”, and “condition” are used interchangeably, to indicate an abnormal state in a subject.

The term “expression” is used herein in its broadest meaning and comprises the production of RNA or of RNA and protein. With respect to RNA, the term “expression” or “translation” relates in particular to the production of peptides or proteins. Expression may be transient or may be stable.

As used herein, an “expression cassette” refers to a nucleic acid molecule which comprises a coding sequence, promoter, and may include other regulatory sequences therefor. In certain embodiments, a vector genome may contain two or more expression cassettes. In other embodiments, the term “transgene” may be used interchangeably with “expression cassette”. Typically, such an expression cassette for generating a viral vector contains the coding sequence for the gene product described herein flanked by packaging signals of the viral genome and other expression control sequences such as those described herein.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless” - containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

In many instances, rAAV particles are referred to as DNase resistant. However, in addition to this endonuclease (DNase), other endo- and exo- nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a gene to a host cell and protects these packaged genomic sequences from degradation (digestion) during nuclease incubation steps designed to remove contaminating nucleic acids which may be present from the production process.

As used herein, an “effective amount” refers to the amount of the rAAV composition which delivers and expresses in the target cells an amount of the gene product from the vector genome. An effective amount may be determined based on an animal model, rather than a human patient. Examples of a suitable murine model are described herein.

Unless defined otherwise in this specification, technical and scientific terms used herein have the same meaning as commonly understood by one of ordinary skill in the art and by reference to published texts, which provide one skilled in the art with a general guide to many of the terms used in the present application.

### EXAMPLES

The following examples are illustrative only and are not intended to limit the present invention.

| Abbreviation | Description |
|---|---|
| A | Absorbance |
| aa | Amino Acids |
| AAV | Adeno-Associated Virus |
| AAVhu68 | Adeno-Associated Virus Serotype hu68 |
| Ad5 | Adenovirus Serotype 5 |
| AE | Adverse Events |
| AEX | Anion Exchange |
| AmpR | Ampicillin Resistance (gene) |
| ANOVA | Analysis of Variance |
| AUC | Analytical Ultracentrifugation |
| BA | Chicken β-Actin Promoter |
| BAER | Brainstem Auditory Evoked Response |
| BBB | Blood-Brain Barrier |
| BCA | Bicinchoninic Acid |
| BDS | Bulk Drug Substance |
| BMCB | Bacterial Master Cell Bank |
| bp | Base Pairs |
| BRF | Batch Record Form |
| BSA | Bovine Serum Albumin |
| BSC | Biological Safety Cabinet |
| BWCB | Bacterial Working Cell Bank |
| cap | Capsid (gene) |
| CBC | Complete Blood Count |
| CBER | Center for Biologics Evaluation and Research |
| CFR | Code of Federal Regulations |
| CFU | Colony Forming Units |
| CI | Chimeric Intron |
| CMC | Chemistry Manufacturing and Controls |
| CMO | Contract Manufacturing Organization |
| CMV IE | Cytomegalovirus Immediate-Early Enhancer |
| CNS | Central Nervous System |
| COA | Certificate of Analysis |
| CRL | Charles River Laboratories |
| CRO | Contract Research Organization |
| CSF | Cerebrospinal Fluid |
| CT | Computed Tomography |
| CTL | Cytotoxic T Lymphocyte |
| ddPCR | Droplet Digital Polymerase Chain Reaction |
| DLS | Dynamic Light Scattering |
| DMEM | Dulbecco’s Modified Eagle Medium |
| DMF | Drug Master File |
| DNA | Deoxyribonucleic Acid |
| DO | Dissolved Oxygen |
| DP | Drug Product |
| DRG | Dorsal Root Ganglia |
| DS | Drug Substance |
| DSMB | Data and Safety Monitoring Board |
| E1A | Early Region 1A (gene) |
| ECG | Electrocardiogram |
| EDTA | Ethylenediaminetetraacetic Acid |
| ELISA | Enzyme-Linked Immunosorbent Assay |
| ELISpot | Enzyme-Linked Immunospot |
| ERT | Enzyme Replacement Therapy |
| EU | Endotoxin Units |
| F | Female |
| F/U | Follow-Up |
| FBS | Fetal Bovine Serum |
| FDA | Food and Drug Administration |
| FDP | Final Drug Product |
| FFB | Final Formulation Buffer |
| FIH | First-in-Human |
| GALC | Galactosylceramidase (gene, human) |
| Galc | Galactosylceramidase (gene, mouse) |
| GALC | Galactosylceramidase (protein) |
| GC | Genome Copies |
| GLP | Good Laboratory Practice |
| GMP | Good Manufacturing Practice |
| HCDNA | Host Cell Deoxyribonucleic Acid |
| HCP | Host Cell Protein |
| HEK293 | Human Embryonic Kidney 293 |
| ICH | International Conference on Harmonization |
| ICM | Intra-Cisterna Magna |
| ICV | Intracerebroventricular |
| IDS | Iduronate-2-Sulfatase |
| IFN-γ | Interferon Gamma |
| IT | Intrathecally |
| ITFFB | Intrathecal Final Formulation Buffer |
| ITR | Inverted Terminal Repeat |
| IU | Infectious Unit |
| IV | Intravenous |
| KanR | Kanamycin Resistance (gene) |
| LAL | Limulus Amoebocyte Lysate |
| LFTs | Liver Function Tests |
| LOD | Limit of Detection |
| LTFU | Long-Term Follow-Up |
| M | Male |
| MBR | Master Batch Record |
| MCB | Master Cell Bank |
| MED | Minimum Effective Dose |
| MRI | Magnetic Resonance Imaging |
| mRNA | Messenger Ribonucleic Acid |
| MS | Mass Spectrometry |
| MTD | Maximum Tolerated Dose |
| N | Number of Subjects or Animals |
| N/A | Not Applicable |
| NAbs | Neutralizing Antibodies |
| NBS | Newborn Screening |
| NCV | Nerve Conduction Velocity |
| NGS | Next-Generation Sequencing |
| NHP | Non-Human Primate |
| NHS | Natural History Study |
| OL | Open-Label |
| PAS | Periodic Acid-Schiff |
| PBS | Phosphate-Buffered Saline |
| PEI | Polyethylenimine |
| PES | Polyethersulfone |
| PND | Postnatal Day |
| POC | Proof-of-Concept |
| PolyA | Polyadenylation |
| QA | Quality Assurance |
| QC | Quality Control |
| qPCR | Quantitative Polymerase Chain Reaction |
| rAAV | Recombinant Adeno-Associated Virus |
| rcAAV | Replication-Competent Adeno-Associated Virus |
| rBG | Rabbit β-Globin |
| rDNA | Ribosomal Deoxyribonucleic Acid |
| rep | Replicase (gene) |
| RNA | Ribonucleic Acid |
| RPM | Revolutions Per Minute |
| SA | Single Arm |
| SAE | Serious Adverse Events |
| SD | Standard Deviation |
| SDS | Sodium Dodecyl Sulfate |
| SDS-PAGE | Sodium Dodecyl Sulfate Polyacrylamide Gel Electrophoresis |
| SOP | Standard Operating Procedure |
| SRT | Safety Review Trigger |
| ssDNA | Single-Stranded Deoxyribonucleic Acid |
| TBD | To Be Determined |
| TCID₅₀ | 50% Tissue Culture Infective Dose |
| TE | Tris-EDTA |
| TFF | Tangential Flow Filtration |
| twi | Twitcher Loss-of-Function Allele |
| UPLC | Ultra-Performance Liquid Chromatography |
| US | United States |
| USP | United States Pharmacopeia |
| VEP | Visual Evoked Potential |
| VP1 | Viral Protein 1 |
| VP2 | Viral Protein 2 |
| VUS | Variants of Unknown Significance |
| WCB | Working Cell Bank |
| WHO | World Health Organization |
| WT | Wild Type |

### Example 1 - Recombinant AAVhu68.hGALC

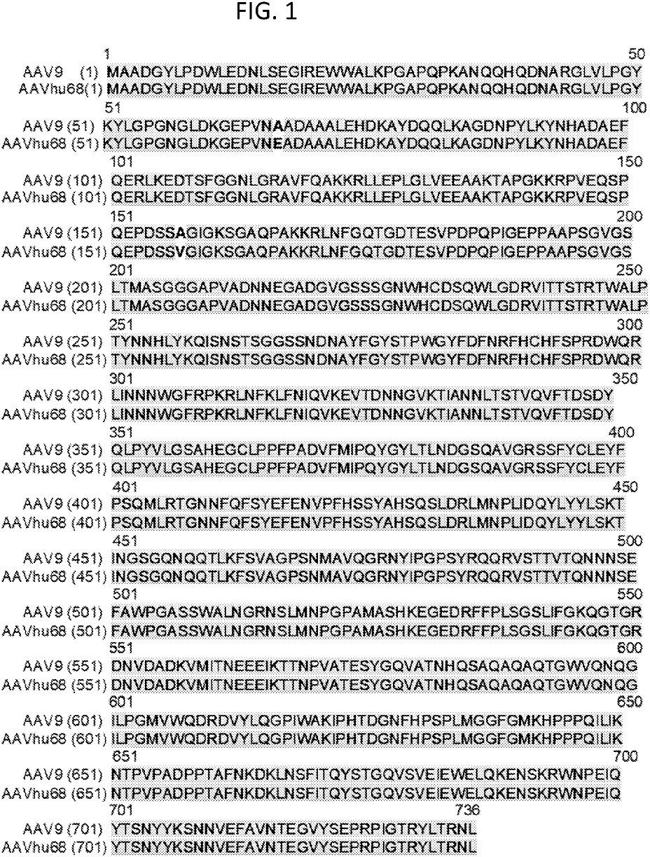

rAAVhu68.hGALC is an AAV that carries an engineered sequence encoding a human GALC. The AAVhu68 capsid of rAAVhu68.hGALC is 99% identical at the amino acid level to AAV9. The two amino acids that differ between the AAV9 [SEQ ID NO: 4] and AAVhu68 capsids [SEQ ID NO: 2] are located in the VP1 (67 and 157) and VP2 (157) regions of the capsid and are identifies in FIG. 1. See also WO 2018/160852, which is incorporated herein by reference.

rAAVhu68.hGALC is produced by triple plasmid transfection of HEK293 cells with an AAV cis plasmid encoding the transgene cassette flanked by AAV ITRs, the AAV trans plasmid encoding the AAV2 rep and AAVhu68 cap genes (pAAV2/hu68.KanR), and the helper adenovirus plasmid (pAdΔF6.KanR).

### A. AAV Vector Genome Plasmid Sequence Elements

A linear map of the vector genome is shown in FIG. 2. The vector genome contains the following sequence elements:

Inverted Terminal Repeat (ITR): The ITRs are identical, reverse complementary sequences derived from AAV2 (130 bp, GenBank: NC_001401) that flank all components of the vector genome. The ITRs function as both the origin of vector DNA replication and the packaging signal for the vector genome when AAV and adenovirus helper functions are provided in trans. As such, the ITR sequences represent the only cis sequences required for vector genome replication and packaging.

Human Cytomegalovirus Immediate-Early Enhancer (CMV IE): This enhancer sequence obtained from human-derived CMV (382 bp, GenBank: K03104.1) increases expression of downstream transgenes.

Chicken β-Actin (CB) Promoter: This ubiquitous promoter (282 bp, GenBank: X00182.1) was selected to drive transgene expression in any CNS cell type.

Chimeric Intron (CI): The hybrid intron consists of a chicken β-actin splice donor (973 bp, GenBank: X00182.1) and rabbit β-globin splice acceptor element. The intron is transcribed, but removed from the mature mRNA by splicing, bringing together the sequences on either side of it. The presence of an intron in an expression cassette has been shown to facilitate the transport of mRNA from the nucleus to the cytoplasm, thus enhancing the accumulation of the steady level of mRNA for translation. This is a common feature in gene vectors intended for increased levels of gene expression.

Coding sequence: An engineered cDNA of the human GALC gene encodes human galactosylceramidase protein, which is a lysosomal enzyme responsible for the hydrolysis and degradation of myelin galactolipids (2055 bp; 685 amino acids [aa], GenBank: EAW81361.1).

Rabbit β-Globin Polyadenylation Signal (rBG PolyA): The rBG PolyA signal (127 bp, GenBank: V00882.1) facilitates efficient polyadenylation of the transgene mRNA in cis. This element functions as a signal for transcriptional termination, a specific cleavage event at the 3′ end of the nascent transcript and the addition of a long polyadenyl tail.

### B. Trans Plasmid: pAAV2/1.KanR (p0068)

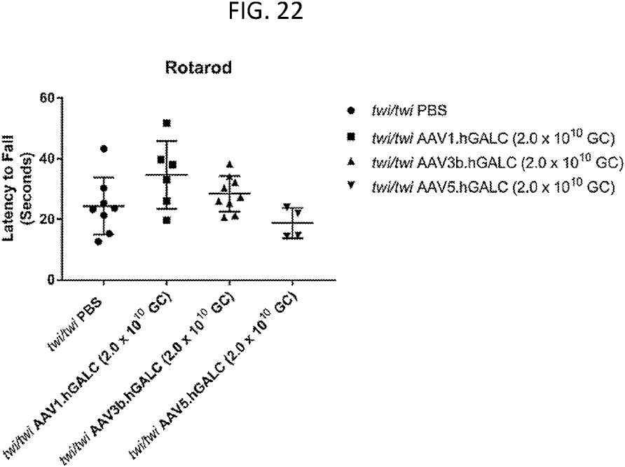

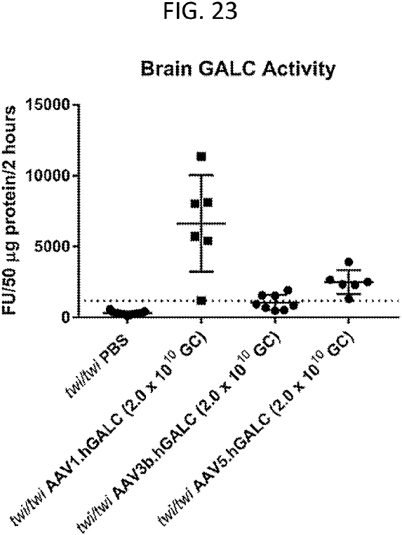

The AAV2/hu68 trans plasmid pAAV2/hu68.KanR (p0068) is presented in FIG. 21.

The AAV2/hu68 trans plasmid is pAAV2/hu68.KanR (p0068). The pAAV2/hu68.KanR plasmid is 8030 bp in length and encodes four wild type AAV2 replicase (Rep) proteins required for the replication and packaging of the AAV vector genome. The pAAV2/hu68.KanR plasmid also encodes three WT AAVhu68 virion protein capsid (Cap) proteins, which assemble into a virion shell of the AAV serotype hu68 to house the AAV vector genome.

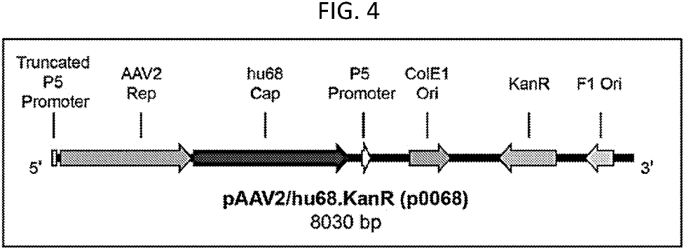

To create the pAAV2/hu68.KanR trans plasmid, the AAV9 cap gene from plasmid pAAV2/9n (p0061-2) (which encodes the wild type AAV2 rep and AAV9 cap genes on a plasmid backbone derived from the pBluescript KS vector) was removed and replaced with the AAVhu68 cap gene. The ampicillin resistance (AmpR) gene was also replaced with the kanamycin resistance (KanR) gene, yielding pAAV2/hu68.KanR (p0068). This cloning strategy relocated the AAV p5 promoter sequence (which normally drives rep expression) from the 5′ end of rep to the 3′ end of cap, leaving behind a truncated p5 promoter upstream of rep. This truncated promoter serves to down-regulate expression of rep and, consequently, maximize vector production (FIG. 4).

All component parts of the plasmid have been verified by direct sequencing.

### C. Adenovirus Helper Plasmid: pAdDeltaF6(KanR)

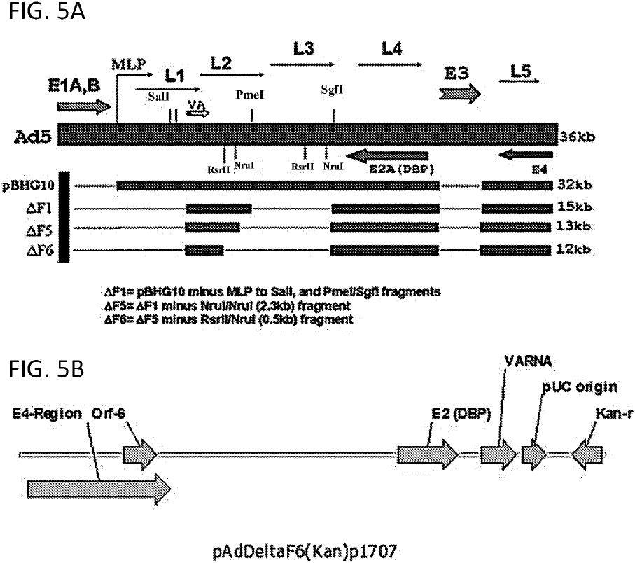

The adenovirus helper plasmid pAdDeltaF6(KanR) is presented in (FIG. 5B) Plasmid pAdDeltaF6(KanR) is 15,770 bp in size. The plasmid contains the regions of adenovirus genome that are important for AAV replication; namely, E2A, E4, and VA RNA (the adenovirus E1 functions are provided by the HEK293 cells). However, the plasmid does not contain other adenovirus replication or structural genes. The plasmid does not contain the cis elements critical for replication, such as the adenoviral ITRs; therefore, no infectious adenovirus is expected to be generated. The plasmid was derived from an E1, E3-deleted molecular clone of Ad5 (pBHG10, a pBR322-based plasmid). Deletions were introduced into Ad5 to eliminate expression of unnecessary adenovirus genes and reduce the amount of adenovirus DNA from 32 kb to 12 kb (FIG. 5A). Finally, the ampicillin resistance gene was replaced by the kanamycin resistance gene to create pAdeltaF6(KanR) (FIG. 5B). The E2, E4, and VAI adenoviral genes that remain in this plasmid, along with E1, which is present in HEK293 cells, are necessary for AAV vector production.

### Example 2 - Material and Methods

### Neuromotor Function Assessment (RotaRod)

Coordination and balance were measured using the RotaRod test (Ugo Basile; Gemonio, Italy) by personnel blinded to the treatment group. Briefly, mice were first habituated to the RotaRod by placing up to five mice per trial in a lane of the RotaRod device facing the wall. Mice were allowed to stabilize themselves on the fixed (non-rotating) rod for 2 minutes. Two habituation trials were then performed with the rod rotating for 1 minute at a constant speed of 5 revolutions per minute (RPM). Between each habituation trial, mice were allowed to rest in the RotaRod collecting box for approximately 1 minute. If a mouse fell during the habituation phase, it was immediately placed back on the rod.

Immediately following habituation, testing trials were performed to measure how long each mouse could remain on the rotating rod while it was accelerating. The mice were placed in a lane of the RotaRod device facing the wall and allowed to equilibrate on the fixed (non-rotating) rod to establish a firm grip. The rod was then set to spin at a constant speed of 5 RPM for a few seconds to allow the mice to equilibrate. Once equilibrated, the rod was set to accelerate from 5 RPM to 40 RPM over 120 seconds. For each animal, the testing trial was considered terminated when the mouse fell off the rod, completed two passive revolutions, or 120 seconds had elapsed. The fall latency (defined as the time between the initiation of rod acceleration and trial termination) was recorded. A total of three sequential test replicates were performed for the mice in each trial, with a 1-3 minute pause in between runs to allow the animals to rest in the collecting box.,

### Histological Processing and Evaluation

### Brain

The whole brain was removed from the skull of necropsied animals and cut through the mid-sagittal into two pieces by using a razor blade. The left hemisphere of the brain frozen on dry ice and stored at ≤-60° C. for evaluation of transgene expression (GALC enzyme activity assay) and the right hemisphere of the brain was placed in 10% NBF for histology.

### Sciatic Nerve

The sciatic nerves were collected and immediately placed in fixed in 2.5% glutaraldehyde + 2% paraformaldehyde in PBS for a minimum of approximately 24 hours at room temperature. After fixation the samples were washed in PBS, postfixed with 1% aqueous osmium tetroxide for 2 hours, washed in water, and dehydrated through an ascending ethanol series followed by propylene oxide, mixtures of propylene oxide and embedding resin, and embedding resin alone. The embedding resin was prepared by mixing its components (LX-122, DDSA, NMA, and DMP-30) before use according to the manufacturer’s instructions (Ladd Research Industries). Samples were then placed into molds with embedding medium and cured at 70° C. for 48 hours, sectioned at 1 µm thickness with an ultramicrotome, stained with Toluidine Blue, and after drying coverslipped with Permount mounting medium.

### Paraffin Embedding and Sectioning

Tissue samples were embedded in paraffin and sectioned according to SOP 4004 and SOP 4006. Briefly, cassettes containing fixed tissues were placed into a Leica ASP300 tissue processor, and the processor was allowed to run overnight. Tissues were embedded in the appropriate orientation using an embedding center, and samples were hardened on a cooling plate. Blocks were then trimmed and cooled on ice for at least 10 minutes before sectioning. Sections were prepared using a microtome at a thickness of 5-7 µm. Sections were transferred and floated in a water bath preheated to 38-52° C. to remove wrinkles. Sections were transferred onto slides and were dried at room temperature. Slides were then deparaffinized by heating for at least 15 minutes (or until paraffin melted) at a temperature of 60-65° C. Slides were incubated in xylene (3 times, 3-5 minutes), rehydrated through an ethanol series, and washed twice in distilled water. Slides containing a minimum of three serial sections were then stained LFB, PAS (myelination and globoid cell infiltration, respectively, or IBA1 IHC (globoid cell size measurements).

### LFB/PAS Staining (Evaluating Myelination and Globoid Cell Infiltration)

Following deparaffinization, sections of the brain, spinal cord, and sciatic nerve were stained with LFB/PAS staining. Briefly, slides were incubated with LFB solution (SLMP, LLC; Catalog number: STLFBPT) overnight at 65oC. Sections were differentiated in a 0.05% lithium carbonate solution and 70% ethanol, and monitored under the microscope until differentiation was completed. Slides were then placed in 0.5% periodic acid for 5 minutes (Sigma; Catalog number: 395B-1Kit). After washing with running tap water, the slides were transferred into Schiff’s reagent (Sigma; Catalog number: 395B-1Kit) for 15 minutes. Slides were washed with running tap water for 5 minutes, counterstained briefly with hematoxylin for nuclei identification, and coverslipped. Histopathological evaluation was performed.

### IBA1 Immunohistochemistry (Neuroinflammation)

Following deparaffinization, immunohistochemical staining for IBA1 was performed on sections of the brain, spinal cord, and sciatic nerve. Briefly, antigen retrieval was performed in a pressure cooker at 100oC for 20 minutes using a citric acid-based antigen unmasking solution (Vector Laboratories; Catalog number: H-3300). Slides were incubated with 3% hydrogen peroxide for 10 minutes, blocked using avidin/biotin reagents for 15 minutes each (Vector Laboratory; Catalog number: SP-2001), and incubated with 1% donkey serum with 0.2% Triton-X for 15 minutes at room temperature. Slides were then incubated with a rabbit anti-IBA1 primary antibody (Abcam; Catalog number: ab178846) diluted 1:2000 at 4oC overnight. Slides were incubated with a biotinylated donkey anti-rabbit IgG secondary antibody (Jackson; Catalog number: 711-065-152) at a dilution of 1:1000 for 30 minutes at room temperature. Slides were washed and then incubated with Vectastain ABC reagent (Vector Laboratories; Catalog number: PK-6100). Colorimetric development was performed using a DAB kit (Vector Laboratories; Catalog number: SK-4100) followed by counterstaining with hematoxylin and coverslipping.

### GALC Immunohistochemistry

Tissue samples were fixed in formalin for a minimum of 24 hour and paraffin embedded. Sections (6 µm) were then deparaffinized through a xylene and ethanol series and brought to water. Antigen retrieval was performed in a citrate buffer-based (pH 6.0) antigen unmasking solution (Vector Laboratories) using a pressure cooker. Next, sections were sequentially treated with 2% H₂O₂ (15 minutes), avidin/biotin blocking reagents (15 minutes each; Vector Laboratories), and blocking buffer (1% donkey serum in PBS + 0.2% Triton, 1 hour). Sections were then incubated with primary antibody (rabbit anti-human GALC, ThermoFisher PA5-72315 at 1:100, overnight at 4° C.) and, after washing in PBS/0.02% Tween-20, biotinylated secondary antibody from donkey (30 minutes; Jackson ImmunoResearch, 1:500). All antibodies were diluted in blocking buffer. After washing, a Vectastain Elite ABC kit (Vector Laboratories) was used according to the manufacturer’s instructions with DAB as substrate (5 minute development time) to stain bound antibodies as brown precipitate. Sections were counterstained with hematoxylin to show nuclei and coverslipped.

### Evaluating Transgene Expression (GALC Activity Assay)

### Processing of Tissue Samples

Frozen brains were homogenized in 0.9% NaCl, pH 4.0 containing 0.05% Triton-X100 using a Qiagen TissueLyzer for 2 minutes and 30 seconds at 30 Hz. Samples were frozen on dry ice for 20 minutes, thawed at room temperature, and briefly vortexed. Lysates were clarified by centrifugation for 10 minutes at 10,000 RPM in a tabletop centrifuge. The clarified lysate was transferred to a new tube for analysis. Protein content was measured by a bicinchoninic acid (BCA) assay.

### Measuring GALC Enzyme Activity

GALC activity was measured in brain and serum using a commercial kit from Marker Gene Technologies, Inc. (Catalog Number: M2774). For this assay 50 µg of total protein from the brain or 10 µL of serum were mixed with the reaction buffer included in the kit to a final volume of 100 µL. A tube containing 100 µL of reaction buffer served as a blank. Samples were incubated at 37° C. for 2 hours and the reaction was stopped by adding 1 mL of the stop solution supplied in kit. Finally, 300 µL of each reaction were transferred to a 96-well black plate, and fluorescence was measured using a plate reader at an emission wavelength of 454 nm upon excitation at 365 nm.

### Example 3 - AAVhu68.hGALC Delivery in the GALC-Deficient Twitcher Mouse Model

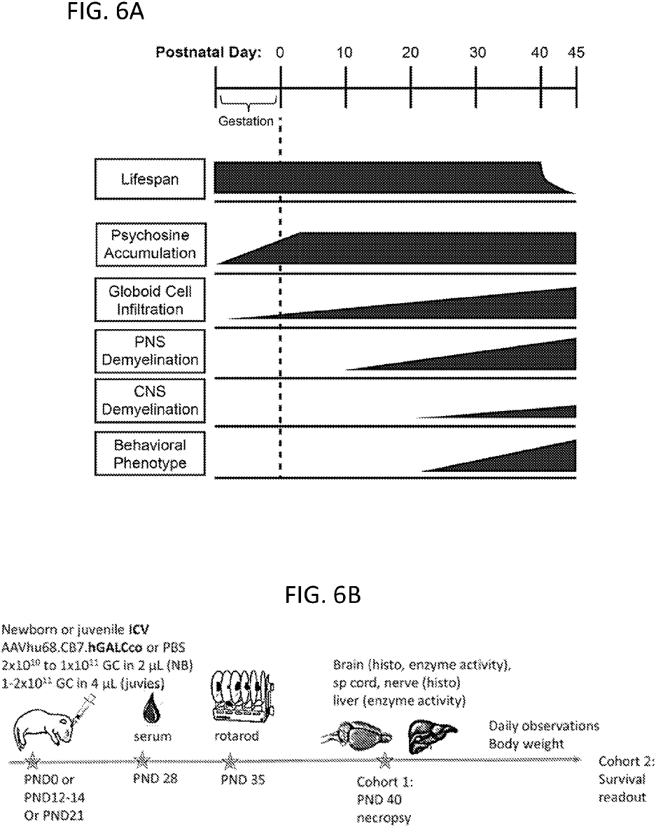

The studies described below used the Twitcher mouse model to establish the potential for delivery AAVhu68.CB7.CI.GALC.rBG (AAVhu68 vector containing an engineered human GALC cDNA (SEQ ID NO: 9) under the control of a CB7 promoter and a BG polyA sequence flanked by AAV2 ITRs, also referred to as rAAVhu68.hGALC) to achieve therapeutic levels of GALC expression levels and rescue several biomarkers of the disease. An overview of the Twitcher mouse studies is provided in FIG. 6B.

The Twitcher mouse is a naturally occurring inbred model of Krabbe disease that was identified as a spontaneous mutation at the Jackson Laboratory in 1976 (Kobayashi T., et al. (1980) Brain Research. 202(2):479-483). Affected mice are homozygous for the twitcher loss-of-function allele (twi), which consists of a G to A mutation in the Galcgene. This mutation causes an early stop codon (W339X). The truncated GALC protein has residual enzymatic activity close to 0%, which is similar to GALC activity levels observed in patients with the infantile form of Krabbe disease. Heterozygous carrier mice (twi/+) are phenotypically normal.

The progression of disease in the Twitcher mouse is well-documented (FIG. 6A), and a variety of neuropathological and behavioral defects phenocopy infantile Krabbe disease. As in infantile Krabbe patients, GALC deficiency in mice results in the accumulation of the cytotoxic lipid intermediate, psychosine. Twitcher mice likewise display massive infiltration of PNS and CNS white matter by phagocytic, psychosine-filled globoid cells, which are thought to be derived from macrophage and/or microglial lineages (Tanaka K., et al. (1988) Brain Research. 454(1):340-346; Levine S.M., et al. (1994) Intl J Dev Neuro. 12(4):275-288). This results in demyelination, which is one of the key hallmarks of disease in Krabbe patient. After an initial period of normal myelination, affected Twitcher mice lose myelin after 10 days of age in the PNS due to the death of myelin-forming Schwann cells (Jacobs J.M., et al. (1982) J Neurol Sci. 55(3):285-304) and 20 days of age in the CNS due to the death of myelin-forming oligodendrocytes. It is likely because of this delay that demyelination is more severe in the peripheral nerves than in the CNS of these mice (Suzuki K. & Suzuki K. (1983) The American journal of pathology. 111(3):394-397). Finally, Twitcher mice display consistent and rapid neurological deterioration after the onset of symptoms, which is similarly observed in infantile Krabbe patients upon symptom onset. Behavioral symptoms in these mice include motor phenotypes reminiscent of those observed in human patients, including tremors, twitching, and hind leg weakness, which present at approximately 20 days of age. Mice ultimately progress to a humane endpoint characterized by severe weight loss and paralysis by around 40 days of age (Wenger D.A. (2000) Molec Med Today. 6(11):449-451). We selected the humane endpoint as the end of the study to assess efficacy of the treatment to rescue survival in mice. At the humane endpoint, CNS and PNS were collected for histopathology to observe the demyelination and globoid cells infiltration which are hallmarks of Krabbe disease in mice and humans, hGALC expression, and transgene expression (GALC enzyme activity).

Infantile Krabbe patients exhibit similar clinical features as the Twitcher mice. Thus, the Twitcher mouse model is adequate to assess the efficacy (rescue of enzyme activity to improve survival, motor function, and brain and nerve pathology) of rAAVhu68.hGALC to support an infantile Krabbe indication. Studies using the Twitcher mouse, described below, demonstrated the efficacy of rAAVhu68.hGALC to express active GALC enzyme in the relevant tissues, rescue survival, ameliorate motor function, and ameliorate CNS and PNS histopathology after a single ICV administration (the most efficient route of administration in mouse models where ICM is not technically feasible).

Newborn Twitcher (twi/twi) mice (PND 0) received a single IV administration of rAAVhu68.hGALC at a dose of 1.0 × 10¹¹GC (6.7 × 10¹¹ GC/g brain weight) or phosphate-buffered saline (PBS). Wild type, heterozygous (twi/+), and homozygous (twi/twi) mice (PND 0) were administered PBS IV and served as comparators (see table below). In-life assessments included viability checks performed daily and monitoring for survival. Necropsies were performed on animals at a humane endpoint. Brains were collected at necropsy for evaluation of transgene expression (GALC enzyme activity).

| Group | N | Sex | Genotype^{a} | Treatment | Dose (GC/Animal)^{b} | Dose (GC/g Brain)^{c} | Dose Volume (µL) |
|---|---|---|---|---|---|---|---|
| 1 | 12 | both | +/+ and twi/+ | PBS | N/A | N/A | 50.0 |
| 2 | 8 | both | twi/twi | PBS | N/A | N/A | 50.0 |
| 3 | 6 | both | twi/twi | AAVhu68.CB7. hGALCco.rBG | 1.0 × 10¹¹ | 6.7 × 10¹¹ | 50.0 |

^{a}+/+, wild type mice; twi/+, heterozygous genotype; twi/twi, knockout mice; The twi allele consist of a loss-of-function mutation in the Galc gene.
^{b}1.0 × 10¹⁴ GC/kg body weight
^{c}Values were calculated using a brain mass of 0.15 g for a newborn mouse

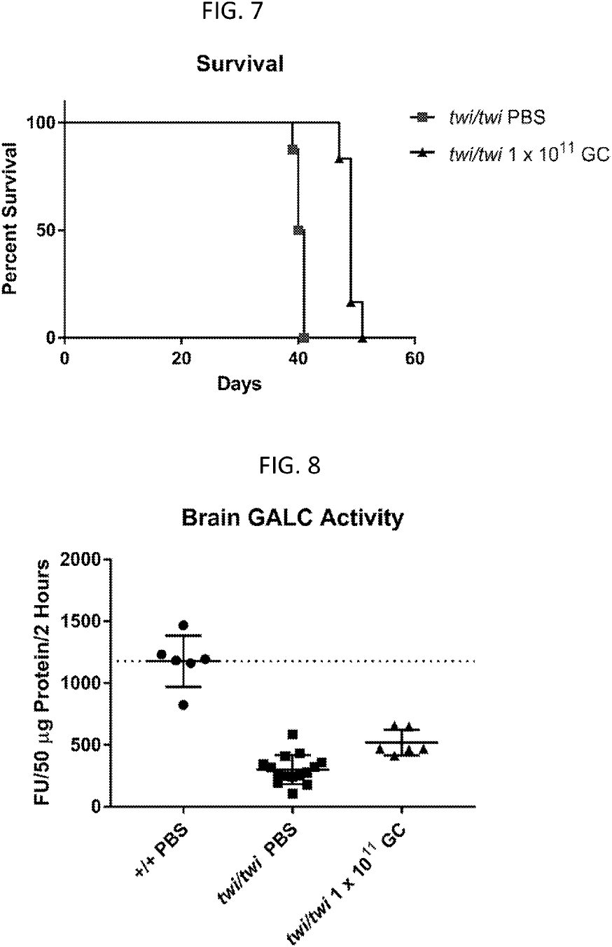

A minimal increase of brain GALC activity was observed in rAAVhu68.hGALC twi/twi treated mice. All twi/twi animals were euthanized upon reaching humane endpoint. Three twi/twi animals were found dead and included in the survival analysis. Intravenous administration of 1 × 10¹¹ GC of rAAVhu68.hGALC led to a small statistically significant increase in survival from 40.5 days median survival in twi/twi PBS mice to 49 days median survival in twi/twi mice (FIG. 7). Intravenous administration of rAAVhu68.hGALC led to minimal increase of brain GALC activity that remained below wild type levels. Mean GALC activity was 44% of wild type levels in mice that received rAAVhu68.hGALC (FIG. 8).

Next, studies were performed to determine the efficacy of rAAVhu68.hGALC (AAVhu68.CB7.hGALCco.rBG) in the Twitcher mouse model following intracerebroventricular (ICV) administration. Presymptomatic Twitcher (twi/twi) mice were administered rAAVhu68.hGALC ICV because direct administration into the CSF is known to facilitate CNS transduction at lower doses. Twitcher (twi/twi) mice (PND 0) received a single ICV administration of rAAVhu68.hGALC at one of three dose levels (2.0 × 10¹⁰ GC, 5.0 × 10¹⁰ GC, or 1.0 × 10¹¹GC) or phosphate-buffered saline (PBS). Wild type, heterozygous (twi/+), and homozygous (twi/twi) mice (PND 0) were administered PBS ICV and served as comparators. In-life assessments included viability checks, body weight monitoring, neuromotor assessments (RotaRod), and monitoring for survival. Necropsies were performed on animals at a humane endpoint. At humane endpoint, CNS and PNS were collected for histopathology to observe the demyelination and globoid cells infiltration, hGALC expression, and transgene expression (GALC enzyme activity).

| Group a | N | Sex | Genotype | Treatment | Dose (GC/Animal) | Dose (GC/g Brain)^{a} | Dose Volume (µL) | ROA | Dosin g Day | Necropsy Day |
|---|---|---|---|---|---|---|---|---|---|---|
| 1 | 15 | both | +/+ and twi/+ | PBS | N/A | N/A | 2.0 | ICV | 0 | 21 |
| 2 | 8 | both | twi/twi | PBS | N/A | N/A | 2.0 | ICV | 0 | Humane endpoint^{b} |
| 3 | 10 | both | twi/twi | AAVhu68.CB 7.hGALCco.r BG | 2.0 × 10¹⁰ | 1.3 × 10¹¹ | 2.0 | ICV | 0 | Humane endpoint |
| 4 | 12 | both | twi/twi | AAVhu68.CB 7.hGALCco.r BG | 5.0 × 10¹⁰ | 3.3 × 10¹¹ | 2.0 | ICV | 0 | Humane endpoint |
| 5 | 12 | both | twi/twi | AAVhu68.CB 7.hGALCco.r BG | 1.0 × 10¹¹ | 6.7 × 10¹¹ | 2 × 2.0 | ICV bilatera l | 0 | Humane endpoint |

^{a}Values were calculated using a brain mass of 0.15 g for a newborn mouse
^{b} PBS-treated wild type and heterozygous mice were used for rotarod and body weight controls and did not reach humane endpoint. They were euthanized at study completion.

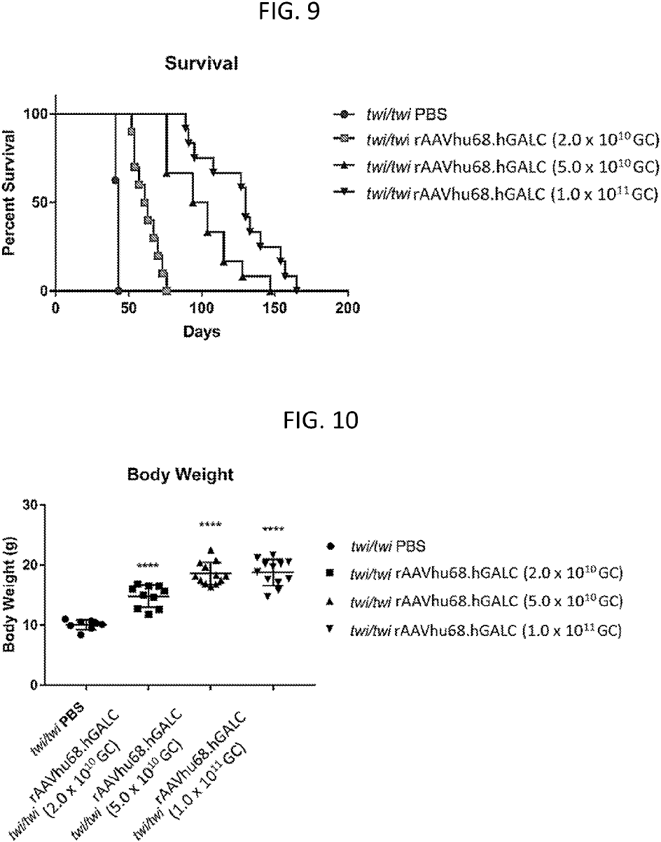

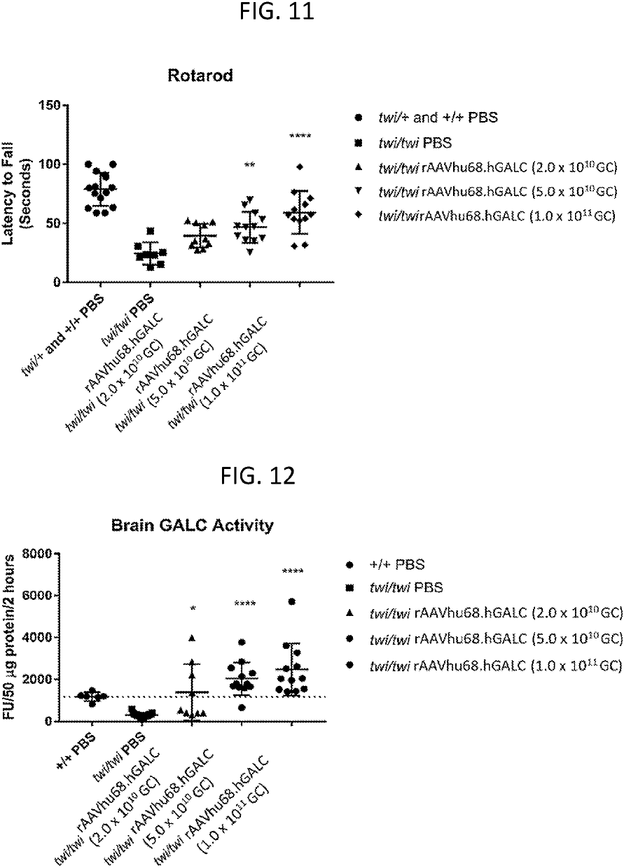

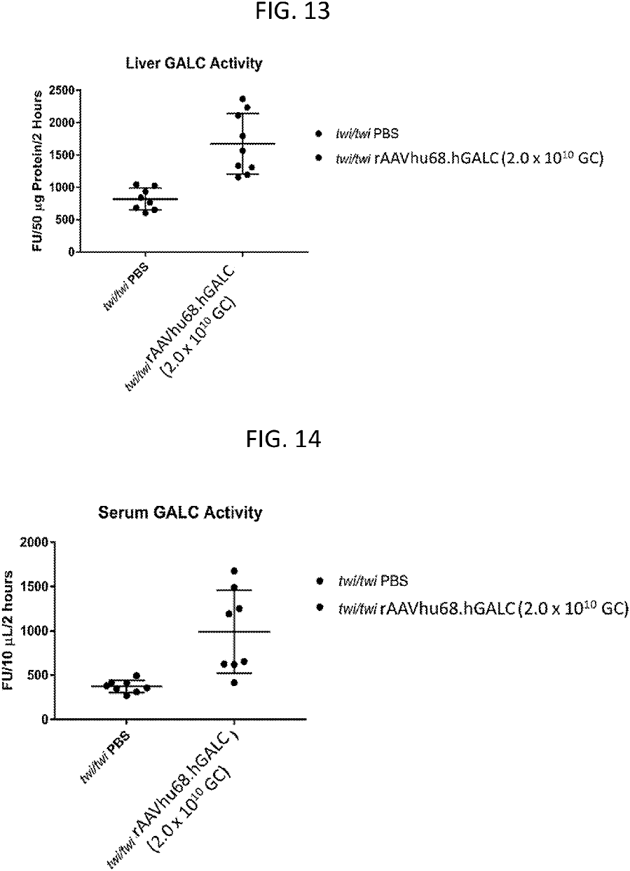

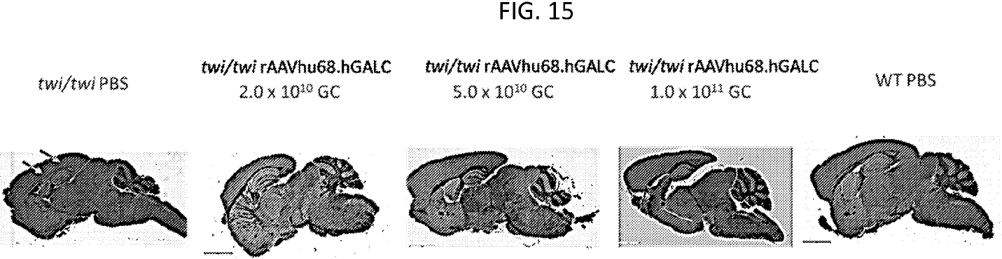

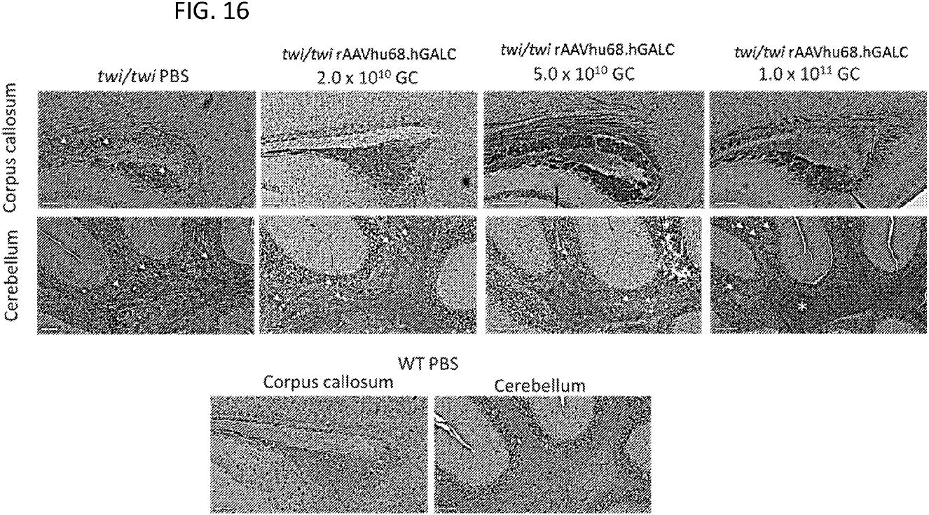

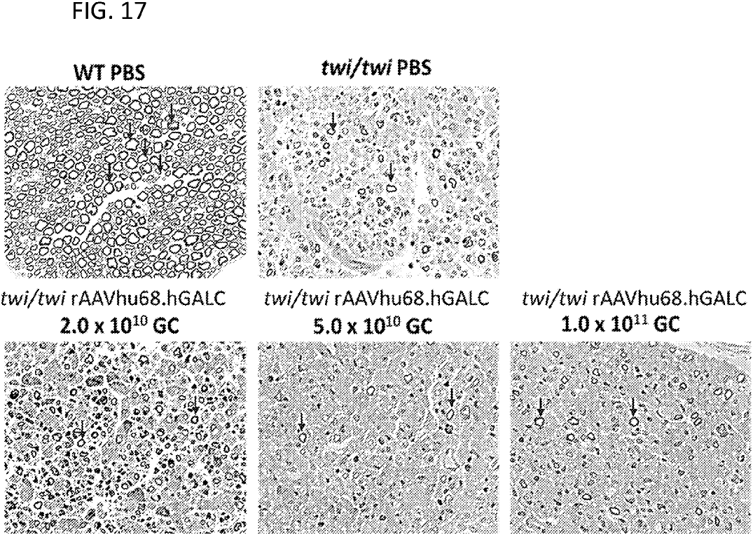

rAAVhu68.hGALC led to a statistically significant dose-dependent increase in survival from 43 days median survival in PBS-treated twi/twi mice to 62 days at a dose of 2 × 10¹⁰ GC, 99 days at a dose of 5 × 10¹⁰ GC, and 130 days at a dose of 1 × 10¹¹ GC (FIG. 9). Administration of rAAVhu68.hGALC lead to a statistically significant rescue of body weight loss in twi/twi mice when compared to the body weights of the PBS-treated twi/twi mice at all dose levels. Body weights in mice give rAAVhu68.hGALC were the same in the two highest dose groups (5.0 × 10¹⁰ GC and 1.0 × 10¹¹ GC) (FIG. 10). Neuromotor function was assessed by the RotaRod test, which evaluates coordination and balance by measuring the time to fall for mice running on a spinning rod that progressively accelerates. A decrease in fall latency indicates neuromotor impairment, while an increase in fall latency indicates improved neuromotor function.rAAVhu68.hGALC administered ICV to twi/twi mice led to dose-dependent increase in fall latency compared to PBS-treated twi/twi mice. The increase in fall latency was statistically significant at the two highest doses (5.0 × 10¹⁰ and 1.0 × 10¹¹ GC) (FIG. 11). Brain GALC enzyme activity was increased in a dose-dependent manner compared to wild type PBS-treated mice. GALC activity in the rAAVhu68.hGALC dosed groups was 117% in the 2.0 × 10¹⁰ GC group, 173% in the 5.0 × 10¹⁰ GC group, and 210% in the 1.0 × 10¹¹ GC group compared the wild type PBS-treated group. When compared to PBS-treated twi/twi mice, rAAVhu68.hGALC -treated mice also had increased GALC activity (FIG. 12).

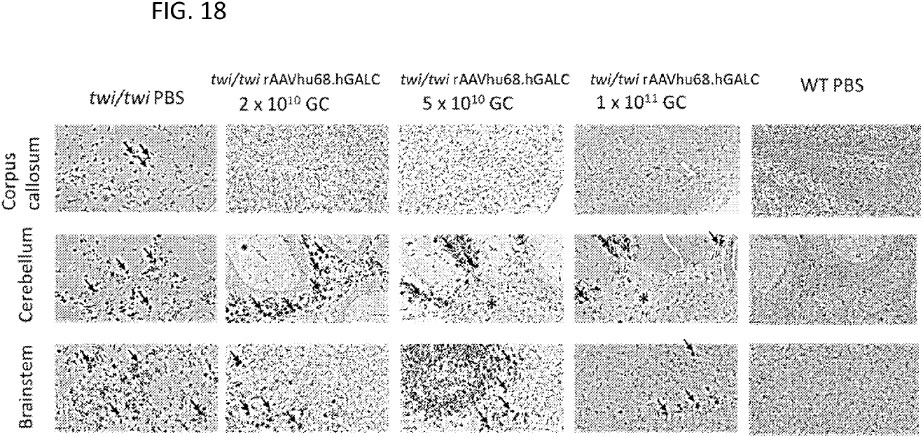

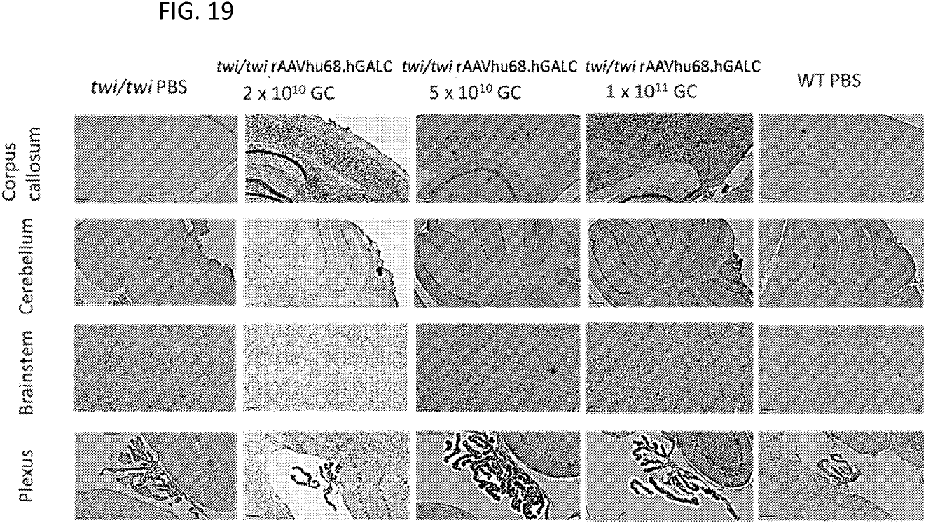

The brain of twi/twi PBS-treated animals showed minimal to moderate brain demyelination and all treatment groups show normal WT-like corpus callosum myelin intensity (FIG. 15). Decreased myelin staining and infiltration were observed in white matter of the corpus callosum and cerebellum of twi/twi PBS-treated mice. Treatment at all dose levels reduced demyelination and suppressed globoid cells in the corpus callosum (FIG. 16). rAAVhu68.hGALC was less effective in the cerebellum where demyelination and globoid cell infiltration was present in all rAAVhu68.hGALC-treated groups. Similar demyelination and globoid cell infiltration was observed in the PBS-treated twi/twi animals and in rAAVhu68.hGALC -treated animals at doses of 2.0 × 10¹⁰ GC and 5.0 × 10¹⁰ GC groups. The most central white matter of cerebellar folia appeared normal in most at a rAAVhu68.hGALC dose of 1.0 × 10¹¹ GC treated animals with dark blue myelin staining and no globoid cells while the tip of the folia contained abundant globoid cells (FIG. 16). Wild type mice displayed abundant myelinated nerve fibers that are tightly packed and no inflammatory cells while twi/twi mice displayed a decreased number of myelinated nerve fibers and numerous enlarged mononuclear cells (globoid cells) seen between nerve fibers (FIG. 17). Sciatic nerves from all rAAVhu68.hGALC -treated twi/twi mice showed similar pathology to PBS-treated twi/twi group with severe demyelination and globoid cell infiltration (FIG. 17). This finding is likely due to the fact that the humane endpoint was defined as hindleg paralysis which would occur when sciatic nerve is severely affected.

IBA1-positive cells were normal in PBS-treated wild type mice and all rAAVhu68.hGALC-treated mice in the corpus callosum and cortex. Neuroinflammation persisted in the cerebellum and brainstem where enlarged globoid cells (FIG. 18). Partial treatment effect was observed in the cerebellum as evidenced by fewer and smaller IBA1-positive cells in animals administered rAAVhu68.hGALC at doses of 5.0 × 10¹⁰ GC and 1.0 × 10¹¹ GC. A dose-dependent expression of hGALC in neurons of the cerebral cortex, hippocampus, cerebellum, and in cells of the choroid plexus while brainstem was not transduced (FIG. 19).

### Example 4 - Evaluation of AAV1.CB7.CI.hGALCco.rBG, AAV3B.CB7.CI.hGALCco.rBG, and AAV5.CB7.CI.hGALCco.rBG in Newborn Twitcher (Twi/Twi) Mice Following Intracerebroventricular Administration

Studies were performed to evaluate three additional clinical vectors (AAV1.CB7.CI.hGALCco.rBG, AAV3B.CB7.CI.hGALCco.rBG, AAV5.CB7.CI.hGALCco.rBG) to determine efficacy in a mouse model of infantile Krabbe disease following intracerebrovascular (ICV) administration. The potential candidates had different serotypes, but all contained an engineered human GALC cDNA under the control of a CB7 promoter and an BG polyA sequence flanked by AAV2 ITRs.

Newborn Twitcher (twi/twi) mice (PND 0) received a single ICV administration of AAV1.CB7.CI.hGALCco.rBG, AAV3B.CB7.CI.hGALCco.rBG, or AAV5.CB7.CI.hGALCco.rBG at a dose of 2.0 × 10¹⁰ GC (1.3 × 10¹¹ GC/g brain weight). The ICV route (involving administration of AAV vector directly into the CSF of the cerebral ventricles) was chosen to evaluate the potential for delivering GALC enzyme to the CNS and PNS, which are the targets for the treatment for infantile Krabbe disease. In-life assessments included viability checks, body weight monitoring, neuromotor assessments (RotaRod), and monitoring for survival. Necropsies were performed on animals at a humane endpoint. At humane endpoint, CNS and PNS were collected for histopathology to observe the demyelination and globoid cells infiltration, hGALC expression, and transgene expression (GALC enzyme activity).

TABLE

Group Designations, Dose Levels, and Route of Administration

| Group^{a} | N | Sex | Genotype^{a} | Treatment | Dose (GC/Anim al) | Dose (GC/g Brain)^{b} | Dose Volume (µL) |
|---|---|---|---|---|---|---|---|
| 1 | 6 | both | twi/twi | AAV1.CB7.CI.hGALCco. rBG | 2.0 × 10¹⁰ | 1.3 × 10¹¹ | 2.0 |
| 2 | 9 | both | twi/twi | AAV3B.CB7.CI.hGALCc o.rBG | 2.0 × 10¹⁰ | 1.3 × 10¹¹ | 2.0 |
| 3 | 6 | both | twi/twi | AAV5.CB7.CI.hGALCco. rBG | 2.0 × 10¹⁰ | 1.3 × 10¹¹ | 2.0 |

^{a} The twi allele consist of a loss-of-function mutation in the Galcgene.twi/twi refers to knockout mice
^{b}Values were calculated using a brain mass of 0.15 g for a newborn mouse (Gu et al., 2012) Abbreviations: GC, genome copies; ICV, intracerebroventricular; ID, identification number; N, number of animals; N/A, not applicable; PBS, phosphate-buffered saline; ROA, route of administration.

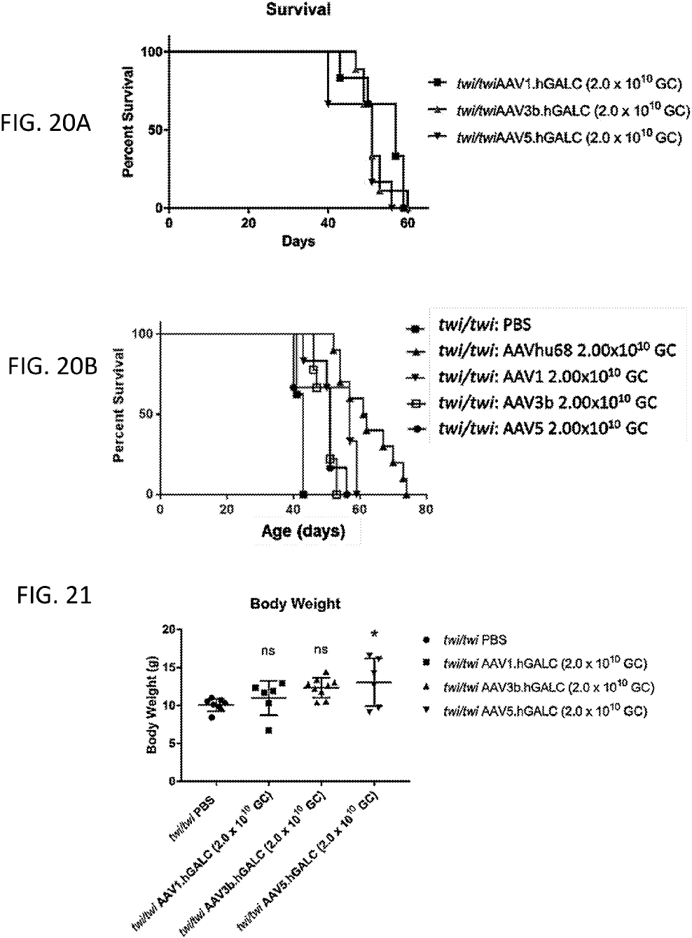

All twi/twi animals were euthanized upon reaching their humane endpoint. One twi/twi animal was found dead (AAV3B.hGALC group) and included in the survival analysis. Median survival was 57 days following administration of AAV1.hGALC and 51 days with AAV3B.hGALC and AAV5.hGALC in twi/twi animals (FIG. 20A and FIG. 20B).

Administration of AAV5.hGALC lead to rescue of body weight loss in twi/twi mice. The rescue of body weight was statistically significant for AAV5.hGALC when compared to PBS-treated twi/twi mice (FIG. 21).

Neuromotor function was assessed by the RotaRod test, which evaluates coordination and balance by measuring the time to fall for mice running on a spinning rod that progressively accelerates. A decrease in fall latency indicates neuromotor impairment, while an increase in fall latency indicates improved neuromotor function (FIG. 22). No significant change in fall latency was observed for any capsid tested.

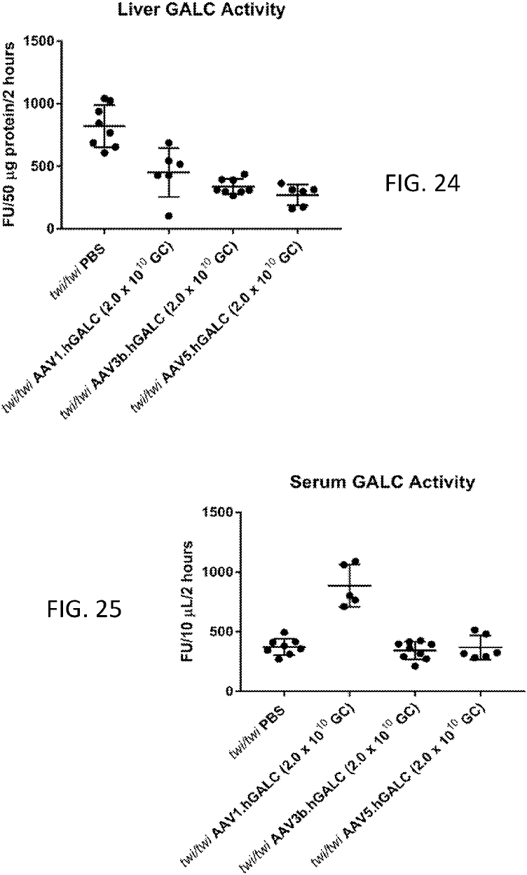

GALC enzyme activity levels in the brain was increased following administration of AAV1.hGALC compared to the other capsids and the twi/twi PBS-treated group (FIG. 23). GALC activity was similar between AAV3B.hGALC and AAV5.hGALC and higher than the twi/twi PBS-treated group. In the liver, GALC enzyme activity levels was lower in the AAV-treated groups compared to the twi/twi PBS-treated group (FIG. 24). In the serum, GALC enzyme activity levels was lower in the AAV-treated groups compared to the twi/twi PBS-treated group (FIG. 25).

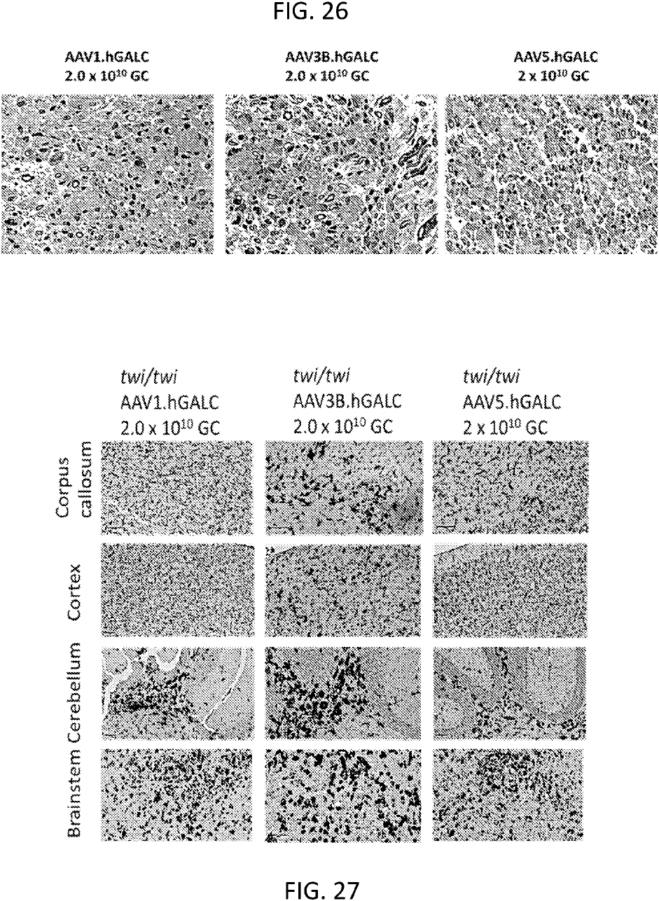

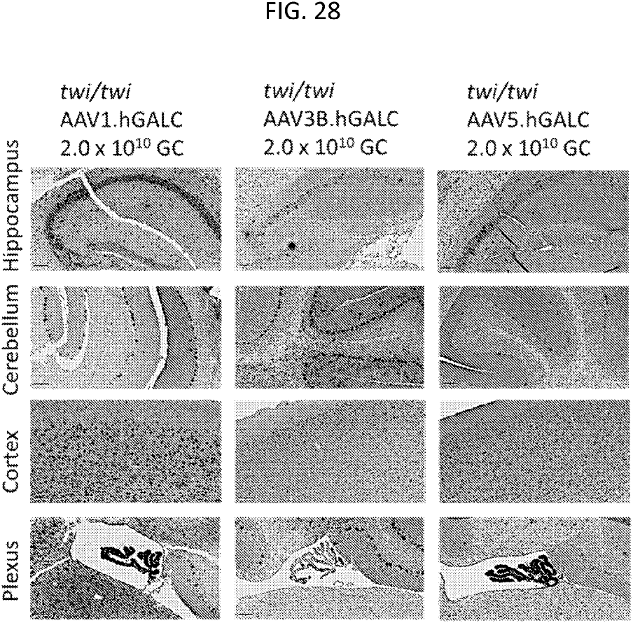

Sciatic nerve (FIG. 26) IBA1-positive cells were normalized in the cerebral cortex and hippocampus after ICV administration of AAV1.hGALC (FIG. 27). Numerous IBA1-positive globoid cells were observed in the corpus callosum of animals treated with AAV3B.hGACL and AAV5.hGALC indicating these capsids were less efficacious. AAV1.hGALC administration produced robust transduction of neurons in the hippocampus, cerebral cortex, cerebellum Purkinje cells, plexus choroid cells, medulla oblongata in close proximity to the pontine cistern (FIG. 28). The more robust transduction of plexus choroid and some medulla neurons may account for the higher GALC activity measured in brains from animals injected with AAV1.hGALC. AAV3B.hGALC transduced neurons mainly in the cerebellum with very few transduced neurons elsewhere. AAV5.hGALC led to low neuronal transduction overall and mainly transduced cells in the choroid plexus (FIG. 28).

### Example 5 - Evaluation of rAAVhu68.hGALC Juvenile Twitcher (Twi/Twi) Mice Following Intracerebroventricular Administration

Studies were performed to determine the efficacy of rAAVhu68.hGALC (AAVhu68.CB7.hGALCco.rBG) in the Twitcher mouse model of infantile Krabbe disease following intracerebroventricular (ICV) administration.rAAVhu68.hGALC is a recombinant adeno-associated virus (AAV) serotype hu68 vector expressing the human galactocerebrosidase (GALC) gene. Juvenile Twitcher (twi/twi) mice at PND 12 received a single ICV administration of rAAVhu68.hGALC at a dose of 1.0 × 10¹¹ GC or 2.0 × 10¹¹ GC (2.5 × 10¹¹ or 5.0 × 10¹¹ GC/g brain weight, respectively). Alternatively, rAAVhu68.hGALC was administered on PND 21 at a dose of 2.0 × 10¹¹ GC (5.0 × 10¹¹ GC/g brain weight). The ages of the animals were selected as PND 12 is an age prior to the onset of behavioral symptoms (“early-symptomatic”) and PND21 is an age when a mice display behavioral symptoms (“later-symptomatic”). Moreover, PND 12 and PND 21 translate to a 2 month old and a 9 month old, respectively in humans (www.translatingtime.org), which is similar to the intended infantile population for the FIH trial.

In-life assessments included viability checks performed daily, body weight monitoring, neuromotor assessments (RotaRod), and monitoring for survival. Necropsies were performed on animals at a humane endpoint. At the humane endpoint, CNS and PNS were collected for histopathology to observe the demyelination and globoid cells infiltration, hGALC expression, and transgene expression (GALC enzyme activity).

TABLE

Group Designations, Dose Levels, and Route of Administration

| Group | N | Sex | Genotype^{a} | Treatment | Dose (GC/Anima l) | Dose (GC/g Brain)^{b} | Dose Volume (µL) | ROA | Necropsy Day |
|---|---|---|---|---|---|---|---|---|---|
| 1 | 4 | both | twi/twi | PBS | N/A | N/A | 4.0 | IV | Humane endpoint^{c} |
| 2 | 13 | both | +/+ and twi/+ | PBS | N/A | N/A | 4.0 | IV | Humane endpoint |
| 3 | 12 | both | twi/twi | AAVhu68.CB7. hGALCco.rBG | 1.0 × 10¹¹ | 2.5 × 10¹¹ | 4.0 | IV | Humane endpoint |
| 4 | 11 | both | twi/twi | AAVhu68.CB7. hGALCco.rBG | 2.0 × 10¹¹ | 5.0 × 10¹¹ | 2×4.0 (n=3); 4.0 (n=8) | ICV | Humane endpoint |
| 1 | 16 | both | twi/twi | AAVhu68.CB7. hGALCco.rBG | 2.0 × 10¹¹ | 5.0 × 10¹¹ | 5.0 | ICV | Humane endpoint |

^{a}+/+, wild-type mice; twi/+ heterozygous genotype; twi/twi knockout mice; The twi allele consist of a loss-of-function mutation in the Galcgene.
^{b}Values were calculated using a brain mass of 0.4 g for a newborn mouse (Gu et al., 2012)
^{c} PBS-treated wild type and heterozygous mice were used for rotarod and body weight controls and did not reach humane endpoint. They were euthanized at study completion. Abbreviations: GC, genome copies; ICV, intracerebroventricular; ID, identification number; N, number of animals; N/A, not applicable; PBS, phosphate-buffered saline; ROA, route of administration.

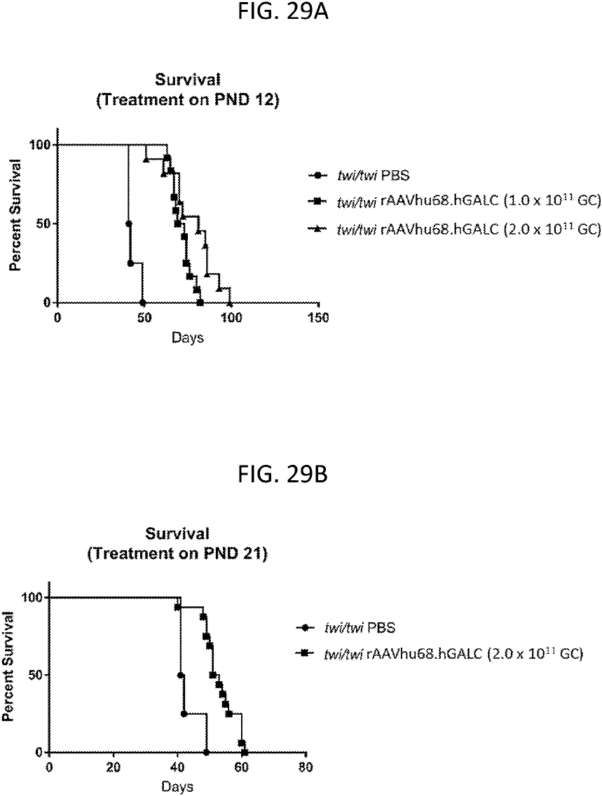

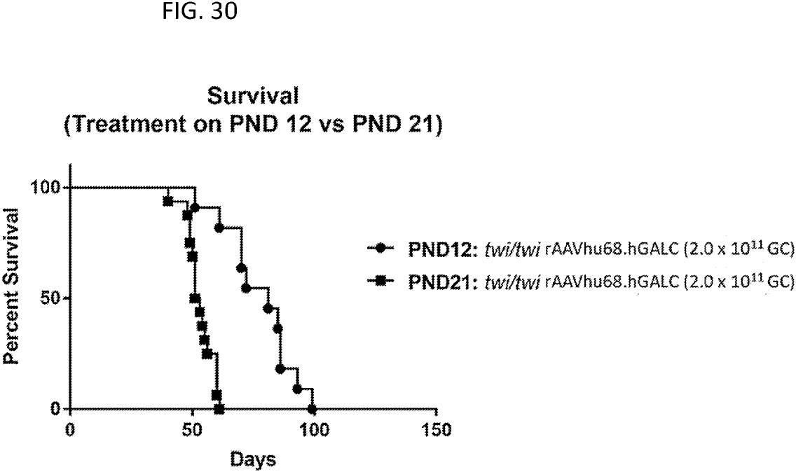

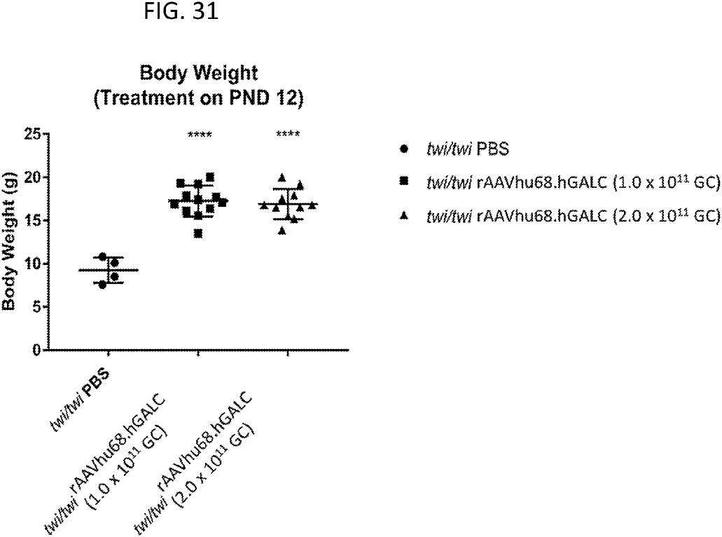

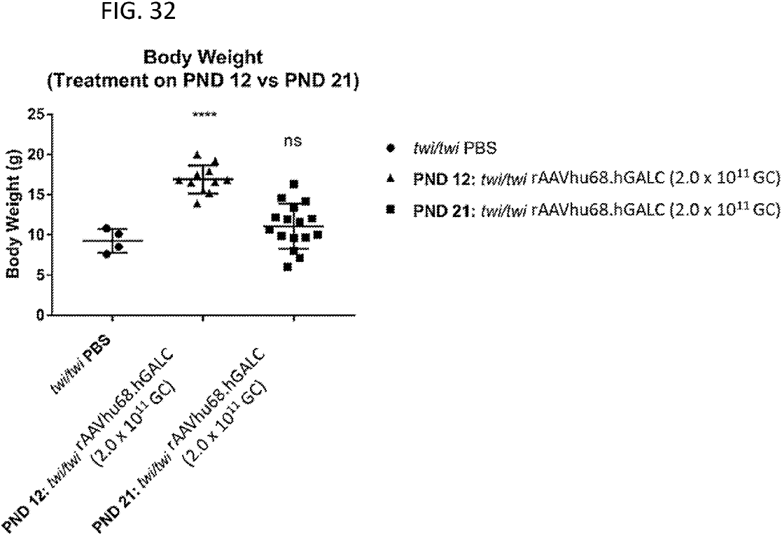

All twi/twi animals were euthanized upon reaching humane endpoint. A statistically significant increase in survival in twi/twi mice was observed when rAAVhu68.hGALC was administered on PND 12 and PND 21 (FIG. 29A and FIG. 29B). Median survival for mice treated on PND12 was 41.5 days in PBS-treated mice, 71 days in mice treated at a rAAVhu68.hGALC dose of 1.0 × 10¹¹ GC and 81 day in mice treated at a rAAVhu68.hGALC dose of 2.0 × 10¹¹ GC. In mice treated with rAAVhu68.hGALC at a dose of 2.0 × 10¹¹ GC on PND21 median survival was 52 days. Survival was statistically significantly improved in animals treated with rAAVhu68.hGALC at a dose 2.0 × 10¹¹ GC on PND12 compared to PND21 (FIG. 30). Median survival was 81 days and 52 days in mice treated on PND12 and PND21, respectively. Administration of rAAVhu68.hGALC at a dose of 1.0 × 10¹¹ GC or 2.0 × 10¹¹ GC on PND12 lead to a statistically significant rescue of body weight loss in twi/twi mice when compared to PBS-treated mice (FIG. 31). rAAVhu68.hGALC treatment at a dose of 2.0 × 10¹¹ GC on PND 21 treatment did not significantly rescue weight loss when compared to vehicle-treated animals (FIG. 32).

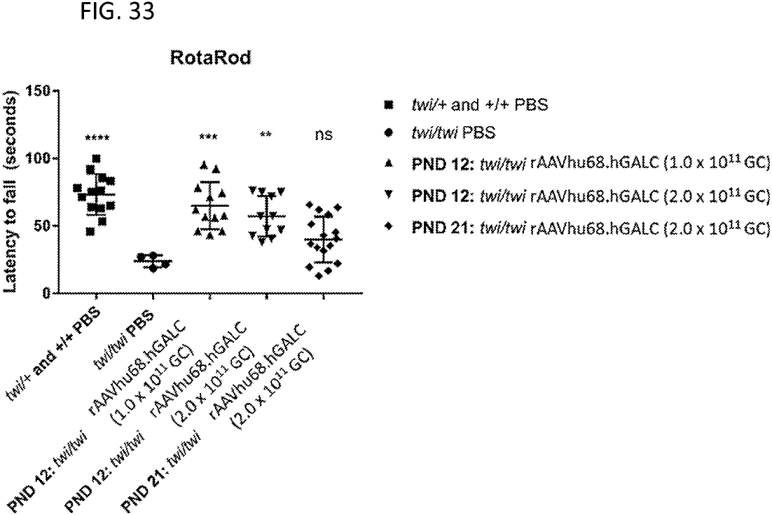

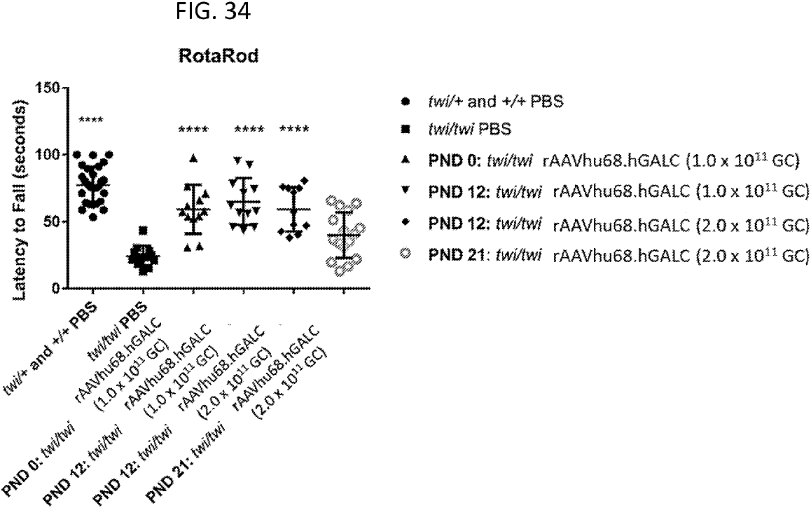

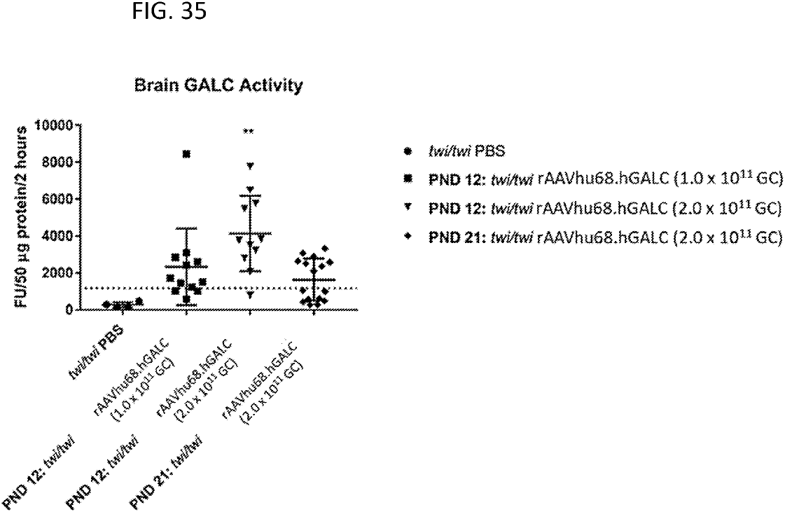

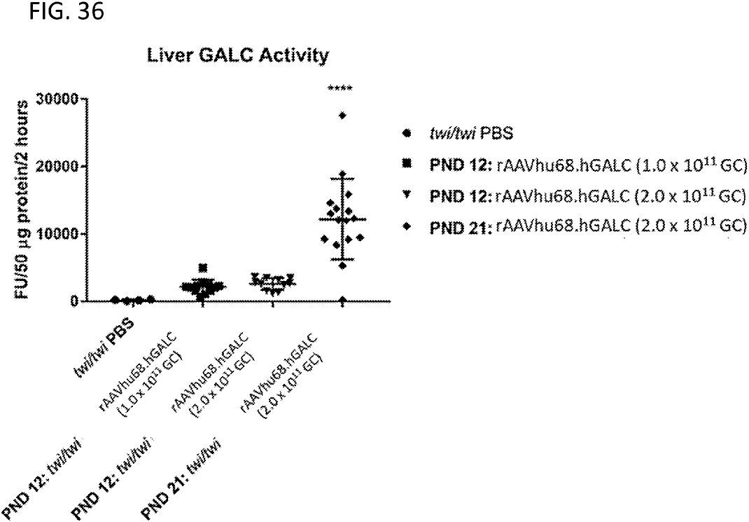

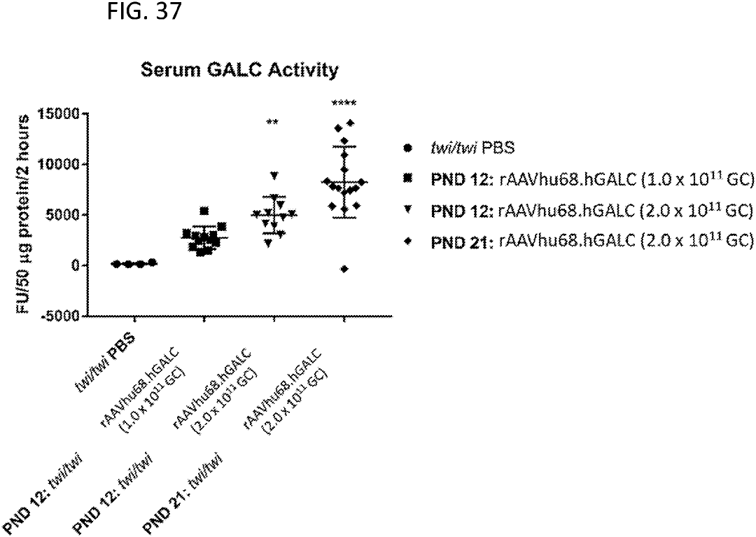

Neuromotor function was assessed by the RotaRod test, which evaluates coordination and balance by measuring the time to fall for mice running on a spinning rod that progressively accelerates. A decrease in fall latency indicates neuromotor impairment, while an increase in fall latency indicates improved neuromotor function. rAAVhu68.hGALC administered ICV on PND12 to twi/twi mice led to a statistically significant increase in fall latency when compared to PBS-treated mice (FIG. 33). Fall latency was similar in the PBS treated wild type and twi/+ mice.rAAVhu68.hGALC administered at a dose of 2.0 × 10¹¹ GC to twi/twi mice on PND21 did not improve fall latency (FIG. 34).

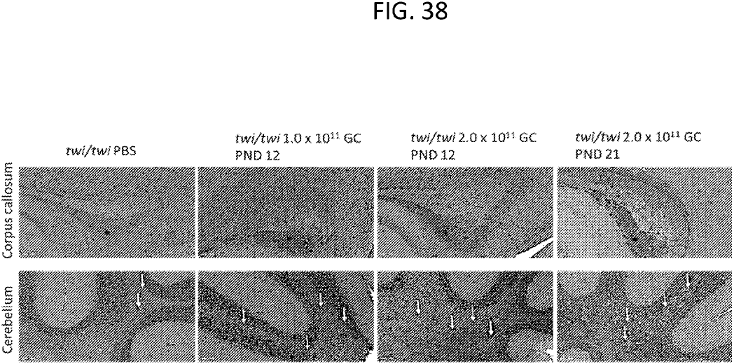

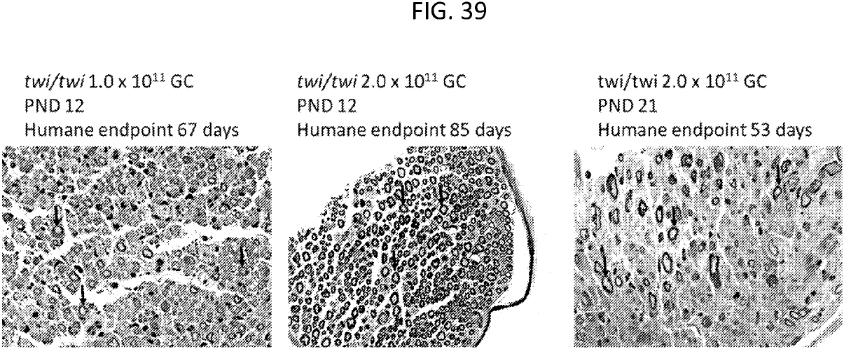

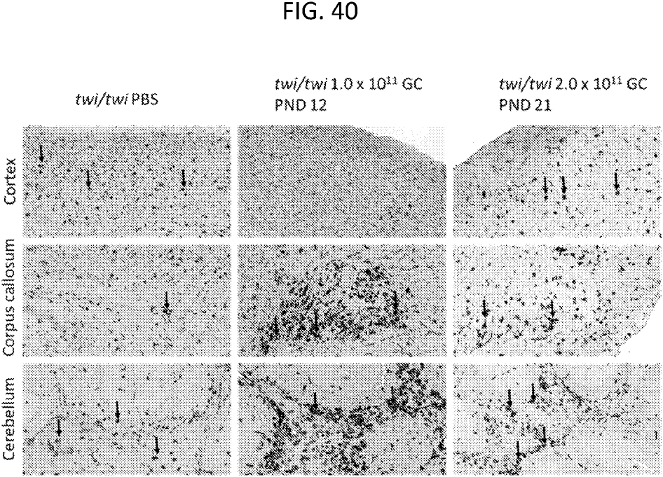

Brain GALC enzyme activity was increased in a dose-dependent manner in twi/twi mice administered rAAVhu68.hGALC on PND12 (FIG. 35). When mice were treated with rAAVhu68.hGALC at a dose of 2.0 × 10¹¹ GC on PND 21, the GALC enzyme activity increase was less pronounced. Liver and serum GALC enzyme activity was higher in twi/twi mice treated with rAAVhu68.hGALC at a dose of 2.0 × 10¹¹ GC on PND 21 compared to mice treated on PND 12 (FIG. 36 and FIG. 37). The higher GALC activity in the PND21-treated mice may be a result of shorter survival post treatment would lead to less transgene loss following liver growth in juvenile animals. The brain of twi/twi PBS-treated animals showed decreased myelin staining in white matter of the corpus callosum and cerebellum and abundant PAS stained globoid cells were observed in the cerebellar folia (FIG. 38). rAAVhu68.hGALC -treated mice at all dose groups administered on PND 12 and PND21 reduced demyelination in the corpus callosum but was less effective at correcting demyelination in the cerebellum where globoid cell infiltration was observed.

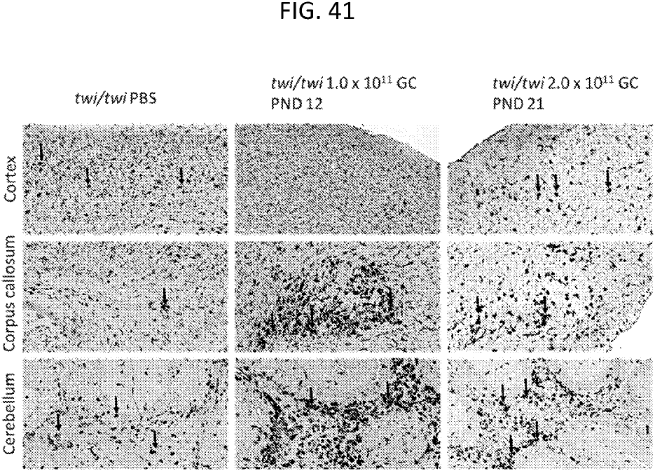

Wild type mice displayed abundant myelinated nerve fibers that are tightly packed and no inflammatory cells while twi/twi mice displayed a decreased number of myelinated nerve fibers and numerous enlarged mononuclear cells (globoid cells) seen between nerve fibers. Sciatic nerves from all twi/twi mice treated with rAAVhu68.hGALC on PND 12 or PND21 showed moderate to severe demyelination of myelin fibers (FIG. 39). This finding is likely due to the fact that the humane endpoint was defined as hindleg paralysis which would occur when sciatic nerve is severely affected.rAAVhu68.hGALC treated mice that survived the longest had more myelin fibers.

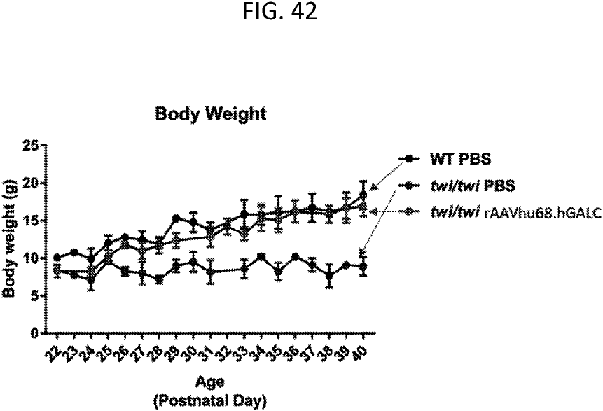

IBA1-positive cells were similar to wild type in the cortex of twi/twi treated with rAAVhu68.hGALC on PND12 while mice treated on PND21 exhibited globoid cells and enlarged activated microglia in all analyzed brain regions (FIG. 40). Neuroinflammation and enlarged globoid cells persisted in the corpus callosum, cerebellum (FIG. 40) and brainstem (data not shown). Robust expression of hGALC in neurons of the cerebral cortex and hippocampus (in proximity from the injection site) was observed in twi/twi mice treated with rAAVhu68.hGALC on PND12 or PND21 (FIG. 41). Purkinje cells in the cerebellum were positive for hGALC only in animals treated with rAAVhu68.hGALC on PND 12. The brainstem was not transduced rAAVhu68.hGALC -treated regarding of age of administration.

### Example 6 - Evaluation of Delivery of rAAVhu68.hGALC to Juvenile Twitcher (Twi/Twi) Mice

Additional studies were performed to determine the efficacy of rAAVhu68.hGALC (AAVhu68.CB7.hGALCco.rBG) in the Twitcher mouse following intracerebroventricular (ICV) administration. Juvenile Twitcher (twi/twi) mice at PND 12 received a single ICV administration of rAAVhu68.hGALC at a dose of 2.0 × 10¹¹ GC (1.3 ×10¹² GC/g brain weight). The age of the animals was selected as PND12 is an age prior to the onset of behavioral symptoms (“early-symptomatic”) and translates to a 2 month old human (www.translatingtime.org) and is similar to the intended infantile population for the FIH trial.

In-life assessments included viability checks performed daily, body weight monitoring and neuromotor assessments (RotaRod). Necropsies were performed on animals on PND40. At necropsy, CNS and PNS were collected for histopathology to observe the demyelination and globoid cells infiltration, hGALC expression, and transgene expression (GALC enzyme activity).

TABLE

Group Designations, Dose Levels, and Route of Administration

| Group a | N | Sex | Genotype^{a} | Treatment | Dose (GC/Anima l) | Dose (GC/g Brain)^{c} | Dose Volume (µL) | ROA | Dosing Day | Necropsy Day |
|---|---|---|---|---|---|---|---|---|---|---|
| 1 | 7 | both | +/+ | PBS | N/A | N/A | 4.0 | ICV | 12±1 | PND40 |
| 2 | 8 | both | twi/twi | PBS | N/A | N/A | 4.0 | ICV | 12±1 | PND40 |
| 3 | 10 | both | twi/twi | AAVhu68. CB7.hGAL Cco.rBG | 2.0 × 10¹¹ | 1.33 × 10¹² | 4.0 | ICV | 12±1 | PND40 |

^{a}+/+, wild-type mice; twi/+ heterozygous genotype; twi/twi knockout mice; The twi allele consist of a loss-of-function mutation in the Galcgene.
^{b}Values were calculated using a brain mass of 0.4 g for a newborn mouse (Gu et al., 2012) Abbreviations: GC, genome copies; ICV, intracerebroventricular; ID, identification number; N, number of animals; N/A, not applicable; PBS, phosphate-buffered saline; ROA, route of administration.

Clinical signs were scored three times per week by personnel blinded to the treatment group using an unpublished assessment of clasping ability, gait, tremor, kyphosis, and fur quality (table below). These measures were chosen to assess clinical status based on the symptoms typically exhibited by twi/twi mice. Scores above 0 indicated clinical deterioration.

| Assessment Category | Observation | Clinical Score |
|---|---|---|
| Hind Limb Clasping | No clasping | 0 |
| | Non-permanent clasping | 1 |
| | Permanent Clasping | 2 |
| Gait | Normal | 0 |
| | Slightly abnormal gait but mouse moves easily and spontaneously | 1 |
| | Markedly abnormal gait, reduced spontaneous mobility | 2 |
| | Severe difficulty moving forward, dragging of hind legs | 3 |
| Tremors | Normal | 0 |
| | Minimal tremors, only visible when mice are immobile | 1 |
| | Moderate tremors, noticeable at rest and while moving. Twitching | 2 |
| | Marked tremors and twitching, obvious both at rest and while moving. | 3 |
| Spinal Curvature | Normal | 0 |
| | Mild kyphosis (curved spine), but able to straighten spine completely | 1 |
| | Unable to straighten spine completely; maintenance of persistent mild kyphosis | 2 |
| | Maintenance of pronounced kyphosis while walking or sitting | 3 |
| Fur Quality | Normal | 0 |
| | Any abnormality (scruffed, alopecia, etc.) | 1 |

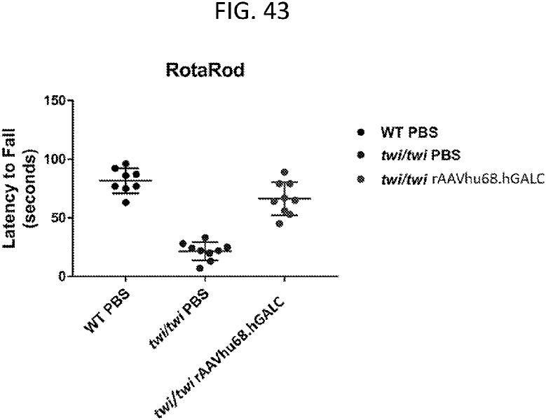

Administration of rAAVhu68.hGALC at a dose of 2.0 × 10¹¹ GC rescued body weight loss with values be comparable to the wild type mice administered vehicle (FIG. 42).

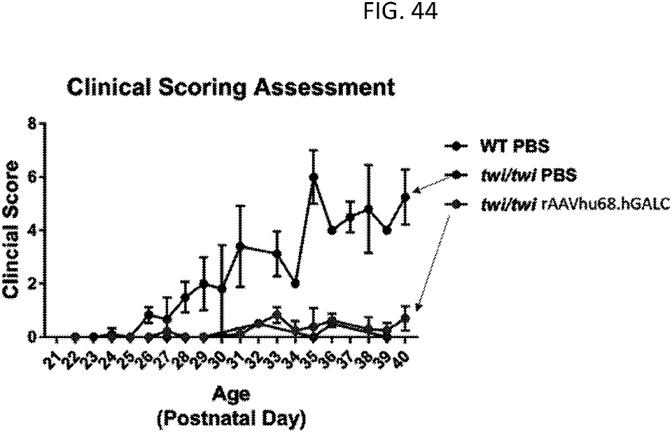

Neuromotor function was assessed by the RotaRod test, which evaluates coordination and balance by measuring the time to fall for mice running on a spinning rod that progressively accelerates. A decrease in fall latency indicates neuromotor impairment, while an increase in fall latency indicates improved neuromotor function. rAAVhu68.hGALC administered to twi/twi mice led to a statistically significant increase in fall latency when compared to PBS-treated twi/twi mice (FIG. 43). Administration of rAAVhu68.hGALC to twi/twi mice reduced the total clinical scores compared to the PBS-treated twi/twi mice (FIG. 44).rAAVhu68.hGALC -treated twi/twi mice had similar clinical scores to wild type PBS-treated mice.

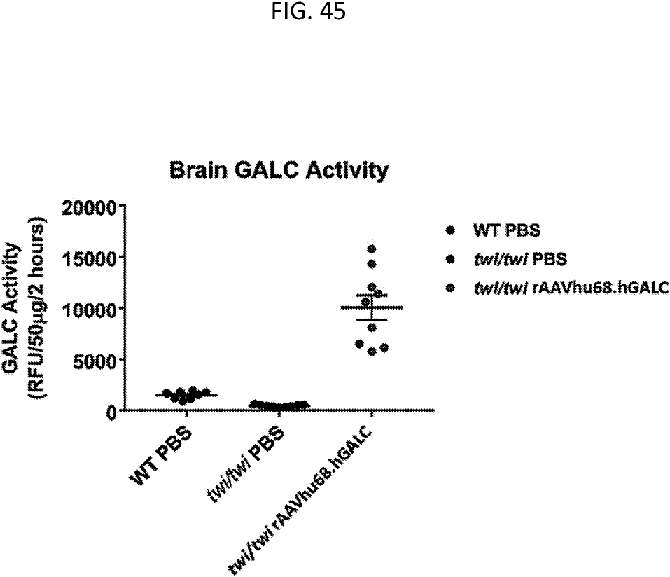

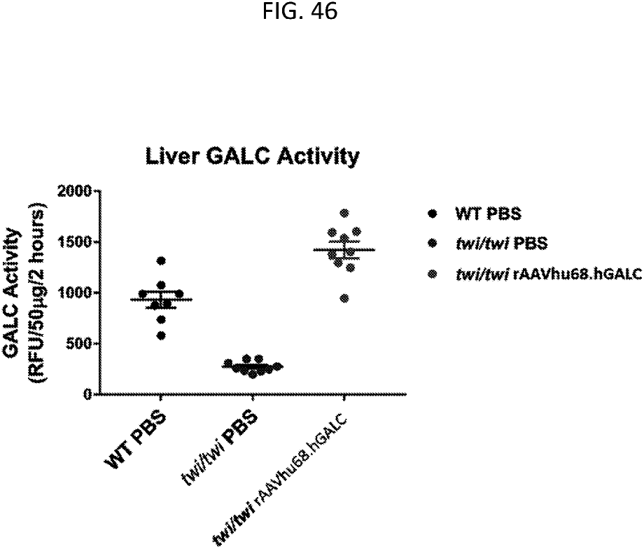

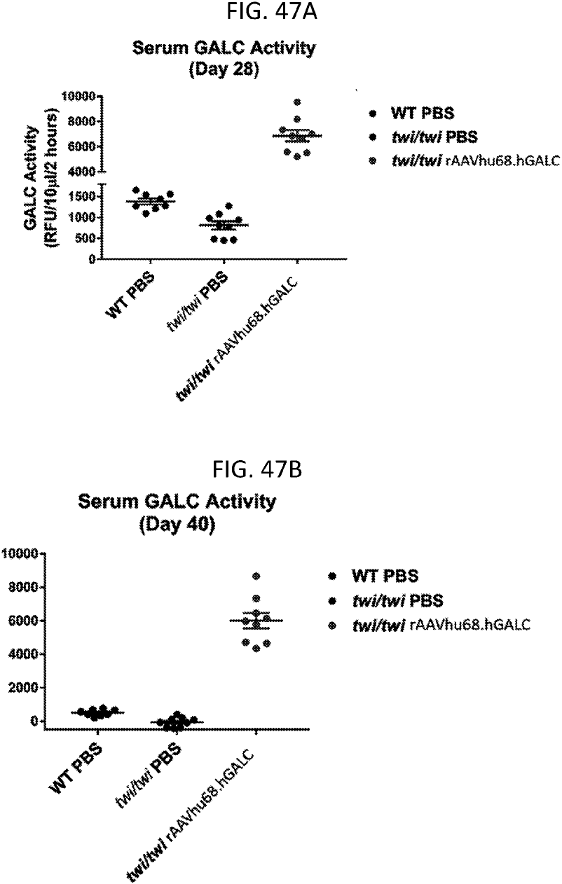

Brain GALC enzyme activity was increased in rAAVhu68.hGALC -treated mice compared to wild type or twi/twi PBS-treated mice (FIG. 45). Liver GALC enzyme activity was increased in rAAVhu68.hGALC -treated mice compared to wild type or twi/twi PBS-treated mice (FIG. 46). GALC enzyme activity in the serum on PND28 and PND 40 was increased in rAAVhu68.hGALC -treated mice compared to wild type or twi/twi PBS-treated mice (FIG. 47A and FIG. 47B).

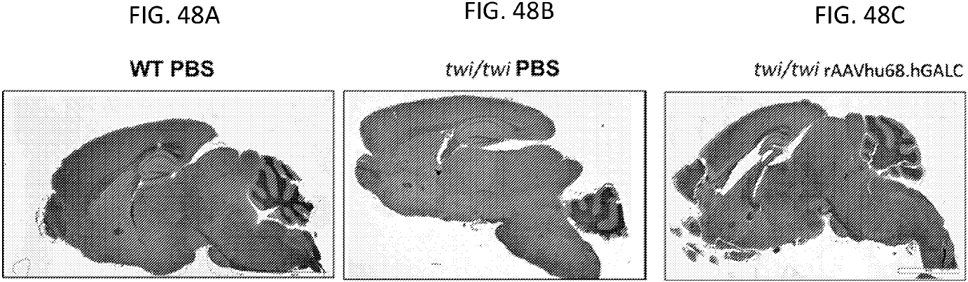

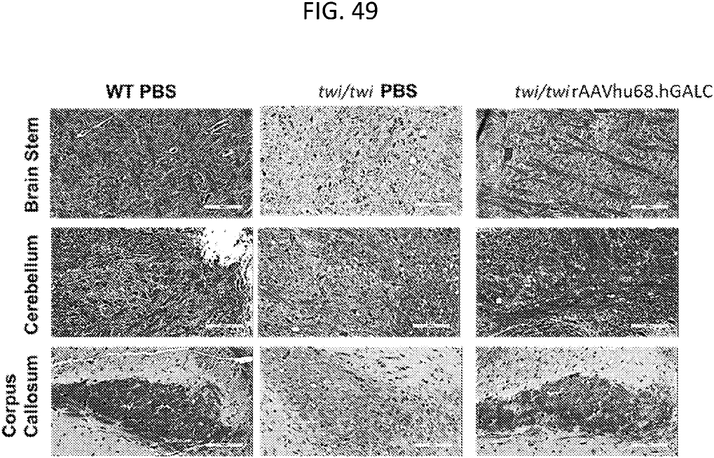

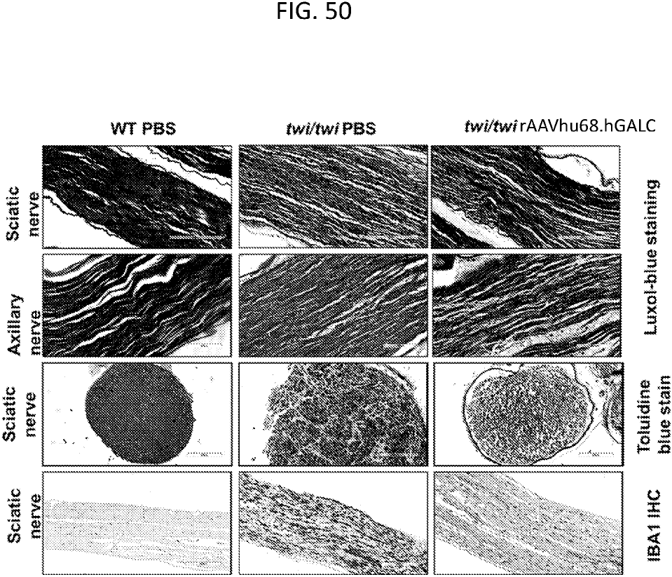

rAAVhu68.hGALC administration to twi/twi mice had no signs of demyelination (FIG. 48). Demyelination levels in the rAAVhu68.hGALC treated mice was similar to wild type PBS-treated mice. Decreased myelin staining and infiltration with abundant PAS stained globoid cells were observed in white matter of the brain stem, corpus callosum and cerebellum of twi/twi PBS-treated mice (FIG. 49). Treatment of rAAVhu68.hGALC to twi/twi mice reduced demyelination and suppressed globoid cells in these myelin-rich areas (FIG. 48). In PBS-treated twi/twi mice peripheral nerves had significant loss of the amount of myelin fibers and the nerve fiber structure appeared disorganized (FIG. 50). In contrast, peripheral nerves from twi/twi treated with rAAVhu68.hGALC showed some demyelination but the number of nerve fibers and the structure was not as disorganized compared to the PBS-treated mice (FIG. 50). A similar finding was observed for the sciatic nerve (FIG. 50).

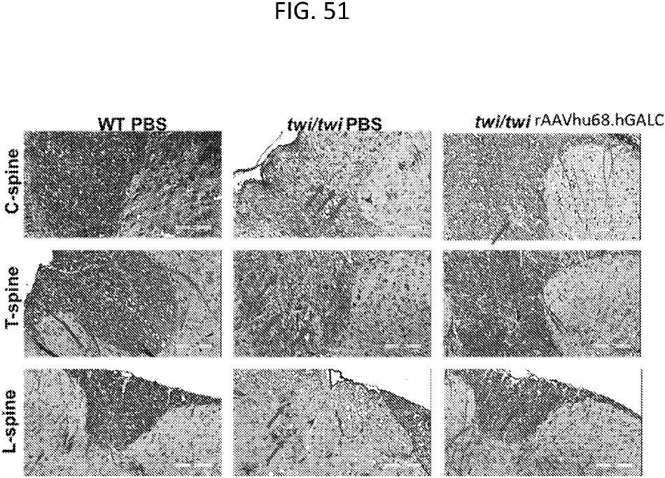

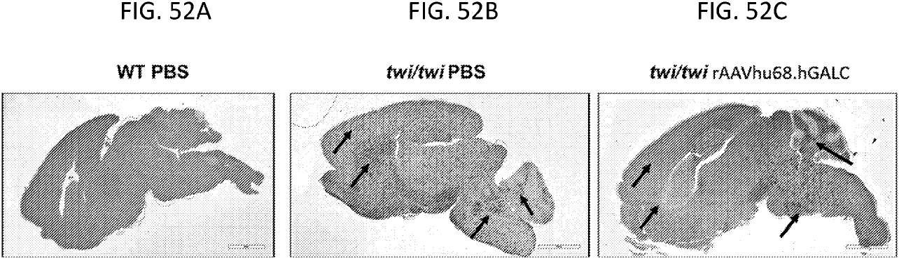

rAAVhu68.hGALC administration reduced demyelination in the dorsal tract of the spinal cord when compared to PBS-treated twi/twi mice showed severe demyelination (FIG. 51). IBA1-positive cells in the wild type PBS-treated mice were small and evenly distributed through the whole brain, while in the PBS-treated twi/twi mice the microglia cells were large and gave a patchy coarse staining appearance in the cortex, corpus callosum, brainstem and cerebellum (FIG. 52A - FIG. 52C). In twi/twi treated with rAAVhu68.hGALC there were more consistent staining of IBA1-positive cells in cortex, corpus callosum, but the patchy staining, meaning neuroinflammation, persisted in the cerebellum and brainstem (FIG. 52A -FIG. 52C). rAAVhu68.hGALC administration dramatically reduce the IBA1-positive cells in the cortical cortex, hippocampus, corpus callosum (FIG. 53). However, it remained at the same level in cerebellum and brain stem.

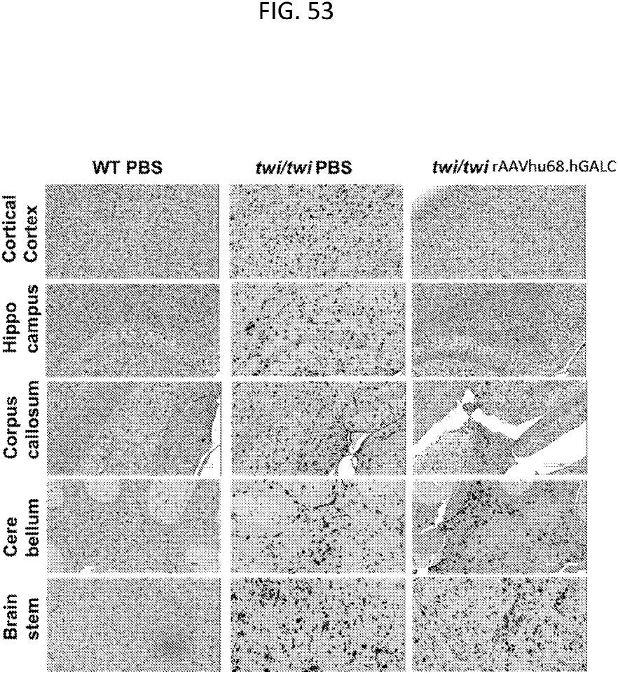

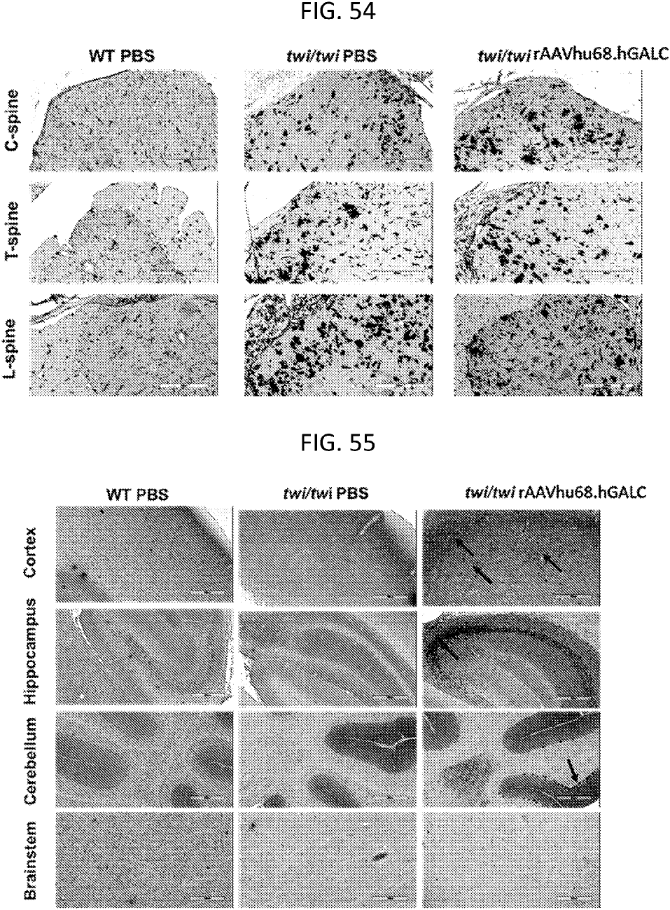

rAAVhu68.hGALC administration reduced neuroinflammation in the peripheral nerves (FIG. 50). rAAVhu68.hGALC administration did not inhibit neuroinflammation in the spinal cord when compared to PBS-treated twi/twi mice (FIG. 54). Robust expression of hGALC in neurons in the cortex, hippocampus, and a few positive staining in Purkinje neurons in cerebellum but no obvious staining in the brainstem of twi/twi mice administered rAAVhu68.hGALC (FIG. 55). No positive staining was observed in the brains of wild type and twi/twi PBS-treated mice.

### Example 7 - Effect of Bone Marrow Transplant in Combination With rAAVhu68.hGALC Administration

This study investigated the potential benefit of a dual therapy of rAAVhu68.hGALC and bone marrow transplant (BMT). We investigated this combination therapy because of the prominent neuroinflammatory component of Krabbe disease. In theory, there is a synergistic effect because the HSCT provides an additional source of GALC enzyme in the CNS (from macrophage/microglial cells derived from the transplanted cells and rAAVhu68.hGALC-transduced neurons), while rAAVhu68.hGALC provides correction to the PNS, which is not affected by HSCT. Moreover, different combination treatment designs were examined in this study to assess whether rAAVhu68.hGALC might be efficacious in 1) patients who receive a HSCT first through NBS programs followed by gene therapy and/or 2) patients who receive gene therapy first followed by HSCT, if eligible.

The combination therapy study is summarized in the table below.

TABLE

Study of AAV and BMT Combination Therapy in Mice

| Group | Genotype | N | Treatment #1 | Time Point | Treatment #2 | Time Point | Rationale |
|---|---|---|---|---|---|---|---|
| 1 | twi/twi | 13 | BMT | PND 10 | - | - | Assess efficacy of BMT monotherapy |
| 2 | twi/twi | 7 | rAAVhu68.hGALC | PND 0 | BMT | PND 10 | Assess efficacy of rAAVhu68.hGALC treatment followed by BMT as early as possible in newborn pre-symptomatic mice |
| 3 | twi/twi | 7 | BMT | PND 10 | rAAVhu68.hGALC | PND 12 | Assess efficacy of BMT followed by rAAVhu68.hGALC treatment; PND 10 was found to be earliest time point for BMT in pilot experiments because busulfan conditioning is toxic to mice weighing less than 4 g |
| 4 | twi/twi | TBD | rAAVhu68.hGALC | PND 12 | BMT | PND 28 | Assess efficacy of rAAVhu68.hGALC treatment followed by BMT in early-symptomatic mice at a later stage of disease progression |
| 5 | twi/twi | 12 | rAAVhu68.hGALC | PND 0* | - | - | Control: rAAVhu68.hGALC monotherapy in pre-symptomatic mice |
| 6 | twi/twi | 13 | rAAVhu68.hGALC | PND 12* | - | - | Control: rAAVhu68.hGALC monotherapy in early-symptomatic mice |
| 7 | twi/twi | 12 | PBS | * | - | - | Control: Vehicle-only treatment |

rAAVhu68.hGALC was administered at a dose of 1.00 × 10¹¹ GC.
All mice receiving a BMT also undergo myeloablative conditioning with busulfan 1-2 days prior to the BMT procedure to reduce the quantity of endogenous bone marrow cells.
*Historical controls are used for these groups. Group 5 is a historical control from Study 1. Group 6 is a historical control from Study 2. Group 7 is a historical control consisting of N=8 mice from Study 1 (PND 0) and N=4 mice from Study 2 (PND 12).
Abbreviations: AAV, adeno-associated virus; BMT, bone marrow transplant; GC, genome copies; PBS, phosphate-buffered saline; PND, postnatal day; TBD, to be determined.

The rAAVhu68.hGALC dose of 1.00 × 10¹¹ GC was utilized because we anticipate a better response due to the combination therapy, which permits a lower dose of rAAVhu68.hGALC than was used in previous studies of rAAVhu68.hGALC monotherapy. Efficacy of rAAVhu68.hGALC is assessed in terms of survival, body weight, and neurologic observations (e.g., presence of tremor and abnormal clasping reflex).

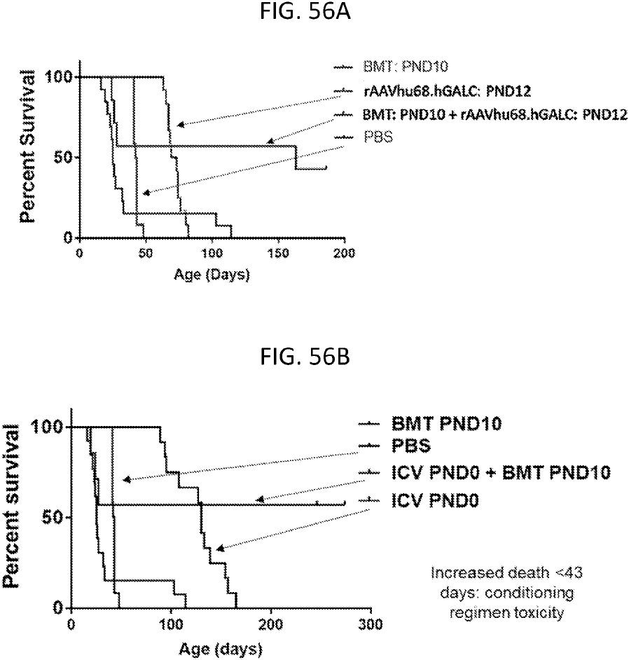

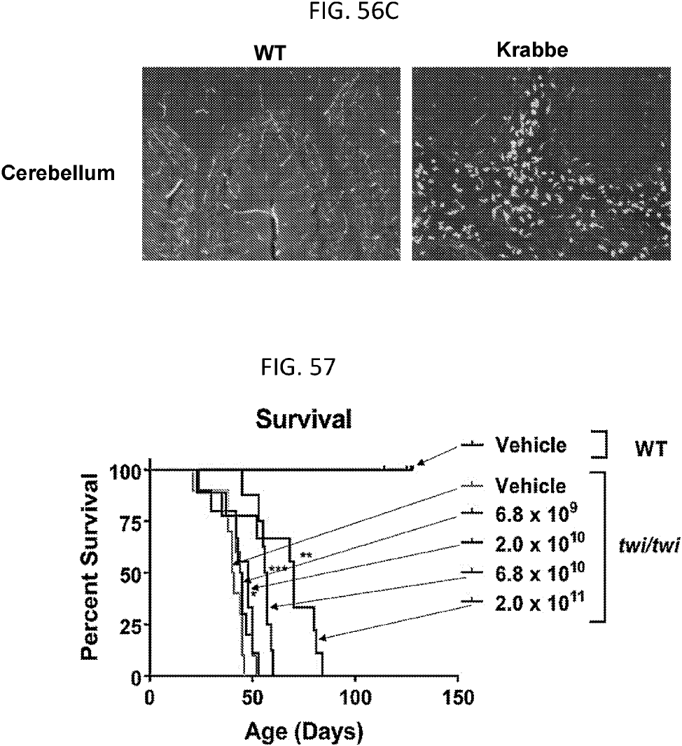

The survival data for Groups 1-3 are shown in FIG. 56A and FIG. 56B. Thus far, the best survival was achieved with the combination of treating presymptomatic Twitcher mice (twi/twi) with ICV-administered rAAVhu68.hGALC on PND 0 followed by BMT on PND 10 (Group 2). Survival was extended to >300 days in the absence of overt signs. These mice appear to be in better physical condition based upon the previously described clinical assessment, displaying a slight tremor with no noticeable gait abnormalities and no clasping (analysis still ongoing; data not shown). Mice that received BMT before rAAVhu68.hGALC (Group 3) are currently still alive (N=3/7), but they display marked tremor, some gait abnormality, and lower body weight. However, the busulfan conditioning regimen coupled with BMT is toxic in mice younger than 10 days of age, and mice in both Groups 2 and 3 displayed increased mortality either before or shortly after BMT, regardless of the order of the combination therapies. Group 4 are being injected to mimic a clinically relevant situation of gene therapy administered to early symptomatic patients followed by BMT.

Cumulatively, these data suggest that combining rAAVhu68.hGALC treatment with a subsequent BMT may provide more efficacy than each treatment alone in the murine model of Krabbe disease.

### Example 8 - Efficacy of rAAVhu68.hGALC Following Intracerebroventricular Administration in Twitcher (Twi/Twi) Mice to Determine the Minimum Effective Dose (MED)

The purpose of this pharmacology study was to determine the minimum effective dose (MED) and transgene expression levels in the Twitcher mouse model of infantile Krabbe disease following intracerebroventricular (ICV) administration of AAVhu68.CB7.CI.GALC.rBG (rAAVhu68.hGALC).

In-life assessments included observations performed daily, monitoring for survival, body weight measurements, neurological exams, neuromotor function assessments (rotarod), and evaluation of serum transgene expression (GALC enzyme activity). Necropsies were performed on untreated mice on the day of dosing (PND 12-14 [untreated baseline cohort]), 4 weeks post dosing (PND 40-42 [PND 40 cohort]), and at a humane endpoint to evaluate survival (up to 10 weeks post dosing in twi/twi mice [survival cohort]). At necropsy, a comprehensive list of tissues was collected for histopathological evaluation. Samples of the brain, spinal cord, and sciatic nerves were collected for evaluation of myelination (Luxol Fast Blue [LFB] staining) along with globoid cell infiltration and neuroinflammation (Periodic Acid-Schiff [PAS] staining and IBA1 immunohistochemistry). Brain, peripheral organs, and serum were collected for a transgene expression assay (GALC enzyme activity). Blood was collected for complete blood counts (CBCs) with differentials and serum clinical chemistry analysis.

At the time of enrollment (PND 12-14), litters of mice were randomly assigned to treatment groups. The age of the animals was selected to model the disease stage of early-symptomatic patients. Due to the size of the study and the unpredictable day of litter birth, litters were injected as they become available, and the randomization process was as follows: when litters became available, the mating cage card numbers were entered in a random list generator. The randomized list was then assigned to study groups by ascending number (the first number was assigned to Group 1, the second number was assigned to Group 2, and so on until all groups were enrolled). Randomization lists were saved in the study binder. Earlier-enrolled animals were assigned to the survival cohort within each group while later-enrolled mice were assigned to the PND 40 cohort. Animal were replaced at the discretion of the Study Director in cases of pre-weaning mortality (with appropriate justification).

Following group assignment, each animal (except the untreated baseline controls) received a single ICV injection of one of the following treatments:
rAAVhu68.hGALC (test article) at a dose of 6.8 × 10⁹ GC/animal
rAAVhu68.hGALC (test article) at a dose of 2.0 × 10¹⁰ GC/animal
rAAVhu68.hGALC (test article) at a dose of 6.8 × 10¹⁰ GC/animal
rAAVhu68.hGALC (test article) at a dose of 2.0 × 10¹¹ GC/animal
ITFFB (control article)

Group designations, dose levels, and the route of administration (ROA) are presented in the table below.

TABLE

Group Designations, Dose Levels, and Route of Administration

| Group Number | N and Sex | Genotype^{a} | Treatment | Dose (GC/Animal) | Dose (GC/g Brain)^{b} | Dose Volume (µL) | ROA | Dosing Day | Necropsy Cohort |
|---|---|---|---|---|---|---|---|---|---|
| 1 | 3 M, 3 F | twi/twi | Untreated | N/A | N/A | N/A | N/A | N/A | PND 12-14 |
| 2 | 3 M, 3 F | WT | Untreated | N/A | N/A | N/A | N/A | N/A | PND 12-14 |
| 3a | 4 M, 5 F | twi/twi | ITFFB | N/A | N/A | 4.0 | ICV | PND 12-14 | PND 40-42 |
| 3b | 4M, 5F | twi/twi | ITFFB | N/A | N/A | 4.0 | ICV | PND 12-14 | Survival |
| 4a | 2M, 7F | WT | ITFFB | N/A | N/A | 4.0 | ICV | PND 12-14 | PND 40-42 |
| 4b | 5M, 3F | WT | ITFFB | N/A | N/A | 4.0 | ICV | PND 12-14 | Survival |
| 5a | 5 M, 3 F | twi/twi | rAAVhu68. hGALC | 2.0 × 10¹¹ | 5.0 × 10¹¹ | 4.0 | ICV | PND 12-14 | PND 40-42 |
| 5b | 6M, 3F | twi/twi | rAAVhu68. hGALC | 2.0 × 10¹¹ | 5.0 × 10¹¹ | 4.0 | ICV | PND 12-14 | Survival |
| 6a | 6 M, 3 F | twi/twi | rAAVhu68. hGALC | 6.8 × 10¹⁰ | 1.7 × 10¹¹ | 4.0 | ICV | PND 12-14 | PND 40-42 |
| 6b | 5M, 3F | twi/twi | rAAVhu68. hGALC | 6.8 × 10¹⁰ | 1.7 × 10¹¹ | 4.0 | ICV | PND 12-14 | Survival |
| 7a | 3 M, 5 F | twi/twi | rAAVhu68. hGALC | 2.0 × 10¹⁰ | 5.0 × 10¹⁰ | 4.0 | ICV | PND 12-14 | PND 40-42 |
| 7b | 5M,4F | twi/twi | rAAVhu68. hGALC | 2.0 × 10¹⁰ | 5.0 × 10¹⁰ | 4.0 | ICV | PND 12-14 | Survival |
| 8a | 5 M, 3 F | twi/twi | rAAVhu68. hGALC | 6.8 × 10⁹ | 1.7 × 10¹⁰ | 4.0 | ICV | PND 12-14 | PND 40-42 |
| 8b | 5M, 5F | twi/twi | rAAVhu68. hGALC | 6.8 × 10⁹ | 1.7 × 10¹⁰ | 4.0 | ICV | PND 12-14 | Survival |

^{a}The twi allele consist of a loss-of-function mutation in the Galc gene.
^{b}Values were calculated using 0.4 g brain mass for an juvenile/adult mouse.
Abbreviations: F, female; Galc, galactosylceramidase (gene, mouse); GC, genome copies; ICV, intracerebroventricular; ID, identification number; ITFFB, intrathecal final formulation buffer; M, male; N, number of animals; N/A, not applicable; PND, postnatal day; ROA, route of administration; twi, Twitcher allele; WT, wild type.

In the PND 40 cohort, one twi/twi mouse administered vehicle (Animal 1074; Group 3a; N=⅑) was euthanized on PND 38 due to disease progression upon reaching 20% body weight loss in accordance with study-defined euthanasia criteria. All other animals in the PND 40 cohort survived to the scheduled necropsy.

In the survival cohort, all vehicle-treated wild type mice (N=8/8) survived until study termination, while the majority of rAAVhu68.hGALC -treated twi/twi mice (N=34/36) and vehicle-treated twi/twi controls (N=8/9) were euthanized in accordance with study-defined euthanasia criteria. All euthanized twi/twi mice exhibited signs of disease progression. Of the remaining mice, 2/36 rAAVhu68.hGALC -treated twi/twi mice and ⅑ vehicle-treated twi/twi mice were found dead. Animal 1071 (rAAVhu68.hGALC, 2.0 × 10¹⁰ GC; Group 7b) was found dead on PND 23 (11 days post treatment), likely due to a failure to thrive after weaning. Animal 1053 (rAAVhu68.hGALC, 2.0 × 10¹¹ GC; Group 5b) was found dead on PND 23 (11 days post treatment), and Animal 1062 (ITFFB; Group 3b) was found dead on PND 21 (7 days post treatment); however, the cause of death for both of these mice was unable to be determined.

Cumulatively, administration of rAAVhu68.hGALC at the three highest doses (2.0 × 10¹⁰ GC, 6.8 × 10¹⁰ GC, or 2.0 × 10¹¹ GC) resulted in a significant dose-dependent increase in survival of twi/twi mice compared to that of vehicle-treated twi/twi controls (FIG. 57). Median survival age was 40.5 days for the vehicle-treated twi/twi control group, while rAAVhu68.hGALC -treated mice exhibited a dose-related increase in survival. In rAAVhu68.hGALC -treated animals, the median survival age was 44.5 days (6.8 × 10⁹ GC), 48 days (2.0 × 10¹⁰ GC), 56.5 days (6.8 × 10¹⁰ GC), or 70 days (2.0 × 10¹¹ GC). All vehicle-treated WT mice survived to the study endpoint, and were euthanized at a median age of 125 days.

No clinical abnormalities related to rAAVhu68.hGALC were noted throughout the study.

The following three twi/twi mice were found dead during the study:
Animal 1071 (rAAVhu68.hGALC, 2.0 × 10¹⁰ GC; Group 7b) was found dead on PND 23 (11 days post dosing). One day prior on PND 22 (10 days post doing), this animal was noted to exhibit a smaller body size compared to its cagemate (<5 g), lethargy, and a hunched posture. Since no disease-related neurological signs were observed (e.g., tremors or ataxia) and histopathology revealed no significant finding, the cause of death was considered due to a failure to thrive post weaning.
Animal 1053 (rAAVhu68.hGALC, 2.0 × 10¹¹ GC; Group 5b) was found dead on PND 23 (11 days post treatment) after exhibiting seizure-like behavior (tonic-clonic activity with hyperactivity) the previous day. There were no gross or microscopic findings on histopathology for Animal 1053 besides the expected disease-related demyelination and globoid cell infiltration in the sciatic nerve and cerebellar white matter. Since seizure-like activity is not a documented phenotype of twi/twi mice, the cause of death for Animal 1053 was undetermined, although a possible ICV procedure-related etiology could not be ruled out.
Animal 1062 (ITFFB; Group 3b identified as M34-3F in the histopathology report as it died prior to microchip implantation) was found dead on PND 21 (7 days post treatment). No clinical abnormalities were noted, and histopathology revealed no significant finding besides the expected disease-related demyelination in the CNS. The cause of death for Animal 1062 was undetermined. The remaining rAAVhu68.hGALC -treated (N=67/69) and vehicle-treated (N=17/18) twi/twi mice in the PND 40 and survival cohorts exhibited clinical signs related to the Krabbe disease phenotype, including tremors, ataxia, hind limb weakness, hind limb paralysis, kyphosis, and/or body weight loss. These animals were either euthanized at the scheduled necropsy on PND 40 or euthanized for humane reasons due to disease progression upon meeting study-defined euthanasia criteria.

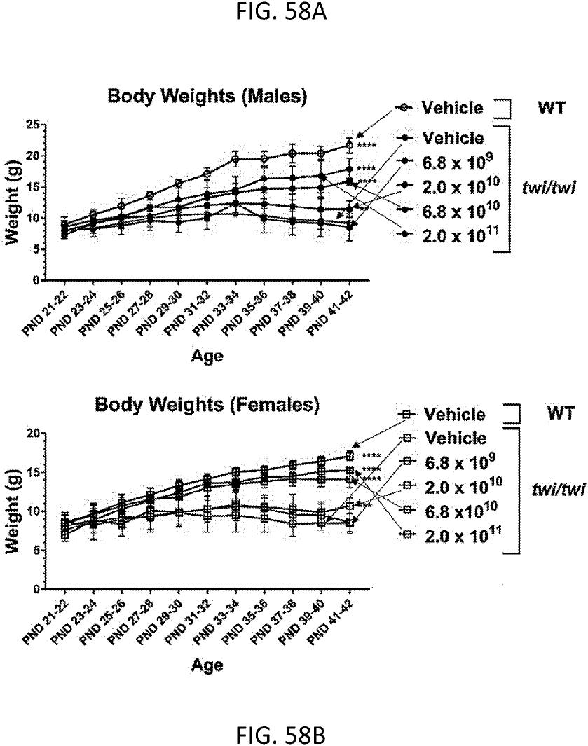

Administration of rAAVhu68.hGALC at the three highest doses (2.0 × 10¹⁰ GC, 6.8 × 10¹⁰ GC, or 2.0 × 10¹¹ GC) led to a significant dose-dependent rescue of body weight loss in both male and female twi/twi mice when compared to the body weights of sex-matched vehicle-treated twi/twi mice from PND 21-22 (weaning) to PND 41-42 (FIG. 58A and FIG. 58B).

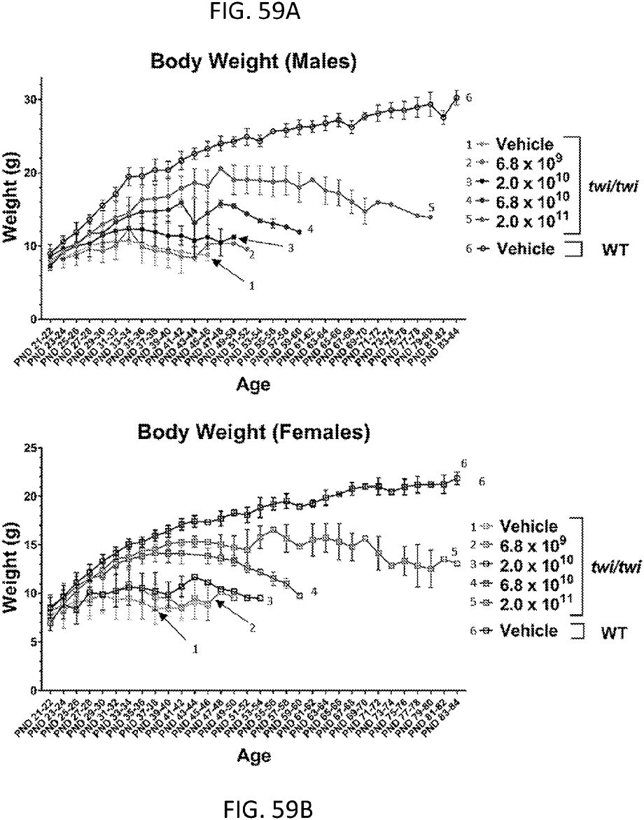

In the survival cohort, while all groups of twi/twi mice exhibited a decline in body weights after PND 41-42, the rate of body weight loss was inversely correlated to dose, with higher doses of rAAVhu68.hGALC resulting in a generally slower body weight loss in twi/twi mice. No gender differences related to body weights were noted in the study (FIG. 59A and FIG. 59B).

Administration of rAAVhu68.hGALC at the three highest doses (2.0 × 10¹⁰ GC, 6.8 × 10¹⁰ GC, or 2.0 × 10¹¹ GC) to twi/twi mice led to a significant dose-dependent reduction in total clinical scores compared to that of vehicle-treated twi/twi mice from PND 21-22 (weaning) to PND 41-42. The significant reduction in the clinical severity scores indicated an improvement in Krabbe disease-related clinical phenotypes following rAAVhu68.hGALC administration (FIG. 60).

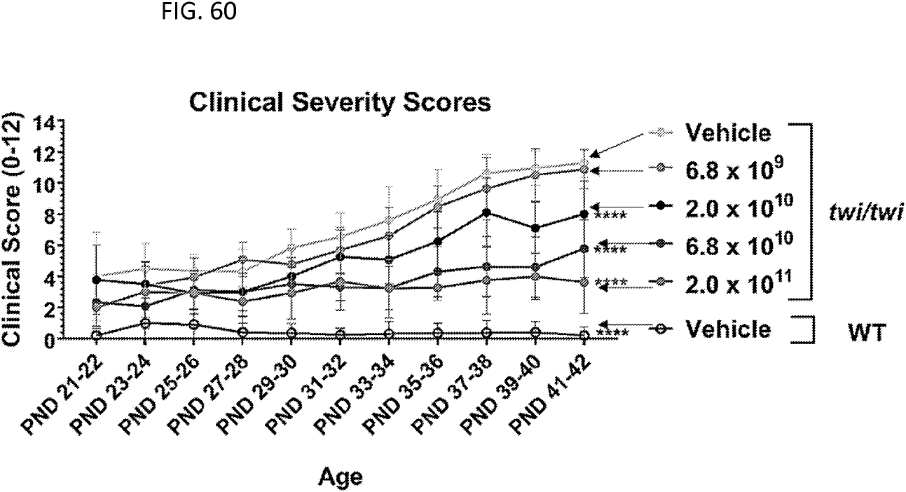

In the survival cohort, while all groups of twi/twi mice exhibited a progressive increase in clinical severity scores after PND 41-42 prior to humane euthanasia, the rate of increase was inversely correlated to dose, with higher doses of rAAVhu68.hGALC resulting in a generally slower increase in clinical severity scores in twi/twi mice. At the highest dose of rAAVhu68.hGALC (2.0 × 10¹¹ GC), twi/twi mice reached a significantly lower peak clinical severity score compared to that of vehicle-treated twi/twi mice at the time of humane euthanasia, suggesting a better overall clinical condition at the humane endpoint (FIG. 61).

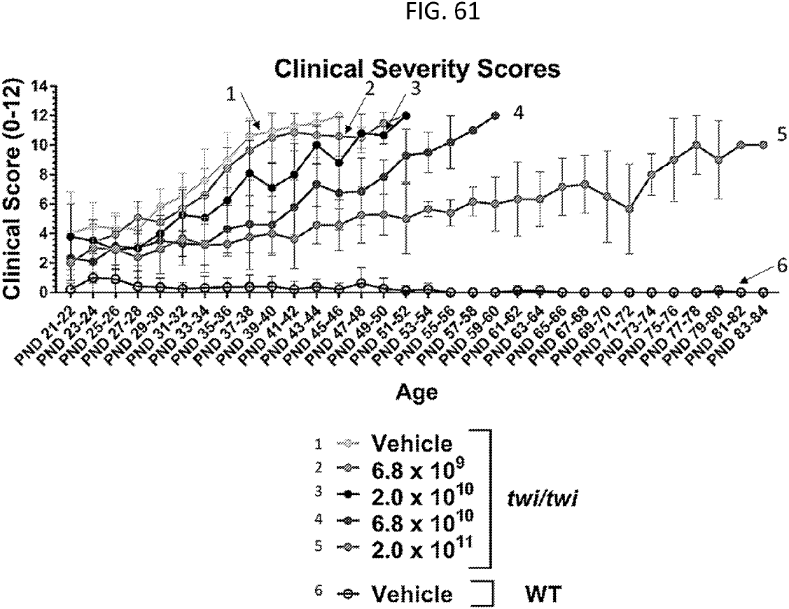

Neuromotor function was assessed by the RotaRod test, which evaluates coordination and balance by measuring the time to fall for mice running on a spinning rod that progressively accelerates. A decrease in fall latency indicates neuromotor impairment, while an increase in fall latency indicates improved neuromotor function. Administration of rAAVhu68.hGALC at a dose of 6.8 × 10¹⁰ GC or 2.0 × 10¹¹ GC to twi/twi mice led to a dose-dependent increase in fall latency compared to that of vehicle-treated twi/twi mice on PND 35-37 (3 weeks post treatment), which was indicative of improved neuromotor function following rAAVhu68.hGALC administration (FIG. 62).

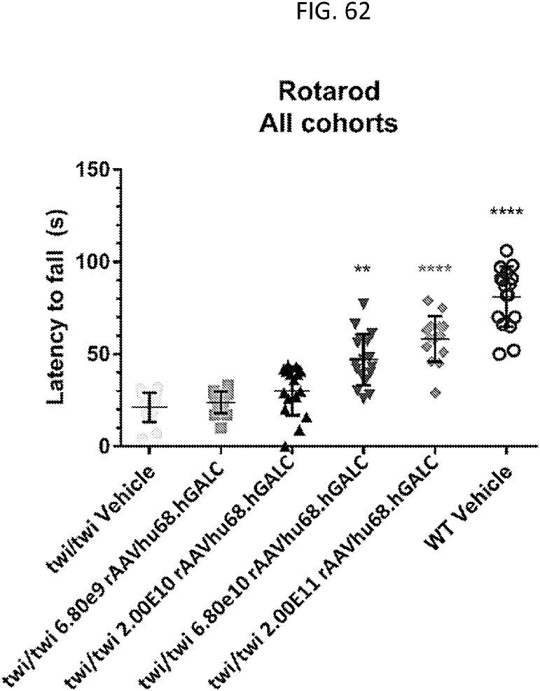

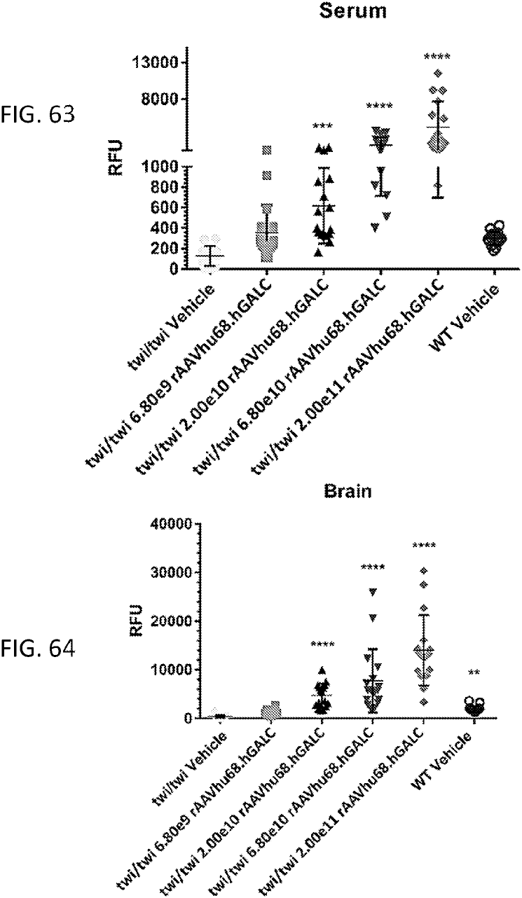

In serum, administration of rAAVhu68.hGALC at the three highest doses (2.0 × 10¹⁰ GC, 6.8 × 10¹⁰ GC, or 2.0 × 10¹¹ GC) to twi/twi mice led to a significant dose-dependent increase in GALC enzyme activity compared to that of vehicle-treated twi/twi mice on PND 35-37 (3 weeks post treatment). Furthermore, all doses of rAAVhu68.hGALC (6.8 × 10⁹ GC, 2.0 × 10¹⁰ GC, 6.8 × 10¹⁰ GC, or 2.0 × 10¹¹ GC) increased GALC enzyme activity to vehicle-treated wild type levels or higher (FIG. 63).

In the brain, administration of rAAVhu68.hGALC at the three highest doses (2.0 × 10¹⁰ GC, 6.8 × 10¹⁰ GC, or 2.0 × 10¹¹ GC) to twi/twi mice resulted in a significant dose-dependent increase in GALC enzyme activity compared to that of vehicle-treated twi/twi mice. Furthermore, these doses of rAAVhu68.hGALC (2.0 × 10¹⁰ GC, 6.8 × 10¹⁰ GC, or 2.0 × 10¹¹ GC) increased average GALC enzyme activity in twi/twi mice to levels higher than that of vehicle-treated wild type controls (FIG. 64).

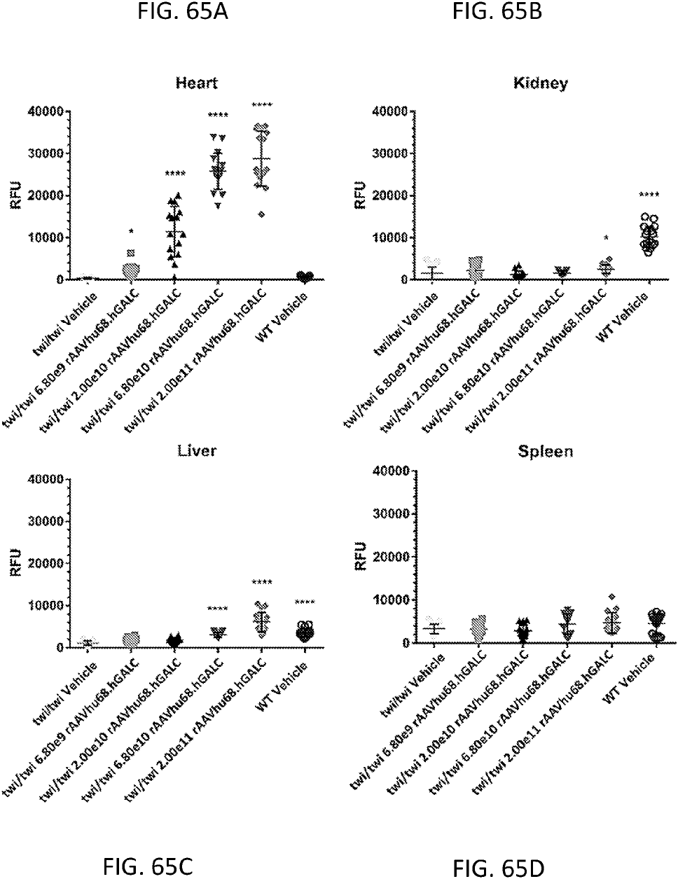

In the heart, administration of rAAVhu68.hGALC at all doses (6.8 × 10⁹ GC, 2.0 × 10¹⁰ GC, 6.8 × 10¹⁰ GC, or 2.0 × 10¹¹ GC) resulted in a significant dose-dependent increase in GALC enzyme activity compared to that of vehicle-treated twi/twi mice (FIG. 65A).

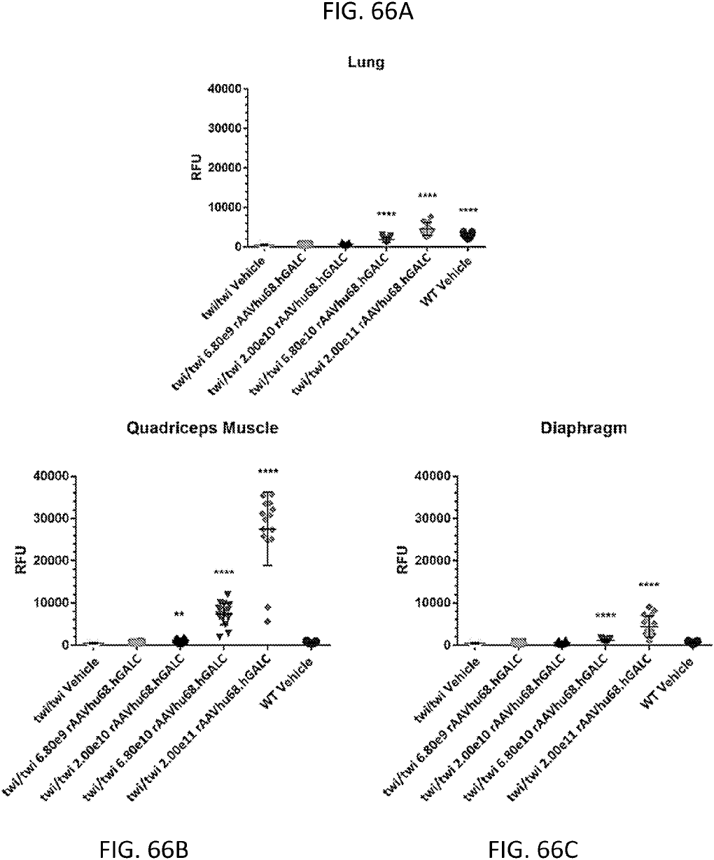

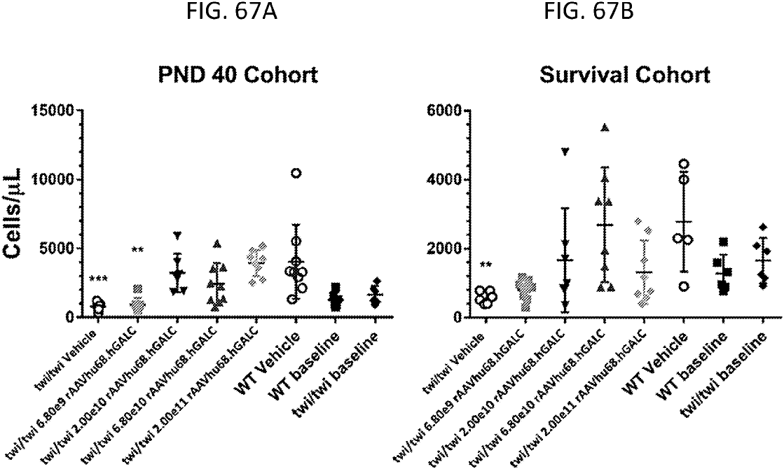

In the quadriceps muscle, administration of rAAVhu68.hGALC at the three highest doses (2.0 × 10¹⁰ GC, 6.8 × 10¹⁰ GC, or 2.0 × 10¹¹ GC) to twi/twi mice resulted in a significant dose-dependent increase in GALC enzyme activity compared to that of vehicle-treated twi/twi mice (FIG. 67A and FIG. 67B). In the liver (FIG. 65C), lung, and diaphragm (FIG. 66C), administration of rAAVhu68.hGALC at the two highest doses (6.8 × 10¹⁰ GC or 2.0 × 10¹¹ GC) to twi/twi mice resulted in a significant dose-dependent increase in GALC enzyme activity compared to that of vehicle-treated twi/twi mice.

In the kidney, administration of rAAVhu68.hGALC at the highest dose of 2.0 × 10¹¹ GC to twi/twi mice resulted in a significant increase in GALC enzyme activity compared to that of vehicle-treated twi/twi mice (FIG. 65B).

No increase in GALC enzyme activity was observed in the spleen of rAAVhu68.hGALC -treated twi/twi mice at any dose when compared to that of vehicle-treated twi/twi mice (FIG. 65D). However, it should be noted that the artificial fluorogenic substrate used in this assay is known to react with other lysosomal enzymes, such as β-galactosidase. Since background GALC enzyme activity was high in both the spleen and kidney of vehicle-treated twi/twi mice, there is the possibility of non-specific activity in these organs.

When comparing transgene product expression in rAAVhu68.hGALC -treated twi/twi mice to that of vehicle-treated wild type controls, average GALC enzyme activity in twi/twi mice was restored to wild type levels or higher in the heart and quadriceps muscle at all doses of rAAVhu68.hGALC (6.8 × 10⁹ GC, 2.0 × 10¹⁰ GC, 6.8 × 10¹⁰ GC, or 2.0 × 10¹¹ GC), in the brain and diaphragm at the three highest doses (2.0 × 10¹⁰ GC, 6.8 × 10¹⁰ GC, or 2.0 × 10¹¹ GC), in the spleen at the two highest doses (6.8 × 10¹⁰ GC or 2.0 × 10¹¹ GC), and in the liver and lungs at the highest dose (2.0 × 10¹¹ GC) (FIG. 65C and FIG. 66A).

No toxicity associated with rAAVhu68.hGALC treatment was observed on clinical pathology. Due to the phenotype of the twi/twi mouse model that leads to severe ambulation difficulties due to paresis of the hind legs (paralysis/dragging of both hind legs), these animals exhibited body wasting and failure to thrive as a result of inability to access food and water. Several abnormalities related to the twi/twi phenotype were noted in clinical pathology parameters, including lymphocyte counts, aspartate aminotransferase (AST), bilirubin, alanine aminotransferase (ALT), glucose, amylase, and triglycerides. In addition some abnormalities were corrected by rAAVhu68.hGALC administration. Only phenotype-or rAAVhu68.hGALC -corrected parameters are discussed in this section.

At baseline, untreated twi/twi mice exhibited lymphocyte counts similar to that of untreated WT controls. However, at PND 40 and the humane endpoint, vehicle-treated twi/twi mice exhibited a significant reduction in lymphocytes (lymphopenia) compared to that of vehicle-treated wild type controls as expected. In contrast, rAAVhu68.hGALC administration at a dose of 2.0 × 10¹⁰ GC, 6.8 × 10¹⁰ GC, or 2.0 × 10¹¹ GC to twi/twi mice normalized lymphocyte counts to levels similar to that of vehicle-treated wild type controls by PND 40-42 (4 weeks post treatment). At the humane endpoint, all doses of rAAVhu68.hGALC (6.8 × 10⁹ GC, 2.0 × 10¹⁰ GC, 6.8 × 10¹⁰ GC, or 2.0 × 10¹¹ GC) normalized lymphocyte counts in twi/twi mice to levels similar to that of vehicle-treated wild type controls (FIG. 67A and FIG. 67B).

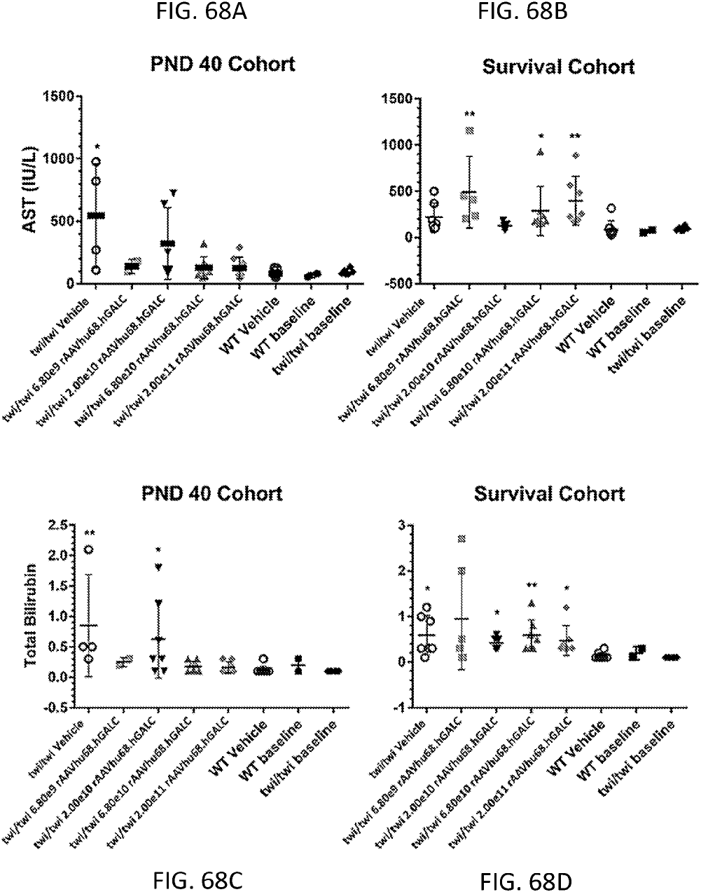

At baseline, serum AST levels appeared similar between untreated twi/twi mice and untreated WT controls, although the low sample numbers in the untreated twi/twi group precluded statistical analysis. At PND 40, AST levels were increased in vehicle-treated twi/twi mice when compared to that of vehicle-treated WT controls, while all rAAVhu68.hGALC -treated groups exhibited similar AST levels as vehicle-treated WT controls. At the humane endpoint, AST levels in vehicle-treated twi/twi mice were comparable to that of vehicle-treated WT controls, while AST levels were increased for twi/twi mice administered a rAAVhu68.hGALC dose of 6.8 × 10⁹ GC, 6.8 × 10¹⁰ GC, or 2.0 × 10¹¹ GC when compared to that of vehicle-treated WT controls (FIG. 68A and FIG. 68B). The observed AST elevations at both PND 40 and the humane endpoint appeared to be due largely to a small number of outliers. While no animals in the study, including the outliers with elevated AST levels, exhibited liver lesions on histopathology that would account for these elevations, a treatment-related effect could not be ruled out. However, because vehicle-treated twi/twi mice also displayed increased AST levels at PND 40, the most likely explanation is that this is a phenotype-related abnormality.

At baseline, total bilirubin levels appeared similar between untreated twi/twi mice and untreated WT controls, although the low sample numbers in the untreated twi/twi group precluded statistical analysis. At PND 40, bilirubin levels were increased for vehicle-treated twi/twi mice and rAAVhu68.hGALC -treated twi/twi mice at a dose of 2.0 × 10¹⁰ GC when compared to that of vehicle-treated WT controls. At the humane endpoint, bilirubin levels were increased for vehicle-treated twi/twi mice and rAAVhu68.hGALC -treated twi/twi mice at the three highest doses (2.0 × 10¹⁰ GC, 6.8 × 10¹⁰ GC, and 2.0 × 10¹¹ GC) when compared to that of vehicle-treated WT controls (FIG. 68C and FIG. 68D). No animals in the study exhibited liver lesions on histopathology that could account for elevations in total bilirubin. Therefore, with the caveat that an insufficient number of samples in the PND 40 cohort for the lowest rAAVhu68.hGALC dose group (6.8 × 10⁹ GC) precluded statistical analysis, it is possible that elevated bilirubin levels are related to the twi/twi mouse phenotype, and normalization of bilirubin levels at PND 40 in the two highest rAAVhu68.hGALC dose groups (6.8 × 10¹⁰ GC and 2.0 × 10¹¹ GC) was a dose-related treatment effect.

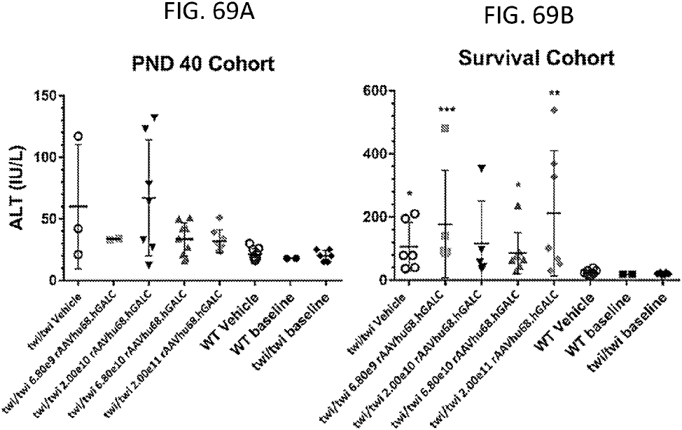

At baseline, alanine aminotransferase (ALT) levels appeared similar between untreated twi/twi mice and untreated WT controls, although the low sample numbers in the untreated twi/twi group precluded statistical analysis. At PND 40, all vehicle-treated and rAAVhu68.hGALC -treated groups of twi/twi mice exhibited ALT levels similar to that of vehicle-treated WT controls. At the humane endpoint, ALT levels were increased in vehicle-treated twi/twi mice and rAAVhu68.hGALC -treated twi/twi mice at doses of 6.8 × 10⁹ GC, 6.8 × 10¹⁰ GC, and 2.0 × 10¹¹ GC when compared to levels in the vehicle-treated WT controls (FIG. 69A and FIG. 69B). No animals in the study exhibited liver lesions on histopathology that could account for the elevations in ALT. Therefore, the ALT elevations observed in twi/twi mice at the humane endpoint are likely related to the twi/twi mouse phenotype and appear unaffected by rAAVhu68.hGALC administration.

At baseline, glucose levels appeared similar between untreated twi/twi mice and untreated WT controls, although the low sample numbers in the untreated twi/twi group precluded statistical analysis. At PND 40, vehicle-treated twi/twi mice and twi/twi mice administered a rAAVhu68.hGALC dose of 2.0 × 10¹⁰ GC exhibited a reduction in glucose levels compared to that of vehicle-treated WT controls, while glucose levels in the two highest rAAVhu68.hGALC dose groups (6.8 × 10¹⁰ GC and 2.0 × 10¹¹ GC) appeared similar to that of the vehicle-treated WT controls. At the humane endpoint, vehicle-treated twi/twi mice and twi/twi mice administered the highest dose of rAAVhu68.hGALC (2.0 × 10¹¹ GC) exhibited reduced glucose levels compared to that of vehicle-treated WT controls, while the remaining rAAVhu68.hGALC dose groups (6.8 × 10⁹ GC, 2.0 × 10¹⁰ GC, 6.8 × 10¹⁰ GC) exhibited glucose levels similar to that of the vehicle-treated WT controls (FIG. 70A and FIG. 70B).

Because mice were not fasted prior to blood collection at necropsy, the significance of this finding is unclear. Since the vehicle-treated twi/twi mice at both PND 40 and the humane endpoint exhibited a reduction in glucose levels compared to that of WT controls, this finding may relate to feeding difficulty, which twi/twi mice exhibit as they approach the humane endpoint due to progressive ataxia. Therefore, with the caveat that an insufficient number of samples in the PND 40 cohort for the lowest rAAVhu68.hGALC dose group (6.8 × 10⁹ GC) precluded statistical analysis, it is possible that normalization of glucose levels at the two highest doses of rAAVhu68.hGALC (6.8 × 10¹⁰ GC and 2.0 × 10¹¹ GC) in twi/twi mice represents a dose-related treatment effect at PND 40. It is unclear whether this treatment effect persists until the humane endpoint in twi/twi mice, as a dose-dependent effect was not evident.

At baseline, amylase levels appeared similar between untreated twi/twi mice and untreated WT controls, although the low sample numbers in the untreated twi/twi group precluded statistical analysis. At PND 40, similar amylase levels were observed for vehicle-treated twi/twi mice and all rAAVhu68.hGALC dose groups when compared to that of vehicle-treated WT controls. At the humane endpoint, vehicle-treated twi/twi mice and twi/twi mice administered a rAAVhu68.hGALC dose of 6.8 × 10¹⁰ GC exhibited elevated amylase levels compared to that of vehicle-treated WT controls, while the remaining rAAVhu68.hGALC dose groups (6.8 × 10⁹ GC, 2.0 × 10¹⁰ GC, and 2.0 × 10¹¹ GC) exhibited amylase levels similar to that of the vehicle-treated WT controls (FIG. 70C and FIG. 70D). Since vehicle-treated twi/twi mice exhibited amylase elevations above WT levels at the humane endpoint, this finding may relate to feeding difficulty, which twi/twi mice exhibit as they approach the humane endpoint due to progressive ataxia. The normalization of amylase levels at the humane endpoint in most rAAVhu68.hGALC dose groups (6.8 × 10⁹ GC, 2.0 × 10¹⁰ GC, or 2.0 × 10¹¹ GC) likely reflects a treatment-related effect.

At baseline, triglyceride levels appeared similar between untreated twi/twi mice and untreated WT controls, although the low sample numbers in the untreated twi/twi group precluded statistical analysis. At PND 40, a reduction in triglyceride levels was observed for twi/twi mice administered a rAAVhu68.hGALC dose of 2.0 × 10¹⁰ GC when compared to that of vehicle-treated WT controls. At the humane endpoint, a reduction in triglyceride levels was observed in vehicle-treated twi/twi mice and twi/twi mice administered the two highest doses of rAAVhu68.hGALC (6.8 × 10¹⁰ GC and 2.0 × 10¹¹ GC). Because mice were not fasted prior to blood collection at necropsy, the significance of this finding is unclear. The observed reduction in triglyceride levels in vehicle-treated twi/twi mice at the humane endpoint when compared to the levels in WT mice may relate to feeding difficulty, which twi/twi mice exhibit as they approach the humane endpoint due to progressive ataxia. No obvious treatment-related effect of rAAVhu68.hGALC on triglyceride levels was observed at either PND 40 or the humane endpoint, as reductions in triglycerides did not correlate with dose.

No toxicity related to rAAVhu68.hGALC was noted in the brain, spinal cord, sciatic nerve, or any of visceral organs evaluated in this study.

Efficacy of rAAVhu68.hGALC in the treatment of the Krabbe disease-related pathology in the twi/twi mouse was demonstrated by significant or trending reduction (correction) of myelin loss and globoid cell infiltration in the nervous system across all rAAVhu68.hGALC -treated cohorts compared to vehicle-treated twi/twi mice. While there was no clear dose effect, mice that received the highest rAAVhu68.hGALC dose (2.0 × 10¹¹ GC) and, to a lesser extent, second highest rAAVhu68.hGALC dose (6.8 × 10¹⁰ GC) generally exhibited more significant correction microscopically of the twi/twi disease phenotype compared to the lower doses.

At baseline (PND 12-14), globoid cells were present in the untreated twi/twi mouse. The severity was generally minimal to mild, and was more pronounced in spinal cord and the peripheral nerves when compared to the brain. This finding suggested that disease-related pathology was already present at the time of rAAVhu68.hGALC administration. At PND 40 (4 weeks post treatment) and humane endpoint in the survival cohort euthanasia (up to 10 weeks post treatment for twi/twi mice), rAAVhu68.hGALC -treated twi/twi mice exhibited reduced demyelination and globoid cell infiltration when compared to that of vehicle-treated twi/twi controls in several neuroanatomical areas (cerebrum, spinal cord, and/or sciatic nerve). Of note, all doses of rAAVhu68.hGALC (6.8 × 10⁹ GC, 2.0 × 10¹⁰ GC, 6.8 × 10¹⁰ GC, and 2.0 × 10¹¹ GC) reduced demyelination and globoid cell infiltration in the cerebral white matter at both PND 40 and the humane endpoint when compared to that of vehicle-treated twi/twi controls. The two highest doses of rAAVhu68.hGALC (6.8 × 10¹⁰ GC and 2.0 × 10¹¹ GC) reduced demyelination and globoid cell infiltration in the sciatic nerve at PND 40 when compared to that of vehicle-treated twi/twi controls. The highest dose of rAAVhu68.hGALC (2.0 × 10¹¹ GC) reduced demyelination globoid cell infiltration in the spinal cord at PND 40 when compared to that of vehicle-treated twi/twi controls.

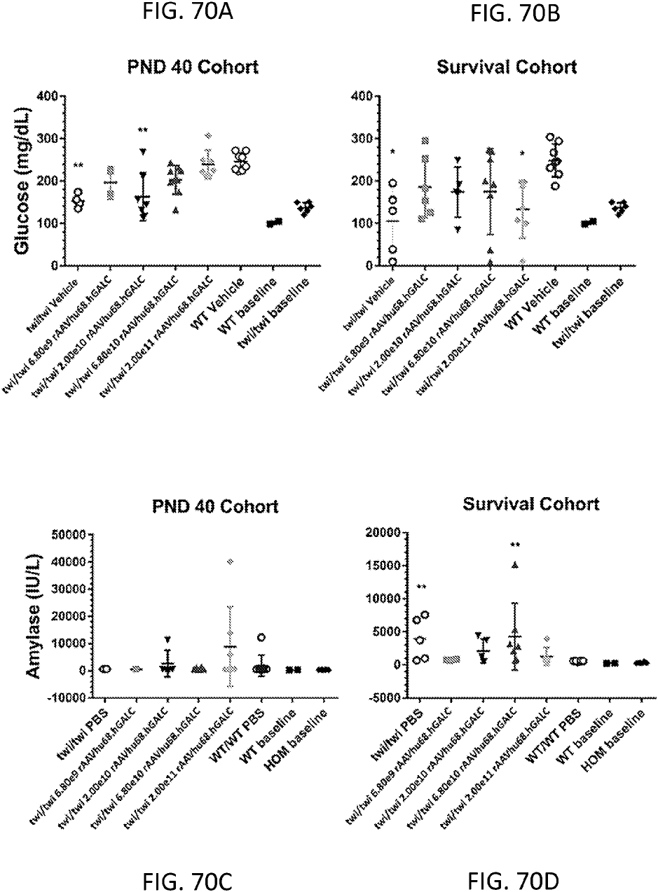

Histopathology did not reveal any significant lesion in the livers of mice that could explain the observed changes in liver-related clinical pathology parameters. The only notable finding in the liver was an improvement in liver microvacuolation following rAAVhu68.hGALC administration. In the survival cohort, vehicle-treated twi/twi mice did not exhibit hepatocellular vacuolation (Grade 0), while all vehicle-treated WT mice exhibited mild to moderate microvesicular vacuolation (Grade 2 to Grade 3). Administration of rAAVhu68.hGALC at all doses (6.8 × 10⁹ GC, 2.0 × 10¹⁰ GC, 6.8 × 10¹⁰ GC, or 2.0 × 10¹¹ GC) to twi/twi mice resulted in increased microvacuolation in the majority of animals to levels comparable to that of vehicle-treated WT controls (FIG. 71). Hepatocellular vacuolation is typically due to the presence of triglycerides and glycogen physiological reserves. The absence of vacuolation in vehicle-treated twi/twi mice could be due to the absence of triglycerides and/or glycogen reserves as a result of the wasting phenotype at the humane endpoint. The increase in vacuolation following rAAVhu68.hGALC treatment likely reflects an improvement in the wasting phenotype of twi/twi mice at all doses of rAAVhu68.hGALC (6.8 × 10⁹ GC, 2.0 × 10¹⁰ GC, 6.8 × 10¹⁰ GC, or 2.0 × 10¹¹ GC). Liver microvacuolation improvements did not appear dose-dependent.

Three mice were found dead, including Animal 1071 (rAAVhu68.hGALC, 2.0 × 10¹⁰ GC; Group 7b; PND 23, 11 days post treatment), Animal 1053 (rAAVhu68.hGALC, 2.0 × 10¹¹ GC; Group 5b; PND 23, 11 days post treatment), and Animal 1062 (ITFFB; Group 3b; PND 21, 7 days post treatment). Aside from findings of demyelination and globoid cell infiltration in the CNS and PNS that are typical of the twi/twi mouse phenotype, no other gross or microscopic abnormalities were noted. The causes of these deaths were undetermined; however, a procedural-related cause could not be ruled out for Animal 1053 (rAAVhu68.hGALC, 2.0 × 10¹¹ GC; Group 5b), which exhibited a seizure 1 day prior to death.

IBA1 immunohistochemistry was performed on the brain (cortex, cerebellum, brainstem), spinal cord (cervical, lumbar, thoracic), and sciatic nerves to visualize globoid cells in the CNS and PNS. The size of individual IBA1-positive cells (mean object area) was measured using image analysis software. A reduction in the size of IBA1-positive cells was expected with an improvement in disease phenotype.

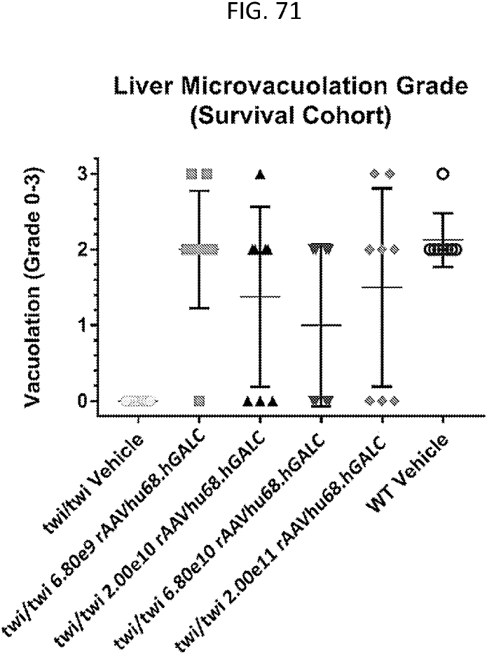

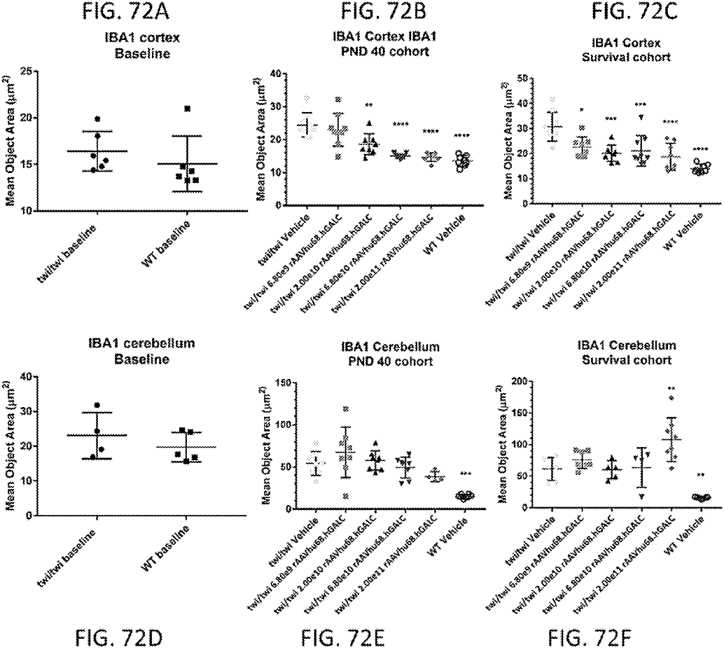

In the cortex, IBA1-positive cells in the untreated twi/twi mice were similar in size to that of the untreated WT mice at baseline. At PND 40, vehicle-treated twi/twi mice exhibited significantly larger IBA-1-positive cells compared to that of the vehicle-treated WT controls. Administration of rAAVhu68.hGALC at the three highest doses (2.0 × 10¹⁰ GC, 6.8 × 10¹⁰ GC, or 2.0 × 10¹¹ GC) to twi/twi mice resulted in a significant dose-dependent reduction in IBA-1-positive cell size compared to that of the vehicle-treated twi/twi mice. Notably, at the two highest doses of rAAVhu68.hGALC (6.8 × 10¹⁰ GC or 2.0 × 10¹¹ GC), IBA-1-positive cells were similar in size to that of the vehicle-treated WT controls. At humane endpoint (survival cohort), vehicle-treated twi/twi mice exhibited significantly larger IBA-1-positive cells compared to that of the vehicle-treated WT controls. Administration of rAAVhu68.hGALC at all doses (6.8 × 10⁹ GC, 2.0 × 10¹⁰ GC, 6.8 × 10¹⁰ GC, or 2.0 × 10¹¹ GC) to twi/twi mice resulted in a significant and generally dose-dependent reduction in IBA-1-positive cell size compared to that of the vehicle-treated twi/twi mice (FIG. 72A - FIG. 72C).

In the cerebellum, IBA1-positive cells in the untreated twi/twi mice were similar in size to that of the untreated WT mice at baseline. At PND 40, vehicle-treated twi/twi mice exhibited significantly larger IBA-1-positive cells compared to that of the vehicle-treated WT controls. Administration of rAAVhu68.hGALC to twi/twi mice did not reduce IBA-1-positive cell size at any dose when compared to that of the vehicle-treated twi/twi mice. At humane endpoint (survival cohort), vehicle-treated twi/twi mice exhibited significantly larger IBA-1-positive cells compared to that of the vehicle-treated WT controls. Administration of rAAVhu68.hGALC at the highest dose (2.0 × 10¹¹ GC) to twi/twi mice resulted in a significant increase in IBA-1-positive cell size compared to that of the vehicle-treated twi/twi mice (FIG. 72D - FIG. 72F).

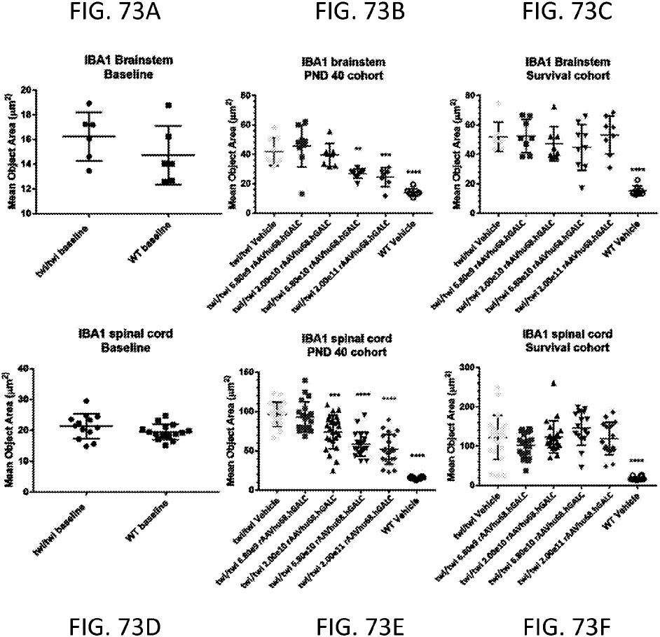

In the brainstem, IBA1-positive cells in the untreated twi/twi mice were similar in size to that of the untreated WT mice at baseline. At PND 40, vehicle-treated twi/twi mice exhibited significantly larger IBA-1-positive cells compared to that of the vehicle-treated WT controls. Administration of rAAVhu68.hGALC at the two highest doses (6.8 × 10¹⁰ GC or 2.0 × 10¹¹ GC) to twi/twi mice resulted in a significant reduction in IBA-1-positive cell size compared to that of the vehicle-treated twi/twi mice. At humane euthanasia, vehicle-treated twi/twi mice exhibited significantly larger IBA-1-positive cells compared to that of the vehicle-treated WT controls. Administration of rAAVhu68.hGALC to twi/twi mice did not reduce IBA-1-positive cell size at any dose when compared to that of the vehicle-treated twi/twi mice (FIG. 73A - FIG. 73C).

In the spinal cord, IBA1-positive cells in the untreated twi/twi mice were similar in size to that of the untreated WT mice at baseline. At PND 40, vehicle-treated twi/twi mice exhibited significantly larger IBA-1-positive cells compared to that of the vehicle-treated WT controls. Administration of rAAVhu68.hGALC at the three highest doses (2.0 × 10¹⁰ GC, 6.8 × 10¹⁰ GC, or 2.0 × 10¹¹ GC) to twi/twi mice resulted in a significant reduction in IBA-1-positive cell size compared to that of the vehicle-treated twi/twi mice. At humane endpoint (survival cohort), vehicle-treated twi/twi mice exhibited significantly larger IBA-1-positive cells compared to that of the vehicle-treated WT controls. Administration of rAAVhu68.hGALC to twi/twi mice did not reduce IBA-1-positive cell size at any dose when compared to that of the vehicle-treated twi/twi mice (FIG. 73D - FIG. 73F).

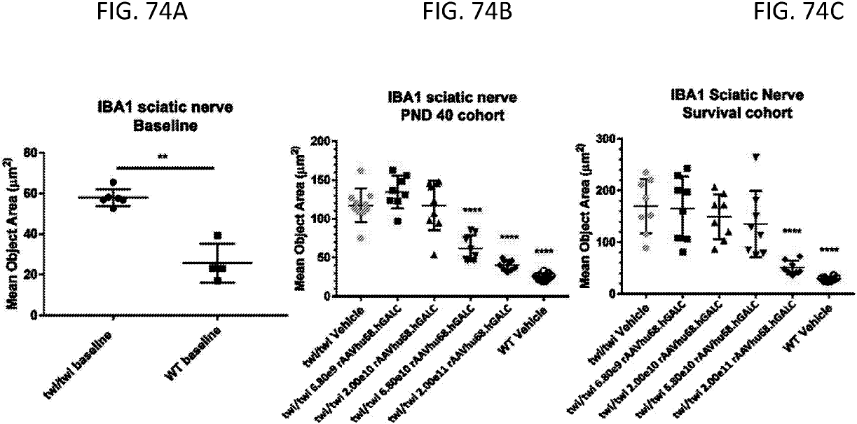

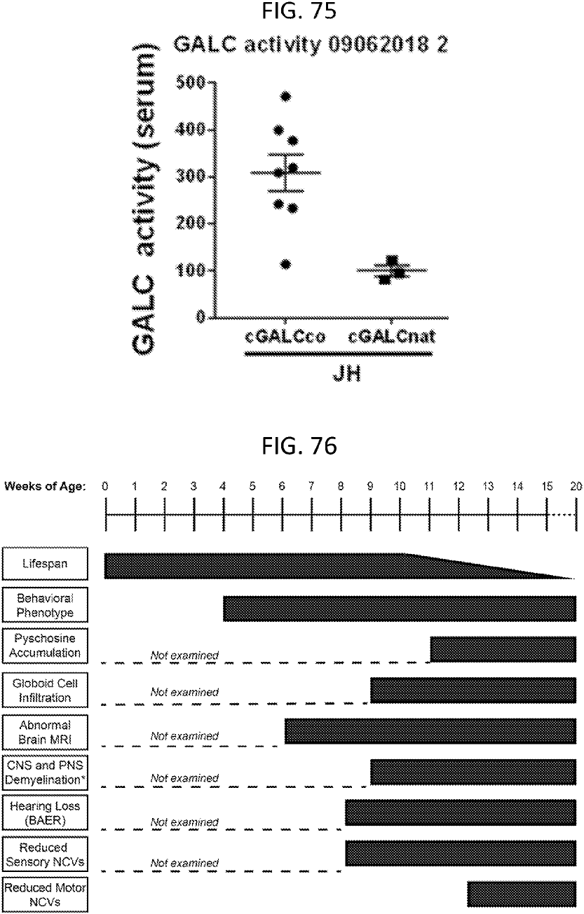

In the sciatic nerve, IBA1-positive cells in the untreated twi/twi mice were significantly larger in size than that of the untreated WT controls at baseline. At PND 40, vehicle-treated twi/twi mice still exhibited significantly larger IBA-1-positive cells compared to that of the vehicle-treated WT controls. Administration of rAAVhu68.hGALC at the two highest doses (6.8 × 10¹⁰ GC or 2.0 × 10¹¹ GC) to twi/twi mice resulted in a significant reduction in IBA-1-positive cell size compared to that of the vehicle-treated twi/twi mice. At humane euthanasia, vehicle-treated twi/twi mice exhibited significantly larger IBA-1-positive cells compared to that of the vehicle-treated WT controls. Administration of rAAVhu68.hGALC at the highest dose (2.0 × 10¹¹ GC) to twi/twi mice resulted in a significant reduction in IBA-1-positive cell size compared to that of the vehicle-treated twi/twi mice. Moreover, at the highest dose of rAAVhu68.hGALC (2.0 × 10¹¹ GC), IBA-1-positive cells approached vehicle-treated WT sizes at both PND 40 and humane euthanasia (FIG. 74A - FIG. 74C).

In summary, the MED was determined to be 2.0 × 10¹⁰ GC (5.0 × 10¹⁰ GC/g brain) because this dose led to significant improvements in survival, body wasting/failure to thrive (body weight loss), Krabbe disease-related clinical symptoms (clinical assessment scoring), phenotypic lymphopenia (which was possibly indicative of a reduction in autonomic neuronal degeneration). This dose reduced demyelination, globoid cell infiltration, and neuroinflammation in the brain (LFB/PAS semi-quantitative scoring), and reduced globoid cell size in the brain and spinal cord (IBA-1 IHC quantification). This dose was also the minimal dose leading to significantly increased transgene product expression (GALC activity) in the brain, which is a key target tissue.

### Example 9 - Efficacy of AAV-mediated Gene Therapy to Treat Krabbe Dogs - Injection of rAAVhu68.CB7.CI.cGALCco.rBG Via the Cisterna Magna

While an informative disease model, the Twitcher mouse does have some limitations. The mice display only mild CNS involvement, which is distinct from infantile Krabbe patients, who present with more severe CNS features of demyelination of brain atrophy. Furthermore, the small size of the mouse poses experimental challenges. The ICV route must be used in mice because their small size makes it difficult to reliably inject AAV vector via the intended clinical route (ICM). Sufficient quantities of serial samples of CSF and blood also cannot be obtained from mice for all of the desired pharmacological assays. Treatment with rAAVhu68.GALC was therefore evaluated in a larger animal, the canine model of Krabbe disease, which can overcome these technical constraints and confirm the scalability of our therapeutic approach.

Like the Twitcher mouse, the Krabbe dog is a naturally occurring autosomal recessive disease model deriving from a spontaneous A to C mutation in the GALC gene that causes a missense mutation (Y158S). The mutant GALC protein has residual enzymatic activity close to 0%, which is similar to GALC activity levels observed in patients with the infantile form of Krabbe disease. While heterozygous dogs do not display symptoms, dogs homozygous for the mutation are affected.

Progression of the Krabbe canine phenotype include elevated psychosine levels, demyelination, and globoid cell infiltration in both the CNS and PNS, along with associated behavioral phenotypes. Krabbe dogs develop hind limb weakness, thoracic limb dysmetria, and tremors at approximately 4-6 weeks of age. Similar to patients with infantile Krabbe disease, Krabbe dogs present consistent and rapid neurologic deterioration after the onset of symptoms. Ultimately, these symptoms progress to a humane endpoint characterized by severe ataxia, pelvic limb paralysis, wasting, urinary incontinence, and sensory deficits by around 8-15 weeks of age (Fletcher T.F. & Kurtz H.J. (1972) Am J Pathol. 66(2):375-8; Wenger D.A. (2000) Molec Med Today. 6(11):449-451; Bradbury A.M., et al. (2018) Hum Gene Ther. 29(7):785-801).

TABLE

Murine and canine models of Krabbe disease and comparison with human early infantile presentation

| | Mutation | GALC Activity Levels | Presenting Signs and Evolution | Pathology |
|---|---|---|---|---|
| Twitcher Mouse | Murine: G.A1017 (W339X) | < 10% of normal | Twitching, hind leg weakness at 18-22 days | PNS > CNS |
| | | | Severe weight loss and paralysis at 40-45 days | |
| Krabbe Dog | Canine: A.C 473 (Y158S) | < 10% of normal | Thoracic limb dysmetria, hind limb weakness, tremors at 4-6 weeks | PNS and CNS |
| | | | Pelvic limb paralysis, severe ataxia, wasting, urinary incontinence, sensory deficits at about 15 weeks | |
| Human Early Infantile Krabbe Disease | Various, most frequent is a 30 kb deletion starting near intron 10 (502T/del) | < 10% of normal | Irritability, spasticity, dysphagia before 6 months of age | PNS and CNS |
| | | | Regression and absence of new milestones acquisition, deafness, blindness, seizures, death before 2 years | |

Abbreviations: A, adenine; C, cytosine; CNS, central nervous system; G, guanine; GALC, galactosylceramidase; kb, kilobase; PNS, peripheral nervous system; W339X, tryptophan changed to a stop codon at position 339; Y158S, tyrosine to serine substitution at position 158.

The purpose of this study was to evaluate the efficacy, pharmacology, safety, and biodistribution of AAVhu68.CB7.CI.cGALCco.rBG, a recombinant adeno-associated virus (AAV) serotype hu68 vector expressing canine galactocerebrosidase (GALC) enzyme, following intra-cisterna magna (ICM) administration in a canine model of infantile Krabbe. At the age of 2-3 weeks, Krabbe affected dogs received a single ICM administration of either AAVhu68.cGALCco at a dose of 3.0 × 10¹³ GC or vehicle (intrathecal final formulation buffer [ITFFB]). A wild type littermate was also administered vehicle.

In-life evaluations included cageside observations, body weight monitoring, biweekly behavior and motor function monitoring, physical exams, standardized neurological exams, evaluation of brain myelination (assessed by magnetic resonance imaging [MRI] and brainstem auditory evoked response [BAER]) and peripheral nerve myelination (assessed by nerve conduction studies [NCS]), CSF sphingolipids quantification to analyze accumulation of psychosine (disease biomarker), transgene product expression in serum and CSF (GALC activity assay). Necropsies were performed at 6 months post treatment (n=2) or at humane endpoint (n=4), except for the healthy wild type untreated littermate control which was euthanized at the same time as the last treated animal. At necropsy, tissues were obtained from each animal for a comprehensive histopathological examination and biodistribution analysis. Samples of brain tissue and peripheral nerves were collected to evaluate myelination and storage (luxol-blue/periodic acid-Schiff [PAS] staining). Samples of brain and spinal cord tissues were also collected to quantify activation of microglia and globoid cell infiltration (IBA1 IHC).

Several relevant biomarkers are accessible in this large animal model as well as the ability to do the intended clinical route of administration (ICM), which makes it an attractive model to study efficacy of gene therapy. To prevent confounding results with exaggerated immune responses to a foreign protein, a vector encoding the canine version of GALC (AAVhu68.CB7.CI.cGALCco.rBG) was administered. While the transgene administered in differed, the vector utilized the same ubiquitous CB7 promoter, AAVhu68 capsid as AAVhu68.CB7.CI.hGALCco.rBG. The age of the animals was selected to ensure that the Krabbe dogs were treated prior to the onset of behavioral symptoms. Furthermore, this age mirrors that of the intended infantile patient population.

Following group assignment, each animal received a single ICM injection of AAVhu68.CB7.cGALCco at a dose of 3 × 10¹³ GC/animal or vehicle (ITFFB). Group designations, dose levels, and the route of administration (ROA) are presented in the table below. A study design is provided in FIG. 77.

| Group | Treatment | Dose (GC) | ROA | GALC Loss-of-Function Mutation Status^{a} | Animal ID | Sex | Dosing^{b} | Scheduled Necropsy | Actual Necropsy | Age At Necropsy |
|---|---|---|---|---|---|---|---|---|---|---|
| 1 | Vehicle | N/A | ICM | Homozy gous (Krabbe Dogs) | K930 | F | Study Day 0 | Study Day 70±3 | Study Day 35^{c} | 8 weeks |
| | | | | | K948 | M | | | Study Day 66^{c} | 12 weeks |
| 2a | AAVhu68. cGALCco | 3.0 × 10¹³ | ICM | Homozy gous (Krabbe Dogs) | K938 | M | Study Day 0 | Study Day 180±3 | Study Day 181 | 28 weeks |
| | | | | | K939 | F | | | Study Day 182 | 28 weeks |
| 2b | AAVhu68. cGALCco | 3.0 × 10¹³ | ICM | Homozy gous (Krabbe Dogs) | K937 | M | Study Day 0 | Long Term | Study Day 261^{d} | 39 weeks |
| | | | | | K933 | M | | | Study Day 572 | 85 weeks^{e} |
| 3 | Vehicle | N/A | ICM | Wild Type | K928 | F | Study Day 0 | Long Term | Study Day 587 | 87 weeks^{f} |

^{a}Mutation status refers to the canine GALC A.C 473 (Y158S) loss-of-function mutation. Animals homozygous for the GALC loss-of-function mutation exhibit phenotypes mirroring human Krabbe disease and are referred to as Krabbe dogs in the text.
^{b}Animals were 2-3 weeks old at the time of dosing.
^{c}Unscheduled necropsy performed because animal met human euthanasia criteria consistent with disease progression.
^{d}Unscheduled necropsy performed due to an acute episode of hyperthermia and suspected seizures. Animal did not exhibit symptoms consistent with the known natural history of canine Krabbe disease.
^{e}Unscheduled necropsy performed because substantial body weight loss (28%).
^{f}Euthanized when the last treated animal was euthanized.
Abbreviations: AAVhu68.cGALCco, AAVhu68.CB7.CI.cGALCco.rBG; F, female; GALC, galactosylceramidase (gene, canine); GC, genome copies; ICM, intra-cisterna magna; ID, identification number; M, male; N/A, not applicable; ROA, route of administration; TBD, to be determined.

The scheduled 180-day necropsy timepoint was chosen to collect tissues at a fixed timepoint and look for biomarkers of disease 6 months after treatment and 4 months after survival of the last vehicle-treated Krabbe dogs. This timing was considered sufficient duration to measure the eventual progression of meaningful disease-relevant phenotypes and biomarkers while being able to compare to the juvenile untreated dogs that had reached humane endpoint before 12 weeks of age. In order to get longer term follow-up and assess durability of the therapy, two dogs were enrolled in a long-term follow-up (up to 19 months post treatment).

Progression of the behavior and motor function was assessed using bi-weekly videotaping, periodical neurological examination, brain MRI, and electrophysiology (NCV and BAER). Initial timepoints were designed to capture disease apparition and progression in the vehicle Krabbe dog group. Periodic testing was performed every 2 to 3 months in the treated Krabbe dogs. One wild type vehicle-treated dog was included in the long term follow-up for comparison to treated animals.

The two vehicle-treated Krabbe dogs reached the pre-defined humane endpoint characterized by severe hindlimb weakness and inability to stand and walk at the age of 8 weeks (Animal K930), and 12 weeks (Animal K948), consistent with the natural history of the disease.

One vector-treated Krabbe dog, K937, was found laterally recumbent with marked hyperthermia (106.4 degrees) on the morning observation at 9 months (38 weeks) posttreatment. The veterinarians suspected a seizure episode and administered 0.5 mg/kg of valium IV and collected blood for CBC, chemistry, and culture as well as CSF for cytology. Although the rectal temperature returned to normal, the animal remained recumbent and euthanasia was therefore elected. Significant findings on blood work included a neutrophilic leukocytosis (16,647 / µl; 4-fold increase compared to previous measured value), mild lymphopenia (337 / µL), increased D-dimers (>5,400 ng/mL; range <250), mildly increased fibrinogen (455 mg/dl; 150-400 mg/dLrange), AST (184 IU/L; range 15-66 IU / L), and BUN (37 mg/dL; range 6-31 mg /dL). Blood culture revealed the presence of methicillin resistant Staphylococcus epidermidis. CSF showed mildly elevated protein levels (69 mg/dL), a low number of WBC (2 per µL) and no infectious agent. The pathology report revealed Krabbe-related lesions of demyelination and globoid cell infiltration that were less pronounced than vehicle-treated Krabbe dog controls (Animals K930 and K948), and similar to the 2 treated dogs from the 6 months scheduled necropsy timepoint (Animals K938 and K939). The spinal cord and peripheral nerve showed no demyelination suggesting treatment efficacy was maintained and consistent with normal motor function of this animal prior to the acute hyperthermia episode.

The clinical presentation (acute lateral recumbency, hyperthermia, suspected seizures) of animal K937 is not the typical presentation of Krabbe disease. Clinical pathology and pathology report are compatible with an infectious cause. Blood culture revealed growth of an antibiotic resistant Staphylococcus epidermidis. The dog’s record indicates that he was being treated with topical antibiotic ointment due to abrasive lesions on the right front P4 paw pad from 8 days prior to the febrile episode. Suspected seizures related to Krabbe disease brain demyelination and globoid cell infiltration cannot however be ruled out.

Animal K933 (AAVhu68.cGALC.co-treated) was euthanized at 19.5 months of age due to body weight loss. The body weight loss was a result of recurrent vomiting and regurgitations that did not respond to symptomatic treatment. An X-ray performed approximately 4 days before maximal weight loss of 28% at euthanasia revealed a gas-distended esophagus compatible with mega-esophagus. On necropsy, the dog presented bilateral severely enlarged salivary glands which could have explained the swallowing difficulties and regurgitations. Electromyography of the esophagus performed terminally indicated no sign of denervation (lack of spontaneous activity). Behavior and motor function and neurological examination were normal at the time of euthanasia.

The two vehicle-treated Krabbe dogs reached the pre-defined humane endpoint, characterized by severe hind limb weakness and inability to stand and walk, on Day 35 (8 weeks old; Animal K930) or Day 66 (12 weeks old; Animal K948), which was consistent with the natural history of the disease. In contrast, all AAVhu68.cGALCco-treated dogs (N=4/4) maintained normal motor function and did not reach the pre-defined humane endpoint associated with hind limb paralysis.

All the animals ICM-administered AAVhu68.cGALCco or vehicle (ITFFB) tolerated the procedure well and recovered from sedation uneventfully. There were no test-article related adverse events for the duration of the study in the animals administered AAVhu68.cGALCco.

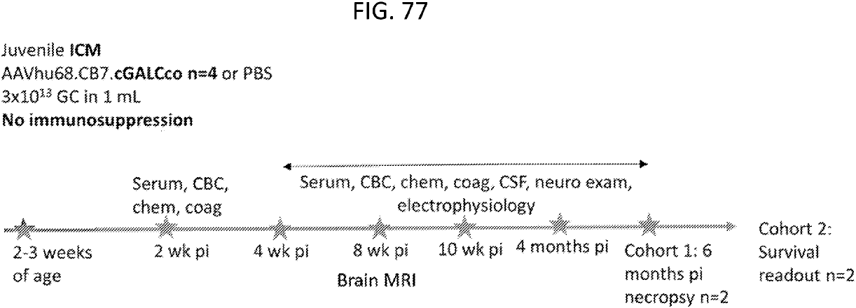

Growth and body weight gain were normal in all treated dogs (FIG. 78). One AAVhu68.cGALC.co-treated dog (K933) was euthanized due to weight body loss at 19.5 months of age (85 weeks).

All animals were videotaped playing in open space to assess their behavior and motor function. Assessments began at 12 weeks of age for Animals K928, K930, K933, and 3 to 4 weeks of age for Animals K937, K938, K939, K948. The two vehicle-treated Krabbe dogs (Animals K930 and K948) displayed the expected Krabbe-related abnormal motor function. Animal K930 developed head and limb tremor, intention tremor, and severe hind limb weakness with muscle atrophy and joint laxity preventing the animal to stand and walk (humane endpoint criteria) at the age of 8 weeks. Animal K948 started to show hind limb weakness at 7 weeks of age and progressed to ataxia, severe weakness, and inability to stand and walk (humane endpoint) at 12 weeks.

All animals that were treated with AAVhu68.cGALCco exhibited normal motor function during open play similar to the vehicle-treated wild type control and demonstrated the ability to walk, run, jump, and stand on hind limbs. All AAVhu68.cGALCco-treated Krabbe dogs (4/4) displayed normal behavior, playing with caretakers and seeing/fetching toys, suggesting that the treatment prevented Krabbe-related phenotype in all of the animals for the duration of study.

Abnormal neurological findings were observed in one of the vehicle-treated Krabbe Dogs (Animal K948) beginning at 11 weeks of age and included general proprioception deficits, ataxia, head tremors/truncal sway, muscle atrophy, the lack of a menace reflex (indicating suspected blindness), and a wide-based posture. This animal was euthanized soon after the observations. The other vehicle-treated Krabbe dog (Animal K930), reached humane endpoint prior to the first scheduled neurological scoring timepoint. All AAVhu68.cGALC.co-treated dogs (4/4) presented similar neurological examination when compared to the wild type vehicle-treated animal throughout the duration of the study.

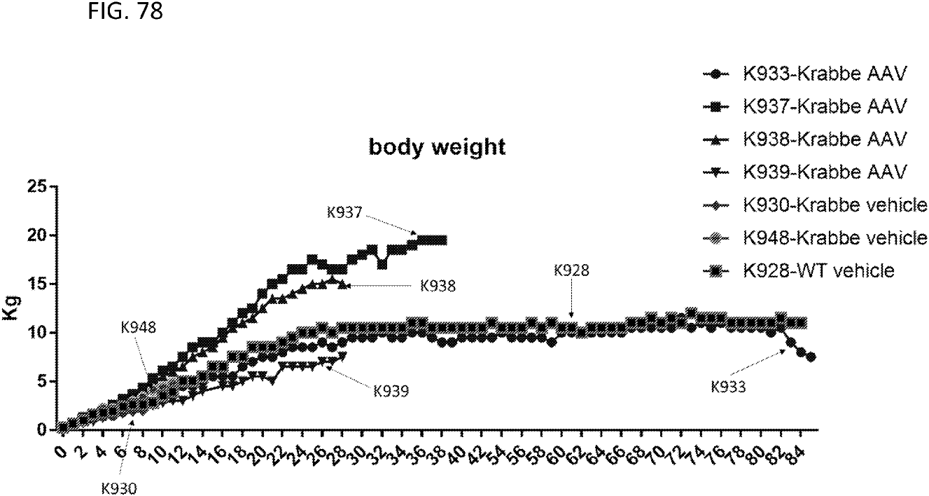

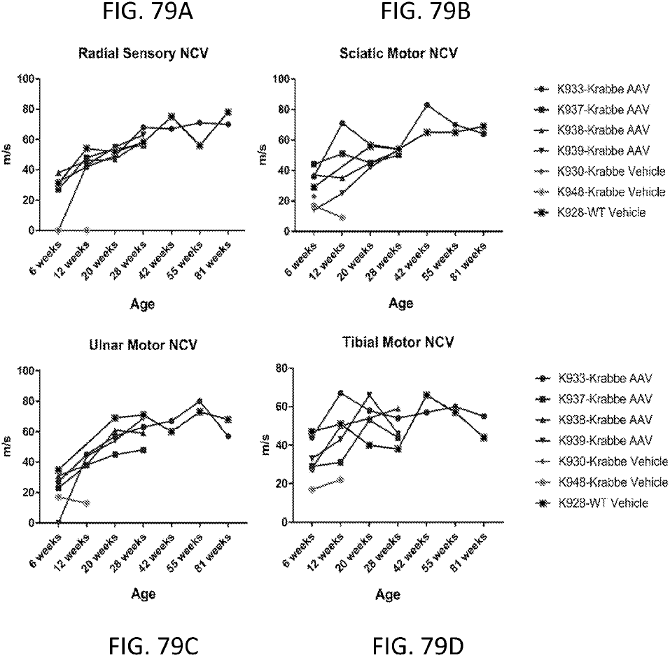

NCVs for the vehicle-treated Krabbe dogs at 6 weeks old (Animals K930 and K948) and 12 weeks old (Animal K948) were generally lower than that of the vehicle-treated wild type controls in all four nerves assessed (FIG. 79A - FIG. 79D). Both vehicle-treated Krabbe dogs notably exhibited a complete loss of NCVs in the radial sensory nerve. In contrast, AAVhu68.cGALCco-treated Krabbe dogs that were necropsied on Day 180 (Animals K938, K939), emergency-necropsied on Day 261 (Animal K937), or are in the ongoing long-term cohort (Animal K933) exhibited NCVs similar to that of the vehicle-treated wild type controls throughout the study. All treated dogs (4/4) had normal NCV, similar to the WT control.

Interpeak latency (IPL) between waves I and V of the BAER recording indicates the conduction latency within the brainstem auditory pathways, thus indicating central nervous conduction. One of the vehicle-treated Krabbe dogs (Animal K948) did not elicit an evoked potential (hearing threshold >90 dB; FIG. 80). The other vehicle-treated Krabbe dog (Animal K930) showed increased I-V IPL (mean 3.275 ms compared to mean 2.275 ms in the vehicle-treated wild type dog). All of the treated Krabbe dogs (4/4) had normal I-V IPL similar to the vehicle-treated wild type dog.

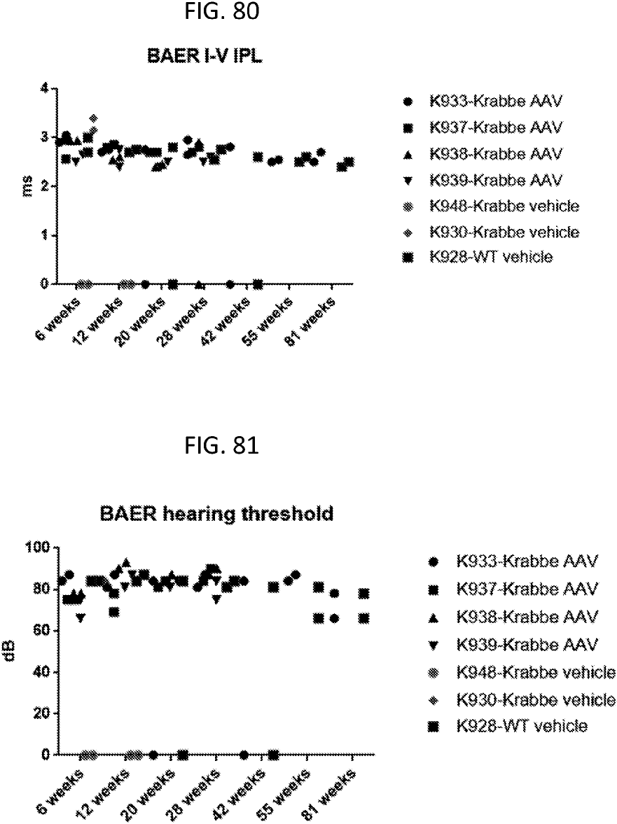

Hearing threshold could not be determined (> 90 dB) in one vehicle-treated Krabbe dog (Animal K948) but were similar to the vehicle-treated wild type dog (Animal K928) in the other vehicle-treated Krabbe dog (Animal K930) (FIG. 81). All the treated Krabbe dogs had hearing thresholds similar to the vehicle-treated wild type dog for the entire duration of the study until euthanized at 19.5 months (81 weeks) of age.

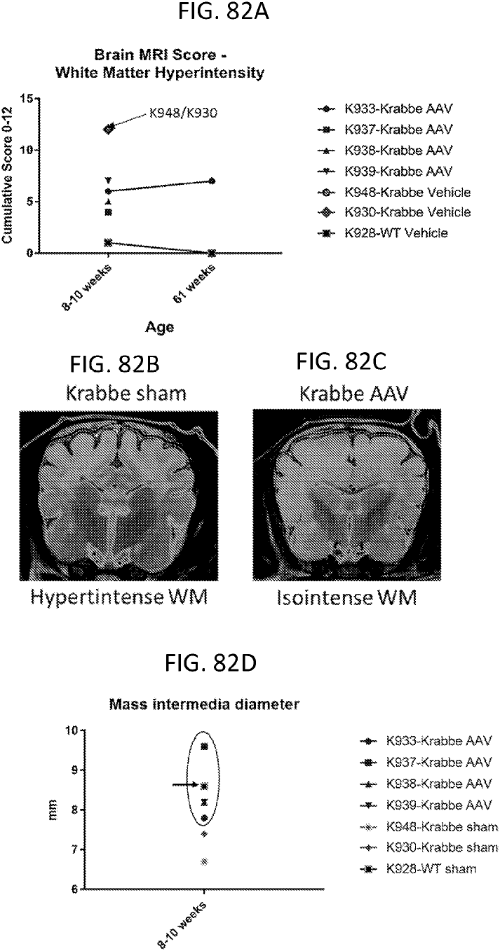

At 8-10 weeks of age, the vehicle-treated Krabbe dogs (Animals K930 and K948) exhibited a higher cumulative white matter hyperintensity score than that of the vehicle-treated wild type control, indicating a loss of myelin. Although AAVhu68.cGALCco administration did not normalize cumulative brain white matter hyperintensity scores to vehicle-treated wild type animal levels, all four AAVhu68.cGALCco-treated Krabbe dogs did exhibit lower cumulative white matter hyperintensity scores than that of the vehicle-treated Krabbe dogs. This result indicates that AAVhu68.cGALCco administration led to a preservation of myelin in the brain of Krabbe dogs. Subsequently, at 61 weeks of age, the AAVhu68.cGALCco-treated Krabbe dog (Animal K933) exhibited a similar cumulative white matter hyperintensity score as it did at 8 weeks of age. The score of the AAVhu68.cGALCco-treated Krabbe dog (Animal K933) remained higher than that of the vehicle-treated wild type control (Animal K928; FIG. 82A).

MRI scores for individual brain regions of each animal revealed that AAVhu68.cGALCco-treated Krabbe dogs exhibited white matter hyperintensity scores comparable to that of the vehicle-treated wild type control in some brain regions (hypointense signal in the corpus callosum and cerebellar white matter indicative of normal myelination), while simultaneously displaying higher scores in other regions (hyperintense signal in the centrum semi-ovale, corona radiata, and occipital white matter indicative of demyelination). This finding suggests that treatment did not equally correct all the white matter regions, possibly due to the distribution of AAVhu68.cGALCco after ICM administration. The corpus callosum and cerebellum, which were both fully corrected, are in close contact with CSF. Furthermore, the brain regions that were well-corrected in the AAVhu68.cGALCco-treated Krabbe dog compared to the vehicle-treated Krabbe dog at 8-10 weeks of age (corpus callosum and cerebellar white matter) remained normal at 61 weeks of age with similar hypointense signal.

The glycosphingolipids in dog CSF, including lactosylceramides (LC), glucosylceramides (GluC), galactosylceramides (GalC), glucosylsphingosine (GluS), galactosylsphingosine (GalS), and lactosylsphingosine (LS) were profiled. Amongst these, only psychosine (aka galactosylsphingosine) revealed a clear increase in Krabbe vehicle-treated dogs compared to WT.

Psychosine was undetectable in the CSF of vehicle-treated wild type dogs from Day 0 through Day 180 post treatment (FIG. 83). While psychosine was undetectable in the CSF of both vehicle-treated Krabbe dogs at baseline (Day 0), psychosine became elevated in both animals on Day 28, and levels increased further by the time of humane euthanasia (Day 35 for Animal K930 and Day 66 for Animal K948). Elevations in psychosine correlated with the onset and progression of neurological symptoms in the vehicle-treated Krabbe dogs.

In contrast, psychosine was undetectable at most time points for all four AAVhu68.cGALCco-treated Krabbe dogs. One animal exhibited undetectable levels of psychosine at all time points evaluated (N=¼; Animal K933), while either mild transient elevations at Day 120 (N=2/4, Animals K937 and K939) or a mild elevation at the last time point evaluated on Day 180 (N=¼; Animal K938) was observed for the other animals. All treated dogs (4/4) maintained undetected to very low CSF psychosine levels for 6 months post treatment.

Three (¾) AAVhu68.cGALCco-treated Krabbe dogs (Animals K938, K938, K939) presented a mild and transient lymphocytosis 2 weeks post-injection (values from 4,674 to 5,590 cells/µL). This finding is possibly treatment-related as it was not observed in the vehicle-treated Krabbe or wild type animals. It is not considered adverse due to low grade elevation and transient nature.

There was no vector related modification of coagulation parameters nor serum clinical chemistry.

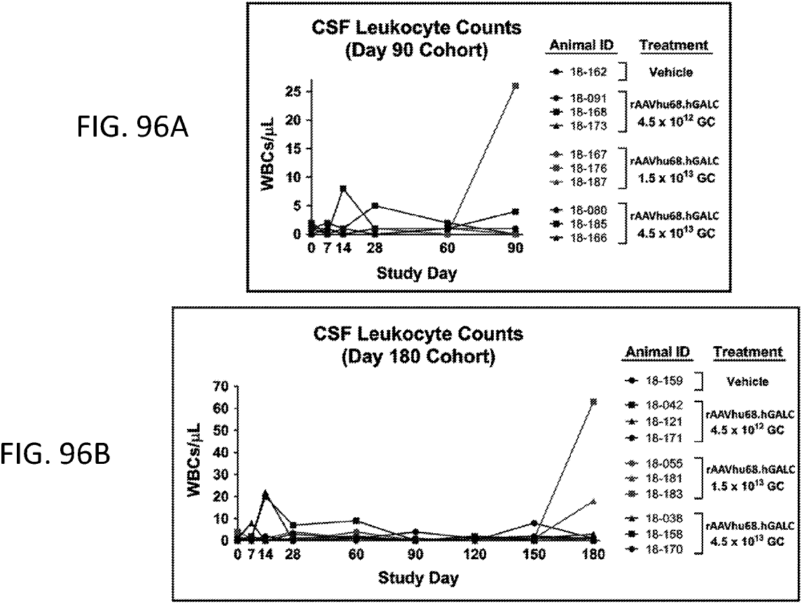

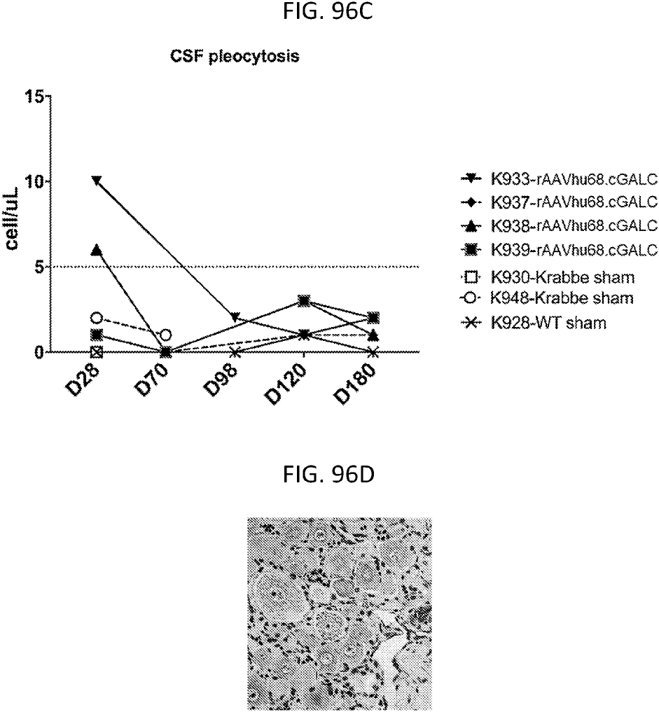

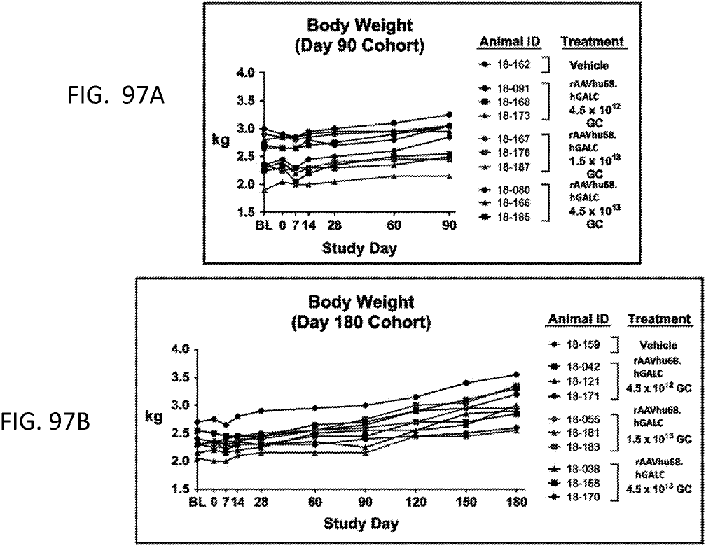

Two AAVhu68.cGALCco-treated Krabbe dogs (Animals K933 and K938) presented transient mild CSF mononuclear pleocytosis 4 weeks post-injection (FIG. 96C). This finding is vector related and not considered adverse as it was self-limited and not accompanied by any neurological sign. No histopathological lesions related to AAVhu68.cGALCco delivery were observed at 6 months and dorsal root ganglia were normal showing no sensory neuron toxicity (FIG. 96D)

### Necropsy and Histopathology

Organ weights for all Krabbe dogs treated with AAVhu68.cGALCco or vehicle were similar to the vehicle-treated wild type dog and published values. Necropsy at humane endpoints or scheduled timepoint revealed no test-article related gross lesions. Krabbe related muscle atrophy was observed in one vehicle-treated dog (Animal K930). Enlarged salivary glands was observed in one AAVhu68.cGALCco-treated dog (Animal K933).

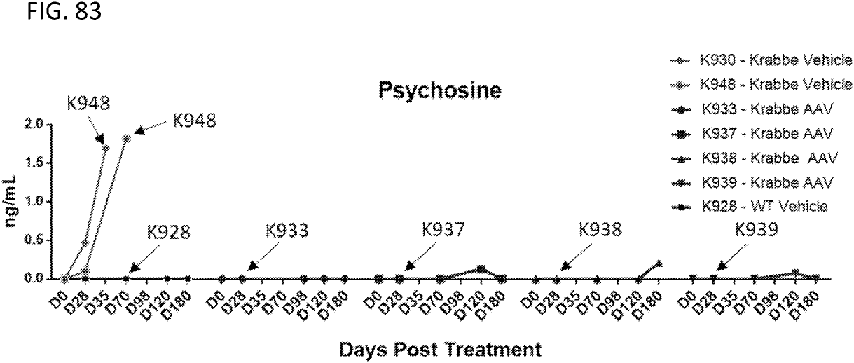

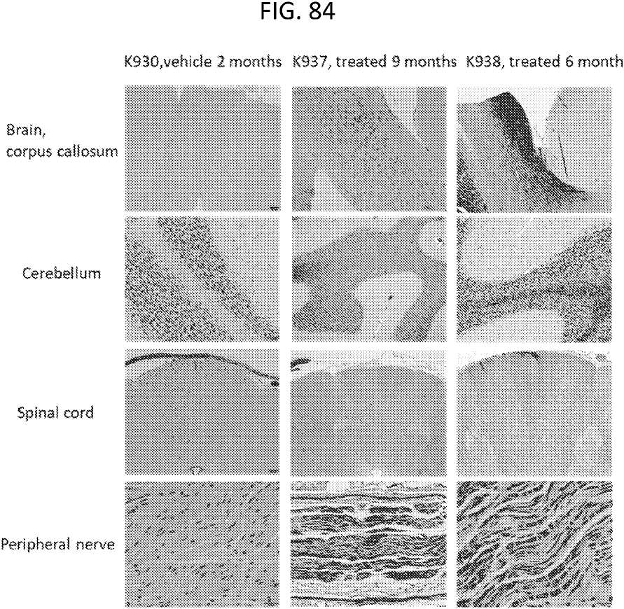

AAVhu68.cGALCco-treated Krabbe dogs (Animals K938, K939, K937) exhibited increased myelination and reduced globoid cell infiltration in the brain, spinal cord, and peripheral nerves by histology when compared to the vehicle-treated Krabbe dogs (FIG. 84).

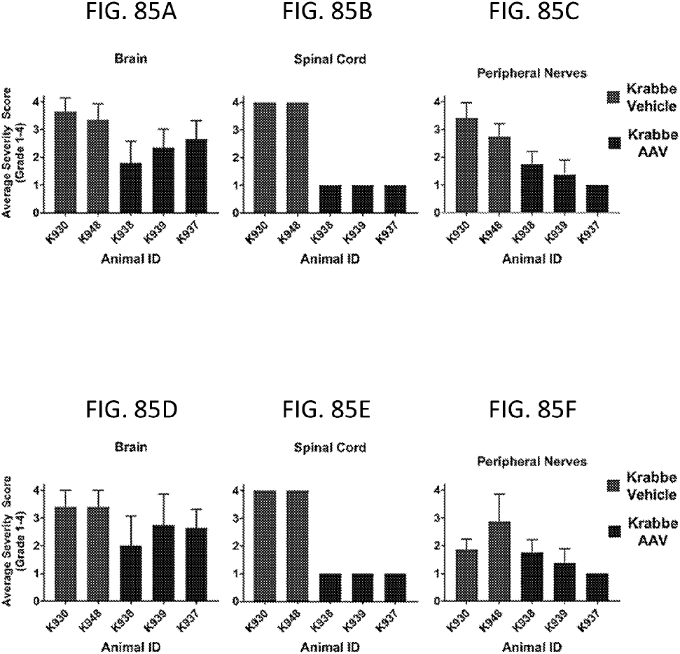

Consistent with the increased myelination and reduced globoid cell infiltration, blinded semi-quantitative scoring of tissue sections revealed that AAVhu68.cGALCco-treated Krabbe dogs (Animals K938, K939, K937) had consistently lower average severity scores for demyelination (FIG. 85A) and globoid cell infiltration (FIG. 85B) in the brain, spinal cord, and peripheral nerves compared to that of the vehicle-treated Krabbe dogs.

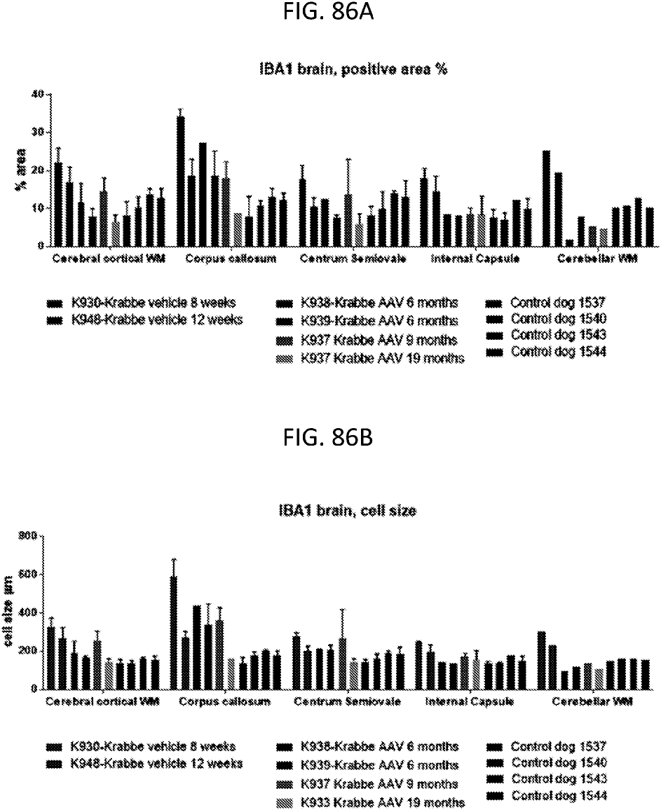

In the brain, neuro-inflammation (indicated by the IBA1 % area), globoid cell infiltration (IBA1 % area), and globoid cell storage (indicated by the IBA1 + cell size), were dramatically reduced in ¾ of the AAVhu68.cGALCco-treated Krabbe dogs (Animals K937, K938, K939) in the cerebral cortex, ¼ of the AAVhu68.cGALCco-treated Krabbe dogs (Animal K938) in the corpus callosum, 2/4 of the AAVhu68.cGALCco-treated Krabbe dogs (Animals K937 and K938) in the centrum semi-ovale, and all 4/4 AAVhu68.cGALCco-treated Krabbe dogs in the internal capsule and the cerebellum (FIG. 86A and FIG. 86B). The levels observed in the AAVhu68.cGALCco-treated were similar to vehicle-treated wild type animal. The AAVhu68.cGALCco-treated dog (Animal K933) that survived to 19.5 months displayed normal levels of IBA1 in all the regions of the brain and cerebellum suggesting durability of the therapeutic efficacy. This observation also indicates that his clinical condition that caused body weight loss and prompted euthanasia was unrelated to CNS disease progression.

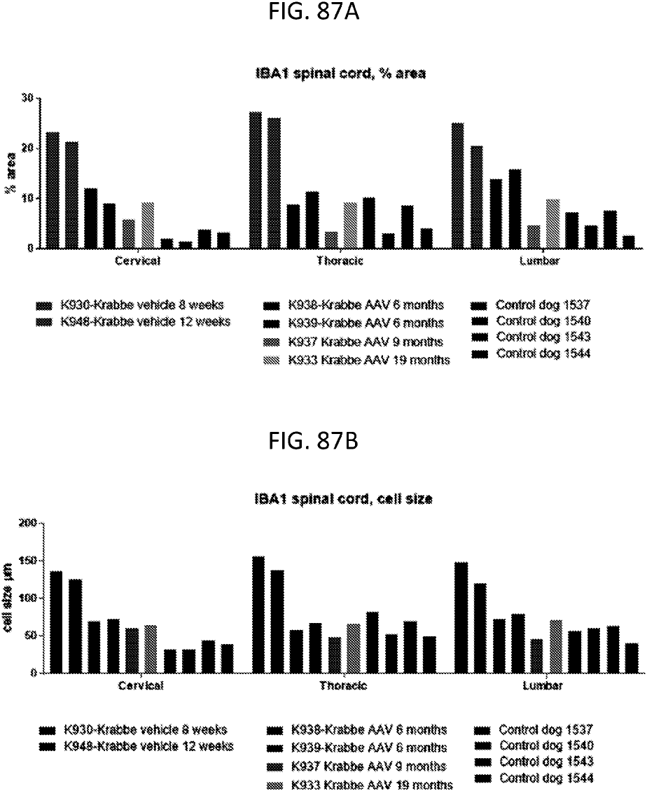

In the spinal cord, neuro-inflammation (indicated by the IBA1 % area), globoid cell infiltration (IBA1 % area), and globoid cell storage (indicated by the IBA1 + cell size), were normalized in all dogs in the thoracic and lumbar segments while the cervical segment was intermediate between vehicle-treated Krabbe dogs and the vehicle-treated wild type dog (FIG. 87A and FIG. 87B). The reason for this finding is unclear but may indicate higher transgene expression in the lumbar and thoracic regions. Correction of the long term 9 months and 19.5 month old dog tissues is similar to that of the other treated dogs euthanized at 6 months showing lack of disease progression and durability of the treatment.

### Transgene Expression

In the sciatic nerve, neuro-inflammation (indicated by the IBA1 % area), globoid cell infiltration (IBA1 % area), and globoid cell storage (indicated by the IBA1 + cell size), were normalized in all dogs and showed no disease progression from 6 months (scheduled necropsy) to 9 and 19.5 months (unscheduled necropsies).

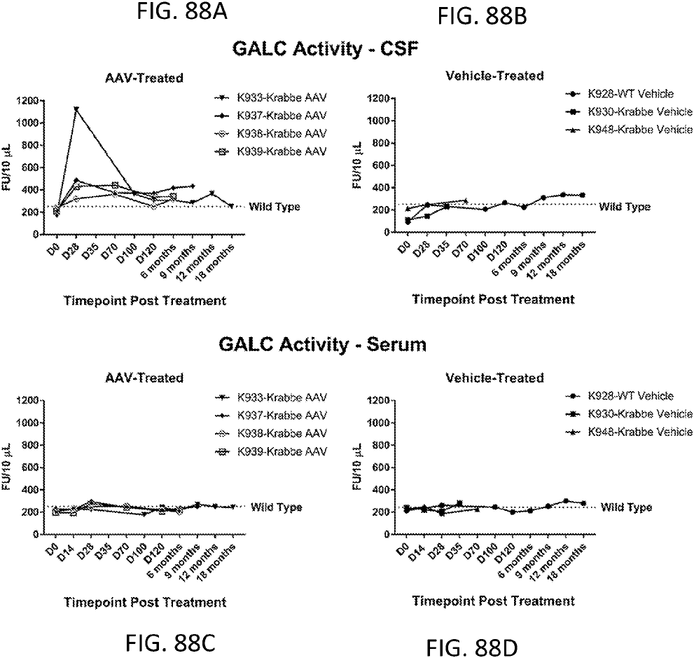

In CSF, GALC enzyme activity was detectable above baseline levels measured on Day 0 in all AAVhu68.cGALCco-treated Krabbe dogs (N=4/4) by 28 days post treatment. Levels were maintained at or above vehicle-treated wild type GALC activity levels for the duration of the study for each AAVhu68.cGALCco-treated Krabbe dog, including up to 19.5 months post treatment. Expression levels in AAVhu68.cGALCco-treated Krabbe dogs remained relatively stable for the duration of the study in most animals except Animal K933, which exhibited a notable peak in GALC enzyme activity on Day 28 followed by a decline to stable levels by Day 100 through 19.5 months post treatment. Finally, at Day 28 and Day 70, GALC enzyme activity levels in all AAVhu68.cGALCco-treated Krabbe dogs (N=4/4) exceeded that of the vehicle-treated controls prior to human euthanasia (FIG. 88A - FIG. 88B).

In serum, AAVhu68.cGALCco-treated Krabbe dogs exhibited GALC activity levels comparable to that of the vehicle-treated Krabbe dogs and vehicle-treated wild type control, and GALC activity levels for all animals in the study remained stable for the duration of follow-up (up to 18 months post treatment) (FIG. 88C and FIG. 88D). The lack of an increase in serum GALC activity levels following AAVhu68.cGALCco administration was likely due to the treatment age (2-3 weeks of age), as it has been shown that AAV transduction is not durable in the liver of immature animals due to cell division and growth of the organ.

In CNS, PNS, and peripheral tissues, variability in GALC enzyme activity was observed, which was possibly due to sampling variations and/or known technical limitations associated with performing this assay on tissues, which express other enzymes that can cleave the fluorescent substrate and produce background signal. Despite these caveats, ICM administration of AAVhu68.cGALCco generally led to increased GALC enzyme activity in key target tissues of the CNS and PNS when compared to vehicle-treated animals.

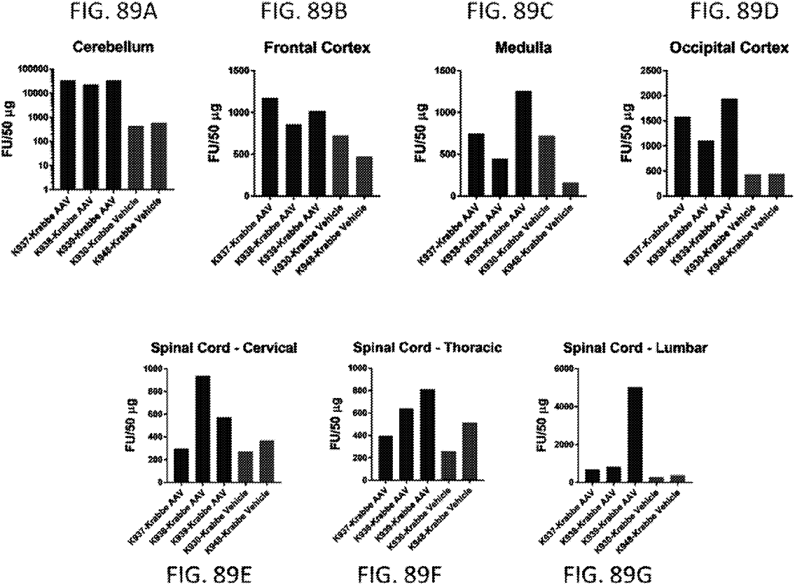

In the CNS (brain and spinal cord), GALC activity levels were increased in the majority of AAVhu68.cGALCco-treated Krabbe dogs in the cerebellum, frontal cortex, medulla, occipital cortex, and spinal cord (cervical, thoracic, and lumbar) compared to that of vehicle-treated Krabbe dogs (FIG. 89A - FIG. 89G).

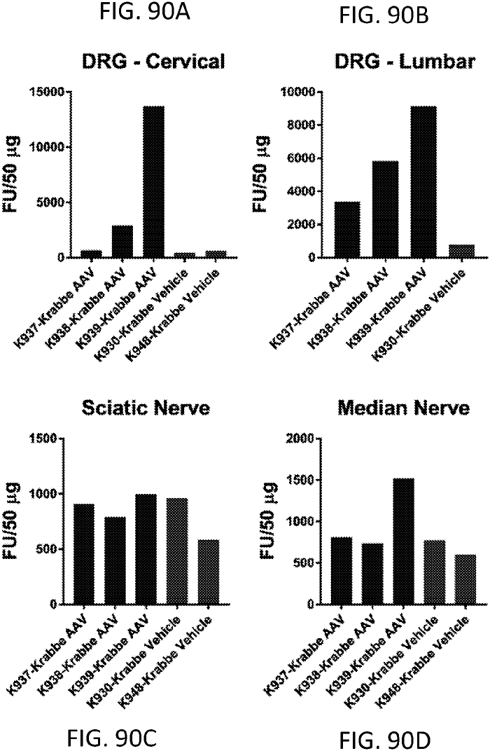

In the PNS, GALC activity levels were increased in the majority of AAVhu68.cGALCco-treated Krabbe dogs in the cervical and lumbar DRG, along with the median nerve when compared to that of vehicle-treated Krabbe dogs. A slight increase in GALC activity levels in the sciatic nerve was detected in ⅓ AAVhu68.cGALCco-treated dogs (Animal K939) when compared to that of vehicle-treated dogs (FIG. 90A - FIG. 90D).

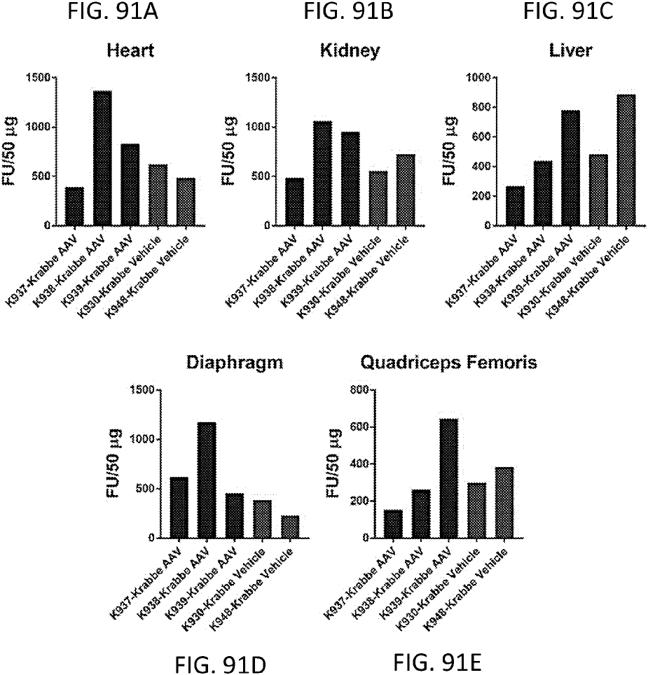

In peripheral organs, AAVhu68.cGALCco administration increased GALC activity levels in the diaphragm, heart, and kidney in the majority of animals when compared to that of vehicle-treated Krabbe dogs. Increased GALC activity was observed in the quadriceps femoris in ⅓ AAVhu68.cGALCco-treated dogs (Animal K939) when compared to that of vehicle-treated Krabbe dogs. AAVhu68.cGALCco administration did not appear to increase GALC enzyme activity in the liver compared that of to the vehicle-treated Krabbe dogs, which was consistent with the observed results for GALC enzyme activity in serum (FIG. 91C).

### Biodistribution

Vector genomes were detected in AAVhu68.cGALCco-treated Krabbe Dogs with the highest transduction levels observed in the spinal cord, brain and dorsal root ganglia (FIG. 92). Peripheral organs (liver, heart, kidney, muscle) showed the lowest transduction.

In summary, ICM administration of AAVhu68.cGALCco to 2-3-week old pre-symptomatic Krabbe dogs increased transgene product expression (GALC activity) in key target tissues for the treatment of Krabbe disease (CNS and PNS tissues). Transgene product was also expressed in the CSF, which suggests the possibility of cross-correction in the CNS and PNS. AAVhu68.cGALCco administration substantially increased survival, preserved both neuromotor function and peripheral nerve function, and reduced CSF levels of a disease-relevant biomarker (psychosine). AAVhu68.cGALCco administration also prevented demyelination and globoid cell infiltration in the brain, spinal cord, and peripheral nerves.

### Example 10 -Toxicology Study in Nonhuman Primates

A toxicology study was conducted using the same rAAVhu68.hGALC vector lot as was used in the mouse MED study and was conducted in NHPs because they better replicate the size and CNS anatomy of humans and can be treated using the clinical ROA (ICM). It is expected that the similarity in size, anatomy, and ROA results in representative vector distribution and transduction profiles, which allows for more accurate assessment of toxicity than is possible in mice or dogs. In addition, more rigorous neurological assessments can be performed in NHPs than in rodent or canine models, allowing for more sensitive detection of CNS toxicity.

ICM vector administration results in immediate vector distribution within the CSF compartment. Doses were scaled by brain mass, which provides an approximation of the size of the CSF compartment. Dose conversions are based on a brain mass of 0.15 g for a newborn mouse (Gu Z., et al. (2012) PLoS One. 7(7):e41542.), 0.4 g for a juvenile-adult mouse (Gu Z., et al. (2012) PLoS One. 7(7):e41542.), 90 g for a juvenile and adult rhesus macaque (Herndon J.G., et al. (1998) Neurobiol Aging. 19(3):267-72), 60 g for a dog, 800 g for 4-12-month-old infants, and 1300 g for adult humans (Dekaban A.S. (1978) Ann Neurol. 4(4):345-56). Doses for the NHP toxicology study, the murine MED study, and the equivalent human doses are shown below.

TABLE

Vector doses for the murine MED study, NHP toxicology study, and equivalent dog and human doses

| Dose (GC/g brain mass) | Juvenile Mouse MED study (GC) | Juvenile NHP toxicology study (GC) | Dog (GC) | Human (GC) |
|---|---|---|---|---|
| 5.00 × 10¹¹ | 2.00 × 10¹¹ | 4.50 × 10¹³ | 3.00 × 10¹³ | 4.00 × 10¹⁴ |
| 1.70 × 10¹¹ | 6.80 × 10¹⁰ | 1.50 × 10¹³ | - | 1.40 × 10¹⁴ |
| 5.00 × 10¹⁰ | 2.00 × 10¹⁰ | 4.50 × 10¹² | - | 4.00 × 10¹³ |
| 1.70 × 10¹⁰ | 6.80 × 10⁹ | - | - | 1.40 × 10¹³ |

Abbreviations: GC, genome copies; MED, minimum effective dose; NHP, nonhuman primate.

The juvenile NHP was selected because the dimensions of the NHP central nervous system (CNS) act as a representative model of our target clinical population and allow us to administer rAAVhu68.hGALC using the proposed clinical route of administration (ROA) via an ICM injection. This study was designed to provide critical data on ROA-related safety of rAAVhu68.hGALC. Juvenile animals were selected to model the pediatric population that will be enrolled in the planned clinical trial.

The total number of NHPs used in this study was considered to be the minimum number necessary to provide assessment of the toxicity of rAAVhu68.hGALC at three dose levels and two necropsy time points and to account for variability among NHPs.

A cohort of 22 animals (12 males and 10 females) were used for this study. Animals were assigned to this study in accordance with SOP 7006. Briefly, animals underwent additional testing prior to study initiation, including medical record review, physical exam, body weight update, and any tests/procedures requested by the Study Director. Once an animal was included in the study, all medical records for the animal were archived as part of the study documentation. In the event that an animal did not qualify for assignment to the study or was removed from the study, they were replaced with an alternate animal at the discretion and approval of the Study Director.

Following group assignment, each animal received a single ICM injection of one of the following treatments of either control article (ITFFB) or test article (rAAVhu68.hGALC):
1.) ITFFB (control article)
2.) a low dose of rAAVhu68.hGALC (4.5 × 10¹² GC; test article)
3.) a mid-dose of rAAVhu68.hGALC (1.5 × 10¹³ GC; test article)
4.) a high dose of rAAVhu68.hGALC (4.5 × 10¹³ GC; test article)

The day of dose administration (Day 0) was staggered with animals representing as many study groups as possible across administration dates. The study design is summarized below.

TABLE

Group Designations, Dose Levels, and Route of Administration

| Group | Treatment (Dose) | Dose (GC) | Dose (GC/g Brain) | Anim al ID | Sex | ROA | Administratio n Volume (mL) | Day of Dosin g | Day of Necropsy |
|---|---|---|---|---|---|---|---|---|---|
| 1 | ITFFB | N/A | N/A | 18-162 | Femal e | ICM | 1.0 | 0 | 90±4 |
| 2 | vector (Low Dose) | 4.5 × 10¹² | 5.0 × 10¹⁰ | 18-168 | Femal e | | | | |
| | | | | 18-173 | Femal e | | | | |
| | | | | 18-091 | Male | | | | |
| 3 | vector (Mid-Dose) | 1.5 × 10¹³ | 1.7 × 10¹¹ | 18-167 | Male | | | | |
| | | | | 18-176 | Male | | | | |
| | | | | 18-187 | Femal e | | | | |
| 4 | vector (High Dose) | 4.5 × 10¹³ | 5.0 × 10¹¹ | 18-080 | Male | | | | |
| | | | | 18-166 | Male | | | | |
| | | | | 18-185 | Femal e | | | | |
| 5 | ITFFB | N/A | N/A | 18-159 | Male | ICM | 1.0 | 0 | 180±5 |
| 6 | vector (Low Dose) | 4.5 × 10¹² | 5.0 × 10¹⁰ | 18-042 | Femal e | | | | |
| | | | | 18-121 | Femal e | | | | |
| | | | | 18-171 | Male | | | | |
| 7 | vector (Mid-Dose) | 1.5 × 10¹³ | 1.7 × 10¹¹ | 18-055 | Male | | | | |
| | | | | 18-181 | Femal e | | | | |
| | | | | 18-183 | Femal e | | | | |
| 8 | vector (High Dose) | 4.5 × 10¹³ | 5.0 × 10¹¹ | 18-038 | Femal e | | | | |
| | | | | 18-158 | Femal e | | | | |
| | | | | 18-170 | Male | | | | |

Abbreviations: GC, genome copies; ICM, intra-cisterna magna; ID, identification number; ITFFB, intrathecal final formulation buffer; N/A, not applicable; ROA, route of administration.

The study events are summarized in the table below.

TABLE

Study Events

| | | | | | | | | | | | |
|---|---|---|---|---|---|---|---|---|---|---|---|
| Study Day | Baseline (Day -28 to -1) | Day 0 | Day 5±2 | Day 7±1 | Day 14±2 | Day 28±3 | Day 60±3 | Day 90±4 | Day 120±4^{a} | Day 150±4^{a} | Day 180±5^{a} |
| Dosing (vector or ITFFB) | | X | | | | | | | | | |
| Body Weight, Temperature, Respiratory Rate, Heart Rate | X | X | | X | X | X | X | X | X | X | X |
| Neurological Examinations | X | | | | X | X | X | X | X | X | X |
| Sensory Nerve Conduction Study | X | | | | | X | X | X | X | X | X |
| Biomarker (blood)^{c} - Transgene Product Expression | X | | | | X | X | X | X | X | X | X |
| Biomarker (CSF)^{c}- Transgene Product Expression | | X | | X | X | X | X | X | X | X | X |
| Clinical Pathology (blood) | X | X | | X | X | X | X | X | X | X | X |
| Clinical Pathology (CSF) | | X | | X | X | X | X | X | X | X | X |
| Immunology: NAbs against capsid | X | | | | | X | X | X | X | X | X |
| Immunology: T cell response to capsid or transgene (IFN-γ ELISpot on PBMCs) | X | | | | | X | X | X | X | X | X |
| Immunology: T cell response to capsid or transgene (IFN-γ ELISpot on lymphocytes^{d}) | | | | | | | | X^{b} | | | X |
| Vector Pharmacokinetics (blood and CSF) | | X | | X | X | X | X | X | | | X |
| Vector Excretion (urine and feces) | X | | X | | | X | X | X | X | X | X |
| Necropsy and Sample Collection^{e} | | | | | | | | X^{b} | | | X |

^{a}Groups 5-8 only (animals for Day 180±5 necropsy time point).
^{b}Groups 1-4 only (animals for Day 90±4 necropsy time point).
^{c}Blood and CSF were collected for evaluation of transgene product expression (GALC enzyme activity assay) and antibodies against the transgene product (anti-human GALC antibody ELISA).
^{d}Lymphocytes were obtained from the liver, spleen, and bone marrow.
^{e}Samples were collected for evaluation of histopathology, transgene product expression (GALC enzyme activity assay). Tissues were collected and stored for a future vector biodistribution analysis. Abbreviations: CSF, cerebrospinal fluid; ELISA, enzyme-linked immunosorbent assay; ELISpot, enzyme-linked immunosorbent spot; GALC, galactosylceramidase (protein); IFN-y, interferon gamma; ITFFB, intrathecal final formulation buffer; NAbs, neutralizing antibodies; PBMC, peripheral blood mononuclear cells.

### OBSERVATIONS

### Viability Assessments (In-Cage)

Animals were observed daily visually for general appearance or signs of toxicity, which included but were not limited to neurologic signs or lethargy, distress, and changes in behavior. The clinical veterinarian or designee and the study director was notified of any unusual conditions. Treatment was conducted only after approval by the clinical veterinarian or designee and study director, except in cases of emergency imperiling the NHP or for humanely euthanizing the NHP if the clinical veterinarian and/or the study director could not be contacted promptly.

### In-Life Examinations

### Physical Examination

All animals were physically examined each time they were anesthetized. Animals were observed for any abnormality in vital signs, muscular tones, coat and skin, eyes, genitalia, etc. All abnormal findings were promptly communicated to the Study Director and recorded. At the time of necropsy, the animals were examined for gross abnormalities. All changes were noted. As part of preventative health and colony maintenance, animals also received routine semiannual physical examinations in accordance with SOP 7016.

### Neurological Monitoring

Animals underwent neurological monitoring at the time points indicated in the table above. Briefly, the assessment was divided into five sections evaluating the following: mentation, posture and gait, proprioception, cranial nerves, and spinal reflexes. The tests for each assessment were performed in the same order each time. Assessors were not formally blinded to the treatment group; however, assessors typically remained unaware of treatment group at the time of assessment. Numerical scores were given for each assessment category as applicable and recorded (normal: 1; abnormal: 2; decreased: 3; increased: 4; none: 5; N/A: not applicable).

### Mentation

To assess mentation, NHPs were assessed cage-side prior to manipulation by the examiner by noting how the animal interacted with the examiner and the environment. Any changes, such as depressed, dull, disoriented, or comatose behavior, were recorded in addition to respiratory character and effort and any excessive lacrimation or salivation.

### Posture and Gait

To assess posture and gait, NHPs were assessed cage-side prior to manipulation by the examiner by observing how the animal moved around in the cage. Any impairments, such as ataxia, paresis, paralysis, or stumbling/falling/tremors/convulsions/uncoordinated movement were recorded. The examiner also observed the animal’s posture, head position (head tilt, head or neck turn), wide-based stance, ability to perch, tremors, or unintentional movements, and any abnormalities were recorded.

### Proprioception

Proprioceptive assessments were optional as they can only be performed on a restrained animal. Proprioceptive positioning was assessed by standing the animal on a flat surface, such as a table top, and flipping the dorsal aspect of each of the hind feet (one at a time) onto the tabletop. The animal should correct the placement of the foot immediately, and any delayed responses and/or failures to correct placement of the foot were recorded. Visual placing was assessed by slowly moving the primate towards a flat surface with a ledge (such a table top) and allowing the dorsal aspect of the hind feet to touch the surface. The primate should respond by placing the plantar aspect of both feet on the tabletop. Tactile placing was assessed in the same way as visual placing except that the primate’s eyes were covered by the examiner’s hand.

### Cranial Nerves

The cranial nerve assessment was performed in the cage by utilizing the squeeze-back mechanism or outside the cage in a chair while restrained by noting facial/head symmetry as well as facial and cranial muscle tone. Any abnormalities were recorded. The menace reflex was assessed by advancing the hand of the handler toward each eye of the primate, using caution so as not to create an air current or touch any part of the primate’s face. The menace reflex test determined whether the animal blinked each eye as the examiner’s hand approached the face, and any abnormalities were recorded. Each eye was examined for symmetry (placement, pupil size and shape). Pupillary light reflex was assessed in both eyes using either a trans-illuminator or pen light by covering the eyes with both hands for 5 seconds, removing hand, and then shining light directly into the eye to assess pupillary constriction. The symmetry of pupillary response (speed of contraction and overall degree of constriction) was noted. The palpebral reflex was assessed in both eyes by touching a cotton tip applicator to the lateral canthus followed by the medial canthus of the eye. The animal should blink with each touch, and any abnormalities were recorded. Sensation of the nasal septum was assessed by pinching the nasal septum with forceps and determining whether the animal reacted to the noxious stimulus. Eye positioning was assessed in chaired or in manually restrained animals in which the nose can be elevated while the eyes stayed in a normal position. When the head was gently moved side to side, the eyes should have followed the movement of the head (nystagmus), and any abnormalities were recorded.

### Spinal Reflex

The spinal nerves/spinal reflexes were assessed by evaluating the primate’s muscle strength. If manually restrained, muscle strength was assessed by the handler holding both hind limbs (one in each hand) to assess the primate’s ability to resist manipulation of the limbs. If assessed cage-side, muscle strength was assessed by handing the animal a toy or other appropriate object to grasp in its hand while the examiner continued to hold the object while pulling back. In each case, the animal’s ability to resist the examiner’s action was recorded. The withdrawal reflex was assessed by pinching each of the hind feet with a hemostat, and determining whether the animal quickly flexed the knee and drew its limb up toward the body. The response was recorded. The perineal reflex was assessed by gently stroking the skin around the anus with a cotton-tipped applicator and assessing whether the animal contracted the external sphincter muscles indicated by a puckering of the surrounding skin.

### Sensory Nerve Conduction Study

Animals were sedated with a combination of ketamine/dexmedetomidine. Sedated animals were placed in lateral or dorsal recumbency on a procedure table with heat packs to maintain body temperature. Electronic warming devices were not used due to the potential for interference with electrical signal acquisition.

Sensory nerve conduction studies (NCS), also referred to as sensory nerve conduction velocity (NCV) tests, were performed using the Nicolet EDX® system (Natus Neurology) and Viking® analysis software to measure sensory nerve action potential (SNAP) amplitudes and conduction velocities. Briefly, the stimulator probe was positioned over the median nerve with the cathode closest to the recording site. Two needle electrodes were inserted subcutaneously on digit II at the level of the distal phalanx (reference electrode) and proximal phalanx (recording electrode), while the ground electrode was placed proximal to the stimulating probe (cathode). A WR50 Comfort Plus Probe pediatric stimulator (Natus Neurology) was used. The elicited responses were differentially amplified and displayed on the monitor. The initial acquisition stimulus strength was set to 0.0 mA in order to confirm a lack of background electrical signal. In order to find the optimal stimulus location, the stimulus strength was increased up to 10.0 mA, and a train of stimuli were generated while the probe was moved along the median nerve until the optimal location was found as determined by a maximal definitive waveform. Keeping the probe at the optimal location, the stimulus strength was progressively increased up to 10.0 mA in a step-wise fashion until the peak amplitude response no longer increased. The last thirty stimulus responses were recorded and saved in the software. Up to 10 maximal stimuli responses were averaged and reported for the median nerve. The distance (cm) from the recording site to the stimulation cathode was measured and entered into the software. The conduction velocity was calculated using the onset latency of the response and the distance (cm). Both the conduction velocity and the average of the SNAP amplitude were reported (FIG. 93). The median nerve was tested bilaterally.

### Results

### Clinical Observations

Animals were monitored daily throughout the study. There were no clinical abnormalities attributable to test article administration. Several abnormalities unrelated to test article administration were noted and discussed below. These observations and associated treatment, if needed, did not impact the study because symptoms either fully resolved or improved over time and did not impact the evaluation of in-life end points.

Some animals had positive stool cultures for campylobacter and/or fecal parasites with or without intermittent diarrhea prior to the study start or at baseline and were treated with antibiotics and an antifungal. During the study, three of these animals were housed together (4.5 × 10¹³ GC; Animals 18-080, 18-166, and 18-185; Group 4) and presented with intermittent diarrhea with a reduction in appetite consistent with shigellosis secondary to dysbosis. These findings were noted beginning on Days 1-3, and with veterinary supportive care, resolved by Day 48. Blood hematology values for these animals were affected by diarrhea.

Animal 18-080 (4.5 × 10¹³ GC, Group 4) was noted to have a small rectal prolapse during the neurological examination on Day 59, which resolved without treatment and was not observed later that day when the animal was sedated for NCS and CSF collection. Animal 18-171 (1.5 × 10¹³ GC, Group 7) was noted to have mild dermatitis on Day 6, which resolved without treatment by Day 14. Animal 18-181 (1.5 × 10¹³ GC, Group 7) exhibited mild dermatitis at the CSF collection site on Day 90, which was attributed to a reaction to the antiseptic scrub. The dermatitis resolved following treatment with a topical antibiotic. Animal 18-121 (4.5 × 10¹² GC, Group 6) displayed superficial scratches on arm, ear, face, along with a small hematoma on right ear on Day 76, which was attributed to fighting with a cagemate. Cagemates were reassigned, and the wounds healed following a course of antibiotics. On Day 47, blood was noted on the cage bars, finger board, and grate of Animal 18-183 (1.5 × 10¹³ GC, Group 7) consistent with a laceration the third digit on the animal’s right hand. No pain or swelling were noted, and the injury resolved without treatment.

At baseline, Animal 18-187 (1.5 × 10¹³ GC, Group 3) exhibited inguinal skin reddening attributed to dermatitis. Skin reddening improved with treatment, but did not fully resolve by the time of the scheduled necropsy on Day 85. This animal also exhibited vaginal bleeding on Day 15 and Day 63 that was related to menstrual cycling. Animal 18-042 (4.5 × 10¹² GC, Group 6) was observed to be cycling on Day 95.

### Sensory Nerve Conduction Studies

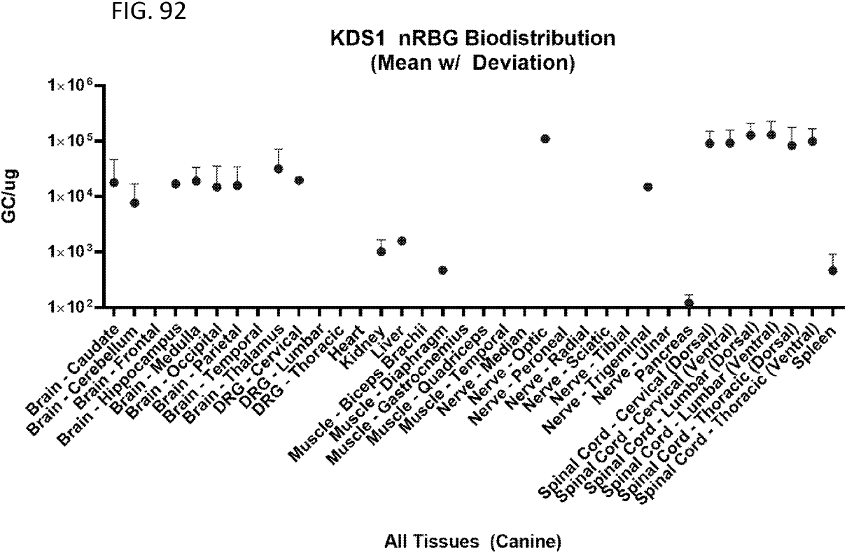

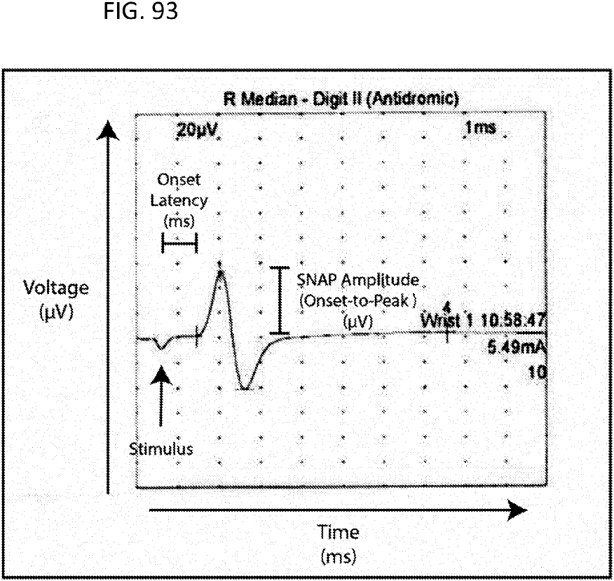

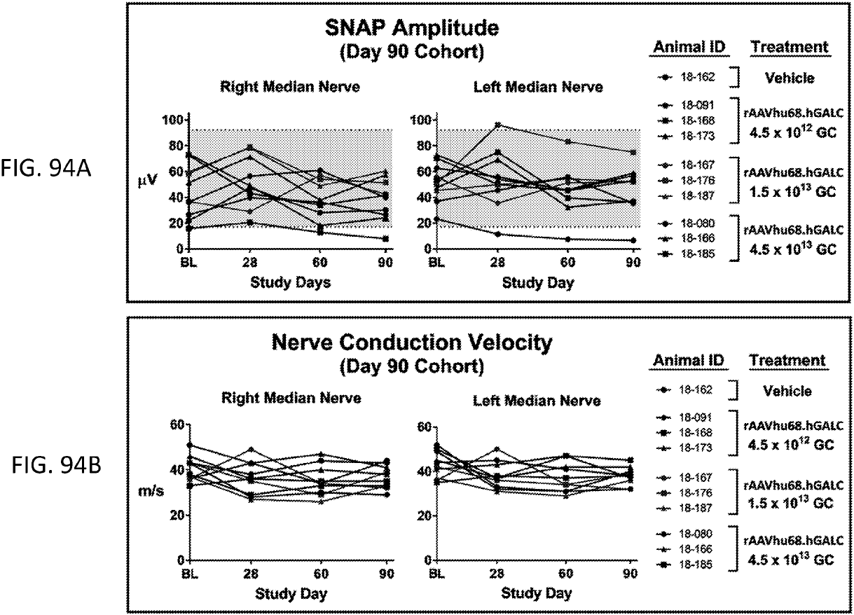

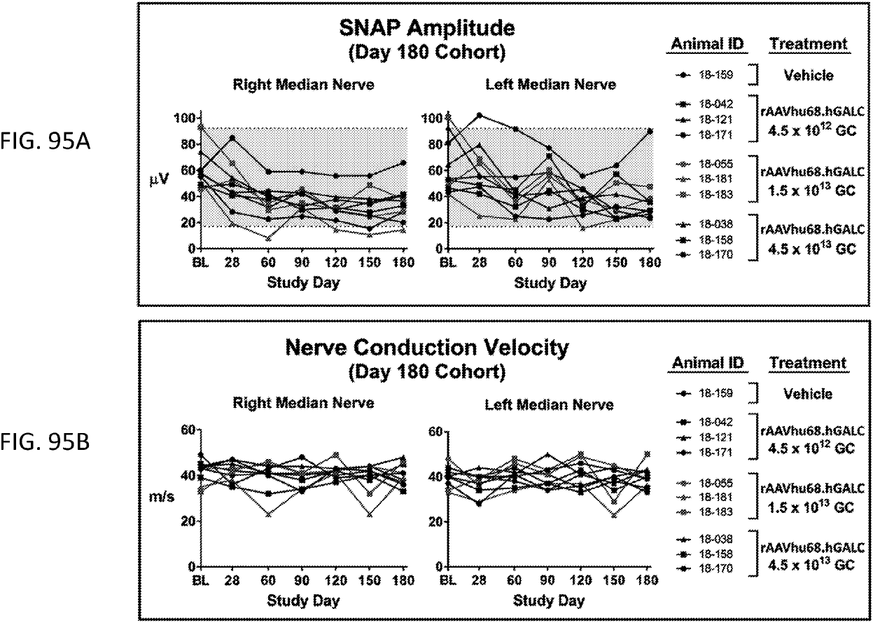

Sensory NCS were performed for all animals at baseline and monthly thereafter to measure bilateral median nerve SNAP amplitudes and conduction velocities (FIG. 94A and FIG. 94B; FIG. 95A and FIG. 95B).

A reduction in SNAP amplitudes from baseline levels that exceeded normal individual animal variability was observed in one vehicle-treated control (Animal 18-159; ITFFB, Group 6) and one animal each in the low dose (Animal 18-168; 4.5 × 10¹² GC, Group 2), mid-dose (Animal 18-181; 1.5 × 10¹³ GC, Group 7), and high dose (Animal 18-080; 4.5 × 10¹³ GC, Group 4) groups (FIG. 94A and FIG. 94B; FIG. 95A and FIG. 95B). The high dose animal (Animal 18-080; 4.5 × 10¹³ GC, Group 4) exhibited decreased SNAP amplitudes in the left median nerve from Day 28 until necropsy on Day 90 that was likely attributable to test-article related sensory neuron toxicity based on correlation with histopathology. SNAP amplitude reductions in the remaining three animals (Animal 18-159 [vehicle: ITFFB], Animal 18-168 [low dose: 4.5 × 10¹² GC], Animal 18-181 [mid-dose: 1.5 × 10¹³ GC]) were considered unrelated to the test article and attributable to technical variability associated with performing NCS in juvenile NHPs. Inter-animal and intra-animal variability in SNAP amplitudes were apparent throughout the study, and the position of the recording needle relative to the nerve can impact SNAP amplitude. Variations in SNAP amplitudes are frequently observed in longitudinal studies, especially in juvenile animals that are still growing and may therefore have slightly modified anatomical landmarks from one time point to another. Nerve conduction velocities are less affected by the electrode positioning and were therefore less variable in the study (FIG. 94 and FIG. 95). No abnormal clinical findings that correlated with reductions in SNAP amplitudes were observed for any of the other animals.

### Body Weights

Animals in both the Day 90 and Day 180 cohorts exhibited weight gain throughout the study. A transient body weight loss was observed at a single time point in some animals, including 9/18 rAAVhu68.hGALC-treated NHPs and 2/2 vehicle-treated controls on Day 7, 1/18 rAAVhu68.hGALC-treated NHPs on Day 28, and 1/18 rAAVhu68.hGALC-treated NHPs on Day 90. The observed weight loss was considered unrelated to the test article.

On Day 7, the majority of animals (6/11) experienced minimal (< 3%) body weight loss from the previously recorded weight, including 3/6 animals administered the low dose of rAAVhu68.hGALC (4.5 × 10¹² GC; Animals 18-091 [-1.72%; Group 2], 18-042 [-2.00%; Group 6], 18-121 [-2.27%; Group 6]), 2/6 animals administered the mid-dose (1.5 × 10¹³ GC; Animals 18-167 [-1.75%; Group 3], 18-187 [-2.44%; Group 3]), and ⅙ animals administered the high dose (4.5 × 10¹³ GC; Animal 18-170 [-2.13%; Group 8]). In the remaining animals, a greater weight loss of approximately 3-10% from the previously recorded weight was observed for 2/2 vehicle-treated controls (ITFFB; Animals 18-162 [-6.12%; Group 1], 18-159 [-3.64%; Group 5]), ⅙ low dose animals (4.5 × 10¹² GC; Animal 18-171 [-6.38%; Group 6]), and 2/6 high dose animals (4.5 × 10¹³ GC; Animals 18-166 [-8.33%; Group 4] and 18-185 [-10.87%; Group 4]). The two animals exhibiting the highest percentage weight loss (Animals 18-166 and 18-185; 4.5 × 10¹³ GC, Group 4) exhibited diarrhea and a reduced appetite from Day 3-10 consistent with shigellosis, which accounted for this temporary weight loss. For the remaining animals, no clinical symptoms were noted that accounted for the observed temporary weight loss. All animals continued to gain and/or maintain weight at all subsequent time points for the duration of the study. Since 2/2 vehicle-treated animals exhibited temporary weight loss on Day 7, this finding was considered unlikely to be test article-related.

On Day 28, one high dose animal (4.5 × 10¹³ GC; Animal 18-080; Group 4) exhibited body weight loss of 3.57% from the previously recorded weight on Day 14. This animal presented with intermittent diarrhea or soft stool accompanied by a loss of appetite on Days 18-25 consistent with shigellosis, which accounted for this temporary weight loss. Following treatment with antibiotics and fiber supplements, the animal gained weight at all subsequent evaluation time points.

On Day 90, one high dose animal (4.5 × 10¹³ GC; Animal 18-038; Group 8) exhibited a weight loss of 4.26% from the previously recorded weight on Day 60. The animal was placed on supplemental feeding and gained weight at all subsequent evaluation time points. No gastrointestinal symptoms were noted that accounted for this weight loss. Since vehicle-treated animals also exhibited transient weight loss during the study, this finding was considered unlikely to be test article-related.

### Cerebrospinal Fluid

### Hematology (Cell Counts)

Mild lymphocytic or neutrophilic pleocytosis (≥6 leukocytes/µL) occurred in 8/18 rAAVhu68.hGALC-treated animals and no vehicle-treated controls (FIG. 96A and FIG. 96C). In some of these cases, the pleocytosis was likely related to hemodilution as >30 red blood cells [RBCs]/µL were observed in some CSF samples as a result of blood contamination during collection. The animals exhibiting lymphocytic pleocytosis that was likely secondary to hemodilution included ⅙ animals in the low dose group (4.5 × 10¹² GC; Animal 18-121 [Group 6]), 3/6 animals in the mid-dose groups (1.5 × 10¹³ GC; Animal 18-176 [Group 3], Animal 18-181, Animal 18-183 [Group 7]), and ⅙ animals in the high dose groups (4.5 × 10¹³ GC; Animal 18-170 [Group 8]). Among these animals, peak CSF leukocyte counts of 8-63 cells/µL were observed 7-180 days after rAAVhu68.hGALC administration. The animals exhibiting lymphocytic or neutrophilic pleocytosis that was not attributable to hemodilution included ⅙ animals in the low dose group (4.5 × 10¹² GC; Animal 18-168 [Group 2]) and 2/6 animals in the high dose group (4.5 × 10¹³ GC; Animal 18-038 and Animal 18-158 [Group 8]). Among these animals, peak CSF leukocyte counts of 8-22 cells/µL were observed 14 days after rAAVhu68.hGALC administration. Mild CSF pleocytosis was considered test article-related and, in all cases, pleocytosis was self-limited and not associated with clinical sequelae.

### Clinical Chemistry

No abnormalities of CSF total protein or glucose were observed in any animals during the study.

### Presence of Neutralizing Antibodies Against AAVhu68 Capsid

NAbs against the AAVhu68 capsid were detectable at baseline in the serum of 12/20 animals in the study, including ½ vehicle-treated controls and 7/18 rAAVhu68.hGALC-treated NHPs.

Among the vehicle-treated controls, the animal that was negative for pre-existing AAVhu68 NAbs (Animal 18-159; ITFFB, Group 5) remained negative throughout the study. The NAb titer in the other vehicle-treated control that was positive for pre-existing AAVhu68 NAbs at baseline (Animal 18-162; ITFFB, Group 1) remained within two dilutions of the pre-existing NAb titer for the duration of the study, which was considered a minimal change.

Among the rAAVhu68.hGALC-treated animals, NAb responses to the AAVhu68 capsid were observed in 18/18 animals by Day 28. AAVhu68 NAbs were detected through Day 180 for all animals administered rAAVhu68.hGALC, with the peak response observed between Day 28 and Day 60 in most animals. Following the peak in NAb titer, a reduction or maintenance of the titer was observed until the end of the study in all animals. While NAb values for the low dose (4.5 × 10¹² GC) and mid-dose (1.5 × 10¹³ GC) groups were comparable during the study, an increase in the magnitude of the NAb response was observed in the high dose groups (4.5 × 10¹³ GC). This difference was most obvious on Day 28 when the geometric mean titers of animals in the high dose groups (4.5 × 10¹³ GC) were approximately 5-fold greater than those in the low dose (4.5 × 10¹² GC) and mid-dose (1.5 × 10¹³ GC) groups. However, by Day 60, the difference in the magnitude of the NAb response decreased. By Day 90, the NAb titers of animals in the high dose groups (4.5 × 10¹³ GC) were approximately 2-fold greater than those of the low dose (4.5 × 10¹² GC) and mid-dose (1.5 × 10¹³ GC) groups, and these titers remained between 2- and 3-fold greater until the end of the study. A statistically significant difference between the titers of the high dose groups (4.5 × 10¹³ GC) and the low dose (4.5 × 10¹² GC) or mid-dose groups (1.5 × 10¹³ GC) was only observed on Day 60 (p=0.03) and Day 150 (p=0.05), respectively. Finally, the magnitude of the AAVhu68 NAb response did not appear influenced by the presence of pre-existing AAVhu68 NAbs prior to rAAVhu68.hGALC administration, regardless of the dose.

NAb responses to AAVhu68 in serum collected throughout the study are summarized below.

TABLE

Presence of Neutralizing Antibodies Against AAVhu68 Capsid in Serum

| Group | Test Article | Dose (GC) | Animal ID | Necropsy Date | AAVhu68 NAb₅₀ | | | | | | |
|---|---|---|---|---|---|---|---|---|---|---|---|
| | | | | | BL | Day 28 | Day 60 | Day 90 | Day 120 | Day 150 | Day 180 |
| 1 | ITFFB | N/A | 18-162 | Day 90 | 80 | 320* | 160 | 80 | | | |
| 2 | vector | 4.5 × 10¹² | 18-091 | Day 90 | < 5 | 320* | 320 | 640 | | | |
| | | | 18-168 | | 10 | 640* | 1280 | 1280 | | | |
| | | | 18-173 | | 80 | 2560* | 1280 | 1280 | | | |
| 3 | vector | 1.5 × 10¹³ | 18-167 | Day 90 | 16 0 | 2560* | 640⁺ | 640 | | | |
| | | | 18-176 | | < 5 | 640* | 320 | 640 | | | |
| | | | 18-187 | | < 5 | 2560* | 5120* | 2560 * | | | |
| 4 | vector | 4.5 × 10¹³ | 18-080 | Day 90 | 10 | 1280* | 2560* | 1280 | | | |
| | | | 18-166 | | 80 | 10240* | 5120* | 1280 | | | |
| | | | 18-185 | | 5 | 10240* | 5120* | 2560 * | | | |
| 5 | ITFFB | N/A | 18-159 | Day 180 | < 5 | < 5 | < 5* | < 5* | <5 | < 5 | < 5 |
| 6 | vector | 4.5 × 10¹² | 18-042 | Day 180 | 80 | 320* | 640 | 160 | 320 | 1280 | 128 0 |
| | | | 18-121 | | 5 | 320* | 640 | 1280 | 640 | 1280 | 128 0 |
| | | | 18-171 | | 5 | 320* | 640 | 640 | 640 | 160 | 160 |
| 7 | vector | 1.5 × 10¹³ | 18-055 | Day 180 | < 5 | 80 | 320 | 320 | 160 | 160 | 160 |
| | | | 18-181 | | < 5 | 320* | 1280 | 320 | 320 | 320 | 640 |
| | | | 18-183 | | < 5 | 320* | 2560* | 1280 | 2560 | 1280 | 128 0 |
| 8 | vector | 4.5 × 10¹³ | 18-038 | Day 180 | < 5 | 320* | 640 | 640 | 1280 | 1280 | 128 0 |
| | | | 18-158 | | 80 | 2560* | 5120* | 1280 | 1280 | 2560 | 256 0 |
| | | | 18-170 | | 40 | 2560* | 1280 | 1280 | 2560 | 2560 | 640 |

Reciprocal serum dilution that inhibited AAVhu68.CMV.LacZ transduction (β-gal expression) by ≥ 50% is shown for each time point. The variability of the assay is ± one 2-fold serum dilution.
*Indicates that the sample was tested twice to determine the end-point NAb titer, and the second dataset is shown in the table.
⁺Indicates that the sample was tested three times to determine the end-point titer, but the second assay result was valid and is shown in the table.
Abbreviations: β-gal, β-galactosidase; BL, baseline; GC, genome copies; ID, identification; ITFFB, intrathecal final formulation buffer; LOD, limit of detection; N/A, not applicable; NAb, neutralizing antibody.

### T Cell Responses to the AAV Capsid and Transgene Product

4/20 NHPs exhibited no IFN-γ T cell response to either the capsid (AAVhu68) or the transgene product (human GALC) during the study. These non-responders included ½ vehicle-treated controls and 3/18 rAAVhu68.hGALC-treated animals. The single vehicle-treated non-responder (ITFFB; Animal 18-162, Group 1) remained negative for T cell responses through necropsy on Day 90. Among rAAVhu68.hGALC-treated animals, 2/6 non-responders in the low dose group (4.5 × 10¹² GC; Animal 18-173, Group 2; Animal 18-121, Group 6) remained negative for T cell responses through necropsy on Day 90 and Day 180, respectively, and ⅙ non-responders in the mid-dose groups (1.5 × 10¹³ GC; Animal 18-167, Group 3) remained negative through Day 90. 16/20 NHPs exhibited IFN-y T cell responses to the capsid and/or the transgene product during the study. Responders included ½ vehicle-treated controls and 15/18 rAAVhu68.hGALC-treated animals. The single vehicle-treated animal (ITFFB; Animal 18-159, Group 5) displayed a T cell response to the transgene product in PBMCs on Day 28 and liver lymphocytes at the Day 180 necropsy. No T cell responses to the capsid were detected in this animal. Since Animal 18-159 was not administered rAAVhu68.hGALC, this low, transient response appeared unrelated to treatment. Responders among the rAAVhu68.hGALC-treated animals included 4/6 animals in the low dose groups (4.5 × 10¹² GC, Groups 2 and 6), ⅚ animals in the mid-dose group (1.5 × 10¹³ GC, Groups 3 and 7), and 6/6 animals in the high dose groups (4.5 × 10¹³ GC, Groups 4 and 8).

Of the 15/18 rAAVhu68.hGALC-treated animals exhibiting a T cell response, the majority (14/15) had a response to either the transgene product only or to both the transgene product and the capsid. Only 1/15 animals exhibited a T cell response to the capsid alone, which was an animal administered the low dose (4.5 × 10¹² GC; Animal 18-091, Group 2).

With regard to response prevalence, T cell responses to the transgene product were more prevalent than responses to the capsid. No difference in the prevalence of the T cell response to the capsid was evident across dose groups or necropsy cohorts. However, T cell responses to the transgene product appeared more prevalent in the mid-dose (1.5 × 10¹³ GC) and high dose groups (4.5 × 10¹³ GC) when compared to responses in the low dose groups (4.5 × 10¹² GC). Across necropsy cohorts, T cell responses to the transgene product in liver lymphocyte were more prevalent at Day 180 than at Day 90, while responses in lymph node and bone marrow lymphocytes appeared more prevalent at Day 90 than at Day 180, with the caveat that the number of animals exhibiting responses in lymph node and bone marrow lymphocytes was low.

With regard to the magnitude of the response, T cell responses to the capsid were of a low magnitude (58-178 spot-forming units [SFU] per million cells) that was similar across all dose groups and cell populations throughout the study. In contrast, T cell responses to the transgene product were of a higher magnitude (58-843 SFU per million cells) with some cell populations exhibiting a greater response. In particular, liver lymphocytes exhibited a higher T cell response to the transgene product than that of PBMCs and other tissue-specific lymphocyte populations. T cell responses to the transgene product in liver lymphocytes were also of a generally higher magnitude in the mid-dose (1.5 × 10¹³ GC) and high dose (4.5 × 10¹³ GC) groups when compared to the responses in the low dose groups (4.5 × 10¹² GC).

T cell responses to the capsid and transgene product were not associated with abnormal clinical observations or changes in hematology, coagulation, and serum chemistry parameters.

### Gross Pathologic Findings

No test article-related gross findings were observed. All gross findings were considered incidental or procedurally related postmortem.

### Histopathologic Findings

Test article-related findings were observed primarily within sensory neurons of the peripheral nervous system in the DRG and trigeminal ganglia (TRG). The DRG/TRG findings consisted of sensory neuronal degeneration. Secondary axonal degeneration (i.e., axonopathy) was seen within the dorsal white matter tracts of the spinal cord and peripheral nerves that contains central and peripheral axons from DRG neurons, respectively. Overall, these findings were observed across all rAAVhu68.hGALC-treated groups for the DRG and only sporadically in the TRG of two mid-dose animals in the Day 90 cohort. The incidence and severity were low. Three DRG segments per animal (cervical, thoracic, lumbar) were analyzed accounting for a total of 54 DRG segments in rAAVhu68.hGALC-treated animals. Grade 1 (minimal) DRG neuronal degeneration was reported in 31% of DRG segments (17/54), while 69% were normal or displayed incidental background lesions.

DRG neuronal degeneration. Test article-related histopathologic findings consisted of neuronal cell body degeneration with mononuclear cell infiltration in the DRG, which project axons centrally into the dorsal white matter tracts of the spinal cord and peripherally to peripheral nerves. The highest severity grade was 1 (minimal), representing neuronal degeneration of sporadic isolated neurons within a DRG section.

At Day 90, the incidence of minimal (grade 1) DRG degeneration was possibly dose-dependent, as this finding was absent in the low dose group (4.5 × 10¹² GC) and observed only in the mid-dose (1.5 × 10¹³ GC; ⅔ animals, 3/9 ganglia, Group 3) and high dose (4.5 × 10¹³ GC; 3/3 animals, 5/9 ganglia, Group 4) groups. Comparing the mid-dose (1.5 × 10¹³ GC) and high dose (4.5 × 10¹³ GC) groups, the incidence and severity (minimal) were similar.

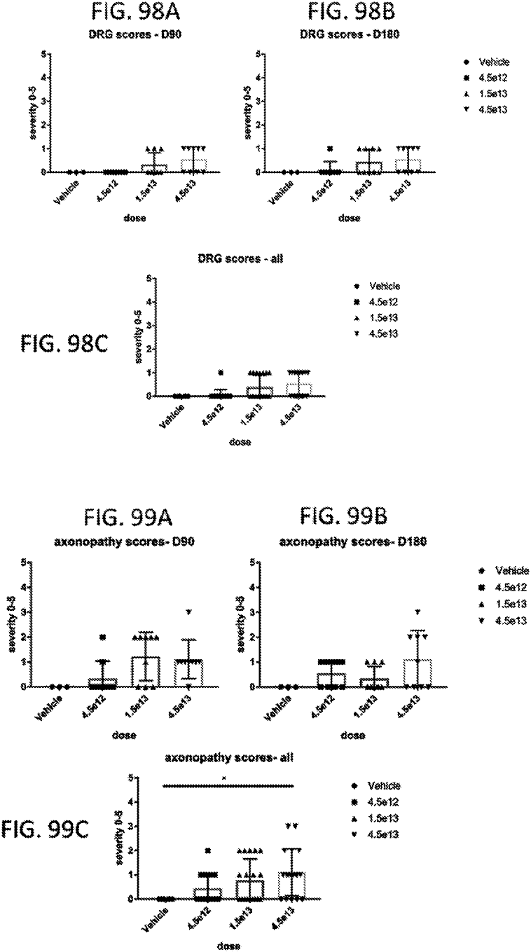

At Day 180, minimal (grade 1) DRG degeneration was observed at all doses, and the incidence appeared dose-dependent. The lowest incidence was observed in the low dose group where a single animal exhibited minimal DRG degeneration in one ganglia (4.5 × 10¹² GC; ⅓ animals, ⅑ ganglia, Group 6). A higher incidence was observed in the mid-dose (1.5 × 10¹³ GC; 3/3 animals, 4/9 ganglia, Group 7) and high dose (4.5 × 10¹³ GC; ⅔ animals, 4/9 ganglia, Group 8) groups. Comparing the mid-dose (1.5 × 10¹³ GC) and high dose (4.5 × 10¹³ GC) groups, the incidence and severity (minimal) were similar. Individual DRG degeneration severity scores observed in the Day 90 and Day 180 necropsy cohorts are presented in FIG. 98A - FIG. 98C.

Axonopathy in spinal cord. DRG neuronal degeneration resulted in axonopathy of the dorsal white matter tracts of the cervical, thoracic, and lumbar spinal cord. The axonopathy was microscopically consistent with axonal degeneration. The incidence was similar across groups, and severity was low overall. Low dose (4.5 × 10¹² GC) and mid-dose (1.5 × 10¹³ GC) groups had similar severity from none to Grade 2 (mild) dorsal axonopathy. Grade 3 (moderate) axonopathy was only observed sporadically in 2/6 animals at the high dose (4.5 × 10¹³ GC).

At Day 90, the incidence of axonopathy was dose-dependent, increasing from ⅓ animals exhibiting axonopathy in the low dose group (4.5 × 10¹² GC; 2/9 segments, Group 2), ⅔ animals exhibiting axonopathy in the mid-dose group (1.5 × 10¹³ GC; 6/9 segments, Group 3), and 3/3 animals exhibiting axonopathy in the high dose group (4.5 × 10¹³ GC; 8/9 segments, Group 4). However, the severity of axonopathy was not clearly dose-dependent. The range of severities increased from minimal to mild in the low dose (4.5 × 10¹² GC) and mid-dose (1.5 × 10¹³ GC) groups to minimal to moderate in the high dose group (4.5 × 10¹³ GC).

At Day 180, the incidence of axonopathy did not appear dose-dependent. The incidence of axonopathy was highest in the high dose (4.5 × 10¹³ GC; ⅔ animals, 5/9 segments, Group 8) and low dose groups (4.5 × 10¹² GC; 3/3 animals, 5/9 segments, Group 6), with a lower incidence observed in the mid-dose group (1.5 × 10¹³ GC; ⅔ animals, 3/9 segments, Group 7). The highest severity was observed at the high dose (4.5 × 10¹³ GC; minimal to moderate, Group 8) followed by low dose group (4.5 × 10¹² GC; minimal, Group 6) and then the mid-dose group (1.5 × 10¹³ GC; minimal, Group 7).

Comparing across time points, a clear time-dependent response was not observed for the axonopathy in dorsal white matter tracts; however, there was no sign of worsening or progression at Day 180 compared to Day 90. At the low dose (4.5 × 10¹² GC), the findings decreased in severity from minimal to mild at Day 90 to minimal on Day 180, but the incidence increased. At the mid-dose (1.5 × 10¹³ GC), both the incidence and severity decreased from minimal to mild at Day 90 to minimal on Day 90. At the high dose (4.5 × 10¹³ GC), the severity increased from mostly minimal at Day 90 to minimal to moderate at Day 180, but the incidence decreased, resulting in a comparable severity score across time points. Definitive interpretation of axonopathy in the dorsal white matter tracts of the spinal cord across time points was therefore difficult and may be reflective of individual animal variability. Severity scores for spinal cord axonopathy observed in the Day 90 and Day 180 necropsy cohorts are presented in FIG. 99A - FIG. 99C.

Axonopathy in peripheral nerves. DRG degeneration resulted in axonopathy of the peripheral nerves, which was microscopically consistent with axonal degeneration and typically observed bilaterally.

At Day 90, both the incidence and severity of peripheral nerve axonopathy appeared dose-dependent. The incidence increased from the low dose (4.5 × 10¹² GC; ⅔ animals, 13/30 nerves; Group 2) to the mid-dose (1.5 × 10¹³ GC; 3/3 animals, 17/30 nerves, Group 3) to the high dose (4.5 × 10¹³ GC; 3/3 animals, 19/30 nerves, Group 4), and the severity increased from minimal in low dose group (4.5 × 10¹² GC; Group 2) to minimal to mild in the mid-dose group (1.5 × 10¹³ GC, Group 3) and high dose group (4.5 × 10¹³ GC; Group 4).

At Day 180, the incidence and severity of the peripheral nerve axonopathy increased across doses, from the low dose (4.5 × 10¹² GC; minimal, ⅓ animals, 1/30 nerves, Group 6) to the mid-dose (1.5 × 10¹³ GC; minimal, ⅔ animals, 4/30 nerves, Group 7) to the high dose (4.5 × 10¹³ GC; minimal to mild, 3/3 animals, 7/30 nerves, Group 8).

Comparing across time points, the incidence and cumulative severity of peripheral nerve axonopathy decreased across all dose groups from Day 90 to 180.

Minimal to mild endoneurial fibrosis (i.e., periaxonal or perineural fibrosis) considered secondary to axonal damage was observed in 2/6 animals in the high dose groups (4.5 × 10¹³ GC), suggesting a possible dose-dependence. At Day 90, mild endoneurial fibrosis was observed in a single nerve (left proximal median nerve) of Animal 18-080 in the high dose group (4.5 × 10¹³ GC; ⅓ animals, ⅒ nerves, Group 4). Endoneurial fibrosis in the left median nerve correlated with a unilateral reduction in SNAP amplitude in the left median nerve, but not the right median nerve, from Day 28 through necropsy on Day 90. At Day 180, minimal to mild endoneurial fibrosis was observed in 10/10 peripheral nerve segments examined (proximal and distal median nerves, peroneal nerves, sciatic nerves, tibial nerves) for Animal 18-038 in the high dose group (4.5 × 10¹³ GC; ⅓ animals; Group 8). Animal 18-038 also exhibited minimal to mild mononuclear cell infiltrates, primarily composed of lymphocytes and plasma cells within some of the peripheral nerves examine (4/10 nerves). Comparing across time points, a clear progression or resolution of fibrosis was not obvious, and this finding may simply represent individual animal variability, as a limited number of nerves and animals at each time point were affected.

Injection site findings. Localized injection site findings within the skeletal muscle and adipose tissue over the ICM/CSF collection site were observed across all groups, including vehicle-treated controls.

Test article-related findings consisted of minimal to moderate chronic inflammation characterized by mononuclear cell infiltrates in the skeletal muscle and/or adipose tissue with associated myofiber changes (i.e., degeneration and regeneration). These findings at the injection site did not worsen from Day 90 to 180 and, in some instances, they improved. At Day 90, the severity and, to a lesser extent, incidence of skeletal muscle inflammation, myofiber regeneration, and myofiber degeneration were dose-dependent, with a higher severity and incidence observed in the high dose group (4.5 × 10¹³ GC; minimal to moderate, 3/3 animals, Group 4) when compared to that of the mid-dose (1.5 × 10¹³ GC; minimal to mild; ⅔ animals, Group 3) or low dose (4.5 × 10¹² GC; minimal to mild; ⅔ animals, Group 2) groups. At Day 180, the incidence and severity of skeletal muscle inflammation (minimal to mild) and myofiber regeneration (minimal) were not dose-dependent, as these findings were similar across all rAAVhu68.hGALC-treated groups. The severity of myofiber degeneration (minimal) was also similar across all dose groups; however, a dose-dependence was observed for the incidence of myofiber degeneration, as more animals in the high dose group (4.5 × 10¹³ GC; 3/3 animals, Group 8) exhibited this finding compared to those in the mid-dose (1.5 × 10¹³ GC; ⅓ animals, Group 7) and low dose (4.5 × 10¹² GC; ⅓ animals, Group 6) groups. Comparing across time points, it was unclear whether the incidence and severity of skeletal muscle inflammation and myofiber changes exhibited a time-dependent response. The incidence of skeletal muscle inflammation was increased in the mid-dose (1.5 × 10¹³ GC) and low dose (4.5 × 10¹² GC) groups from Day 90 (⅔ animals, Group 2; ⅔ animals, Group 3, respectively) to Day 180 (3/3 animals, Group 6; 3/3 animals, Group 7, respectively), but no differences in incidence were observed among the high dose groups (4.5 × 10¹³ GC) from Day 90 (3/3 animals, Group 4) to Day 180 (3/3 animals, Group 8). The severity of skeletal muscle inflammation decreased in the high dose groups (4.5 × 10¹³ GC) from Day 90 (mild to moderate; Group 4) to Day 180 (mild; Group 8), while the low dose (4.5 × 10¹² GC) and mid-dose (1.5 × 10¹³ GC) groups exhibited relatively similar severity (mild) across time points. Across all groups, the incidence and severity of myofiber degeneration/regeneration either improved or remained relatively unchanged from Day 90 to Day 180.

An additional finding of minimal mononuclear cell infiltrates in the skeletal muscle at the injection site was observed at Day 90 in the vehicle-treated control animal (minimal, 1/1 animal, Group 1) and sporadically in animals from the low dose (4.5 × 10¹² GC; mild, ⅓ animals, Group 2) and mid-dose (1.5 × 10¹³ GC; mild, ⅓ animals, Group 3) groups. At Day 180, mononuclear cell infiltrates in the skeletal muscle were not observed in any animals, suggesting resolution of the finding. Given the presence of infiltrates in the vehicle-treated control animal, the significance of this finding was unclear, and it may be incidental or possibly procedurally related to ICM injection and/or repetitive CSF collection.

### Evaluation of Transgene Expression

Transgene product expression (GALC enzyme activity) was evaluated in CNS tissues and major organs (liver and heart). However, because the assay could not distinguish between human GALC enzyme and endogenous rhesus GALC enzyme, the high levels of endogenous rhesus GALC enzyme activity present in normal NHPs made it difficult to detect enzyme activity increases due to the expression of human GALC. In contrast, serum and CSF had substantially lower background levels of endogenous rhesus GALC enzyme activity, enabling evaluation of transgene product expression in these biofluids. However, it should be noted that the analysis was expected to be complicated by the rapid loss of transgene product activity, which is typically attributed to an antibody response to the human transgene product.

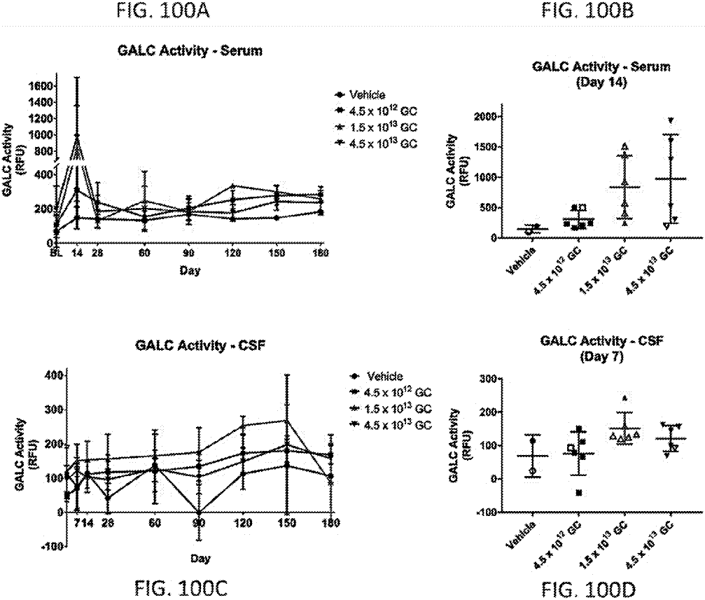

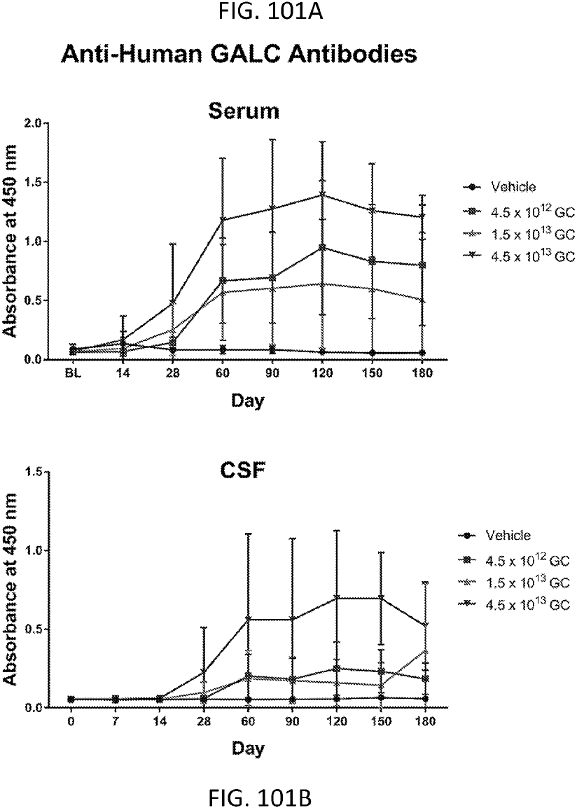

Despite these caveats, GALC enzyme activity was evaluated in serum and CSF (FIG. 100A - FIG. 100D). GALC enzyme activity was detectable in the serum and CSF in animals from all dose groups by the first time point evaluated after rAAVhu68.hGALC administration (Day 14 for serum and Day 7 for CSF) (FIG. 100A - FIG. 100D). In serum, a dose-dependent increase in GALC enzyme activity was observed at the first time point evaluated (Day 14), with an approximately 2-fold, 5.7-fold, and 6.6-fold increase over vehicle-treated control levels at the low dose (4.5 × 10¹² GC), mid-dose, (1.5 × 10¹³ GC), and high dose (4.5 × 10¹³ GC), respectively. As expected, a rapid decline in GALC enzyme activity in serum after Day 14 was observed, which correlated with the onset of anti-human GALC antibody expression around Day 14-28 in both serum and CSF (FIG. 101).

In the CSF, intra- and inter-animal variability in GALC enzyme activity was evident throughout the 180-day study (FIG. 100C). However, at the first time point evaluated (Day 7) prior to the induction of CSF-circulating anti-human GALC antibodies (FIG. 101A), animals receiving the two highest doses (1.5 × 10¹³ GC or 4.5 × 10¹³ GC) displayed GALC enzyme activity levels that were approximately 2-fold and 1.75-fold higher than that of vehicle-treated controls, respectively. The levels at the low dose (4.5 × 10¹² GC) appeared similar to that of the vehicle-treated controls, although an outlier with low levels of GALC enzyme (Animal 18-121, Group 6) was observed in this cohort.

The presence of pre-existing NAbs to the AAVhu68 capsid (denoted by the filled-in shapes in the Serum Day 14 and CSF Day 7 panels of FIG. 100B and FIG. 100D did not appear to impact GALC enzyme activity in the serum or CSF, supporting the potential to achieve therapeutic transgene expression in the target organ system (CNS and PNS) of Krabbe disease patients regardless of NAb status.

### CONCLUSIONS

ICM administration of rAAVhu68.hGALC was well-tolerated at all doses evaluated. rAAVhu68.hGALC produced no adverse effects on clinical and behavioral signs, body weight, or neurologic and physical examinations. There were no abnormalities of blood and CSF clinical pathology related to rAAVhu68.hGALC administration except for an asymptomatic mild transient increase in CSF leukocytes in some animals.
rAAVhu68.hGALC administration resulted in sporadic asymptomatic degeneration of DRG sensory neurons and their associated central and peripheral axons. The severity of these lesions was absent to minimal, and associated axonopathy was mostly minimal to mild. DRG findings were dose-dependent with a trend of higher incidence and/or more severe lesions in the mid-dose (1.5 × 10¹³ GC [1.7 × 10¹¹ GC/g brain]) and high dose (4.5 × 10¹³ GC [5.0 × 10¹¹ GC/g brain]) groups. The DRG findings and corresponding axonopathy were similar at Day 90 and Day 180, suggesting a lack of progression.
Sensory nerve conduction was monitored to provide a sensitive measurement of sensory neuron DRG pathology. One animal in the high dose group (4.5 × 10¹³ GC [5.0 × 10¹¹ GC/g brain]) had unilateral median nerve endoneurial fibrosis that correlated with unilateral reduction in SNAP amplitudes in the left median nerve by Day 28. All the other animals in the study exhibited either absent or asymptomatic DRG sensory neuron pathology.
Transgene expression (i.e., GALC enzyme activity) in the CSF and serum was detectable in animals from all dose groups by the first time point evaluated (Day 7 for CSF and Day 14 for serum). In the CSF, animals receiving the two higher doses (1.5 × 10¹³ GC [1.7 × 10¹¹ GC/g brain] or 4.5 × 10¹³ GC [5.0 × 10¹¹ GC/g brain]) displayed GALC activity levels that were approximately 2fold and 1.75-fold higher than the levels of vehicle-treated controls, respectively. In the serum, animals in all dose groups (4.5 × 10¹² GC [5.0 × 10¹⁰ GC/g brain], 1.5 × 10¹³ GC [1.7 × 10¹¹ GC/g brain], or 4.5 × 10¹³ GC [5.0 × 10¹¹ GC/g brain]) exhibited GALC activity levels that were approximately 2-fold, 5.7-fold, and 6.6-fold higher than the levels of vehicle-treated controls, respectively. Transgene product expression in the CSF and serum was not affected by the presence of pre-existing NAbs to the vector capsid, supporting the potential to achieve therapeutic activity in the target organ systems (CNS and PNS) in Krabbe disease patients regardless of NAb status.
T cell responses to the vector capsid and/or human transgene product were detectable in the PBMCs and/or tissue lymphocytes (liver, spleen, bone marrow, lymph nodes) in the majority of rAAVhu68.hGALC-treated animals. T cell responses were not generally associated with any abnormal clinical findings.

### Example 11 - Treatment of Krabbe Disease With rAAVhu68.hGALC

The FIH trial is a Phase ½ dose escalation study of a single ICM administration of rAAVhu68.hGALC in pediatric patients with the infantile form of Krabbe disease caused by homozygous or compound heterozygous mutations in the GALC gene. This FIH trial enrolls and treats at least 12 subjects who are followed up for 2 years, with continued long-term follow-up (LTFU) for a total of 5 years post-dose in line with the recommended LTFU for adenoviral vectors described in the draft “FDA Guidance for Industry: Long Term Follow-Up after Administration of Human Gene Therapy Products” (July 2018). The primary objectives are to assess the safety and tolerability of rAAVhu68.hGALC. The secondary objectives of this study are to evaluate the impact of rAAVhu68.hGALC on disease-relevant assessments, including survival, age-appropriate neurocognitive measurements, and age-appropriate motor and/or linguistic assessments. These endpoints are selected in consultation with disease experts and clinicians and based on observations on the disease evolution in untreated patients with infantile Krabbe disease.

Optionally, combination therapy of HSCT and AAV gene therapy can be evaluated. The FIH is an open-label, multi-center, dose escalation study of rAAVhu68.hGALC to evaluate safety, tolerability, and exploratory efficacy endpoints in pediatric subjects with the infantile form of Krabbe disease. The dose-escalation phase assesses the safety and tolerability of a single ICM administration of two dose levels of rAAVhu68.hGALC, with staggered, sequential dosing of subjects. The rAAVhu68.hGALC dose levels are determined based on data from the GLP NHP toxicology study and the murine (MED) study and consist of a low dose (administered to Cohort 1) and a high dose (administered Cohort 2). Both dose levels are anticipated to confer therapeutic benefit, with the understanding that, if tolerated, the higher dose is expected to be advantageous. The sequential evaluation of the low dose followed by the high dose enables the identification of the maximum tolerated dose (MTD) of the doses tested. Finally, an expansion cohort (Cohort 3) receives the MTD of rAAVhu68.hGALC. Rapid GALC enzyme production following administration of the vector (with 1 week post-treatment) provides an extended therapeutic window.

An independent Safety Board conducts a safety review of all accumulated safety data between cohorts and after full enrollment of the second cohort to make a recommendation regarding further conduct of the trial. The Safety Board also conducts a review any time a safety review trigger (SRT) is observed. The 1-month dosing interval between the first and second subject in each cohort allows for evaluation of AEs indicative of acute immune reactions, immunogenicity or other dose-limiting toxicities as well as clinical review of any sensory neuropathy that might present itself consistent with the anticipated time course for development of sensory neuropathology secondary to transduction of DRG, which occurs within 2-4 weeks in non-clinical studies.

Additional subjects are enrolled in an expansion cohort that receives the MTD. Enrollment of these additional subjects does not require a 4-week observation window between subjects. Optionally, this cohort receives combination treatment with HSCT and rAAVhu68.hGALC.

All treated subjects are followed for 2 years to evaluate the safety profile and to characterize the pharmacodynamic and efficacy properties of rAAVhu68.hGALC. Subjects are followed for an additional 3 years (for a total of 5 years post-dose) during the LTFU period of the study to evaluate long-term clinical outcomes, which is in line with draft “FDA Guidance for Industry: Long Term Follow-Up after Administration of Human Gene Therapy Products” (July 2018).

TABLE

First-in-Human Clinical Trial Protocol Synopsis

| Protocol(s) Title: | A Phase ½ Open-Label, Multi-Center, Dose Escalation Study to Assess the Safety and Tolerability of Single Doses of rAAVhu68.hGALC Delivered into the Cisterna Magna (ICM) of Pediatric Subjects with Infantile Globoid Cell Leukodystrophy (Krabbe Disease) |
|---|---|
| Number of Subjects: | Up to 12 evaluable subjects |
| Objectives: | Primary: |
| | To assess the safety and tolerability of rAAVhu68.hGALC through 24 months following administration of a single ICM dose through evaluation of: |
| | Adverse events (AEs) and serious adverse events (SAEs) |
| | Vital signs and physical examinations |
| | Neurological examinations |
| | Electrocardiograms (ECGs) |
| | Sensory nerve conduction studies (for evaluation of DRG toxicity) |
| | Laboratory assessments (serum chemistry, hematology, coagulation studies, liver function tests (LFTs), urinalysis, and CSF chemistry and cytology) |
| | Immunogenicity of the vector and transgene product |
| | Secondary (exploratory efficacy): |
| | To assess the pharmacodynamics and biological activity of rAAVhu68.hGALC over 2 years following administration of a single ICM dose, based on the following endpoints: |
| | Levels of GALC in CSF and serum |
| | To assess the efficacy of rAAVhu68.hGALC through 2 years following administration of a single ICM dose as measured by: |
| | Survival |
| | Disease progression as assess by age at achievement, age at loss and percentage of children maintaining or acquiring age appropriate developmental milestones |
| | Disease progression as assessed by age at achievement, age at loss, and percentage of subjects maintaining motor milestones (as defined by WHO criteria) and percentage of subjects progressing in disease staging. |
| | Exploratory: |
| | To further assess the efficacy of rAAVhu68.hGALC through 2 years following administration of a single ICM dose, as measured by: |
| | Age-at-onset and frequency of seizures as assessed by a seizure dairy |
| | Clinical outcomes as assessed by the Bayley Scale (BSID-III) or the Mullen Scales of early learning (depending on age of the subject), Vineland Adaptive Behavior Scales (Edition III), Peabody Developmental Motor Scales, Infant and Toddler Quality of Life Questionnaire |
| | To further assess the pharmacodynamic effects of rAAVhu68.hGALC through 24 months following administration of a single ICM dose, as measured by: |
| | Central nervous system myelination as measured by MRI and DT-MRI |
| | Nerve conduction velocity (NCV) measurements of the deep peroneal, tibial, ulnar, sural, median nerves (to assess sensory and motor nerve peripheral neuropathy) |
| | Visual evoked potentials (VEPs) |
| | Brainstem auditory evoked responses (BAERs) |
| | CSF and plasma/serum biomarkers of disease, including psychosine and others |
| Study Design: | Phase ½, FIH, multi-center, open-label, single-arm, dose escalation study of rAAVhu68.hGALC administered by a single ICM injection in pediatric subjects with infantile Krabbe disease. Safety and tolerability, pharmacodynamics, and clinical efficacy are assessed over 2 years, and all subjects are followed through 5 years post-administration of rAAVhu68.hGALC for the long-term evaluation of safety and tolerability, pharmacodynamics, disease progression, and clinical outcomes. The study consists of a screening phase to determine eligibility of each potential subject from approximately Day -35 to Day -1. After confirmation of subject eligibility and parent’s/guardian’s willingness to have their child participate in the study, the subject undergoes baseline assessments, which include brain magnetic resonance imaging (MRI), lumbar puncture (LP) for cerebrospinal fluid (CSF) collection, blood draw, urine collection, vitals, ECG, a physical exam, a neurological exam and clinical assessments. Baseline assessments occur on Days -1 and Day 0 and eligibility are reconfirmed at baseline prior to administration of rAAVhu68.hGALC. During the treatment phase, subjects are admitted to the hospital on the morning of Day 0. Subjects receive a single ICM dose of rAAVhu68.hGALC on Day 0 and remain in the hospital for at least 24 hours after dosing for observation. Subsequent study visits occur at Day 7, Day 30, 3 months, and 6 months after dosing, followed by every 6 months for the first 2 years after dosing. LTFU visits occur for an additional 3 years at a frequency of every 12 months, through 5 years post-dosing. |
| | The study consists of the following three cohorts administered rAAVhu68.hGALC as a single ICM injection: |
| | Cohort 1 (Low Dose): Three eligible subjects (subjects #1 to #3) are sequentially enrolled and administered the low dose of rAAVhu68.hGALC with a 4 week safety observation period between the first and second subject. If no safety review triggers (SRTs) are observed, all available safety data are evaluated by a safety board 4 weeks after the third subject in Cohort 1 is administered rAAVhu68.hGALC. |
| | Cohort 2 (High Dose): If the decision is made to proceed, three eligible subjects (Subjects #4 to #6) are sequentially enrolled and administered the high dose of rAAVhu68.hGALC with a 4 week safety observation period between the fourth and fifth subject. If no SRTs are observed, the safety board evaluates all available safety data, including safety data from subjects in Cohort 1, 4 weeks after the third subject Cohort 2 is administered rAAVhu68.hGALC. |
| | Cohort 3 (MTD): Pending a positive recommendation by the safety board, 6 additional subjects (Subjects #7-#12) are enrolled and administered a single ICM dose rAAVhu68.hGALC at the MTD. Dosing for subjects in this cohort is not staggered with a 4 week safety observation period between each subject, and a safety board review is not required following dosing of the first three subjects in this cohort. |
| | Cumulatively, we anticipate a total enrollment of 9 subjects in either the high- or low-dose cohort, and 12 subjects in total (across all doses). |
| Inclusion Criteria | 1. ≥ 1 month and <9 months of age at enrollment, and are presymptomatic or symptomatic with first symptoms of Krabbe disease at ≤6 months of age |
| | 2. Leukocyte GALC activity below the lower limit of normal (LLN) of the testing central laboratory (e.g., <0.30 nmol/hour/mg protein at the Mayo Clinic). |
| | 3. Whole blood psychosine > 10 nM |
| | 4. Biallelic pathogenic GALC gene variants associated with early infantile Krabbe disease or variants classified as likely pathogenic (testing must be done at a Clinical Laboratory Improvement Amendments [CLIA] or CLIA-equivalent laboratory certified per local standard) |
| | 5. Parents or the subject’s LAR provide(s) written informed consent prior to any study-related procedures, including screening evaluations |
| | 6. Symptomatic subjects must exhibit a minimum level of neurological and developmental function that indicates they have the potential to benefit from treatment, at least with slowing or stabilization of their disease. In particular, the subject must demonstrate the following clinical features (when age-appropriate): |
| | a. Thrusting of legs in play (Bayley motor scale, gross motor subset, item 1) |
| | b. Lifting of head (Bayley motor scale, gross motor subset, item 3) |
| | c. Eyes follow moving person (Bayley motor scale, fine motor subset, item 2) |
| | d. Smiles in response to speaker’s attention (Bayley language scale, expressive, item 2) |
| Exclusion Criteria | 1. Any clinically significant neurocognitive deficit not attributable to Krabbe disease or any other condition that may, in the opinion of the Investigator, confound interpretation of study results. |
| | 2. If any subject had an acute illness requiring hospitalization within 30 days of enrollment, the history must be discussed with the Sponsor’s Medical Monitor before allowing the subject to be enrolled. |
| | 3. History of chronic ventilation assisted respiratory support (defined as use of more than 12 hours/day of bilevel positive airway pressure, continuous positive airway pressure, or ventilator) or a need for tracheostomy as a result of their disease. Note: This does not exclude patients who use respiratory vests. |
| | 4. Intractable seizure or uncontrolled epilepsy defined as having had an episode of status epilepticus, or seizures requiring hospitalization. |
| | a. This does not exclude subjects who have a history of staring spells that have not been associated with EEG findings |
| | 5. Family history of seizure disorders or epilepsy of infantile or childhood onset, other than febrile seizures |
| | a. This does not exclude subjects with a family history of Krabbe disease |
| | 6. Any contraindication to ICM administration procedure, including contraindications to fluoroscopic imaging, IT contrast and anesthesia, or any condition that would increase the risk of adverse outcomes from the ICM procedure, including but not limited to the presence of space occupying lesion causing mass effect or signs of increased intracranial pressure, non-communicating hydrocephalus, space-occupying lesion in the posterior fossa or foramen magnum, aberrant vascular anatomy such as a large midline posterior inferior cerebellar artery, venous anomaly such as a large midline cerebellar vein or occipital sinus, congenital anatomical abnormalities such as Chiari malformation. |
| | 7. Any contraindication to MRI or LP. |
| | 8. Prior gene therapy. |
| | 9. Enrollment in any other clinical study with an investigational product within 4 weeks prior to Screening or within 5 half-lives of the investigational product used in that clinical study, whichever is longer |
| | 10. Prior HSCT |
| | 11. Receipt of a vaccine within 14 days prior to or after dosing. |
| | 12. Estimated glomerular filtration rate (eGFR) <60 mL/min/1.73 m² based on creatinine, as determined using the Bedside Schwartz equation (Schwartz and Work, 2009). |
| | 13. Hematological abnormalities: |
| | a. Coagulopathy (INR > 1.5 or activated partial thromboplastin time [aPTT] > 40 seconds). |
| | b. Thrombocytopenia (platelet count < 100,000 per µL). |
| | c. WBC < 5.5 × 10³ cells/ µL |
| | d. Hemoglobin < 10 g/dL |
| | 14. AST or ALT >3 × ULN or total bilirubin >1.5 × ULN. |
| | 15. Abnormal respiratory function: |
| | a. Required suctioning in the absence of upper respiratory tract infection |
| | b. Hypoxemia (oxygen [O₂] saturation awake less than 96% or O₂ saturation asleep less than 96%, without ventilation support) as assessed during screening. Ventilatory support is defined as dependence on supplemental O₂ or use of a ventilator or bilevel positive airway pressure (BiPap) or continuous positive airway pressure (Cpap) machine. |
| | 16. Poor peripheral perfusion or temperature instability in the absence of intercurrent illness |
| | 17. Medical conditions or laboratory or vital sign abnormalities that would increase risk of complications from ICM injection, anesthesia, fluoroscopy, LP, and/or MRI (temperature >38° C., oxygen saturation below 95% on room air or baseline oxygen requirement, heart rate or respiratory rate abnormal for age of the subject, abnormal blood pressure for age, evidence of infection) |
| | 18. Any condition (e.g., history of any disease, evidence of any current disease, any finding upon physical examination, or any laboratory abnormality) that, in the opinion of the Investigator, would interfere with evaluation of the investigational product or interpretation of subject safety or study results. This includes: |
| | a. Abnormal laboratory values considered clinically significant by the Investigator |
| | b. Underlying defect in immune function. |
| | c. History of multiple and severe life-threatening infections |
| Investigational Product | rAAVhu68.hGALC |
| Route of Administration and Procedure | rAAVhu68.hGALC is administered as a single dose to hospitalized subjects on Day 0 via CT-guided sub-occipital injection into the cisterna magna. |
| | On Day 0, a syringe containing 5.6 mL of rAAVhu68.hGALC at the appropriate titer is prepared by the Investigational Pharmacy associated with the study and delivered to the procedure room. |
| | Prior to study drug administration, the subject is anesthetized, intubated, and the injection site is prepped and draped using sterile technique. An LP is performed to remove a predetermined volume of CSF, after which iodinated contrast is IT injected to aid in visualization of relevant anatomy of the cisterna magna. IV contrast may be administered prior to or during needle insertion as an alternative to the IT contrast. The decision to use IV or IT contrast is at the discretion of the interventionalist performing the procedure. A spinal needle (22-25 G) is advanced into the cisterna magna under fluoroscopic guidance. A larger introducer needle may be used to assist with needle placement. After confirmation of needle placement, the extension set is attached to the spinal needle and allowed to fill with CSF. At the discretion of the interventionalist, a syringe containing contrast material may be connected to the extension set and a small amount injected to confirm needle placement in the cisterna magna. After the needle placement is confirmed, the syringe containing rAAVhu68.hGALC is connected to the extension set. The syringe contents are slowly injected over 1-2 minutes, delivering a volume of 5.0 mL. |
| Safety Assessments | Safety assessments, including collection of adverse events (AEs) and serious adverse events (SAEs), physical and neurologic examinations, vital signs, clinical laboratory tests (serum chemistry, hematology, coagulation, LFTs, urinalysis), ECGs, nerve conduction studies and CSF cytology and chemistry (cell counts, protein, glucose) is performed. |
| | The Investigator has primary responsibility for the ongoing medical review of safety data (AEs, SAEs, laboratory data, etc.) throughout the study and prior to enrollment of each subject during the dose escalation phase. A safety board reviews safety data at specified intervals throughout the study and make recommendations to the Sponsor regarding further conduct of the study. Safety evaluations after the first three subjects in Cohort 1 and after the first three subjects in Cohort 2 are conducted. |

### Statistical Methods

No statistical comparisons are planned for safety evaluations; all results are descriptive only. Data are listed and summary tables are produced.

Statistical comparisons are performed for secondary and exploratory endpoints. Measurements at each time point are compared to baseline values for each subject, as well as data from age matched healthy controls and natural history data from Krabbe disease patients with comparable cohort characteristics where available for each endpoint.

All data are presented in subject data listings. Categorical variables are summarized using frequencies and percentages, and continuous variables are summarized using descriptive statistics (number of non-missing observations, mean, SD, median, minimum, and maximum). Graphical displays are presented as appropriate.

### Population Rationale

### Study Population - Pediatric Patients

The FIH focuses on infantile subjects with symptom onset before 9 months of age, who represent the population with the highest unmet need as HSCT is not indicated for these patients. Furthermore, these patients have a singularly devastating disease course with rapid and highly predictable decline that is homogeneous in the presentation of both motor and cognitive impairment (Bascou N., et al. (2018) Orphanet J Rare Dis. 13(1): 126). In fact, patients presenting with symptoms before 9 months of age have a disease course that resembles early infantile Krabbe Disease, with rapid and severe cognitive and motor impairment progression, and failure to gain any functional skills following initial signs and symptoms of disease. The majority of these patients is expected to die within the first few years of life (2 year survival ranges from 26-50% (Duffner P.K., et al. (2011) Pediatr Neurol. 45(3): 141-8; Beltran-Quintero M.L., et al. (2019) Orphanet J Rare Dis. 14(1):46). The phenotype of infants with onset between 9 and 12 months of age is more variable, with some exhibiting the severe early infantile Krabbe Disease phenotype, while others have a less severe disease presentation with (near) normal cognition and markedly better adaptive and fine motor skills, which makes it difficult to predict the phenotype of a newly diagnosed patient with onset between 9 and 12 months (Bascou N., et al. (2018) Orphanet J Rare Dis. 13(1): 126). Consequently, the population is restricted to subjects with symptom onset <9 months of age whose predictable and rapid decline supports a robust study design and evaluation of functional outcomes within a reasonable follow up period. For this group, treatment is expected to stabilize disease progression and prevent loss of skills such as acquired developmental and motor milestones, prolong survival, delay or prevent development of seizures.

Despite a shared underlying pathophysiology, the adult Krabbe phenotype and disease course is notably milder from devastating infantile Krabbe Disease form so demonstration of disease stabilization in adults would not provide reason to believe for the therapy in infantile Krabbe Disease. Importantly, adult Krabbe disease onset is highly variable, and progression is slower and more variable with decline occurring over many years to decades (Jardim L.B., et al. (1999) Arch Neurol. 56(8): 1014-7; Debs R., et al. (2013) J Inherit Metab Dis. 36(5):859-68). It would be very challenging to design a clinical trial that could unequivocally demonstrate efficacy of the investigational therapy in the context of a protracted natural course. The fact that HSCT provides a treatment option able to stabilize or even improve the disease manifestations is another important consideration (Sharp M.E., et al. (2013) JIMD Rep. 10:57-9; Laule C., et al. (2018) Journal of Neuroimaging. 28(3):252-255). Finally, NBS has not been widely adopted in the US and is not available in Europe, and the ambiguous, non-specific clinical presentation means that adult Krabbe disease continues to be underdiagnosed and thus access to such patients remains exceedingly rare (Wasserstein M.P., et al. (2016) Genet Med. 18(12): 1235-1243).

### Study Population - Exclusion of Subjects With Severe Disease

Given the nature of Krabbe disease with CNS injury thought to be largely irreversible and the very rapid disease progression in the infantile population, rAAVhu68.hGALC is expected to confer the greatest potential for benefit in patients with no or mild to moderate disease that do not exhibit signs that are uniquely associated with the latter stages of disease, including deafness, blindness, severe weakness with loss of primitive reflexes (Escolar M.L., et al. (2006) Pediatrics. 118(3):e879-89). Additionally, abnormal pupillary reflexes, jerky eye movement, or visual tracking difficulties are more common in very advanced disease than in patients with moderate signs and symptoms, and are not typically observed in the early disease stages (Escolar M.L., et al. (2006) Pediatrics. 118(3):e879-89). Therefore, evidence of more than one of these signs are considered an indicator of advanced disease and result in exclusion from the trial. Due to the severe disability, these patients would be unlikely to gain substantial benefit from the therapy beyond stabilization of disease at a low level of clinical function are excluded, the benefit/risk profile would not be favorable, and they would exhibit floor effects on various clinical and instrumental assessments that would preclude evaluation of the efficacy of rAAVhu68.hGALC. This population may also present with a higher risk for non-treatment-related safety concerns due to the advanced state of disease sequelae and are excluded from this trial.

Patients with clinical seizures are not excluded from the trial, unless in the opinion of the Investigator the child has other signs of advanced disease and would be unlikely to benefit from treatment. This is because 1) seizures are not uniquely associated with advanced disease and 2) seizures are an endpoint in the trial and excluding patients with seizures might bias the study towards a population that is less prone to experience seizures.

### Study Population - Inclusion of Presymptomatic Subjects

Presymptomatic infantile Krabbe Disease patients are excluded from the dose escalation portion of the study (Cohort 1 and Cohort 2) in which rAAVhu68.hGALC alone is evaluated. For these patients, at least in the US, HSCT is considered a therapeutic option and the treatment of choice, even if it only serves to delay disease progression. The prevailing US KOL opinion is that testing an unproven investigational therapy would be considered unethical in this population because the exceedingly narrow therapeutic window would effectively deprive the patient of access to a treatment shown to provide at least partial benefit (i.e., should gene therapy prove unsuccessful there would unlikely be time to “rescue” with HSCT). Thus, rAAVhu68.hGALC should be reserved for patients with the clearest unmet need (i.e., infantile Krabbe disease patients with signs and symptoms who are not eligible for HSCT).

### Study Population - Justification of the Lower Age Limit

Given that symptom onset can occur perinatally, or even in utero, treatment occurs as early as possible to maximize potential benefit. , thus the minimum age of the study was selected as 1 month old at dosing as current consensus guidelines recommend HSCT before 1 month of age in eligible patients (Kwon J.M., et al. (2018) Orphanet J Rare Dis. 13(1):30). Requiring subjects to be 1 month or older allows subjects and families to consider other forms of standard-of-care treatment prior to their eligibility for this trial.

Another consideration in selecting the lower age limit is to ensure that the treatment, and specifically the ICM procedure can be safely carried out in such a young patient. After careful review of imaging scans from infants as young as 1 or 2 weeks of age, an expert interventional radiologist at the University of Pennsylvania confirmed that there is no specific anatomical concern with performing CT guided ICM administration in a 1-month-old infant, provided the rationale for treatment is supported.

### Endpoints

In addition to measuring safety and tolerability as primary endpoints, secondary and exploratory pharmacodynamic and efficacy endpoints were chosen for this study based on the current literature and in consultation with leading clinicians specializing in Krabbe disease. These endpoints are anticipated to demonstrate meaningful functional and clinical outcomes in this population. Endpoints are measured at 30 days, 90 days and 6 months, and then every 6 months during the 2-year short-term follow-up period, except for those that require sedation and/or a lumbar puncture. During the long-term extension phase, measurement frequency decreases to once every 12 months. These time points were selected to facilitate thorough assessment of the safety and tolerability of rAAVhu68.hGALC. The early time points and 6 monthly interval were also selected in consideration of the rapid rate of disease progression in untreated infantile Krabbe patients. This allows for thorough evaluation of pharmacodynamics and clinical efficacy measures in treated subjects over a period of follow up for which untreated comparator data exist. Subjects continue to be monitored for safety and efficacy for a total of 5 years after rAAVhu68.hGALC administration, in accordance with the draft “FDA Guidance for Industry: Long Term Follow-Up After Administration of Human Gene Therapy Products” (July 2018).

### Disease Progression and Clinical Outcomes

In view of the rapid and homogeneous rate of disease progression in the infantile population (Duffner P.K., et al. (2011) Pediatr Neurol. 45(3): 141-8; Bascou N., et al. (2018) Orphanet J Rare Dis. 13(1): 126) a 2 year follow up for primary outcomes evaluation is considered sufficient to evaluate the impact of rAAVhu68.hGALC over time. In addition, the LTFU to 5 years post-treatment is very informative for assessing long-term outcomes and if the treatment is effective in prolonging survival and stabilizing patients at a level of function similar or superior to the outcomes observed in presymptomatic patients after HSCT.

Administration of rAAVhu68.hGALC stabilizes disease progression as measured by survival, preventing loss of developmental and motor milestone potentially supporting acquisition of new milestones, onset and frequency of seizures. Death typically occurs in the first 3 years of life for a majority patients diagnosed with early infantile Krabbe disease, with median mortality extending to 5 years in the late infantile population which incorporates patients with symptom onset from 7-12 months (Duffner P.K., et al. (2012) Pediatr Neurol. 46(5):298-306). By limiting the inclusion criteria to patients with onset on or before 9 months of age, the population has more severe, early infantile-like phenotype and disease course (Bascou N., et al. (2018) Orphanet J Rare Dis. 13(1): 126). Given the rapid decline seen in cases of untreated infantile Krabbe disease, treatment with rAAVhu68.hGALC extends life expectancy during the follow-up periods. Motor milestone development depends on the age and stage of disease at the time of subject enrollment (Bascou N., et al. (2018) Orphanet J Rare Dis. 13(1): 126; Beltran-Quintero M.L., et al. (2019) Orphanet J Rare Dis. 14(1):46). Given the severity of disease in the target population, subjects may have achieved motor skills by enrollment, developed and subsequently lost other motor milestones, or not yet shown signs of motor milestone development. Assessments therefore track age-at-achievement and age-at-loss for all milestones. Motor milestone achievement is defined for six gross milestones based on the World Health Organization (WHO) criteria outlined below.

TABLE

WHO Performance Criteria for Gross Motor Milestones

| | |
|---|---|
| Gross Motor Milestone | Multicenter Growth Reference Study Performance Criteria |
| Sitting without support | Child sits up straight with the head erect for at least 10 seconds. Child does not use arms or hands to balance body or support position. |
| Hands-and-knees crawling | Child alternately moves forward or backward on hands and knees. The stomach does not touch the supporting surface. There are continuous and consecutive movements, at least three in a row. |
| Standing with assistance | Child stands in upright position on both feet, holding onto a stable object (e.g., furniture) with both hands without leaning on it. The body does not ouch the stable object, and the legs support most of the body weight. Child thus stands with assistance for at least 10 seconds. |
| Walking with assistance | Child is in upright position with the back straight. Child makes sideways or forward steps by holding on a stable objects (e.g., furniture) with one of both hands. One leg moves forward while the other supports part of the body weight. Child takes at least five steps in this manner. |
| Standing alone | Childs stands in upright position on both feed (not on the toes) with the back straight. The legs support 100% of the child’s weigh. There is no contact with a person or objects. Child stands alone for at least 10 seconds. |
| Walking alone | Child takes at least five steps independently in upright position with the back straight. One leg moves forward while the other supports most of the body weight. There is no contact with a person or object. |

Adapted from (Wijnhoven T.M., et al. (2004) Food Nutr Bull. 25(1 Suppl):S37-45). Abbreviations: WHO, World Health Organization.

Given that subjects with infantile Krabbe disease can develop symptoms within the first weeks or months of life, and acquisition of the first WHO motor milestone (sitting without support) typically does not manifest before 4 months of age (median: 5.9 months of age), this endpoint may lack sensitivity to evaluate the extent of therapeutic benefit, especially in subjects who had more overt symptoms at the time of treatment. For this reason, assessment of age appropriate developmental milestones that can be applied to infants are also included (Sharp M.E., et al. (2013) JIMD Rep. 10:57-9). One short-coming is that the published tool is intended for use by clinicians and parents, and organizes skills around the typical age of milestone acquisition without referencing normal ranges. However, the data may be informative to summarize retention, acquisition, or loss of developmental milestones over time, relative to untreated children with infantile Krabbe disease or the typical time of acquisition in neurotypical children.

While seizures are not a presenting symptom for the infantile population, approximately 30-60% of infantile patients will eventually develop seizures in the later stages of the disease (Duffner P.K., et al. (2011) Pediatr Neurol. 45(3): 141-8). The delayed onset of seizure activity enables us to determine if treatment with rAAVhu68.hGALC can either prevent or delay onset of seizures in this population, or decrease the frequency of seizure events. Parents are asked to keep seizure diaries which track onset, frequency, length and type of seizure. These entries are discussed with and interpreted by the clinician at each visit.

As exploratory measures, clinical scales are used to quantity the effects of rAAVhu68.hGALC on development and changes in adaptive behaviors, cognition, language, motor function, and health-related quality of life. Each measure proposed has been used either in the Krabbe population or in a related population. The scale and relevant domains are briefly described below:
Bayley Scales of Infant and Toddler Development (Edition III): Assesses development of infant and toddlers across five domains: cognitive, language, motor, social-emotional, and adaptive behavior. All domains are assessed in the trial.
Vineland Adaptive Behavior Scales (Edition III): Assesses adaptive behavior from birth through adulthood (0-90 years) across five domains: communication, daily living skills, socialization, motor skills, and maladaptive behavior. Improvements from v2 to v3 incorporate questions to enable a better understanding of developmental disabilities.
Peabody Developmental Motor Scales- Second Edition: Measures interrelated motor function from birth to children five years of age. Assessments focus on six domains: reflexes, stationary, locomotion, object manipulation, grasping, and visual-motor integration
Infant Toddler Quality of Life Questionnaire (ITQOL): Parent-reported measure of health-related quality of life designed for infants 2 months of age up to toddlers 5 years of age.
Mullen Scales of Early Learning: Assesses language, motor, and perceptual abilities in infants and toddlers up to 68 months of age.

### Disease Biomarkers

To assess the effect of rAAVhu68.hGALC on disease pathology, changes in myelination, functional outcomes related to myelination, and potential disease biomarkers are measured. As the primary hallmark of disease, central and peripheral demyelination slow or cease in progression with rAAVhu68.hGALC administration. Central demyelination is tracked by diffusion-tensor magnetic resonance imaging (DT-MRI) anisotropy measurements of white matter regions and fiber tracking of corticospinal motors tracts, changes in which are indicators of disease state and progression (McGraw P., et al. (2005) Radiology. 236(1):221-30; Escolar M.L., et al. (2009) AJNR Am J Neuroradiol. 30(5): 1017-21). Peripheral demyelination is measured indirectly via nerve conduction velocity (NCV) studies on the motor nerves (deep peroneal, tibial, and ulnar nerves) and sensory nerves (sural, and median nerves) to monitor for fluctuations indicative of a change in biologically active myelin (i.e., F-wave and distal latencies, amplitude or presence or absence of a response).

Development of visual impairment is common in early infantile Krabbe, with 61.2% of the population developing vision loss at some point in the disease according to one study (Duffner P.K., et al. (2011) Pediatr Neurol. 45(3): 141-8). Similar to seizures, vision loss is not a common presenting symptom. This offers the opportunity to assess the ability of rAAVhu68.hGALC to delay or prevent vision loss for those subjects that have not developed significant vision loss prior to treatment. Measurement of visual evoked potentials (VEPs) is therefore used to objectively measure responses to visual stimuli as an indicator of central visual impairment or loss. Hearing loss is also common during disease progression and early indications of auditory abnormalities are measured via brainstem auditory evoked response (BAER) testing.

GALC is responsible for the hydrolysis of psychosine. Deficiency of GALC in Krabbe disease results in the accumulation of psychosine both centrally and peripherally. Increased levels of psychosine have been proposed as an indicator of Krabbe disease (Escolar M.L., et al. (2017) Mol Genet Metab. 121(3):271-278). While there is evidence to support its use in detection of early and severe cases of infantile Krabbe, interpretation of fluctuations in psychosine levels over time, following treatment may be difficult, as psychosine levels may also decline in late-stage disease. Thus, evidence of decline in psychosine levels alone would not be sufficient evidence of a treatment effect, unless it was accompanied by clinical disease stabilization.

### SEQUENCE LISTING FREE TEXT

The following information is provided for sequences containing free text under numeric identifier <223>.

| SEQ ID NO: (containing free text) | Free text under <223> |
|---|---|
| 1 | <223> AAVhu68 vp1 capsid |
| 2 | <223> Synthetic Construct |
| 3 | <223> AAV9 VP1 capsid |
| | <220> |
| | <221> CDS |
| | <222> (1)..(2208) |
| SEQ ID NO: (containing free text) | Free text under <223> |
| | <223> AAV9 VP1 Capsid |
| 4 | <223> Synthetic Construct |
| 5 | <223> Human GALC coding sequence |
| | <220> |
| | <221> sig_peptide |
| | <222> (1)..(126) |
| | <220> |
| | <221> CDS |
| | <222> (1)..(2058) |
| 6 | <223> Synthetic Construct |
| 7 | <223> Engineered canine GALC |
| | <220> |
| | <221> CDS |
| | <222> (1)..(2007) |
| 8 | <223> Synthetic Construct |
| 9 | <223> Engineered human GALC coding sequence |
| | <220> |
| | <221> CDS |
| | <222> (1)..(2055) |
| | <220> |
| | <221> sig_peptide |
| | <222> (1)..(126) |
| | <220> |
| | <221> mat |
| | <222> (108)..(2055) |
| 10 | <223> Synthetic Construct |
| 11 | <223> AAV2 - 5′ ITR |
| 12 | <223> CMV IE promoter |
| 13 | <223> CB promoter |
| 14 | <223> CB7 promoter |
| SEQ ID NO: (containing free text) | Free text under <223> |
| 15 | <223> chimeric intron |
| 16 | <223> Rabbit globin polyA |
| 17 | <223> AAV2 - 3′ ITR |
| 18 | <223> Vector genome with canine GALC |
| 19 | <223> CB7.CI.hGALC.rBG |
| | <220> |
| | <221> misc_feature |
| | <222> (1)..(130) |
| | <223> 5′ ITR |
| | <220> |
| | <221> misc_feature |
| | <222> (198)..(579) |
| | <223> CMV IE Enhancer |
| | <220> |
| | <221> misc_feature |
| | <222> (582)..(863) |
| | <223> CB promoter |
| | <220> |
| | <221> misc_feature |
| | <222> (836)..(839) |
| | <223> TATA |
| | <220> |
| | <221> misc_feature |
| | <222> (958)..(1930) |
| | <223> chimeric intron |
| | <220> |
| | <221> misc_feature |
| | <222> (1948)..(4002) |
| | <223> hGALCco |
| | <220> |
| | <221> misc_feature |
| SEQ ID NO: (containing free text) | Free text under <223> |
| | <222> (4042)..(4168) |
| | <223> Rabbit globin poly A |
| | <220> |
| | <221> misc_feature |
| | <222> (4257)..(4386) |
| | <223> 3′ ITR |
| 20 | <223> AAV1 VP1 gene |
| | <220> |
| | <221> CDS |
| | <222> (1)..(2208) |
| 21 | <223> Synthetic Construct |
| 22 | <223> AAV5 capsid VP1 gene |
| | <220> |
| | <221> CDS |
| | <222> (1)..(2172) |
| 23 | <223> Synthetic Construct |
| 24 | <223> AAV3B VP1 Capsid |
| 25 | <223> UbC promoter |
| 26 | <223> SV40 late poly A |
| 27 | <223> EF-1a promoter |

All documents cited in this specification are incorporated herein by reference. The sequence listing filed herewith named “21-9660PCT_ST25” and the sequences and text therein are incorporated by reference. U.S. Provisional Pat. Application No. 62/810,708, filed Feb. 26, 2019, U.S. Provisional Pat. Application No. 62/817,482, filed Mar. 12, 2019, U.S. Provisional Pat. Application No. 62/877,707, filed Jul. 23, 2019, U.S. Provisional Pat. Application No. 62/916,652, filed Oct. 17, 2019, U.S. Provisional Pat. Application No. 63/023,459, filed May 12, 2020, U.S. Provisional Pat. Application No. 63/070,653, filed Aug. 26, 2020, U.S. Provisional Pat. Application No. 63/073,756, filed Sep. 2, 2020, and International Patent Application No. PCT/US20/19794, filed Feb. 26, 2020, are incorporated herein by reference. While the invention has been described with reference to particular embodiments, it will be appreciated that modifications can be made without departing from the spirit of the invention. Such modifications are intended to fall within the scope of the appended claims.

1. A pharmaceutical composition comprising a stock of recombinant AAV (rAAV) having an AAV capsid and a vector genome packaged therein, wherein the vector genome comprises:
(a) a 5′ inverted terminal repeat (ITR);
(b) a CB7 promoter;
(c) an intron;
(d) a galactosylceramidase (GALC) coding sequence comprising nucleotides 1 to 2055 of SEQ ID NO: 9, or a sequence at least 95% identical thereto that encodes amino acids 1 to 685 of SEQ ID NO: 10;
(e) a polyA; and
(f) a 3′ ITR,
wherein the composition is formulated for administration of a dose of about 1.7 × 10¹⁰ genome copies (GC)/g brain mass to about 5.0 × 10¹¹ GC/g brain mass.

2. The pharmaceutical composition according to claim 1, wherein the AAV capsid is an AAVhu68 capsid.

3. The pharmaceutical composition according to claim 1 , wherein the vector genome comprises nucleotides 198 to 4168 of SEQ ID NO: 19.

4. The pharmaceutical composition according to claim 1, wherein the composition is formulated for administration of a dose of 1.4 × 10¹³ GC to 4.0 × 10¹⁴ GC.

5. The pharmaceutical composition according to claim 1, wherein the composition is formulated for administration of a dose of 4.0 × 10¹³ GC to 4.0 × 10¹⁴ GC.

6. The pharmaceutical composition according to claim 1, wherein the composition is formulated for intracisternal magna (ICM) administration.

7. The pharmaceutical composition according to claim 1, wherein the total volume is 4.5 mL to 5.5 mL.

8. A method of treating Krabbe disease in a patient in need thereof, the method comprising intracisternal magna (ICM) administration of the pharmaceutical composition according to claim 1 to the patient.

9. The method of claim 8, wherein the pharmaceutical composition is administered at a dose of about 1.7 × 10¹⁰ genome copies (GC)/g brain mass to about 5.0 × 10¹¹ GC/g brain mass.

10. The method according to claim 8, further comprising hematopoietic stem cell transplant or bone marrow transplant before or after administration of the pharmaceutical composition.

11. The method according to claim 10, wherein the hematopoietic stem cell transplant or bone marrow transplant permits a reduced dose of the rAAV to be administered to the patient.

12. A method for increasing GALC expression and enzyme activity in the serum and/or cerebral spinal fluid (CSF) of a patient having Krabbe disease, the method comprising administering a pharmaceutical composition according to claim 1 to the patient at a dose of about 1.7 × 10¹⁰ genome copies (GC)/g brain mass to about 5.0 × 10¹¹ GC/g brain mass.

13. A method for reducing neuroinflammation in peripheral nerves of a patient having Krabbe disease, the method comprising administering a pharmaceutical composition according to claim 1 to the patient at a dose of about 1.7 × 10¹⁰ genome copies (GC)/g brain mass to about 5.0 × 10¹¹ GC/g brain mass.

14. A method for increasing GALC expression and activity in neurons of the cortex and/or hippocampus of a patient having Krabbe disease, the method comprising administering a pharmaceutical composition according to claim 1 to the patient at a dose of about 1.7 × 10¹⁰ genome copies (GC)/g brain mass to about 5.0 × 10¹¹ GC/g brain mass.

15. The method according to claim 8 , wherein the patient is less than two months, less than six months, or less than twelve months of age.

16. The method according to claim 8, wherein the rAAV is administered at a dose of 1.4 × 10¹³ GC to 4.0 × 10¹⁴ GC.

17. The method according to claim 8, wherein the rAAV is administered at a dose of 4.0 × 10¹³ GC to 4.0 × 10¹⁴ GC.

18. The method according to claim 8, further comprising the step of measuring psychosine in the serum and/or cerebral spinal fluid (CSF).

19-25. (canceled)
